(19) United States
(12) Patent Application Publication
(10) Pub. No.: US 2022/0364159 A1
(43) Pub. Date: Nov. 17, 2022

(54) COMPOSITIONS FOR DETECTION OF DNA AND METHODS OF USE THEREOF

(71) Applicant: Mammoth Biosciences, Inc., Brisbane, CA (US)

(72) Inventors: James Paul Broughton, San Francisco, CA (US); Janice Sha Chen, San Francisco, CA (US); Lucas Benjamin Harrington, Berkeley, CA (US)

(21) Appl. No.: 17/629,895

(22) PCT Filed: Jul. 22, 2020
(86) PCT No.: PCT/US2020/043139

§ 371 Date: Jan. 25, 2022

Related U.S. Application Data
(60) Provisional application No. 62/879,315, filed on Jul. 26, 2019.

(51) Int. Cl. C12Q 1/6816; C12N 9/22

(52) U.S. Cl. CPC C12Q 1/6816; C12N 9/22; C12N 2310/20

(57) ABSTRACT

Described herein are methods and systems for direct detection of DNA nucleic acids using a DNA-activated programmable RNA nuclease.

### CROSS-REFERENCE

The present application is a U.S. National Stage Application under 35 U.S.C. § 371 of International PCT Application No. PCT/US2020/043139, filed on Jul. 22, 2020, which claims priority to and benefit from U.S. Provisional Application No. 62/879,315, filed on Jul. 26, 2019, the entire contents of each of which are herein incorporated by reference.

### DESCRIPTION OF THE TEXT FILE SUBMITTED ELECTRONICALLY

The contents of the text file submitted electronically herewith are incorporated herein by reference in its entirety: A computer readable format copy of the Sequence Listing (filename: MABI_004_01US_SubSeqList_ST25.txt, date created: Jul. 21, 2022, file size: ˜1,043,406 bytes.

### BACKGROUND

Detection of specific nucleic acids often requires time- and resource-intensive steps such as sequence amplification or reverse transcription. Simpler methods are needed to increase efficiency and decrease costs of detection methods.

### SUMMARY

Described herein are methods, compositions, reagents, enzymes, and kits for detection of target nucleic acids. The methods, compositions, reagents, enzymes, and kits may comprise reagents of a guide nucleic acid targeting a target nucleic acid, a programmable nuclease, and a single stranded detector nucleic acid with a detection moiety. The target nucleic acid of interest may be indicative of a disease, and the disease may be communicable diseases. The detection of the disease may provide guidance on treatment or intervention to reduce the transmission of the disease.

In various aspects, the present disclosure provides a composition comprising: a) a DNA-activated programmable RNA nuclease; and b) an engineered guide nucleic acid comprising a first segment that is reverse complementary to a segment of a target deoxyribonucleic acid,

wherein the engineered guide nucleic acid comprises a second segment that binds to the DNA-activated programmable RNA nuclease to form a complex.

In some aspects, the composition further comprises a detector nucleic acid. In some aspects, the detector nucleic acid comprises an RNA sequence. In some aspects, the detector nucleic acid is an RNA reporter. In some aspects, the composition further comprises the target deoxyribonucleic acid. In some aspects, the target deoxyribonucleic acid is an amplicon of a nucleic acid. In some aspects, the nucleic acid is a deoxyribonucleic acid or a ribonucleic acid.

In some aspects, the DNA-activated programmable RNA nuclease comprises a HEPN domain. In some aspects, the DNA-activated programmable RNA nuclease comprises two HEPN domains.

In some aspects, the DNA-activated programmable RNA nuclease is a Type VI CRISPR/Cas enzyme. In some aspects, the DNA-activated programmable RNA nuclease is a Cas13 protein. In some aspects, the Cas13 protein comprises a Cas13a polypeptide, a Cas13b polypeptide, a Cas13c polypeptide, a Cas13c polypeptide, a Cas13d polypeptide, or a Cas13e polypeptide. In further aspects, the Cas13 protein is a Cas13a polypeptide. In still further aspects, the Cas13a polypeptide is LbuCas13a or LwaCas13a.

In some aspects, the DNA-activated programmable RNA nuclease has at least 80%, at least 85%, at least 90%, at least 92%, at least 95%, at least 97%, or at least 99% sequence identity to any one of SEQ ID NO: 18-SEQ ID NO: 35. In some aspects, the DNA-activated programmable RNA nuclease is selected from any one of SEQ ID NO: 18-SEQ ID NO: 35.

In some aspects, the composition has a pH from pH 6.8 to pH 8.2. In some aspects, the target deoxyribonucleic acid lacks a guanine at the 3′ end. In some aspects, the terminal 3′ nucleotide in the segment of the target deoxyribonucleic acid is A, C or T. In some aspects, the target deoxyribonucleic acid is a single-stranded deoxyribonucleic acid. In some aspects, the target deoxyribonucleic acid is single stranded deoxyribonucleic acid oligonucleotides. In some aspects, the target deoxyribonucleic acid is genomic single stranded deoxyribonucleic acids. In some aspects, the target deoxyribonucleic acid has a length of from 18 to 100 nucleotides. In further aspects, the target deoxyribonucleic acid has a length of from 18 to 30 nucleotides. In still further aspects, the target deoxyribonucleic acid has a length of 20 nucleotides. In some aspects, the composition is present within a support medium.

In some aspects, the composition further comprises a second engineered guide nucleic acid comprising a first segment that is reverse complementary to a segment of a second target deoxyribonucleic acid; and a DNA-activated programmable DNA nuclease, wherein the second engineered guide nucleic acid comprises a second segment that binds to the DNA-activated programmable DNA nuclease to form a complex. In some aspects, the composition further comprises a DNA reporter. In some aspects, the DNA-activated programmable DNA nuclease comprises a RuvC catalytic domain. In some aspects, the DNA-activated programmable DNA nuclease comprises a type V CRISPR/Cas enzyme.

In some aspects, the target deoxyribonucleic acid is a reverse transcribed ribonucleic acid. In some aspects, the composition further comprises a reagent for reverse transcription. In some aspects, the composition further comprises a reagent for amplification. In some aspects, the composition further comprises a reagent for in vitro transcription. In some aspects, the reagent for reverse transcription comprises a reverse transcriptase, an oligonucleotide primer, dNTPs, or any combination thereof. In some aspects, the reagent for amplification comprises a primer, a polymerase, dNTPs, or any combination thereof. In some aspects, the reagent for in vitro transcription comprise an RNA polymerase, NTPs, a primer, or any combination thereof.

In various aspects, the present disclosure provides a method of assaying for a target deoxyribonucleic acid in a sample, the method comprising: contacting the sample to the compostions of any of the above compositions; and assaying for a signal produced by cleavage of at least some RNA reporters of a plurality of RNA reporters by the DNA-activated programmable RNA nuclease upon hybridization of the first segment of the engineered guide nucleic acid to the segment of the target deoxyribonucleic acid.

In various aspects, the present disclosure provides a method of assaying for a target ribonucleic acid in a sample, the method comprising: amplifying the target ribonucleic acid in a sample to produce a target deoxyribonucleic acid; contacting the target deoxyribonucleic acid to the composition of any of the above compositions; and assaying for a signal produced by cleavage of at least some RNA reporters of a plurality of RNA reporters by the DNA-activated programmable RNA nuclease upon hybridization of the first segment of the engineered guide nucleic acid to the segment of the target deoxyribonucleic acid.

In various aspects, the present disclosure provides the use of any of the above compositions in a method of assaying for a target deoxyribonucleic acid in a sample.

In various aspects, the present disclosure provides the use of a DNA-activated programmable RNA nuclease in a method of assaying for a target deoxyribonucleic acid in a sample according to any of the above methods.

In various aspects, the present disclosure provides the use of a DNA-activated programmable RNA nuclease in a method of assaying for a target ribonucleic acid in a sample according to any of the above methods.

In some aspects, a composition comprises a DNA-activated programmable RNA nuclease; and a guide nucleic acid comprising a segment that is reverse complementary to a segment of a target deoxyribonucleic acid, wherein the DNA-activated programmable RNA nuclease binds to the guide nucleic acid to form a complex. In some aspects, the composition further comprises an RNA reporter. In some aspects, the composition further comprises the target deoxyribonucleic acid. In some aspects, the target deoxyribonucleic acid is an amplicon of a nucleic acid. In some aspects, the nucleic acid is a deoxyribonucleic acid or a ribonucleic acid.

In some aspects, the DNA-activated programmable RNA nuclease is a Type VI CRISPR/Cas enzyme. In some aspects, the DNA-activated programmable RNA nuclease is a Cas13. In some aspects, the DNA-activated programmable RNA nuclease is a Cas13a. In some aspects, the Cas13a is Lbu-Cas13a or Lwa-Cas13a. In some aspects, the composition has a pH from pH 6.8 to pH 8.2 In some aspects, the target deoxyribonucleic acid lacks a guanine at the 3′ end. In some aspects, the target deoxyribonucleic acid is a single-stranded deoxyribonucleic acid. In some aspects, the composition further comprises a support medium. In some aspects, the composition further comprises a lateral flow assay device. In some aspects, the composition further comprises a device configured for fluorescence detection. In some aspects, the composition further comprises a second guide nucleic acid and a DNA-activated programmable DNA nuclease, wherein the second guide nucleic acid comprises a segment that is reverse complementary to a segment of a second target deoxyribonucleic acid comprising a guide nucleic acid. In some aspects, the composition further comprises a DNA reporter. In some aspects, the DNA-activated programmable DNA nuclease is a Type V CRISPR/Cas enzyme. In some aspects, the DNA-activated programmable DNA nuclease is a Cas12. In some aspects, the Cas12 is a Cas12a, Cas12b, Cas12c, Cas12d, or Cas12e. In some aspects, the DNA-activated programmable DNA nuclease is a Cas14. In some aspects, the Cas14 is a Cas14a, Cas14b, Cas14c, Cas14d, Cas14e, Cas14f, Cas14g, or Cas14h.

In some aspects, a method of assaying for a target deoxyribonucleic acid in a sample comprises contacting the sample to a complex comprising a guide nucleic acid and a DNA-activated programmable RNA nuclease, wherein the guide nucleic acid comprises a segment that is reverse complementary to a segment of the target deoxyribonucleic acid, and assaying for a signal produced by cleavage of at least some RNA reporters of a plurality of RNA reporters.

In some aspects, a method of assaying for a target ribonucleic acid in a sample comprises amplifying a nucleic acid in a sample to produce a target deoxyribonucleic acid, contacting the target deoxyribonucleic acid to a complex comprising a guide nucleic acid and a DNA-activated programmable RNA nuclease, wherein the guide nucleic acid comprises a segment that is reverse complementary to a segment of the target deoxyribonucleic acid, and assaying for a signal produced by cleavage of at least some RNA reporters of a plurality of RNA reporters.

In some aspects, the DNA-activated programmable RNA nuclease is a Type VI CRISPR nuclease. In some aspects, the DNA-activated programmable RNA nuclease is a Cas13. In some aspects, the Cas13 is a Cas13a. In some aspects, the Cas13a is Lbu-Cas13a or Lwa-Cas13a. In some aspects, cleavage of the at least some RNA reporters of the plurality of reporters occurs from pH 6.8 to pH 8.2. In some aspects, the target deoxyribonucleic acid lacks a guanine at the 3′ end. In some aspects, the target deoxyribonucleic acid is a single-stranded deoxyribonucleic acid. In some aspects, the target deoxyribonucleic acid is an amplicon of a ribonucleic acid. In some aspects, the target deoxyribonucleic acid or the ribonucleic acid is from an organism. In some aspects, the organism is a virus, bacteria, plant, or animal. In some aspects, the target deoxyribonucleic acid is produced by a nucleic acid amplification method. In some aspects, the nucleic acid amplification method is isothermal amplification. In some aspects, the nucleic acid amplification method is thermal amplification. In some aspects, the nucleic acid amplification method is recombinase polymerase amplification (RPA), transcription mediated amplification (TMA), strand displacement amplification (SDA), helicase dependent amplification (HDA), loop mediated amplification (LAMP), rolling circle amplification (RCA), single primer isothermal amplification (SPIA), ligase chain reaction (LCR), simple method amplifying RNA targets (SMART), or improved multiple displacement amplification (IMDA), or nucleic acid sequence-based amplification (NASBA). In some aspects, the signal is fluorescence, luminescence, colorimetric, electrochemical, enzymatic, calorimetric, optical, amperometric, or potentiometric. In some aspects, the method further comprises contacting the sample to a second guide nucleic acid and a DNA-activated programmable DNA nuclease, wherein the second guide nucleic acid comprises a segment that is reverse complementary to a segment of a second target deoxyribonucleic acid comprising a guide nucleic acid. In some aspects, the method further comprises assaying for a signal produced by cleavage of at least some DNA reporters of a plurality of DNA reporters. In some aspects, the DNA-activated programmable DNA nuclease is a Type V CRISPR nuclease. In some aspects, the DNA-activated programmable DNA nuclease is a Cas12. In some aspects, the Cas12 is a Cas12a, Cas12b, Cas12c, Cas12d, or Cas12e. In some aspects, the DNA-activated programmable DNA nuclease is a Cas14. In some aspects, the Cas14 is a Cas14a, Cas14b, Cas14c, Cas14d, Cas14e, Cas14f, Cas14g, or Cas14h. In some aspects, the guide nucleic acid comprises a crRNA. In some aspects, the guide nucleic acid comprises a crRNA and a tracrRNA. In some aspects, the signal is present prior to cleavage of the at least some RNA reporters. In some aspects, the signal is absent prior to cleavage of the at least some RNA reporters. In some aspects, the sample comprises blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. In some aspects, the method is carried out on a support medium. In some aspects, the method is carried out on a lateral flow assay device. In some aspects, the method is carried out on a device configured for fluorescence detection.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows Cas13a detection of target RT-LAMP DNA amplicon.

FIG. 1A shows a schematic of the workflow including providing DNA/RNA, LAMP/RT-LAMP, and Cas13a detection.

FIG. 1B shows Cas13a specific detection of target RT-LAMP DNA amplicon with a first primer set as measured by background subtracted fluorescence on the y-axis.

FIG. 1C shows Cas13a specific detection of target RT-LAMP DNA amplicon with a second primer set as measured by background subtracted fluorescence on the y-axis.

FIG. 2 shows experimental results from a Cas13 detection assay.

FIG. 2A shows a Cas13 detection assay using 2.5 nM RNA, single-stranded DNA (ssDNA), or double-stranded (dsDNA) as target nucleic acids, where detection was measured by fluorescence for each of the target nucleic acid tested.

FIG. 2B shows Cas12 detection assay using 2.5 nM RNA, ssDNA, and dsDNA as target nucleic acids, where detection was measured by fluorescence for each of the target target nucleic acid tested.

FIG. 2C shows the performance of Cas13 and Cas12 on target RNA, target ssDNA, and target dsDNA at various concentrations, where detection was measured by fluorescence for each of the target nucleic acids tested.

FIG. 3 shows an Lbu-Cas13a (SEQ ID NO: 19) detection assay using 2.5 nM target ssDNA with 170 nM of various reporter substrates, wherein detection was measured by fluorescence for each of the reporter substrates tested.

FIG. 4 shows experimental results of a Cas13 detection assay.

FIG. 4A shows the results of Cas13 detection assays for Lbu-Cas13a (SEQ ID NO: 19) and Lwa-Cas13a (SEQ ID NO: 25) using 10 nM of target RNA or no target RNA (shown as 0 nM), where detection was measured by fluorescence resulting from cleavage of reporters over time.

FIG. 4B shows the results of Cas13 detection assays for Lbu-Cas13a (SEQ ID NO: 19) and Lwa-Cas13a (SEQ ID NO: 25) using 10 nM of target ssDNA or no target ssDNA (shown as 0 nM), where detection was measured by fluorescence resulting from cleavage of reporters over time.

FIG. 5 shows Lbu-Cas13a (SEQ ID NO: 19) detection assay using 1 nM target RNA (at left) or target ssDNA (at right) in buffers with various pH values ranging from 6.8 to 8.2.

FIG. 6 shows setup and experimental results of a Cas13 detection assay.

FIG. 6A shows guide RNAs (gRNAs) tiled along a target sequence at 1 nucleotide intervals.

FIG. 6B shows Cas13M26 detection assays using 0.1 nM target RNA or 2 nM target ssDNA with gRNAs tiled at 1 nucleotide intervals and an off-target gRNA.

FIG. 6C shows data from FIG. 6B ranked by performance of target ssDNA.

FIG. 6D shows performance of gRNAs for each nucleotide on a 3′ end of a target RNA.

FIG. 6E shows performance of gRNAs for each nucleotide on a 3′ end of a target ssDNA.

FIG. 7 shows experimental results from a Lbu-Cas13a (SEQ ID NO: 19) detection assays.

FIG. 7A shows Lbu-Cas13a (SEQ ID NO: 19) detection assays using 1 μL of target DNA amplicon from various LAMP isothermal nucleic acid amplification reactions.

FIG. 7B shows Cas13M26 detection assays using various amounts of PCR reaction as a target DNA.

FIG. 8 shows results from detection assays using a Cas13a DNA-activated programmable RNA nuclease, ssDNA target oligonucleotides, guide RNAs, and a reporter.

FIG. 8A shows results from assays in which ssDNA oligonucleotides were present at 2 nM.

FIG. 8B shows results from assays in which ssDNA oligonucletoides were not present (shown as 0 μM).

FIG. 9 shows results from detection assays using a Cas13a DNA-activated programmable RNA nuclease, ssDNA genome from the bacteriophage M13mp18, guide RNAs, and a reporter.

FIG. 9A shows results from assays in which the R1490 guide was used.

FIG. 9B shows results from assays in which the R1488 guide was used.

FIG. 9C shows results from assays in which the R1491 guide was used.

FIG. 10 illustrates the raw HMM for PF07282.

FIG. 11 illustrates the raw HMM for PF18516.

### DETAILED DESCRIPTION

The capability to quickly and accurately detect the presence of a target nucleic acid can provide valuable information associated with the presence of the target nucleic acid. For example, the capability to quickly and accurately detect the presence of an ailment provides valuable information and leads to actions to reduce the progression or transmission of the ailment. Detection of a target nucleic acid molecule encoding a specific sequence using a programmable nuclease provides a method for efficiently and accurately detecting the presence of the nucleic acid molecule of interest. There exists a need for direct sequence detection methods, in particular methods to directly and robustly detect DNA encoding a specific sequence. Such direct detection methods may reduce reagent and labor costs and decrease the time to result of the detection assay.

Provided herein are programmable nucleases capable of directly detecting DNA in a sample. In some embodiments, the present disclosure provides a composition comprising a DNA-activated programmable RNA nuclease. In some embodiments, the present disclosure provides a composition comprising a DNA-activated programmable RNA nuclease, an engineered guide nucleic acid comprising a segment that is reverse complementary to a segment of a target deoxyribonucleic acid, wherein the DNA-activated programmable RNA nuclease binds to the engineered guide nucleic acid to form a complex, and a RNA reporter, and optionally, further comprising a target deoxyribonucleic acid. In some embodiments, the present disclosure provides methods, systems, enzymes, and kits for direct detection of DNA with a programmable nuclease. The programmable nuclease may be a DNA-activated programmable RNA nuclease. The DNA-activated programmable RNA nuclease may be a Type VI CRISPR/Cas enzyme. For example, in some embodiments, the present disclosure provides a Cas13 protein for direct detection of DNA in a sample. In particular embodiments, the Cas13 protein can be a Cas13a protein. In some embodiments, a DNA-activated programmable RNA nuclease is multiplexed with a DNA-activated programmable RNA nuclease for detection of two target deoxynucleic acids that encode different sequences.

The detection of the disease in an individual, especially at the early stages of the disease, may provide guidance on treatments or interventions to reduce the progression of the disease. Additionally, the detection of traits of the disease, such as resistance to an antibiotic, can be useful for informing treatment of the disease. The detection of the disease in the environment may provide guidance on interventions to reduce or minimize a potential epidemic or transmission of the disease. The capability to quickly and accurately detect the presence of a disease in a biological or environmental sample can provide valuable information and lead to actions to reduce the transmission of the disease.

Additionally, early detection of cancers and genetic disorders can be important for initiating treatment. Individuals with cancer or genetic disorders may have poor outcomes, including severe symptoms that can lead to death, if left undetected. The detection of the cancer or genetic disorder in an individual, especially at the early stages of the cancer or genetic disorder, may provide guidance on treatments or interventions to reduce the progression of the cancer or maladies associated with progression of the genetic disorder.

The present disclosure provides various methods, reagents, enzymes, and kits for rapid lab tests, which may quickly assess whether a target nucleic acid is present in a sample by using a DNA-activated programmable RNA nuclease that can detect the presence of a nucleic acid of interest (e.g., a deoxyribonucleic acid or a deoxyribonucleic acid amplicon of the nucleic acid of interest, which can be the target deoxyribonucleic acid) and generating a detectable signal indicating the presence of said nucleic acid of interest. The methods and programmable nucleases disclosed herein can be used as a companion diagnostic with any of the diseases disclosed herein (e.g., RSV, sepsis, flu), or can be used in reagent kits, point-of-care diagnostics, or over-the-counter diagnostics. The methods or reagents may be used as a point of care diagnostic or as a lab test for detection of a target nucleic acid and, thereby, detection of a condition in a subject from which the sample was taken. The methods or reagents may be used in various sites or locations, such as in laboratories, in hospitals, in physician offices/laboratories (POLs), in clinics, at remotes sites, or at home. Sometimes, the present disclosure provides various devices, systems, fluidic devices, and kits for consumer genetic use or for over the counter use.

Furthermore, detection of a target nucleic acid can provide genetic information of the sample, which is consistent with the methods, compositions, reagents, enzymes, and kits described herein. A target nucleic acid that provides genetic information can include, but is not limited to, a nucleic acid encoding a sequence associated with organism ancestry (e.g., a nucleic acid comprising a sequence encoding a single nucleotide polymorphism that identifies geographical ancestry, ancestry from an ethnic group, etc.); a sequence for trait not associated with a communicable disease, cancer, or genetic disorder; a sequence for a phenotypic trait (e.g., a sequence from a gene for blue eyes, brown hair color, fast or slow metabolism of a drug such as caffeine, an intolerance such as lactose intolerance, etc.), or a sequence for genotyping (e.g., a sequence for a gene that is recessive, such as the gene for Taye-Sachs disease).

Described herein are methods, compositions, reagents, enzymes, and kits for detecting the presence of a target nucleic acid in a sample. The methods, compositions, reagents, enzymes, and kits for detecting the presence of a target nucleic acid in a sample can be used in a rapid lab tests for direct detection of a target nucleic acid encoding a sequence of interest. In particular, provided herein are methods, reagents, enzymes, and kits which may enable the direct detection of target DNA sequences. Also disclosed herein are devices comprising the reagents, enzymes (e.g., a DNA-activated programmable RNA nuclease), and kits of this disclosure. A device of this disclosure may comprise a fluidic device, reagents for detecting a target nucleic acid in a sample, and a solid support.

In one aspect, described herein, is a method for detecting a target nucleic acid, such as a single-stranded DNA, in a sample. The method may comprise contacting the sample with an engineered guide nucleic acid capable of binding a target nucleic acid sequence; a programmable nuclease capable of being activated when complexed with the engineered guide nucleic acid and the target sequence; and a single stranded detector nucleic acid comprising a detection moiety, wherein the detector nucleic acid is capable of being cleaved by the activated nuclease, thereby generating a first detectable signal. In some embodiments, the programmable nuclease is a DNA-activated programmable RNA nuclease. In some embodiments, the method comprises a DNA-activated programmable RNA nuclease for detecting a first target deoxyribonucleic acid and a a DNA-activated programmable RNA nuclease for detecting a second deoxyribonucleic acid. In some embodiments, the first deoxyribonucleic acid and the second deoxyribonucleic acid encode different sequences. In some embodiments, the first deoxyribonucleic acid and the second deoxyribonucleic acid encode the same sequence.

In another aspect, described herein are reagents for detecting a target nucleic acid, such as a single-stranded DNA reporter, the reagents comprising a reagent chamber and a support medium for detection of the first detectable signal. The reagent chamber comprises an engineered guide nucleic acid comprising a segment that is reverse complementary to the target nucleic acid; a programmable nuclease capable of being activated when complexed with the engineered guide nucleic acid and the target nucleic acid; and a single stranded detector nucleic acid comprising a detection moiety, wherein the detector nucleic acid is capable of being cleaved by the activated nuclease, thereby generating a first detectable signal. In some embodiments, the programmable nuclease is a DNA-activated programmable RNA nuclease.

Also described herein is a kit for detecting a target nucleic acid. The kit may comprise an engineered guide nucleic acid that binds to a target nucleic acid, preferably DNA; a programmable nuclease capable of being activated when complexed with the engineered guide nucleic acid and the target nucleic acid; and a single stranded detector nucleic acid comprising a detection moiety, wherein the detector nucleic acid is capable of being cleaved by the activated nuclease, thereby generating a first detectable signal.

A sample can be a biological sample or an environmental sample. A biological sample can be from an individual and can be tested to determine whether the individual has a communicable disease. The biological sample can be tested to detect the presence or absence of at least one target nucleic acid from a bacterium or a virus or a pathogen responsible for the disease. The at least one target nucleic acid from a bacterium or a pathogen responsible for the disease that is detected can also indicate that the bacterium or pathogen is wild-type or comprises a mutation that confers resistance to treatment, such as antibiotic treatment. The biological sample can be tested to detect the presence or absence of at least one target nucleic acid expressed in a cancer or genetic disorder. An environmental sample can comprise a biological material and can be tested to determine whether the content of the biological material. For example, the environmental sample can be tested to detect the presence or absence of at least one target nucleic acid from a bacterium or a virus or a pathogen, which in some cases, can be responsible for a disease (e.g., a human pathogenic disease or an agricultural disease). The at least one target nucleic acid from a bacterium or a pathogen responsible for the disease that is detected can also indicate that the bacterium or pathogen is wild-type or comprises a mutation that confers resistance to treatment, such as antibiotic treatment. A sample from an individual or from an environment is applied to the reagents described herein. If the target nucleic acid is present in the sample, the target nucleic acid binds to the engineered guide nucleic acid to activate the DNA-activated programmable RNA nuclease. The activated DNA-activated programmable RNA nuclease cleaves the detector RNA and generates a detectable signal that can be visualized, for example on a support medium, by eye, or using a spectrometer. If the target nucleic acid is absent in the sample or below the threshold of detection, the engineered guide nucleic acid remains unbound, the DNA-activated programmable RNA nuclease remains inactivated, and the detector RNA remains uncleaved.

Such methods, compositions, reagents, enzymes, and kits described herein may allow for direct detection of target deoxyribonucleic acid, such as a target single-stranded DNA, and in turn the pathogen and disease associated with the target nucleic acid or the cancer or genetic disorder associated with the target nucleic acid, in remote regions or low resource settings without specialized equipment. Also, such methods, compositions, reagents, enzymes, and kits described herein may allow for detection of target nucleic acid, and in turn the pathogen and disease associated with the target nucleic acid or the cancer or genetic disorder associated with the target nucleic acid, in healthcare clinics or doctor offices without specialized equipment. In some cases, this provides a point of care testing for users to easily test for a disease, cancer, or genetic disorder at home or quickly in an office of a healthcare provider. Assays that deliver results in under an hour, for example, in 15 to 60 minutes, are particularly desirable for at home testing for many reasons. Antivirals can be most effective when administered within the first 48 hours and improve antibiotic stewardship. Thus, the systems and assays disclosed herein, which are capable of delivering results in under an hour can will allow for the delivery of anti-viral therapy at an optimal time. Additionally, the systems and assays provided herein, which are capable of delivering quick diagnoses and results, can help keep or send a patient at home, improve comprehensive disease surveillance, and prevent the spread of an infection. In other cases, this provides a test, which can be used in a lab to detect a nucleic acid sequence of interest in a sample from a subject. Also provided herein are devices, compositions, systems, fluidic devices, and kits, wherein the rapid lab tests can be performed in a single system. In some cases, this may be valuable in detecting diseases and pathogens, cancer, or a genetic disorder in a developing country and as a global healthcare tool to detect the spread of a disease or efficacy of a treatment or provide early detection of a cancer or genetic disorder.

The methods as described herein in some instances comprise obtaining a cell-free DNA sample, amplifying DNA from the sample, using a DNA-activated programmable RNA nuclease to cleave detector RNA, and reading the output of the cleavage. In other instances, the method comprises obtaining a fluid sample from a patient, and without amplifying a nucleic acid of the fluid sample, using a DNA-activated programmable RNA nuclease to cleave detector RNA, and detecting the cleavage of the detector RNA. A number of samples, engineered guide nucleic acids, DNA-activated programmable RNA nuclease, support mediums, target nucleic acids, single-stranded detector nucleic acids, and reagents are consistent with the devices, systems, fluidic devices, kits, and methods disclosed herein. Furthermore, these can be multiplexed with a second programmable nuclease, such a DNA-activated programmable DNA nuclease.

Also disclosed herein are detector nucleic acids and methods detecting a target nucleic using the detector nucleic acids. Reporter and detector as used herein are interchangeably with reporter nucleic acid (e.g., RNA, DNA) or detector nucleic acid (e.g., RNA, DNA). Often, the detector nucleic acid is a protein-nucleic acid. For example, a method of assaying for a target nucleic acid in a sample comprises contacting the sample to a complex comprising an engineered guide nucleic acid comprising a segment that is reverse complementary to a segment of the target nucleic acid and a programmable nuclease that exhibits sequence independent cleavage upon forming a complex comprising the segment of the engineered guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. Often, the protein-nucleic acid is an enzyme-nucleic acid or a enzyme substrate-nucleic acid. Sometimes, the protein-nucleic acid is attached to a solid support. The nucleic acid can be DNA, RNA, or a DNA/RNA hybrid. The methods described herein use a programmable nuclease, such as a DNA-activated programmable RNA nuclease, to detect a target nucleic acid. A method of assaying for a target nucleic acid in a sample, for example, comprises: a) contacting the sample to a complex comprising an engineered guide nucleic acid comprising a segment that is reverse complementary to a segment of the target nucleic acid and a programmable nuclease that exhibits sequence independent cleavage upon forming a complex comprising the segment of the engineered guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Cleavage of the protein-nucleic acid produces a signal. For example, cleavage of the protein-nucleic acid produces a calorimetric signal, a potentiometric signal, an amperometric signal, an optical signal, or a piezo-electric signal. Various devices can be used to detect these different types of signals, which indicate whether a target nucleic acid is present in the sample.

### Sample

A number of samples are consistent with the methods, reagents, enzymes, and kits disclosed herein. In particular, described herein are sample that contain deoxyribonucleic acid (DNA), which can be directly detected by a DNA-activated programmable RNA nuclease, such as a type VI CRISPR enzyme, for example Cas13a, Cas13b, Cas13c, Cas13d, or Cas13e. As described herein, nucleic acid comprising DNA may be directly detected using a Cas13 programmable nuclease. Direct DNA detection using Cas13 can eliminate the need for intermediate steps, for example reverse transcription or amplification, required by existing Cas13-based sequence detection methods. Elimination of said intermediate steps decreases time to assay result and reduces labor and reagent costs.

These samples can comprise a target nucleic acid. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample can be taken from any place where a nucleic acid can be found. Samples can be taken from an individual/human, a non-human animal, or a crop or an environmental sample can be obtained to test for presence of a disease, virus, pathogen, cancer, genetic disorder, or any mutation or pathogen of interest. A biological sample can be blood, serum, plasma, lung fluid, exhaled breath condensate, saliva, spit, urine, stool, feces, mucus, lymph fluid, peritoneal, cerebrospinal fluid, amniotic fluid, breast milk, gastric secretions, bodily discharges, secretions from ulcers, pus, nasal secretions, sputum, pharyngeal exudates, urethral secretions/mucus, vaginal secretions/mucus, anal secretion/mucus, semen, tears, an exudate, an effusion, tissue fluid, interstitial fluid (e.g., tumor interstitial fluid), cyst fluid, tissue, or, in some instances, a combination thereof. A sample can be an aspirate of a bodily fluid from an animal (e.g. human, animals, livestock, pet, etc.) or plant. A tissue sample can be from any tissue that may be infected or affected by a pathogen (e.g., a wart, lung tissue, skin tissue, and the like). A tissue sample (e.g., from animals, plants, or humans) can be dissociated or liquified prior to application to detection system of the present disclosure. A sample can be from a plant (e.g., a crop, a hydroponically grown crop or plant, and/or house plant). Plant samples can include extracellular fluid, from tissue (e.g., root, leaves, stem, trunk etc.). A sample can be taken from the environment immediately surrounding a plant, such as hydroponic fluid/water, or soil. A sample from an environment may be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system or be applied neat to the detection system. Sometimes, the sample is contained in no more 20 μl. The sample, in some cases, is contained in no more than 1, 5, 10, 15, 20, 25, 30, 35 40, 45, 50, 55, 60, 65, 70, 75, 80, 90, 100, 200, 300, 400, 500 μl, or any of value from 1 μl to 500 μl, preferably from 10 μL to 200 μL, or more preferably from 50 μL to 100 μL. Sometimes, the sample is contained in more than 500 μl.

In some embodiments, the target nucleic acid is single-stranded DNA. The methods, reagents, enzymes, and kits disclosed herein may enable the direct detection of a DNA encoding a sequence of interest, in particular a single-stranded DNA encoding a sequence of interest, without transcribing the DNA into RNA, for example, by using an RNA polymerase. The methods, reagents, enzymes, and kits disclosed herein may enable the detection of target nucleic acid that is an amplified nucleic acid of a nucleic acid of interest. In some embodiments, the target nucleic acid is a cDNA, genomic DNA, an amplicon of genomic DNA or a DNA amplicon of an RNA. In some cases, the target nucleic acid that binds to the engineered guide nucleic acid is a portion of a nucleic acid. A portion of a nucleic acid can encode a sequence from a genomic locus. A portion of a nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A portion of a nucleic acid can be from 10 to 90, from 20 to 80, from 30 to 70, or from 40 to 60 nucleotides in length. A portion of a nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target nucleic acid can encode a sequence is reverse complementary to an engineered guide nucleic acid sequence.

In some instances, the sample is taken from a single-cell eukaryotic organisms; a plant or a plant cell; an algal cell; a fungal cell; an animal or an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

The sample used for disease testing may comprise at least one target nucleic acid that can bind to an engineered guide nucleic acid of the reagents described herein. The sample used for disease testing may comprise at least nucleic acid of interest that is amplified to produce a target nucleic acid that can bind to an engineered guide nucleic acid of the reagents described herein. The nucleic acid of interest can comprise DNA, RNA, or a combination thereof.

The target nucleic acid can be a nucleic acid or portion of a nucleic acid from a pathogen, virus, bacterium, fungi, protozoa, worm or other agents or organism responsible or related to a a disease or condition in living organisms (e.g. humans, animals, plants, crops and the like). The target nucleic acid can be portions of sequences that are agricultural targets (e.g., nucleic acids from plants). The target nucleic acid (e.g., a target DNA) may be a portion of a nucleic acid from a virus or a bacterium or other agents responsible for a disease in the sample. The target nucleic acid may be a portion of a nucleic acid from a gene expressed in a cancer or genetic disorder in the sample. The target nucleic acid may comprise a genetic variation (e.g., a single nucleotide polymorphism), with respect to a standard sample, associated with a disease phenotype or disease predisposition. The target nucleic acid may be an amplicon of a portion of an RNA, may be a DNA, or may be a DNA amplicon from any organism in the sample. The target nucleic acid can be a portion of any genomic sequence associated with a phenotype, trait, or disease status (e.g., eye color, a genetic disease or disorder). A target nucleic acid for determining genetic information can include, but is not limited to, a nucleic acid associated with organism ancestry (e.g., a nucleic acid comprising a single nucleotide polymorphism that identifies geographical ancestry, ancestry from an ethnic group, etc.); a nucleic acid for trait not associated with a communicable disease, cancer, or genetic disorder; a nucleic acid for a phenotypic trait (e.g., a nucleic acid from a gene for blue eyes, brown hair color, fast or slow metabolism of a drug such as caffeine, an intolerance such as lactose intolerance, etc.), or a nucleic acid for genotyping (e.g., a nucleic acid for a gene that is recessive, such as the gene for Taye-Sachs disease).

In some embodiments, target nucleic acid may comprise DNA that was reverse transcribed from RNA using a reverse transcriptase prior to detection by a DNA-activated programmable RNA nuclease (e.g., a Cas13a) using the compositions, systems, and methods disclosed herein.

In some cases, the target nucleic acid is a portion of a nucleic acid from a virus or a bacterium or other agents responsible for a disease in the sample. The target nucleic acid can be a portion of a nucleic acid associated with an infection, where the infection may be caused by a bacterium, virus, or other disease-causing agent. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is a portion of a nucleic acid from sepsis, in the sample. These diseases can include but are not limited to respiratory viruses (e.g., COVID-19, SARS, MERS, influenza and the like), human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to: respiratory viruses (e.g., adenoviruses, parainfluenza viruses, severe acute respiratory syndrome (SARS), coronavirus, MERS), gastrointestinal viruses (e.g., noroviruses, rotaviruses, some adenoviruses, astroviruses), exanthematous viruses (e.g. the virus that causes measles, the virus that causes rubella, the virus that causes chickenpox/shingles, the virus that causes roseola, the virus that causes smallpox, the virus that causes fifth disease, chikungunya virus infection); hepatic viral diseases (e.g., hepatitis A, B, C, D, E); cutaneous viral diseases (e.g. warts (including genital, anal), herpes (including oral, genital, anal), molluscum contagiosum); hemmorhagic viral diseases (e.g. Ebola, Lassa fever, dengue fever, yellow fever, Marburg hemorrhagic fever, Crimean-Congo hemorrhagic fever); neurologic viruses (e.g., polio, viral meningitis, viral encephalitis, rabies), sexually transmitted viruses (e.g., HIV, HPV, and the like), immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

The sample used for cancer testing or cancer risk testing may comprise at least one target nucleic acid that can bind to an engineered guide nucleic acid of the reagents described herein. The target nucleic acid, in some cases, is comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some cases, the assay can be used to detect “hotspots” in target nucleic acids that can be predictive of cancer, such as lung cancer, cervical cancer, in some cases, the cancer can be a cancer that is caused by a virus. Some non-limiting examples of viruses that cause cancers in humans include Epstein-Barr virus (e.g., Burkitt's lymphoma, Hodgkin's Disease, and nasopharyngeal carcinoma); papillomavirus (e.g., cervical carcinoma, anal carcinoma, oropharyngeal carcinoma, penile carcinoma); hepatitis B and C viruses (e.g., hepatocellular carcinoma); human adult T-cell leukemia virus type 1 (HTLV-1) (e.g., T-cell leukemia); and Merkel cell polyomavirus (e.g., Merkel cell carcinoma). One skilled in the art will recognize that viruses can cause or contribute to other types of cancers. In some cases, the target nucleic acid comprises a portion of a nucleic acid that is associated with a blood fever. In some cases, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICERI, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCEl, STK11, SUFU, TERC, TERT, TMEM127, TP53, TSC1, TSC2, VHL, WRN, and WT1.

The sample used for genetic disorder testing may comprise at least one target nucleic acid that can bind to an engineered guide nucleic acid of the reagents described herein. In some embodiments, the genetic disorder is hemophilia, sickle cell anemia, β-thalassemia, Duchene muscular dystrophy, severe combined immunodeficiency, Huntington's disease, or cystic fibrosis. The target nucleic acid, in some cases, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid is a nucleic acid from a genomic locus, reverse transcribed mRNA, a DNA amplicon of or a cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRElC, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHEl, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLSl, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHDI, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

In some embodiments, the target nucleic acid sequence comprises a nucleic acid sequence of a virus, a bacterium, or other pathogen responsible for a disease in a plant (e.g., a crop). Methods and compositions of the disclosure can be used to treat or detect a disease in a plant. For example, the methods of the disclosure can be used to target a viral nucleic acid sequence in a plant. A programmable nuclease of the disclosure can cleave the viral nucleic acid. In some embodiments, the target nucleic acid sequence comprises a nucleic acid sequence of a virus or a bacterium or other agents (e.g., any pathogen) responsible for a disease in the plant (e.g., a crop). In some embodiments, the target nucleic acid comprises DNA that is reverse transcribed from RNA using a reverse transcriptase prior to detection by a programmable nuclease using the compositions, systems, and methods disclosed herein. The target nucleic acid, in some cases, is a portion of a nucleic acid from a virus or a bacterium or other agents responsible for a disease in the plant (e.g., a crop). In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant can be an RNA virus. A virus infecting the plant can be a DNA virus. Non-limiting examples of viruses that can be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

The plant can be a monocotyledonous plant. The plant can be a dicotyledonous plant. Non-limiting examples of orders of dicotyledonous plants include Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales.

Non-limiting examples of orders of monocotyledonous plants include Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales. A plant can belong to the order, for example, Gymnospermae, Pinales, Ginkgoales, Cycadales, Araucariales, Cupressales and Gnetales.

Non-limiting examples of plants include plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses, wheat, maize, rice, millet, barley, tomato, apple, pear, strawberry, orange, acacia, carrot, potato, sugar beets, yam, lettuce, spinach, sunflower, rape seed, Arabidopsis, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. A plant can include algae.

The sample used for phenotyping testing may comprise at least one target nucleic acid that can bind to an engineered guide nucleic acid of the reagents described herein. The target nucleic acid, in some cases, is a portion of a nucleic acid encoding a sequence associated with a phenotypic trait.

The sample used for genotyping testing may comprise at least one target nucleic acid that can bind to an engineer guide nucleic acid of the reagents described herein. The target nucleic acid, in some cases, is a portion of a nucleic acid encoding a sequence associated with a genotype of interest.

The sample used for ancestral testing may comprise at least one target nucleic acid that can bind to an engineer guide nucleic acid of the reagents described herein. The target nucleic acid, in some cases, is a portion of a nucleic acid encoding a sequence associated with a geographic region of origin or ethnic group.

The sample can be used for identifying a disease status. For example, a sample is any sample described herein, and is obtained from a subject for use in identifying a disease status of a subject. The disease can be a cancer or genetic disorder. Sometimes, a method comprises obtaining a serum sample from a subject; and identifying a disease status of the subject. Often, the disease status is prostate disease status.

In some instances, the target nucleic acid is a single stranded nucleic acid. Alternatively or in combination, the target nucleic acid is a double stranded nucleic acid and is prepared into single stranded nucleic acids before or upon contacting the reagents. The target nucleic acid may be a reverse transcribed RNA, DNA, DNA amplicon, synthetic nucleic acids, or nucleic acids found in biological or environmental samples. Preferably, the target nucleic acid is single-stranded DNA (ssDNA). In some cases, the target nucleic acid is from a virus, a parasite, or a bacterium described herein. In some cases, the target nucleic acid is transcribed from a gene as described herein and then reverse transcribed into a DNA amplicon.

A number of target nucleic acids are consistent with the methods and compositions disclosed herein. Some methods described herein can detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample as from 1 to 10,000, from 100 to 8000, from 400 to 6000, from 500 to 5000, from 1000 to 4000, or from 2000 to 3000 target nucleic acids. In some cases, the method detects target nucleic acid present at least at one copy per 10¹ non-target nucleic acids, 10² non-target nucleic acids, 10′ non-target nucleic acids, 104 non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

In some embodiments, the sample comprises a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid sequence at a concentration of from 1 nM to 2 nM, from 2 nM to 3 nM, from 3 nM to 4 nM, from 4 nM to 5 nM, from 5 nM to 6 nM, from 6 nM to 7 nM, from 7 nM to 8 nM, from 8 nM to 9 nM, from 9 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 2 μM, from 2 μM to 3 μM, from 3 μM to 4 μM, from 4 μM to 5 μM, from 5 μM to 6 μM, from 6 μM to 7 μM, from 7 μM to 8 μM, from 8 μM to 9 μM, from 9 μM to 10 μM, from 10 μM to 100 μM, from 100 μM to 1 mM, from 1 nM to 10 nM, from 1 nM to 100 nM, from 1 nM to 1 μM, from 1 nM to 10 μM, from 1 nM to 100 μM, from 1 nM to 1 mM, from 10 nM to 100 nM, from 10 nM to 1 μM, from 10 nM to 10 μM, from 10 nM to 100 μM, from 10 nM to 1 mM, from 100 nM to 1 μM, from 100 nM to 10 μM, from 100 nM to 100 μM, from 100 nM to 1 mM, from 1 μM to 10 μM, from 1 μM to 100 μM, from 1 μM to 1 mM, from 10 μM to 100 μM, from 10 μM to 1 mM, or from 100 μM to 1 mM. In some embodiments, the sample comprises a target nucleic acid at a concentration of from 20 nM to 200 μM, from 50 nM to 100 μM, from 200 nM to 50 μM, from 500 nM to 20 μM, or from 2 μM to 10 μM. In some embodiments, the target nucleic acid is not present in the sample.

In some embodiments, the sample comprises fewer than 10 copies, fewer than 100 copies, fewer than 1000 copies, fewer than 10,000 copies, fewer than 100,000 copies, or fewer than 1,000,000 copies of a target nucleic acid sequence. In some embodiments, the sample comprises from 10 copies to 100 copies, from 100 copies to 1000 copies, from 1000 copies to 10,000 copies, from 10,000 copies to 100,000 copies, from 100,000 copies to 1,000,000 copies, from 10 copies to 1000 copies, from 10 copies to 10,000 copies, from 10 copies to 100,000 copies, from 10 copies to 1,000,000 copies, from 100 copies to 10,000 copies, from 100 copies to 100,000 copies, from 100 copies to 1,000,000 copies, from 1,000 copies to 100,000 copies, or from 1,000 copies to 1,000,000 copies of a target nucleic acid sequence. In some embodiments, the sample comprises from 10 copies to 500,000 copies, from 200 copies to 200,000 copies, from 500 copies to 100,000 copies, from 1000 copies to 50,000 copies, from 2000 copies to 20,000 copies, from 3000 copies to 10,000 copies, or from 4000 copies to 8000 copies. In some embodiments, the target nucleic acid is not present in the sample.

A number of target nucleic acid populations are consistent with the methods and compositions disclosed herein. Some methods described herein can detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has from 3 to 50, from 5 to 40, or from 10 to 25 target nucleic acid populations. In some cases, the method detects target nucleic acid populations that are present at least at one copy per 10¹ non-target nucleic acids, 10² non-target nucleic acids, 10′ non-target nucleic acids, 104 non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

Additionally, target nucleic acid can be an amplified nucleic acid of interest, which can bind to the engineered guide nucleic acid of a programmable nuclease, such as a DNA-activated programmable RNA nuclease. The nucleic acid of interest may be any nucleic acid disclosed herein or from any sample as disclosed herein. This amplification can be thermal amplification (e.g., using PCR) or isothermal amplification. This nucleic acid amplification of the sample can improve at least one of sensitivity, specificity, or accuracy of the detection the target nucleic acid. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C. The nucleic acid amplification reaction can be performed at a temperature of at least 20° C., 25° C., 30° C., 35° C., 37° C., 40° C., or 45° C.

In some embodiments, the target nucleic acid as disclosed herein can be activate the programmable nuclease to initiate trans cleavage of a nucleic acid-based reporter (e.g., a reporter, such as an RNA reporter or DNA reporter). For example, a DNA-activated programmable RNA nuclease herein is activated by a target DNA nucleic acid to cleave RNA reporter molecules. For example, a DNA-activated programmable DNA nuclease disclosed herein is activated by a target DNA nucleic acid to cleave DNA reporter molecules. The RNA reporter can comprise a single-stranded RNA labelled with a reporter or can be any RNA-based reporter as disclosed herein. The DNA reporter can comprise a single-stranded DNA labelled with a reporter or can be any DNA-based reporter as disclosed herein. In some embodiments, a Cas13a recognizes and detects a target single-stranded DNA and, further, trans-cleaves RNA reporters.

Any of the above disclosed samples are consistent with the methods, compositions, reagents, enzymes, and kits disclosed herein and can be used as a companion diagnostic with any of the diseases disclosed herein (e.g., RSV, sepsis, flu), or can be used in reagent kits, point-of-care diagnostics, or over-the-counter diagnostics.

### Reagents

A number of reagents are consistent with the methods, compositions, reagents, enzymes, and kits disclosed herein. The reagents described herein for detecting a disease, cancer, or genetic disorder comprise an engineered guide nucleic acid targeting the target nucleic acid segment indicative of a disease, cancer, or genetic disorder. The reagents disclosed herein may include programmable nucleases, engineered guide nucleic acids, target nucleic acids, and buffers. As described herein, target nucleic acid comprising DNA may be directly detected (e.g., the target DNA hybridizes to the guide nucleic) using a DNA-activated programmable RNA nuclease (e.g., a Cas13a) and other reagents disclosed herein. Direct DNA detection using Cas13 may eliminate the need for intermediate steps, for example reverse transcription or amplification, required by existing programmable nuclease-based sequence detection methods. Elimination of said intermediate steps decreases time to assay result and reduces labor and reagent costs. As described herein, target nucleic acid comprising DNA may be an amplicon of a nucleic acid of interest and the amplicon can be detected (e.g., the target DNA hybridizes to the guide nucleic) using a DNA-activated programmable RNA nuclease (e.g., a Cas13a) and other reagents disclosed herein. Additionally, detection by a DNA-activated programmable RNA nuclease, which can cleave RNA reporters, allows for multiplexing with DNA programmable DNA nuclease that can cleave DNA reporters (e.g., Type V programmable nucleases).

### Guide Nucleic Acids

The reagents of this disclosure may comprise a guide nucleic acid. The guide nucleic acid is an engineered guide nucleic acid. Engineered guide nucleic acids are non-naturally occurring and can be synthetically made. Engineered guide nucleic acids can be encoded for using vectors or can be chemically synthesized. The engineered guide nucleic acid can bind to a single stranded target nucleic acid or portion thereof as described herein. For example, the engineered guide nucleic acid can bind to a target nucleic acid such as nucleic acid from a virus or a bacterium or other agents responsible for a disease, or an amplicon thereof, as described herein. The engineered guide nucleic acid can bind to a target nucleic acid such as a nucleic acid from a bacterium, a virus, a parasite, a protozoa, a fungus or other agents responsible for a disease, or an amplicon thereof, as described herein and further comprising a mutation, such as a single nucleotide polymorphism (SNP), which can confer resistance to a treatment, such as antibiotic treatment. The engineered guide nucleic acid can bind to a target nucleic acid such as a nucleic acid, preferably DNA, from a cancer gene or gene associated with a genetic disorder, or an amplicon thereof, as described herein. The engineered guide nucleic acid comprises a segment of nucleic acids that are reverse complementary to the target nucleic acid. Often the engineered guide nucleic acid binds specifically to the target nucleic acid. The target nucleic acid may be a reversed transcribed RNA, DNA, DNA amplicon, or synthetic nucleic acids. The target nucleic acid can be a single-stranded DNA or DNA amplicon of a nucleic acid of interest.

An engineered guide nucleic acid can comprise a sequence that is reverse complementary to the sequence of a target nucleic acid. An engineered guide nucleic acid can include a crRNA. Sometimes, an engineered guide nucleic acid comprises a crRNA and tracrRNA. The crRNA can have a spacer sequence that is reverse complementary or sufficiently reverse complementary to allow for hybridization to a target nucleic acid. The engineered guide nucleic acid can bind specifically to the target nucleic acid. In some cases, the engineered guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. In some cases, the segment of an engineered guide nucleic acid that comprises a sequence that is reverse complementary to the target nucleic acid is 20 nucleotides in length. The segment of the engineered guide nucleic acid that comprises a sequence that is reverse complementary to the target nucleic acid may have a length of at least 10, 11, 12, 13, 14, 15, 16, 17, 18, 19, 20, 21, 22, 23, 24, 25, 26, 27, 28, 29, or 30 nucleotides in length. In some instances, the segment of the engineered guide nucleic acid that comprises a sequence that is reverse complementary to the target nucleic acid is 10, 11, 12, 13, 14, 15, 16, 17, 18, 19, 20, 21, 22, 23, 24, 25, 26, 27, 28, 29, or 30 nucleotides in length. In some cases, the segment of an engineered guide nucleic acid that comprises a sequence that is reverse complementary to the target nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the segment of an engineered guide nucleic acid that comprises a sequence that is reverse complementary to the target nucleic acid has a length of from about 10 nt to about 60 nt, from about 20 nt to about 50 nt, or from about 30 nt to about 40 nt. It is understood that the sequence of a polynucleotide need not be 100% complementary to that of its target nucleic acid to be specifically hybridizable or hybridizable or bind specifically. The engineered guide nucleic acid can have a sequence comprising at least one uracil in a region from nucleic acid residue 5 to 20 that is reverse complementary to a modification variable region in the target nucleic acid. The engineered guide nucleic acid, in some cases, has a sequence comprising at least one uracil in a region from nucleic acid residue 5 to 9, 10 to 14, or 15 to 20 that is reverse complementary to a modification variable region in the target nucleic acid. The engineered guide nucleic acid can have a sequence comprising at least one uracil in a region from nucleic acid residue 5 to 20 that is reverse complementary to a methylation variable region in the target nucleic acid. The engineered guide nucleic acid, in some cases, has a sequence comprising at least one uracil in a region from nucleic acid residue 5 to 9, 10 to 14, or 15 to 20 that is reverse complementary to a methylation variable region in the target nucleic acid. The engineered guide nucleic acid can hybridize with a target nucleic acid.

The engineered guide nucleic acid can be selected from a group of engineered guide nucleic acids that have been tiled against the nucleic acid sequence of a strain of an infection or genomic locus of interest. The engineered guide nucleic acid can be selected from a group of engineered guide nucleic acids that have been tiled against the nucleic acid sequence of a strain of HPV 16 or HPV18. Often, engineered guide nucleic acids that are tiled against the nucleic acid of a strain of an infection or genomic locus of interest can be pooled for use in a method described herein. Often, these engineered guide nucleic acids are pooled for detecting a target nucleic acid in a single assay. The pooling of engineered guide nucleic acids that are tiled against a single target nucleic acid can enhance the detection of the target nucleic using the methods described herein. The pooling of engineered guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled engineered guide nucleic acids along the target nucleic acid. In some instances the tiling of the engineered guide nucleic acids is non-sequential. Often, a method for detecting a target nucleic acid comprises contacting a target nucleic acid to a pool of engineered guide nucleic acids and a programmable nuclease, wherein an engineered guide nucleic acid sequence of the pool of engineered guide nucleic acids has a sequence selected from a group of tiled engineered guide nucleic acid that correspond to nucleic acid sequence of a target nucleic acid; and assaying for a signal produce by cleavage of at least some detector nucleic acids of a population of detector nucleic acids. Pooling of engineered guide nucleic acids can ensure broad spectrum identification, or broad coverage, of a target species within a single reaction. This can be particularly helpful in diseases or indications, like sepsis, that may be caused by multiple organisms.

### Programmable Nucleases

The programmable nucleases disclosed herein (e.g., a DNA-activated programmable RNA nuclease such as a type VI CRISPR enzyme) enable the detection of target nucleic acids (e.g., DNA). Additionally, detection by a DNA-activated programmable RNA nuclease, which can cleave RNA reporters, allows for multiplexing with other programmable nucleases, such as a a DNA-activated programmable DNA nuclease (e.g., a Type V CRISPR enzyme).

In some embodiments, the Type VI CRISPR/Cas enzyme is a Cas13 nuclease. The general architecture of a Cas13 protein includes an N-terminal domain and two HEPN (higher eukaryotes and prokaryotes nucleotide-binding) domains separated by two helical domains (Liu et al., Cell 2017 Jan. 12;168(1-2):121-134.e12). The HEPN domains each comprise aR-X₄-H motif Shared features across Cas13 proteins include that upon binding of the crRNA of the engineered guide nucleic acid to a target nucleic acid, the protein undergoes a conformational change to bring together the HEPN domains and form a catalytically active RNase. (Tambe et al., Cell Rep. 2018 Jul. 24; 24(4): 1025-1036.). Thus, two activatable HEPN domains are characteristic of a Cas13 nuclease of the present disclosure. However, Cas13 nucleases also consistent with the present disclosure include Cas13 nucleases comprising mutations in the HEPN domain that enhance the Cas13 proteins cleavage efficiency or mutations that catalytically inactivate the HEPN domains. Cas13 nucleases consistent with the present disclosure also Cas13 nucleases comprising catalytic

A Cas13 nuclease can be a Cas13a protein (also referred to as “c2c2”), a Cas13b protein, a Cas13c protein, a Cas13d protein, or a Cas13e protein. Example C2c2 proteins are set forth as SEQ ID NO: 18-SEQ ID NO: 35. In some cases, a subject C2c2 protein includes an amino acid sequence having 80% r more (e.g., 85% r more, 90% r more, 95% r more, 98% r more, 99% r more, 99.5% r more, or 100%) amino acid sequence identity with the amino acid sequence set forth in any one of SEQ ID NO: 18-SEQ ID NO: 35. In some cases, a suitable C2c2 polypeptide comprises an amino acid sequence having at least 80%, at least 85%, at least 90%, at least 95%, at least 98%, at least 99%, or 100%, amino acid sequence identity to the Listeria seeligeri C2c2 amino acid sequence set forth in SEQ ID NO: 18. In some cases, a suitable C2c2 polypeptide comprises an amino acid sequence having at least 80%, at least 85%, at least 90%, at least 95%, at least 98%, at least 99%, or 100%, amino acid sequence identity to the Leptotrichia buccalis C2c2 amino acid sequence set forth in SEQ ID NO: 19. In some cases, a suitable C2c2 polypeptide comprises an amino acid sequence having at least 80%, at least 85%, at least 90%, at least 95%, at least 98%, at least 99%, or 100%, amino acid sequence identity to the Rhodobacter capsulatus C2c2 amino acid sequence set forth in SEQ ID NO: 21. In some cases, a suitable C2c2 polypeptide comprises an amino acid sequence having at least 80%, at least 85%, at least 90%, at least 95%, at least 98%, at least 99%, or 100%, amino acid sequence identity to the Carnobacterium gallinarum C2c2 amino acid sequence set forth in SEQ ID NO: 22. In some cases, a suitable C2c2 polypeptide comprises an amino acid sequence having at least 80%, at least 85%, at least 90%, at least 95%, at least 98%, at least 99%, or 100%, amino acid sequence identity to the Herbinix hemicellulosilytica C2c2 amino acid sequence set forth in SEQ ID NO: 23. In some cases, the C2c2 protein includes an amino acid sequence having 80% r more amino acid sequence identity with the Leptotrichia buccalis (Lbu) C2c2 amino acid sequence set forth in SEQ ID NO: 19. In some cases, the C2c2 protein is a Leptotrichia buccalis (Lbu) C2c2 protein (e.g., see SEQ ID NO: 19). In some cases, the C2c2 protein includes the amino acid sequence set forth in any one of SEQ ID NO: 18-SEQ ID NO: 35. In some cases, a C2c2 protein used in a method of the present disclosure is not a C2c2 polypeptide having at least 80%, at least 85%, at least 90%, at least 95%, at least 98%, at least 99%, or 100%, amino acid sequence identity to the Lsh C2c2 polypeptide set forth in SEQ ID NO: 20. Exemplary Cas13 protein sequences are set forth in SEQ ID NO: 18-SEQ ID NO: 35. TABLE 1, below, shows exemplary Cas13 DNA-activated programmable nuclease sequences of the present disclsorue.

TABLE 1
Cas 13 Protein Sequences

| SEQ ID NO | Description | Sequence |
|---|---|---|
| SEQ ID NO: 18 | Listeria | MWISIKTLIHHLGVLFFCDYMYNRREKKIIEVKTMRITKVE |
| | seeligeri C2c2 | VDRKKVLISRDKNGGKLVYENEMQDNTEQIMHHKKSSFY |
| | amino acid | KSVVNKTICRPEQKQMKKLVHGLLQENSQEKIKVSDVTKL |
| | sequence | NISNFLNHRFKKSLYYFPENSPDKSEEYRIEINLSQLLEDSL |
| | | KKQQGTFICWESFSKDMELYINWAENYISSKTKLIKKSIRN |
| | | NRIQSTESRSGQLMDRYMKDILNKNKPFDIQSVSEKYQLE |
| | | KLTSALKATFKEAKKNDKEINYKLKSTLQNHERQIIEELKE |
| | | NSELNQFNIEIRKHLETYFPIKKTNRKVGDIRNLEIGEIQKIV |
| | | NHRLKNKIVQRILQEGKLASYEIESTVNSNSLQKIKIEEAFA |
| | | LKFINACLFASNNLRNMVYPVCKKDILMIGEFKNSFKEIKH |
| | | KKFIRQWSQFFSQEITVDDIELASWGLRGAIAPIRNEIIHLK |
| | | KHSWKKFFNNPTFKVKKSKIINGKTKDVTSEFLYKETLFK |
| | | DYFYSELDSVPELIINKMESSKILDYYSSDQLNQVFTIPNFE |
| | | LSLLTSAVPFAPSFKRVYLKGFDYQNQDEAQPDYNLKLNI |
| | | YNEKAFNSEAFQAQYSLFKMVYYQVFLPQFTTNNDLFKSS |
| | | VDFILTLNKERKGYAKAFQDIRKMNKDEKPSEYMSYIQSQ |
| | | LMLYQKKQEEKEKINHFEKFINQVFIKGFNSFIEKNRLTYIC |
| | | HPTKNTVPENDNIEIPFHTDMDDSNIAFWLMCKLLDAKQL |
| | | SELRNEMIKFSCSLQSTEEISTFTKAREVIGLALLNGEKGCN |
| | | DWKELFDDKEAWKKNMSLYVSEELLQSLPYTQEDGQTPV |
| | | INRSIDLVKKYGTETILEKLFSSSDDYKVSAKDIAKLHEYD |
| | | VTEKIAQQESLHKQWIEKPGLARDSAWTKKYQNVINDISN |
| | | YQWAKTKVELTQVRHLHQLTIDLLSRLAGYMSIADRDFQF |
| | | SSNYILERENSEYRVTSWILLSENKNKNKYNDYELYNLKN |
| | | ASIKVSSKNDPQLKVDLKQLRLTLEYLELFDNRLKEKRNNI |
| | | SHFNYLNGQLGNSILELFDDARDVLSYDRKLKNAVSKSLK |
| | | EILSSHGMEVTFKPLYQTNHHLKIDKLQPKKIHHLGEKSTV |
| | | SSNQVSNEYCQLVRTLLTMK |
| SEQ ID NO: 19 | Leptotrichia | MKVTKVGGISHKKYTSEGRLVKSESEENRTDERLSALLNM |
| | buccalis (Lbu) | RLDMYIKNPSSTETKENQKRIGKLKKFFSNKMVYLKDNTL |
| | C2c2 amino | SLKNGKKENIDREYSETDILESDVRDKKNFAVLKKIYLNEN |
| | acid sequence | VNSEELEVFRNDIKKKLNKINSLKYSFEKNKANYQKINEN |
| | | NIEKVEGKSKRNIIYDYYRESAKRDAYVSNVKEAFDKLYK |
| | | EEDIAKLVLEIENLTKLEKYKIREFYHEIIGRKNDKENFAKII |
| | | YEEIQNVNNMKELIEKVPDMSELKKSQVFYKYYLDKEELN |
| | | DKNIKYAFCHFVEIEMSQLLKNYVYKRLSNISNDKIKRIFE |
| | | YQNLKKLIENKLLNKLDTYVRNCGKYNYYLQDGEIATSDF |
| | | IARNRQNEAFLRNIIGVSSVAYFSLRNILETENENDITGRMR |
| | | GKTVKNNKGEEKYVSGEVDKIYNENKKNEVKENLKMFYS |
| | | YDFNMDNKNEIEDFFANIDEAISSIRHGIVHFNLELEGKDIF |
| | | AFKNIAPSEISKKMFQNEINEKKLKLKIFRQLNSANVFRYL |
| | | EKYKILNYLKRTRFEFVNKNIPFVPSFTKLYSRIDDLKNSLG |
| | | IYWKTPKTNDDNKTKEIIDAQIYLLKNIYYGEFLNYFMSNN |
| | | GNFFEISKEIIELNKNDKRNLKTGFYKLQKFEDIQEKIPKEY |
| | | LANIQSLYMINAGNQDEEEKDTYIDFIQKIFLKGFMTYLAN |
| | | NGRLSLIYIGSDEETNTSLAEKKQEFDKFLKKYEQNNNIKIP |
| | | YEINEFLREIKLGNILKYTERLNMFYLILKLLNHKELTNLK |
| | | GSLEKYQSANKEEAFSDQLELINLLNLDNNRVTEDFELEA |
| | | DEIGKFLDFNGNKVKDNKELKKFDTNKIYFDGENIIKHRAF |
| | | YNIKKYGMLNLLEKIADKAGYKISIEELKKYSNKKNEIEKN |
| | | HKMQENLHRKYARPRKDEKFTDEDYESYKQAIENIEEYTH |
| | | LKNKVEFNELNLLQGLLLRILHRLVGYTSIWERDLRFRLK |
| | | GEFPENQYIEEIFNFENKKNVKYKGGQIVEKYIKFYKELHQ |
| | | NDEVKINKYSSANIKVLKQEKKDLYIRNYIAHFNYIPHAEIS |
| | | LLEVLENLRKLLSYDRKLKNAVMKSVVDILKEYGFVATFK |
| | | IGADKKIGIQTLESEKIVHLKNLKKKKLMTDRNSEELCKLV |
| | | KIMFEYKMEEKKSEN |
| SEQ ID NO: 20 | Leptotrichia | MGNLFGHKRWYEVRDKKDFKIKRKVKVKRNYDGNKYIL |
| | shahii (Lsh) | NINENNNKEKIDNNKFIRKYINYKKNDNILKEFTRKFHAGN |
| | C2c2 protein | ILFKLKGKEGIIRIENNDDFLETEEVVLYIEAYGKSEKLKAL |
| | | GITKKKIIDEAIRQGITKDDKKIEIKRQENEEEIEIDIRDEYTN |
| | | KTLNDCSIILRIIENDELETKKSIYEIFKNINMSLYKIIEKIIEN |
| | | ETEKVFENRYYEEHLREKLLKDDKIDVILTNFMEIREKIKS |
| | | NLEILGFVKFYLNVGGDKKKSKNKKMLVEKILNINVDLTV |
| | | EDIADFVIKELEFWNITKRIEKVKKVNNEFLEKRRNRTYIK |
| | | SYVLLDKHEKFKIERENKKDKIVKFFVENIKNNSIKEKIEKI |
| | | LAEFKIDELIKKLEKELKKGNCDTEIFGIFKKHYKVNFDSK |
| | | KFSKKSDEEKELYKIIYRYLKGRIEKILVNEQKVRLKKMEK |
| | | IEIEKILNESILSEKILKRVKQYTLEHIMYLGKLRHNDIDMT |
| | | TVNTDDFSRLHAKEELDLELITFFASTNMELNKIFSRENINN |
| | | DENIDFFGGDREKNYVLDKKILNSKIKIIRDLDFIDNKNNIT |
| | | NNFIRKFTKIGTNERNRILHAISKERDLQGTQDDYNKVINII |
| | | QNLKISDEEVSKALNLDVVFKDKKNIITKINDIKISEENNND |
| | | IKYLPSFSKVLPEILNLYRNNPKNEPFDTIETEKIVLNALIYV |
| | | NKELYKKLILEDDLEENESKNIFLQELKKTLGNIDEIDENIIE |
| | | NYYKNAQISASKGNNKAIKKYQKKVIECYIGYLRKNYEEL |
| | | FDFSDFKMNIQEIKKQIKDINDNKTYERITVKTSDKTIVIND |
| | | DFEYIISIFALLNSNAVINKIRNRFFATSVWLNTSEYQNIIDIL |
| | | DEIMQLNTLRNECITENWNLNLEEFIQKMKEIEKDFDDFKI |
| | | QTKKEIFNNYYEDIKNNILTEFKDDINGCDVLEKKLEKIVIF |
| | | DDETKFEIDKKSNILQDEQRKLSNINKKDLKKKVDQYIKD |
| | | KDQEIKSKILCRIIFNSDFLKKYKKEIDNLIEDMESENENKF |
| | | QEIYYPKERKNELYIYKKNLFLNIGNPNFDKIYGLISNDIKM |
| | | ADAKFLFNIDGKNIRKNKISEIDAILKNLNDKLNGYSKEYK |
| | | EKYIKKLKENDDFFAKNIQNKNYKSFEKDYNRVSEYKKIR |
| | | DLVEFNYLNKIESYLIDINWKLAIQMARFERDMHYIVNGL |
| | | RELGIIKLSGYNTGISRAYPKRNGSDGFYTTTAYYKFFDEE |
| | | SYKKFEKICYGFGIDLSENSEINKPENESIRNYISHFYIVRNP |
| | | FADYSIAEQIDRVSNLLSYSTRYNNSTYASVFEVFKKDVNL |
| | | DYDELKKKFKLIGNNDILERLMKPKKVSVLELESYNSDYI |
| | | KNLIIELLTKIENTNDTL |
| SEQ ID NO: 21 | Rhodobacter | MQIGKVQGRTISEFGDPAGGLKRKISTDGKNRKELPAHLSS |
| | capsulatus | DPKALIGQWISGIDKIYRKPDSRKSDGKAIHSPTPSKMQFD |
| | C2c2 amino | ARDDLGEAFWKLVSEAGLAQDSDYDQFKRRLHPYGDKFQ |
| | acid sequence | PADSGAKLKFEADPPEPQAFHGRWYGAMSKRGNDAKELA |
| | | AALYEHLHVDEKRIDGQPKRNPKTDKFAPGLVVARALGIE |
| | | SSVLPRGMARLARNWGEEEIQTYFVVDVAASVKEVAKAA |
| | | VSAAQAFDPPRQVSGRSLSPKVGFALAEHLERVTGSKRCS |
| | | FDPAAGPSVLALHDEVKKTYKRLCARGKNAARAFPADKT |
| | | ELLALMRHTHENRVRNQMVRMGRVSEYRGQQAGDLAQS |
| | | HYWTSAGQTEIKESEIFVRLWVGAFALAGRSMKAWIDPM |
| | | GKIVNTEKNDRDLTAAVNIRQVISNKEMVAEAMARRGIYF |
| | | GETPELDRLGAEGNEGFVFALLRYLRGCRNQTFHLGARAG |
| | | FLKEIRKELEKTRWGKAKEAEHVVLTDKTVAAIRAIIDND |
| | | AKALGARLLADLSGAFVAHYASKEHFSTLYSEIVKAVKDA |
| | | PEVSSGLPRLKLLLKRADGVRGYVHGLRDTRKHAFATKLP |
| | | PPPAPRELDDPATKARYIALLRLYDGPFRAYASGITGTALA |
| | | GPAARAKEAATALAQSVNVTKAYSDVMEGRSSRLRPPND |
| | | GETLREYLSALTGETATEFRVQIGYESDSENARKQAEFIEN |
| | | YRRDMLAFMFEDYIRAKGFDWILKIEPGATAMTRAPVLPE |
| | | PIDTRGQYEHWQAALYLVMHFVPASDVSNLLHQLRKWEA |
| | | LQGKYELVQDGDATDQADARREALDLVKRFRDVLVLFLK |
| | | TGEARFEGRAAPFDLKPFRALFANPATFDRLFMATPTTARP |
| | | AEDDPEGDGASEPELRVARTLRGLRQIARYNHMAVLSDLF |
| | | AKHKVRDEEVARLAEIEDETQEKSQIVAAQELRTDLHDKV |
| | | MKCHPKTISPEERQSYAAAIKTIEEHRFLVGRVYLGDHLRL |
| | | HRLMMDVIGRLIDYAGAYERDTGTFLINASKQLGAGADW |
| | | AVTIAGAANTDARTQTRKDLAHFNVLDRADGTPDLTALV |
| | | NRAREMMAYDRKRKNAVPRSILDMLARLGLTLKWQMKD |
| | | HLLQDATITQAAIKHLDKVRLTVGGPAAVTEARFSQDYLQ |
| | | MVAAVFNGSVQNPKPRRRDDGDAWHKPPKPATAQSQPD |
| | | QKPPNKAPSAGSRLPPPQVGEVYEGVVVKVIDTGSLGFLA |
| | | VEGVAGNIGLHISRLRRIREDAIIVGRRYRFRVEIYVPPKSN |
| | | TSKLNAADLVRID |
| SEQ ID NO: 22 | Carnobacterium | MRITKVKIKLDNKLYQVTMQKEEKYGTLKLNEESRKSTAE |
| | gallinarum | ILRLKKASFNKSFHSKTINSQKENKNATIKKNGDYISQIFEK |
| | C2c2 amino | LVGVDTNKNIRKPKMSLTDLKDLPKKDLALFIKRKFKNDD |
| | acid sequence | IVEIKNLDLISLFYNALQKVPGEHFTDESWADFCQEMMPY |
| | | REYKNKFIERKIILLANSIEQNKGFSINPETFSKRKRVLHQW |
| | | AIEVQERGDFSILDEKLSKLAEIYNFKKMCKRVQDELNDLE |
| | | KSMKKGKNPEKEKEAYKKQKNFKIKTIWKDYPYKTHIGLI |
| | | EKIKENEELNQFNIEIGKYFEHYFPIKKERCTEDEPYYLNSE |
| | | TIATTVNYQLKNALISYLMQIGKYKQFGLENQVLDSKKLQ |
| | | EIGIYEGFQTKFMDACVFATSSLKNIIEPMRSGDILGKREFK |
| | | EAIATSSFVNYHHFFPYFPFELKGMKDRESELIPFGEQTEAK |
| | | QMQNIWALRGSVQQIRNEIFHSFDKNQKFNLPQLDKSNFE |
| | | FDASENSTGKSQSYIETDYKFLFEAEKNQLEQFFIERIKSSG |
| | | ALEYYPLKSLEKLFAKKEMKFSLGSQVVAFAPSYKKLVKK |
| | | GHSYQTATEGTANYLGLSYYNRYELKEESFQAQYYLLKLI |
| | | YQYVFLPNFSQGNSPAFRETVKAILRINKDEARKKMKKNK |
| | | KFLRKYAFEQVREMEFKETPDQYMSYLQSEMREEKVRKA |
| | | EKNDKGFEKNITMNFEKLLMQIFVKGFDVFLTTFAGKELL |
| | | LSSEEKVIKETEISLSKKINEREKTLKASIQVEHQLVATNSAI |
| | | SYWLFCKLLDSRHLNELRNEMIKFKQSRIKFNHTQHAELIQ |
| | | NLLPIVELTILSNDYDEKNDSQNVDVSAYFEDKSLYETAPY |
| | | VQTDDRTRVSFRPILKLEKYHTKSLIEALLKDNPQFRVAAT |
| | | DIQEWMHKREEIGELVEKRKNLHTEWAEGQQTLGAEKRE |
| | | EYRDYCKKIDRFNWKANKVTLTYLSQLHYLITDLLGRMV |
| | | GFSALFERDLVYFSRSFSELGGETYHISDYKNLSGVLRLNA |
| | | EVKPIKIKNIKVIDNEENPYKGNEPEVKPFLDRLHAYLENVI |
| | | GIKAVHGKIRNQTAHLSVLQLELSMIESMNNLRDLMAYDR |
| | | KLKNAVTKSMIKILDKHGMILKLKIDENHKNFEIESLIPKEII |
| | | HLKDKAIKTNQVSEEYCQLVLALLTTNPGNQLN |
| SEQ ID NO: 23 | Herbinix | MKLTRRRISGNSVDQKITAAFYRDMSQGLLYYDSEDNDCT |
| | hemicellulosilytica | DKVIESMDFERSWRGRILKNGEDDKNPFYMFVKGLVGSN |
| | C2c2 | DKIVCEPIDVDSDPDNLDILINKNLTGFGRNLKAPDSNDTL |
| | amino acid | ENLIRKIQAGIPEEEVLPELKKIKEMIQKDIVNRKEQLLKSIK |
| | sequence | NNRIPFSLEGSKLVPSTKKMKWLFKLIDVPNKTFNEKMLE |
| | | KYWEIYDYDKLKANITNRLDKTDKKARSISRAVSEELREY |
| | | HKNLRTNYNRFVSGDRPAAGLDNGGSAKYNPDKEEFLLF |
| | | LKEVEQYFKKYFPVKSKHSNKSKDKSLVDKYKNYCSYKV |
| | | VKKEVNRSIINQLVAGLIQQGKLLYYFYYNDTWQEDFLNS |
| | | YGLSYIQVEEAFKKSVMTSLSWGINRLTSFFIDDSNTVKFD |
| | | DITTKKAKEAIESNYFNKLRTCSRMQDHFKEKLAFFYPVY |
| | | VKDKKDRPDDDIENLIVLVKNAIESVSYLRNRTFHFKESSL |
| | | LELLKELDDKNSGQNKIDYSVAAEFIKRDIENLYDVFREQI |
| | | RSLGIAEYYKADMISDCFKTCGLEFALYSPKNSLMPAFKN |
| | | VYKRGANLNKAYIRDKGPKETGDQGQNSYKALEEYRELT |
| | | WYIEVKNNDQSYNAYKNLLQLIYYHAFLPEVRENEALITD |
| | | FINRTKEWNRKETEERLNTKNNKKHKNFDENDDITVNTYR |
| | | YESIPDYQGESLDDYLKVLQRKQMARAKEVNEKEEGNNN |
| | | YIQFIRDVVVWAFGAYLENKLKNYKNELQPPLSKENIGLN |
| | | DTLKELFPEEKVKSPFNIKCRFSISTFIDNKGKSTDNTSAEA |
| | | VKTDGKEDEKDKKNIKRKDLLCFYLFLRLLDENEICKLQH |
| | | QFIKYRCSLKERRFPGNRTKLEKETELLAELEELMELVRFT |
| | | MPSIPEISAKAESGYDTMIKKYFKDFIEKKVFKNPKTSNLY |
| | | YHSDSKTPVTRKYMALLMRSAPLHLYKDIFKGYYLITKKE |
| | | CLEYIKLSNIIKDYQNSLNELHEQLERIKLKSEKQNGKDSL |
| | | YLDKKDFYKVKEYVENLEQVARYKHLQHKINFESLYRIFR |
| | | IHVDIAARMVGYTQDWERDMHFLFKALVYNGVLEERRFE |
| | | AIFNNNDDNNDGRIVKKIQNNLNNKNRELVSMLCWNKKL |
| | | NKNEFGAIIWKRNPIAHLNHFTQTEQNSKSSLESLINSLRIL |
| | | LAYDRKRQNAVTKTINDLLLNDYHIRIKWEGRVDEGQIYF |
| | | NIKEKEDIENEPIIHLKHLHKKDCYIYKNSYMFDKQKEWIC |
| | | NGIKEEVYDKSILKCIGNLFKFDYEDKNKSSANPKHT |
| SEQ ID NO: 24 | Paludibacter | MRVSKVKVKDGGKDKMVLVHRKTTGAQLVYSGQPVSNE |
| | propionicigenes | TSNILPEKKRQSFDLSTLNKTIIKFDTAKKQKLNVDQYKIV |
| | C2c2 | EKIFKYPKQELPKQIKAEEILPFLNHKFQEPVKYWKNGKEE |
| | amino acid | SFNLTLLIVEAVQAQDKRKLQPYYDWKTWYIQTKSDLLK |
| | sequence | KSIENNRIDLTENLSKRKKALLAWETEFTASGSIDLTHYHK |
| | | VYMTDVLCKMLQDVKPLTDDKGKINTNAYHRGLKKALQ |
| | | NHQPAIFGTREVPNEANRADNQLSIYHLEVVKYLEHYFPIK |
| | | TSKRRNTADDIAHYLKAQTLKTTIEKQLVNAIRANIIQQGK |
| | | TNHHELKADTTSNDLIRIKTNEAFVLNLTGTCAFAANNIRN |
| | | MVDNEQTNDILGKGDFIKSLLKDNTNSQLYSFFFGEGLSTN |
| | | KAEKETQLWGIRGAVQQIRNNVNHYKKDALKTVFNISNFE |
| | | NPTITDPKQQTNYADTIYKARFINELEKIPEAFAQQLKTGG |
| | | AVSYYTIENLKSLLTTFQFSLCRSTIPFAPGFKKVFNGGINY |
| | | QNAKQDESFYELMLEQYLRKENFAEESYNARYFMLKLIY |
| | | NNLFLPGFTTDRKAFADSVGFVQMQNKKQAEKVNPRKKE |
| | | AYAFEAVRPMTAADSIADYMAYVQSELMQEQNKKEEKV |
| | | AEETRINFEKFVLQVFIKGFDSFLRAKEFDFVQMPQPQLTA |
| | | TASNQQKADKLNQLEASITADCKLTPQYAKADDATHIAFY |
| | | VFCKLLDAAHLSNLRNELIKFRESVNEFKFHHLLEIIEICLLS |
| | | ADVVPTDYRDLYSSEADCLARLRPFIEQGADITNWSDLFV |
| | | QSDKHSPVIHANIELSVKYGTTKLLEQIINKDTQFKTTEANF |
| | | TAWNTAQKSIEQLIKQREDHHEQWVKAKNADDKEKQER |
| | | KREKSNFAQKFIEKHGDDYLDICDYINTYNWLDNKMHFV |
| | | HLNRLHGLTIELLGRMAGFVALFDRDFQFFDEQQIADEFK |
| | | LHGFVNLHSIDKKLNEVPTKKIKEIYDIRNKIIQINGNKINES |
| | | VRANLIQFISSKRNYYNNAFLHVSNDEIKEKQMYDIRNHIA |
| | | HFNYLTKDAADFSLIDLINELRELLHYDRKLKNAVSKAFID |
| | | LFDKHGMILKLKLNADHKLKVESLEPKKIYHLGSSAKDKP |
| | | EYQYCTNQVMMAYCNMCRSLLEMKK |
| SEQ ID NO: 25 | Leptotrichia | MYMKITKIDGVSHYKKQDKGILKKKWKDLDERKQREKIE |
| | wadei (Lwa) | ARYNKQIESKIYKEFFRLKNKKRIEKEEDQNIKSLYFFIKEL |
| | C2c2 amino | YLNEKNEEWELKNINLEILDDKERVIKGYKFKEDVYFFKE |
| | acid sequence | GYKEYYLRILFNNLIEKVQNENREKVRKNKEFLDLKEIFKK |
| | | YKNRKIDLLLKSINNNKINLEYKKENVNEEIYGINPTNDRE |
| | | MTFYELLKEIIEKKDEQKSILEEKLDNFDITNFLENIEKIFNE |
| | | ETEINIIKGKVLNELREYIKEKEENNSDNKLKQIYNLELKK |
| | | YIENNFSYKKQKSKSKNGKNDYLYLNFLKKIMFIEEVDEK |
| | | KEINKEKFKNKINSNFKNLFVQHILDYGKLLYYKENDEYIK |
| | | NTGQLETKDLEYIKTKETLIRKMAVLVSFAANSYYNLFGR |
| | | VSGDILGTEVVKSSKTNVIKVGSHIFKEKMLNYFFDFEIFD |
| | | ANKIVEILESISYSIYNVRNGVGHFNKLILGKYKKKDINTN |
| | | KRIEEDLNNNEEIKGYFIKKRGEIERKVKEKFLSNNLQYYY |
| | | SKEKIENYFEVYEFEILKRKIPFAPNFKR11KKGEDLFNNKN |
| | | NKKYEYFKNFDKNSAEEKKEFLKTRNFLLKELYYNNFYK |
| | | EFLSKKEEFEKIVLEVKEEKKSRGNINNKKSGVSFQSIDDY |
| | | DTKINISDYIASIHKKEMERVEKYNEEKQKDTAKYIRDFVE |
| | | EIFLTGFINYLEKDKRLHFLKEEFSILCNNNNNVVDFNININ |
| | | EEKIKEFLKENDSKTLNLYLFFNMIDSKRISEFRNELVKYK |
| | | QFTKKRLDEEKEFLGIKIELYETLIEFVILTREKLDTKKSEEI |
| | | DAWLVDKLYVKDSNEYKEYEEILKLFVDEKILSSKEAPYY |
| | | ATDNKTPILLSNFEKTRKYGTQSFLSEIQSNYKYSKVEKENI |
| | | EDYNKKEEIEQKKKSNIEKLQDLKVELHKKWEQNKITEKE |
| | | IEKYNNTTRKINEYNYLKNKEELQNVYLLHEMLSDLLARN |
| | | VAFFNKWERDFKFIVIAIKQFLRENDKEKVNEFLNPPDNSK |
| | | GKKVYFSVSKYKNTVENIDGIHKNFMNLIFLNNKFMNRKI |
| | | DKMNCAIWVYFRNYIAHFLHLHTKNEKISLISQMNLLIKLF |
| | | SYDKKVQNHILKSTKTLLEKYNIQINFEISNDKNEVFKYKI |
| | | KNRLYSKKGKMLGKNNKFEILENEFLENVKAMLEYSE |
| SEQ ID NO: 26 | Bergeyella | MENKTSLGNNIYYNPFKPQDKSYFAGYFNAAMENTDSVF |
| | zoohelcum | RELGKRLKGKEYTSENFFDAIFKENISLVEYERYVKLLSDY |
| | Cas13b | FPMARLLDKKEVPIKERKENFKKNFKGIIKAVRDLRNFYT |
| | | HKEHGEVEITDEIFGVLDEMLKSTVLTVKKKKVKTDKTKE |
| | | ILKKSIEKQLDILCQKKLEYLRDTARKIEEKRRNQRERGEK |
| | | ELVAPFKYSDKRDDLIAAIYNDAFDVYIDKKKDSLKESSK |
| | | AKYNTKSDPQQEEGDLKIPISKNGVVFLLSLFLTKQEIHAF |
| | | KSKIAGFKATVIDEATVSEATVSHGKNSICFMATHEIFSHL |
| | | AYKKLKRKVRTAEINYGEAENAEQLSVYAKETLMMQML |
| | | DELSKVPDVVYQNLSEDVQKTFIEDWNEYLKENNGDVGT |
| | | MEEEQVIHPVIRKRYEDKFNYFAIRFLDEFAQFPTLRFQVH |
| | | LGNYLHDSRPKENLISDRRIKEKITVFGRLSELEHKKALFIK |
| | | NTETNEDREHYWEIFPNPNYDFPKENISVNDKDFPIAGSILD |
| | | REKQPVAGKIGIKVKLLNQQYVSEVDKAVKAHQLKQRKA |
| | | SKPSIQNIIEEIVPINESNPKEAIVFGGQPTAYLSMNDIHSILY |
| | | EFFDKWEKKKEKLEKKGEKELRKEIGKELEKKIVGKIQAQI |
| | | QQIIDKDTNAKILKPYQDGNSTAIDKEKLIKDLKQEQNILQ |
| | | KLKDEQTVREKEYNDFIAYQDKNREINKVRDRNHKQYLK |
| | | DNLKRKYPEAPARKEVLYYREKGKVAVWLANDIKRFMPT |
| | | DFKNEWKGEQHSLLQKSLAYYEQCKEELKNLLPEKVFQH |
| | | LPFKLGGYFQQKYLYQFYTCYLDKRLEYISGLVQQAENFK |
| | | SENKVFKKVENECFKFLKKQNYTHKELDARVQSILGYPIFL |
| | | ERGFMDEKPTIIKGKTFKGNEALFADWFRYYKEYQNFQTF |
| | | YDTENYPLVELEKKQADRKRKTKIYQQKKNDVFTLLMAK |
| | | HIFKSVFKQDSIDQFSLEDLYQSREERLGNQERARQTGERN |
| | | TNYIWNKTVDLKLCDGKITVENVKLKNVGDFIKYEYDQR |
| | | VQAFLKYEENIEWQAFLIKESKEEENYPYVVEREIEQYEKV |
| | | RREELLKEVHLIEEYILEKVKDKEILKKGDNQNFKYYILNG |
| | | LLKQLKNEDVESYKVFNLNTEPEDVNINQLKQEATDLEQK |
| | | AFVLTYIRNKFAHNQLPKKEFWDYCQEKYGKIEKEKTYAE |
| | | YFAEVFKKEKEALIK |
| SEQ ID NO: 27 | Prevotella | MEDDKKTTDSIRYELKDKHFWAAFLNLARHNVYITVNHI |
| | intermedia | NKILEEGEINRDGYETTLKNTWNEIKDINKKDRLSKLIIKHF |
| | Cas 13b | PFLEAATYRLNPTDTTKQKEEKQAEAQSLESLRKSFFVFIY |
| | | KLRDLRNHYSHYKHSKSLERPKFEEGLLEKMYNIFNASIRL |
| | | VKEDYQYNKDINPDEDFKHLDRTEEEFNYYFTKDNEGNIT |
| | | ESGLLFFVSLFLEKKDAIWMQQKLRGFKDNRENKKKMTN |
| | | EVFCRSRMLLPKLRLQSTQTQDWILLDMLNELIRCPKSLYE |
| | | RLREEDREKFRVPIEIADEDYDAEQEPFKNTLVRHQDRFPY |
| | | FALRYFDYNEIFTNLRFQIDLGTYHFSIYKKQIGDYKESHH |
| | | LTHKLYGFERIQEFTKQNRPDEWRKFVKTFNSFETSKEPYI |
| | | PETTPHYHLENQKIGIRFRNDNDKIWPSLKTNSEKNEKSKY |
| | | KLDKSFQAEAFLSVHELLPMMFYYLLLKTENTDNDNEIET |
| | | KKKENKNDKQEKHKIEEIIENKITEIYALYDTFANGEIKSID |
| | | ELEEYCKGKDIEIGHLPKQMIAILKDEHKVMATEAERKQE |
| | | EMLVDVQKSLESLDNQINEEIENVERKNSSLKSGKIASWLV |
| | | NDMMRFQPVQKDNEGKPLNNSKANSTEYQLLQRTLAFFG |
| | | SEHERLAPYFKQTKLIESSNPHPFLKDTEWEKCNNILSFYRS |
| | | YLEAKKNFLESLKPEDWEKNQYFLKLKEPKTKPKTLVQG |
| | | WKNGFNLPRGIFTEPIRKWFMKHRENITVAELKRVGLVAK |
| | | VIPLFFSEEYKDSVQPFYNYHFNVGNINKPDEKNFLNCEER |
| | | RELLRKKKDEFKKMTDKEKEENPSYLEFKSWNKFERELRL |
| | | VRNQDIVTWLLCMELFNKKKIKELNVEKIYLKNINTNTTK |
| | | KEKNTEEKNGEEKNIKEKNNILNRIMPMRLPIKVYGRENFS |
| | | KNKKKKIRRNTFFTVYIEEKGTKLLKQGNFKALERDRRLG |
| | | GLFSFVKTPSKAESKSNTISKLRVEYELGEYQKARIEIIKDM |
| | | LALEKTLIDKYNSLDTDNFNKMLTDWLELKGEPDKASFQ |
| | | NDVDLLIAVRNAFSHNQYPMRNRIAFANINPFSLSSANTSE |
| | | EKGLGIANQLKDKTHKTIEKIIEIEKPIETKE |
| SEQ ID NO: 28 | Prevotella | MQKQDKLFVDRKKNAIFAFPKYITIMENKEKPEPIYYELTD |
| | buccae | KHFWAAFLNLARHNVYTTINHINRRLEIAELKDDGYMMGI |
| | Cas13b | KGSWNEQAKKLDKKVRLRDLIMKHFPFLEAAAYEMTNSK |
| | | SPNNKEQREKEQSEALSLNNLKNVLFIFLEKLQVLRNYYS |
| | | HYKYSEESPKPIFETSLLKNMYKVFDANVRLVKRDYMHH |
| | | ENIDMQRDFTHLNRKKQVGRTKNIIDSPNFHYHFADKEGN |
| | | MTIAGLLFFVSLFLDKKDAIWMQKKLKGFKDGRNLREQM |
| | | TNEVFCRSRISLPKLKLENVQTKDWMQLDMLNELVRCPKS |
| | | LYERLREKDRESFKVPFDIFSDDYNAEEEPFKNTLVRHQDR |
| | | FPYFVLRYFDLNEIFEQLRFQIDLGTYHFSIYNKRIGDEDEV |
| | | RHLTHHLYGFARIQDFAPQNQPEEWRKLVKDLDHFETSQE |
| | | PYISKTAPHYHLENEKIGIKFCSAHNNLFPSLQTDKTCNGR |
| | | SKFNLGTQFTAEAFLSVHELLPMMFYYLLLTKDYSRKESA |
| | | DKVEGIIRKEISNIYAIYDAFANNEINSIADLTRRLQNTNILQ |
| | | GHLPKQMISILKGRQKDMGKEAERKIGEMIDDTQRRLDLL |
| | | CKQTNQKIRIGKRNAGLLKSGKIADWLVNDMMRFQPVQK |
| | | DQNNIPINNSKANSTEYRMLQRALALFGSENFRLKAYFNQ |
| | | MNLVGNDNPHPFLAETQWEHQTNILSFYRNYLEARKKYL |
| | | KGLKPQNWKQYQHFLILKVQKTNRNTLVTGWKNSFNLPR |
| | | GIFTQPIREWFEKHNNSKRIYDQILSFDRVGFVAKAIPLYFA |
| | | EEYKDNVQPFYDYPFNIGNRLKPKKRQFLDKKERVELWQ |
| | | KNKELFKNYPSEKKKTDLAYLDFLSWKKFERELRLIKNQD |
| | | IVTWLMFKELFNMATVEGLKIGEIHLRDIDTNTANEESNNI |
| | | LNRIMPMKLPVKTYETDNKGNILKERPLATFYIEETETKVL |
| | | KQGNFKALVKDRRLNGLFSFAETTDLNLEEHPISKLSVDLE |
| | | LIKYQTTRISIFEMTLGLEKKLIDKYSTLPTDSFRNMLERWL |
| | | QCKANRPELKNYVNSLIAVRNAFSHNQYPMYDATLFAEV |
| | | KKFTLFPSVDTKKIELNIAPQLLEIVGKAIKEIEKSENKN |
| SEQ ID NO: 29 | Porphyromonas | MNTVPASENKGQSRTVEDDPQYFGLYLNLARENLIEVESH |
| | gingivalis | VRIKFGKKKLNEESLKQSLLCDHLLSVDRWTKVYGHSRR |
| | Cas 13b | YLPFLHYFDPDSQIEKDHDSKTGVDPDSAQRLIRELYSLLD |
| | | FLRNDFSHNRLDGTTFEHLEVSPDISSFITGTYSLACGRAQS |
| | | RFAVFFKPDDFVLAKNRKEQLISVADGKECLTVSGFAFFIC |
| | | LFLDREQASGMLSRIRGFKRTDENWARAVHETFCDLCIRH |
| | | PHDRLESSNTKEALLLDMLNELNRCPRILYDMLPEEERAQ |
| | | FLPALDENSMNNLSENSLDEESRLLWDGSSDWAEALTKRI |
| | | RHQDRFPYLMLRFIEEMDLLKGIRFRVDLGEIELDSYSKKV |
| | | GRNGEYDRTITDHALAFGKLSDFQNEEEVSRMISGEASYP |
| | | VRFSLFAPRYAIYDNKIGYCHTSDPVYPKSKTGEKRALSNP |
| | | QSMGFISVHDLRKLLLMELLCEGSFSRMQSDFLRKANRIL |
| | | DETAEGKLQFSALFPEMRHRFIPPQNPKSKDRREKAETTLE |
| | | KYKQEIKGRKDKLNSQLLSAFDMDQRQLPSRLLDEWMNI |
| | | RPASHSVKLRTYVKQLNEDCRLRLRKFRKDGDGKARAIPL |
| | | VGEMATFLSQDIVRMIISEETKKLITSAYYNEMQRSLAQYA |
| | | GEENRRQFRAIVAELRLLDPSSGHPFLSATMETAHRYTEGF |
| | | YKCYLEKKREWLAKIFYRPEQDENTKRRISVFFVPDGEAR |
| | | KLLPLLIRRRMKEQNDLQDWIRNKQAHPIDLPSHLFDSKV |
| | | MELLKVKDGKKKWNEAFKDWWSTKYPDGMQPFYGLRR |
| | | ELNIHGKSVSYIPSDGKKFADCYTHLMEKTVRDKKRELRT |
| | | AGKPVPPDLAADIKRSFHRAVNEREFMLRLVQEDDRLML |
| | | MAINKMMTDREEDILPGLKNIDSILDEENQFSLAVHAKVL |
| | | EKEGEGGDNSLSLVPATIEIKSKRKDWSKYIRYRYDRRVP |
| | | GLMSHFPEHKATLDEVKTLLGEYDRCRIKIFDWAFALEGA |
| | | IMSDRDLKPYLHESSSREGKSGEHSTLVKMLVEKKGCLTP |
| | | DESQYLILIRNKAAHNQFPCAAEMPLIYRDVSAKVGSIEGS |
| | | SAKDLPEGSSLVDSLWKKYEMIIRKILPILDPENRFFGKLLN |
| | | NMSQPINDL |
| SEQ ID NO: 30 | Bacteroides | MESIKNSQKSTGKTLQKDPPYFGLYLNMALLNVRKVENHI |
| | pyogenes | RKWLGDVALLPEKSGFHSLLTTDNLSSAKWTRFYYKSRKF |
| | Cas13b | LPFLEMFDSDKKSYENRRETAECLDTIDRQKISSLLKEVYG |
| | | KLQDIRNAFSHYHIDDQSVKHTALIISSEMHRFIENAYSFAL |
| | | QKTRARFTGVFVETDFLQAEEKGDNKKFFAIGGNEGIKLK |
| | | DNALIFLICLFLDREEAFKFLSRATGFKSTKEKGFLAVRETF |
| | | CALCCRQPHERLLSVNPREALLMDMLNELNRCPDILFEML |
| | | DEKDQKSFLPLLGEEEQAHILENSLNDELCEAIDDPFEMIAS |
| | | LSKRVRYKNRFPYLMLRYIEEKNLLPFIRFRIDLGCLELASY |
| | | PKKMGEENNYERSVTDHAMAFGRLTDFHNEDAVLQQITK |
| | | GITDEVRFSLYAPRYAIYNNKIGFVRTSGSDKISFPTLKKKG |
| | | GEGHCVAYTLQNTKSFGFISIYDLRKILLLSFLDKDKAKNI |
| | | VSGLLEQCEKHWKDLSENLFDAIRTELQKEFPVPLIRYTLP |
| | | RSKGGKLVSSKLADKQEKYESEFERRKEKLTEILSEKDFDL |
| | | SQIPRRMIDEWLNVLPTSREKKLKGYVETLKLDCRERLRV |
| | | FEKREKGEHPLPPRIGEMATDLAKDIIRMVIDQGVKQRITS |
| | | AYYSEIQRCLAQYAGDDNRRHLDSIIRELRLKDTKNGHPFL |
| | | GKVLRPGLGHTEKLYQRYFEEKKEWLEATFYPAASPKRVP |
| | | RFVNPPTGKQKELPLIIRNLMKERPEWRDWKQRKNSHPID |
| | | LPSQLFENEICRLLKDKIGKEPSGKLKWNEMFKLYWDKEF |
| | | PNGMQRFYRCKRRVEVFDKVVEYEYSEEGGNYKKYYEA |
| | | LIDEVVRQKISSSKEKSKLQVEDLTLSVRRVFKRAINEKEY |
| | | QLRLLCEDDRLLFMAVRDLYDWKEAQLDLDKIDNMLGEP |
| | | VSVSQVIQLEGGQPDAVIKAECKLKDVSKLMRYCYDGRV |
| | | KGLMPYFANHEATQEQVEMELRHYEDHRRRVFNWVFAL |
| | | EKSVLKNEKLRRFYEESQGGCEHRRCIDALRKASLVSEEE |
| | | YEFLVHIRNKSAHNQFPDLEIGKLPPNVTSGFCECIWSKYK |
| | | AIICRIIPFIDPERRFFGKLLEQK |
| SEQ ID NO: 31 | Cas13c | MTEKKSIIFKNKSSVEIVKKDIFSQTPDNMIRNYKITLKISEK |
| | | NPRVVEAEIEDLMNSTILKDGRRSARREKSMTERKLIEEKV |
| | | AENYSLLANCPMEEVDSIKIYKIKRFLTYRSNMLLYFASIN |
| | | SFLCEGIKGKDNETEEIWHLKDNDVRKEKVKENFKNKLIQ |
| | | STENYNSSLKNQIEEKEKLLRKESKKGAFYRTIIKKLQQERI |
| | | KELSEKSLTEDCEKIIKLYSELRHPLMHYDYQYFENLFENK |
| | | ENSELTKNLNLDIFKSLPLVRKMKLNNKVNYLEDNDTLFV |
| | | LQKTKKAKTLYQIYDALCEQKNGFNKFINDFFVSDGEENT |
| | | VFKQIINEKFQSEMEFLEKRISESEKKNEKLKKKFDSMKAH |
| | | FHNINSEDTKEAYFWDIHSSSNYKTKYNERKNLVNEYTEL |
| | | LGSSKEKKLLREEITQINRKLLKLKQEMEEITKKNSLFRLE |
| | | YKMKIAFGFLFCEFDGNISKFKDEFDASNQEKIIQYHKNGE |
| | | KYLTYFLKEEEKEKFNLEKMQKIIQKTEEEDWLLPETKNN |
| | | LFKFYLLTYLLLPYELKGDFLGFVKKHYYDIKNVDFMDEN |
| | | QNNIQVSQTVEKQEDYFYHKIRLFEKNTKKYEIVKYSIVPN |
| | | EKLKQYFEDLGIDIKYLTGSVESGEKWLGENLGIDIKYLTV |
| | | EQKSEVSEEKIKKFL |
| SEQ ID NO: 32 | Cas13c | MEKDKKGEKIDISQEMIEEDLRKILILFSRLRHSMVHYDYE |
| | | FYQALYSGKDFVISDKNNLENRMISQLLDLNIFKELSKVKL |
| | | IKDKAISNYLDKNTTIHVLGQDIKAIRLLDIYRDICGSKNGF |
| | | NKFINTMITISGEEDREYKEKVIEHFNKKMENLSTYLEKLE |
| | | KQDNAKRNNKRVYNLLKQKLIEQQKLKEWFGGPYVYDIH |
| | | SSKRYKELYIERKKLVDRHSKLFEEGLDEKNKKELTKINDE |
| | | LSKLNSEMKEMTKLNSKYRLQYKLQLAFGFILEEFDLNIDT |
| | | FINNFDKDKDLIISNFMKKRDIYLNRVLDRGDNRLKNIIKE |
| | | YKFRDTEDIFCNDRDNNLVKLYILMYILLPVEIRGDFLGFV |
| | | KKNYYDMKHVDFIDKKDKEDKDTFFHDLRLFEKNIRKLEI |
| | | TDYSLSSGFLSKEHKVDIEKKINDFINRNGAMKLPEDITIEE |
| | | FNKSLILPIMKNYQINFKLLNDIEISALFKIAKDRSITFKQAI |
| | | DEIKNEDIKKNSKKNDKNNHKDKNINFTQLMKRALHEKIP |
| | | YKAGMYQIRNNISHIDMEQLYIDPLNSYMNSNKNNITISEQ |
| | | IEKIIDVCVTGGVTGKELNNNIINDYYMKKEKLVFNLKLRK |
| | | QNDIVSIESQEKNKREEFVFKKYGLDYKDGEINIIEVIQKVN |
| | | SLQEELRNIKETSKEKLKNKETLFRDISLINGTIRKNINFKIK |
| | | EMVLDIVRMDEIRHINIHIYYKGENYTRSNIIKFKYAIDGEN |
| | | KKYYLKQHEINDINLELKDKFVTLICNMDKHPNKNKQTIN |
| | | LESNYIQNVKFIIP |
| SEQ ID NO: 33 | Cas13c | MENKGNNKKIDFDENYNILVAQIKEYFTKEIENYNNRIDNI |
| | | IDKKELLKYSEKKEESEKNKKLEELNKLKSQKLKILTDEEI |
| | | KADVIKIIKIFSDLRHSLMHYEYKYFENLFENKKNEELAEL |
| | | LNLNLFKNLTLLRQMKIENKTNYLEGREEFNIIGKNIKAKE |
| | | VLGHYNLLAEQKNGFNNFINSFFVQDGTENLEFKKLIDEHF |
| | | VNAKKRLERNIKKSKKLEKELEKMEQHYQRLNCAYVWDI |
| | | HTSTTYKKLYNKRKSLIEEYNKQINEIKDKEVITAINVELLR |
| | | IKKEMEEITKSNSLFRLKYKMQIAYAFLEIEFGGNIAKFKDE |
| | | FDCSKMEEVQKYLKKGVKYLKYYKDKEAQKNYEFPFEEI |
| | | FENKDTHNEEWLENTSENNLFKFYILTYLLLPMEFKGDFL |
| | | GVVKKHYYDIKNVDFTDESEKELSQVQLDKMIGDSFFHKI |
| | | RLFEKNTKRYEIIKYSILTSDEIKRYFRLLELDVPYFEYEKG |
| | | TDEIGIFNKNIILTIFKYYQIIFRLYNDLEIHGLFNISSDLDKIL |
| | | RDLKSYGNKNINFREFLYVIKQNNNSSTEEEYRKIWENLEA |
| | | KYLRLHLLTPEKEEIKTKTKEELEKLNEISNLRNGICHLNY |
| | | KEIIEEILKTEISEKNKEATLNEKIRKVINFIKENELDKVELG |
| | | FNFINDFFMKKEQFMFGQIKQVKEGNSDSITTERERKEKNN |
| | | KKLKETYELNCDNLSEFYETSNNLRERANSSSLLEDSAFLK |
| | | KIGLYKVKNNKVNSKVKDEEKRIENIKRKLLKDSSDIMGM |
| | | YKAEVVKKLKEKLILIFKHDEEKRIYVTVYDTSKAVPENIS |
| | | KEILVKRNNSKEEYFFEDNNKKYVTEYYTLEITETNELKVI |
| | | PAKKLEGKEFKTEKNKENKLMLNNHYCFNVKIIY |
| SEQ ID NO: 34 | Cas13c | MEEIKHKKNKSSIIRVIVSNYDMTGIKEIKVLYQKQGGVDT |
| | | FNLKTIINLESGNLEIISCKPKEREKYRYEFNCKTEINTISITK |
| | | KDKVLKKEIRKYSLELYFKNEKKDTVVAKVTDLLKAPDKI |
| | | EGERNHLRKLSSSTERKLLSKTLCKNYSEISKTPIEEIDSIKI |
| | | YKIKRFLNYRSNFLIYFALINDFLCAGVKEDDINEVWLIQD |
| | | KEHTAFLENRIEKITDYIFDKLSKDIENKKNQFEKRIKKYKT |
| | | SLEELKTETLEKNKTFYIDSIKTKITNLENKITELSLYNSKES |
| | | LKEDLIKIISIFTNLRHSLMHYDYKSFENLFENIENEELKNLL |
| | | DLNLFKSIRMSDEFKTKNRTNYLDGTESFTIVKKHQNLKK |
| | | LYTYYNNLCDKKNGFNTFINSFFVTDGIENTDFKNLIILHFE |
| | | KEMEEYKKSIEYYKIKISNEKNKSKKEKLKEKIDLLQSELIN |
| | | MREHKNLLKQIYFFDIHNSIKYKELYSERKNLIEQYNLQIN |
| | | GVKDVTAINHINTKLLSLKNKMDKITKQNSLYRLKYKLKI |
| | | AYSFLMIEFDGDVSKFKNNFDPTNLEKRVEYLDKKEEYLN |
| | | YTAPKNKFNFAKLEEELQKIQSTSEMGADYLNVSPENNLF |
| | | KFYILTYIMLPVEFKGDFLGFVKNHYYNIKNVDFMDESLL |
| | | DENEVDSNKLNEKIENLKDSSFFNKIRLFEKNIKKYEIVKYS |
| | | VSTQENMKEYFKQLNLDIPYLDYKSTDEIGIFNKNMILPIFK |
| | | YYQNVFKLCNDIEIHALLALANKKQQNLEYAIYCCSKKNS |
| | | LNYNELLKTFNRKTYQNLSFIRNKIAHLNYKELFSDLFNNE |
| | | LDLNTKVRCLIEFSQNNKFDQIDLGMNFINDYYMKKTRFIF |
| | | NQRRLRDLNVPSKEKIIDGKRKQQNDSNNELLKKYGLSRT |
| | | NIKDIFNKAWY |
| SEQ ID NO: 35 | Cas 13c | MKVRYRKQAQLDTFIIKTEIVNNDIFIKSIIEKAREKYRYSF |
| | | LFDGEEKYHFKNKSSVEIVKNDIFSQTPDNMIRNYKITLKIS |
| | | EKNPRVVEAEIEDLMNSTILKDGRRSARREKSMTERKLIEE |
| | | KVAENYSLLANCPIEEVDSIKIYKIKRFLTYRSNMLLYFASI |
| | | NSFLCEGIKGKDNETEEIWHLKDNDVRKEKVKENFKNKLI |
| | | QSTENYNSSLKNQIEEKEKLSSKEFKKGAFYRTIIKKLQQE |
| | | RIKELSEKSLTEDCEKIIKLYSELRHPLMHYDYQYFENLFE |
| | | NKENSELTKNLNLDIFKSLPLVRKMKLNNKVNYLEDNDTL |
| | | FVLQKTKKAKTLYQIYDALCEQKNGFNKFINDFFVSDGEE |
| | | NTVFKQIINEKFQSEMEFLEKRISESEKKNEKLKKKLDSMK |
| | | AHFRNINSEDTKEAYFWDIHSSRNYKTKYNERKNLVNEYT |
| | | KLLGSSKEKKLLREEITKINRQLLKLKQEMEEITKKNSLFR |
| | | LEYKMKIAFGFLFCEFDGNISKFKDEFDASNQEKIIQYHKN |
| | | GEKYLTSFLKEEEKEKFNLEKMQKIIQKTEEEDWLLPETKN |
| | | NLFKFYLLTYLLLPYELKGDFLGFVKKHYYDIKNVDFMDE |
| | | NQNNIQVSQTVEKQEDYFYHKIRLFEKNTKKYEIVKYSIVP |
| | | NEKLKQYFEDLGIDIKYLTGSVESGEKWLGENLGIDIKYLT |
| | | VEQKSEVSEEKNKKVSLKNNGMFNKTILLFVFKYYQIAFK |
| | | LFNDIELYSLFFLREKSEKPFEVFLEELKDKMIGKQLNFGQ |
| | | LLYVVYEVLVKNKDLDKILSKKIDYRKDKSFSPEIAYLRNF |
| | | LSHLNYSKFLDNFMKINTNKSDENKEVLIPSIKIQKMIQFIE |
| | | KCNLQNQIDFDFNFVNDFYMRKEKMFFIQLKQIFPDINSTE |
| | | KQKKSEKEEILRKRYHLINKKNEQIKDEHEAQSQLYEKILS |
| | | LQKIFSCDKNNFYRRLKEEKLLFLEKQGKKKISMKEIKDKI |
| | | ASDISDLLGILKKEITRDIKDKLTEKFRYCEEKLLNISFYNH |
| | | QDKKKEEGIRVFLIRDKNSDNFKFESILDDGSNKIFISKNGK |
| | | EITIQCCDKVLETLMIEKNTLKISSNGKIISLIPHYSYSIDVK |
| | | Y |

The DNA-activated programmable RNA nuclease can be Cas13. Sometimes the Cas13 can be Cas13a, Cas13b, Cas13c, Cas13d, or Cas13e. Sometimes Cas13a can also be also called C2c2. In some cases, the DNA-activated programmable RNA nuclease can be a type VI CRISPR-Cas system. In some cases, the DNA-activated programmable RNA nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the CRISPR enzyme can be activated when the crRNA is complexed with the target nucleic acid. The trans cleavage activity of the CRISPR enzyme can be activated when the engineered guide nucleic acid comprising a tracrRNA and crRNA are complexed with the target nucleic acid. The target nucleic acid can be RNA or DNA.

The detection of the target nucleic acid is facilitated by a programmable nuclease. The programmable nuclease can become activated after binding of an engineered guide nucleic acid to a target nucleic, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety is released from the detector nucleic acid and generates a detectable signal. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or level of the target nucleic acid. A signal can be a calorimetric, potentiometric, amperometric, optical (e.g., fluorescent, colorometric, etc.), or piezo-electric signal. Often, the signal is present prior to detector nucleic acid cleavage and changes upon detector nucleic acid cleavage. Sometimes, the signal is absent prior to detector nucleic acid cleavage and is present upon detector nucleic acid cleavage. The detectable signal can be immobilized on a support medium for detection. The programmable nuclease can be a DNA-activated programmable RNA nuclease. The programmable nuclease can be a Type VI CRISPR enzyme that detects a target deoxyribonucleic acid. The programmable nuclease can be a Cas13 (e.g., Cas13a) tha detects a target deoxyribonucleic acid. The programmable nuclease can be a CRISPR-Cas (clustered regularly interspaced short palindromic repeats—CRISPR associated) nucleoprotein complex with trans cleavage activity, which can be activated by binding of an engineered guide nucleic acid with a target nucleic acid. The CRISPR-Cas nucleoprotein complex can comprise a Cas protein (also referred to as a Cas nuclease) complexed with an engineered guide nucleic acid, which can also be referred to as CRISPR enzyme. An engineered guide nucleic acid can be a CRISPR RNA (crRNA). Sometimes, an engineered guide nucleic acid comprises a crRNA and a trans-activating crRNA (tracrRNA). The CRISPR/Cas system used to detect a modified target nucleic acids can comprise CRISPR RNAs (crRNAs), trans-activating crRNAs (tracrRNAs), Cas proteins, and detector nucleic acids.

The programmable nucleases described herein are capable of being activated when complexed with the engineered guide nucleic acid and the target nucleic acid (e.g., DNA). A programmable nuclease can be capable of being activated when complexed with an engineered guide nucleic acid and the target deoxyribonucleotide. The programmable nuclease can be activated upon binding of the engineered guide nucleic acid to its target nucleic acid and degrades non-specifically nucleic acid in its environment. In some embodiments, an activated DNA-activated programmable RNA nuclease non-specifically degrades RNA in its environment (e.g., exhibits trans-collateral cleavage of RNA, such as RNA reporters). A DNA-activated programmable RNA nuclease can be a Cas protein (also referred to, interchangeably, as a Cas nuclease). A crRNA and Cas protein can form a CRISPR enzyme. In some embodiments, the DNA-activated programmable RNA nuclease is a Type VI CRISPR enzyme. In some embodiments, the DNA-activated programmable RNA nuclease is Cas13. Sometimes the Cas13 is Cas13a, Cas13b, Cas13c, Cas13d, or Cas13e. In some cases, the DNA-activated programmable RNA nuclease is from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the DNA-activated programmable RNA nuclease is at least one of LbuCas13a, LwaCas13a, LbaCas13a, HheCas13a, PprCas13a, EreCas13a, CamCas13a, or LshCas13a.

In some embodiments, a programmable nuclease is capable of being activated by a target RNA to initiate trans cleavage of an RNA reporter and is capable of being activated by a target DNA to initiate trans cleavage of an RNA reporter, such as a Type VI CRISPR protein (e.g., Cas13). For example, Cas13a of the present disclosure can be activated by a target RNA to initiate trans cleavage activity of the Cas13a for the cleavage of an RNA reporter and can be activated by a target DNA to initiate trans cleavage activity of the Cas13a for trans cleavage of an RNA reporter.

The trans cleavage activity of the DNA-activated programmable RNA nuclease can be activated when the crRNA is complexed with the target dexoyribonucleic acid. The trans cleavage activity of the DNA-activated programmable RNA nuclease can be activated when the engineered guide nucleic acid comprising a tracrRNA and crRNA are complexed with the target deoxyribonucleic acid. The target dexoyribonucleic acid can be a DNA or reverse transcribed RNA, or an amplicon thereof. Preferably, the target deoxyribonucleic acid is single-stranded DNA. Thus, a Cas13a nuclease of the present disclosure can be activated by a target DNA to initiate trans cleavage activity of the Cas13a nuclease that cleaves an RNA reporter. For example, Cas13a nucleases disclosed herein are activated by the binding of the engineered guide nucleic acid to a target DNA that was reverse transcribed from an RNA to transcollaterally cleave reporter molecules. For example, Cas13a nucleases disclosed herein are activated by the binding of the engineered guide nucleic acid to a target DNA that was amplified from a DNA to transcollaterally cleave reporter molecules. The reporter molecules can be RNA reporter molecules. In some embodiments, the Cas13a recognizes and detects ssDNA and, further, trans-cleaves RNA reporters. Multiple Cas13a isolates can recognize, be activated by, and detect target DNA as described herein, including ssDNA. For example, trans-collateral cleavage of RNA reporters can be activated in Lbu-Cas13a or Lwa-Cas13a by target DNA. Therefore, a DNA-activated programmable RNA nuclease can be used to detect target DNA by assaying for cleaved RNA reporters.

In some embodiments, the programmable nuclease may be present in the cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the programmable nuclease may be present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the programmable nuclease may be present in the cleavage reaction at a concentration of from 20 nM to 50 μM, from 50 nM to 20 μM, or from 200 nM to 5 μM.

A DNA-activated programmable RNA nuclease can be used to detect DNA at multiple pH values. A DNA-activated programmable RNA nuclease can be used to detect DNA at multiple pH values compared to an RNA-activated programmable RNA nuclease, such as a Cas13a complexed with a guide RNA that detects a target ribonucleic acid. For example, a Cas13 protein that detects a target RNA may exhibit high cleavage activity at pH values from 7.9 to 8.2. A Cas13 protein that detects a target DNA can exhibit consistent cleavage across a wide range of pH conditions, such as from a pH of 6.8 to a pH of 8.2. In some embodiments, Cas13 ssDNA detection may exhibit high cleavage activity at pH values from 6 to 6.5, from 6.1 to 6.6, from 6.2 to 6.7, from 6.3 to 6.8, from 6.4 to 6.9, from 6.5 to 7, from 6.6 to 7.1, from 6.7 to 7.2, from 6.8 to 7.3, from 6.9 to 7.4, from 7 to 7.5, from 7.1 to 7.6, from 7.2 to 7.7, from 7.3 to 7.8, from 7.4 to 7.9, from 7.5 to 8, from 7.6 to 8.1, from 7.7 to 8.2, from 7.8 to 8.3, from 7.9 to 8.4, from 8 to 8.5, from 8.1 to 8.6, from 8.2 to 8.7, from 8.3 to 8.8, from 8.4 to 8.9, from 8.5 to 9, from 6 to 8, from 6.5 to 8, or from 7 to 8.

In some embodiments, a programmable nuclease is capable of being activated by a target RNA to initiate trans cleavage of an RNA reporter and is capable of being activated by a target DNA to initiate trans cleavage of an RNA reporter, such as a Type VI CRISPR protein (e.g., Cas13). For example, Cas13a of the present disclosure can be activated by a target RNA to initiate trans cleavage activity of the Cas13a for the cleavage of an RNA reporter and can be activated by a target DNA to initiate trans cleavage activity of the Cas13a for trans cleavage of an RNA reporter. In some embodiments, target DNA binding preferences of a DNA-activated programmable RNA nuclease can be distinct from target RNA binding preferences of a RNA-activated programmable RNA nuclease. In some embodiments, target DNA binding preferences of an engineered guide nucleic acid complexed with a DNA-activated programmable RNA nuclease can be distinct from target RNA binding preferences of an engineered guide nucleic acid complexed with a RNA-activated programmable RNA nuclease. For example, guide RNA (gRNA) binding to a target DNA, and preferably a target ssDNA, may not necessarily correlate with the binding of the same gRNAs binding to a target RNA. For example, gRNAs can perform at a high level regardless of target nucleotide identity at a 3′ position in a sequence of a target RNA. In some embodiments, gRNAs can perform at a high level in the absence of a G at a 3′ position in a sequence of a target DNA. Furthermore, target DNA detected by a DNA-activated programmable RNA nuclease complexed with an engineered guide nucleic acid as disclosed herein can be directly from organisms, or can be indirectly generated by nucleic acid amplification methods, such as PCR and LAMP of DNA or reverse transcription of RNA. Key steps for the sensitive detection of direct DNA by a DNA-activated programmable RNA nuclease, such as a Cas13a, can include: (1) production or isolation of DNA to concentrations above about 0.1 nM per reaction for in vitro diagnostics, (2) selection of a target DNA with the appropriate sequence features to enable DNA detection as these some of these features are distinct from those required for target RNA detection, and (3) buffer composition that enhances DNA detection. The detection of DNA by a DNA-activated programmable RNA nuclease can be connected to a variety of readouts including fluorescence, lateral flow, electrochemistry, or any other readouts described herein. Multiplexing of a DNA-activated programmable RNA nuclease with a DNA-activated programmable DNA nuclease with RNA and DNA FQ-reporter molecules (each with a different color fluorophore), respectively, can enable detection of ssDNA or a combination of ssDNA and dsDNA, respectively. Multiplexing of different DNA-activated programmable RNA nuclease that have distinct RNA reporter cleavage preferences can enable additional multiplexing, such a first DNA-activated programmable RNA nuclease that preferentially cleaves uracil in an RNA reporter and a second DNA-activated programmable RNA nuclease that preferentially cleaves adenines in an RNA reporter. Methods for the generation of ssDNA for a DNA-activated programmable RNA nuclease-based detection or diagnostics can include (1) asymmetric PCR, (2) asymmetric isothermal amplification, such as RPA, LAMP, SDA, etc. (3) NEAR for the production of short ssDNA molecules, and (4) conversion of RNA targets into ssDNA by a reverse transcriptase followed by RNase H digestion. Thus, a DNA-activated programmable RNA nuclease detection of target DNA is compatible with the various systems, kits, compositions, reagents, and methods disclosed herein.

Cas13a DNA detection can be employed in a DETECTR assay disclosed herein to provide CRISPR diagnostics leveraging Type VI systems (e.g., Cas13) for the detection of a target DNA.

In some embodiments, the Type V CRISPR/Cas enzyme is a programmable Cas12 nuclease. Type V CRISPR/Cas enzymes (e.g., Cas12 or Cas14) lack an HNH domain. A Cas12 nuclease of the present disclosure cleaves a nucleic acids via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. (Murugan et al., Mol Cell. 2017 Oct. 5; 68(1): 15-25). A programmable Cas12 nuclease can be a Cas12a (also referred to as Cpf1) protein, a Cas12b protein, Cas12c protein, Cas12d protein, or a Cas12e protein. In some cases, a suitable Cas12 protein comprises an amino acid sequence having at least 80%, at least 85%, at least 90%, at least 95%, at least 98%, at least 99%, or 100%, amino acid sequence identity to any one of SEQ ID NO: 36-SEQ ID NO: 46.

TABLE 2
Cas12 Protein Sequences

| SEQ | | |
|---|---|---|
| ID NO | Description | Sequence |
| SEQ | Lachnospiraceae | MSKLEKFTNCYSLSKTLRFKAIPVGKTQENIDNKRLLVEDEKRAEDYK |
| ID | bacterium | GVKKLLDRYYLSFINDVLHSIKLKNLNNYISLFRKKTRTEKENKELENL |
| NO: | ND2006 | EINLRKEIAKAFKGNEGYKSLFKKDIIETILPEFLDDKDEIALVNSFNGFT |
| 36 | (LbCas12a) | TAFTGFFDNRENMFSEEAKSTSIAFRCINENLTRYISNMDIFEKVDAIFD |
| | | KHEVQEIKEKILNSDYDVEDFFEGEFFNFVLTQEGIDVYNAIIGGFVTES |
| | | GEKIKGLNEYINLYNQKTKQKLPKFKPLYKQVLSDRESLSFYGEGYTSD |
| | | EEVLEVFRNTLNKNSEIFSSIKKLEKLFKNFDEYSSAGIFVKNGPAISTIS |
| | | KDIFGEWNVIRDKWNAEYDDIHLKKKAVVTEKYEDDRRKSFKKIGSFS |
| | | LEQLQEYADADLSVVEKLKEIIIQKVDEIYKVYGSSEKLFDADFVLEKSL |
| | | KKNDAVVAIMKDLLDSVKSFENYIKAFFGEGKETNRDESFYGDFVLAY |
| | | DILLKVDHIYDAIRNYVTQKPYSKDKFKLYFQNPQFMGGWDKDKETD |
| | | YRATILRYGSKYYLAIMDKKYAKCLQKIDKDDVNGNYEKINYKLLPGP |
| | | NKMLPKVFFSKKWMAYYNPSEDIQKIYKNGTFKKGDMFNLNDCHKLI |
| | | DFFKDSISRYPKWSNAYDFNFSETEKYKDIAGFYREVEEQGYKVSFESA |
| | | SKKEVDKLVEEGKLYMFQIYNKDFSDKSHGTPNLHTMYFKLLFDENN |
| | | HGQIRLSGGAELFMRRASLKKEELVVHPANSPIANKNPDNPKKTTTLSY |
| | | DVYKDKRFSEDQYELHIPIAINKCPKNIFKINTEVRVLLKHDDNPYVIGI |
| | | DRGERNLLYIVVVDGKGNIVEQYSLNEIINNFNGIRIKTDYHSLLDKKEK |
| | | ERFEARQNWTSIENIKELKAGYISQVVHKICELVEKYDAVIALEDLNSG |
| | | FKNSRVKVEKQVYQKFEKMLIDKLNYMVDKKSNPCATGGALKGYQIT |
| | | NKFESFKSMSTQNGFIFYIPAWLTSKIDPSTGFVNLLKTKYTSIADSKKFI |
| | | SSFDRIMYVPEEDLFEFALDYKNFSRTDADYIKKWKLYSYGNRIRIFRN |
| | | PKKNNVFDWEEVCLTSAYKELFNKYGINYQQGDIRALLCEQSDKAFYS |
| | | SFMALMSLMLQMRNSITGRTDVDFLISPVKNSDGIFYDSRNYEAQENAI |
| | | LPKNADANGAYNIARKVLWAIGQFKKAEDEKLDKVKIAISNKEWLEY |
| | | AQTSVKH |
| SEQ | Acidaminococcus sp. | MTQFEGFTNLYQVSKTLRFELIPQGKTLKHIQEQGFIEEDKARNDHYKE |
| ID | BV316 | LKPIIDRIYKTYADQCLQLVQLDWENLSAAIDSYRKEKTEETRNALIEEQ |
| NO: | (AsCas12a) | ATYRNAIHDYFIGRTDNLTDAINKRHAEIYKGLFKAELFNGKVLKQLGT |
| 37 | | VTTTEHENALLRSFDKFTTYFSGFYENRKNVFSAEDISTAIPHRIVQDNF |
| | | PKFKENCHIFTRLITAVPSLREHFENVKKAIGIFVSTSIEEVFSFPFYNQLL |
| | | TQTQIDLYNQLLGGISREAGTEKIKGLNEVLNLAIQKNDETAHIIASLPH |
| | | RFIPLFKQILSDRNTLSFILEEFKSDEEVIQSFCKYKTLLRNENVLETAEA |
| | | LFNELNSIDLTHIFISHKKLETISSALCDHWDTLRNALYERRISELTGKIT |
| | | KSAKEKVQRSLKHEDINLQEIISAAGKELSEAFKQKTSEILSHAHAALDQ |
| | | PLPTTLKKQEEKEILKSQLDSLLGLYHLLDWFAVDESNEVDPEFSARLT |
| | | GIKLEMEPSLSFYNKARNYATKKPYSVEKFKLNFQMPTLASGWDVNKE |
| | | KNNGAILFVKNGLYYLGIMPKQKGRYKALSFEPTEKTSEGFDKMYYDY |
| | | FPDAAKMIPKCSTQLKAVTAHFQTHTTPILLSNNFIEPLEITKEIYDLNNP |
| | | EKEPKKFQTAYAKKTGDQKGYREALCKWIDFTRDFLSKYTKTTSIDLSS |
| | | LRPSSQYKDLGEYYAELNPLLYHISFQRIAEKEIMDAVETGKLYLFQIYN |
| | | KDFAKGHHGKPNLHTLYWTGLFSPENLAKTSIKLNGQAELFYRPKSRM |
| | | KRMAHRLGEKMLNKKLKDQKTPIPDTLYQELYDYVNHRLSHDLSDEA |
| | | RALLPNVITKEVSHEIIKDRRFTSDKFFFHVPITLNYQAANSPSKFNQRV |
| | | NAYLKEHPETPIIGIDRGERNLIYITVIDSTGKILEQRSLNTIQQFDYQKKL |
| | | DNREKERVAARQAWSVVGTIKDLKQGYLSQVIHEIVDLMIHYQAVVV |
| | | LENLNFGFKSKRTGIAEKAVYQQFEKMLIDKLNCLVLKDYPAEKVGGV |
| | | LNPYQLTDQFTSFAKMGTQSGFLFYVPAPYTSKIDPLTGFVDPFVWKTI |
| | | KNHESRKHFLEGFDFLHYDVKTGDFILHFKMNRNLSFQRGLPGFMPAW |
| | | DIVFEKNETQFDAKGTPFIAGKRIVPVIENHRFTGRYRDLYPANELIALL |
| | | EEKGIVFRDGSNILPKLLENDDSHAIDTMVALIRSVLQMRNSNAATGED |
| | | YINSPVRDLNGVCFDSRFQNPEWPMDADANGAYHIALKGQLLLNHLK |
| | | ESKDLKLQNGISNQDWLAYIQELRN |
| SEQ | Francisella | MSIYQEFVNKYSLSKTLRFELIPQGKTLENIKARGLILDDEKRAKDYKK |
| ID | novicida | AKQIIDKYHQFFIEEILSSVCISEDLLQNYSDVYFKLKKSDDDNLQKDFK |
| NO: | U112 | SAKDTIKKQISEYIKDSEKFKNLFNQNLIDAKKGQESDLILWLKQSKDN |
| 38 | (FnCas12a) | GIELFKANSDITDIDEALEIIKSFKGWTTYFKGFHENRKNVYSSNDIPTSII |
| | | YRIVDDNLPKFLENKAKYESLKDKAPEAINYEQIKKDLAEELTFDIDYK |
| | | TSEVNQRVFSLDEVFEIANFNNYLNQSGITKFNTIIGGKFVNGENTKRKG |
| | | INEYINLYSQQINDKTLKKYKMSVLFKQILSDTESKSFVIDKLEDDSDVV |
| | | TTMQSFYEQIAAFKTVEEKSIKETLSLLFDDLKAQKLDLSKIYFKNDKSL |
| | | TDLSQQVFDDYSVIGTAVLEYITQQIAPKNLDNPSKKEQELIAKKTEKA |
| | | KYLSLETIKLALEEFNKHRDIDKQCRFEEILANFAAIPMIFDEIAQNKDN |
| | | LAQISIKYQNQGKKDLLQASAEDDVKAIKDLLDQTNNLLHKLKIFHISQ |
| | | SEDKANILDKDEHFYLVFEECYFELANIVPLYNKIRNYITQKPYSDEKFK |
| | | LNFENSTLANGWDKNKEPDNTAILFIKDDKYYLGVMNKKNNKIFDDK |
| | | AIKENKGEGYKKIVYKLLPGANKMLPKVFFSAKSIKFYNPSEDILRIRNH |
| | | STHTKNGSPQKGYEKFEFNIEDCRKFIDFYKQSISKHPEWKDFGFRFSDT |
| | | QRYNSIDEFYREVENQGYKLTFENISESYIDSVVNQGKLYLFQIYNKDFS |
| | | AYSKGRPNLHTLYWKALFDERNLQDVVYKLNGEAELFYRKQSIPKKIT |
| | | HPAKEAIANKNKDNPKKESVFEYDLIKDKRFTEDKFFFHCPITINFKSSG |
| | | ANKFNDEINLLLKEKANDVHILSIDRGERHLAYYTLVDGKGNIIKQDTF |
| | | NIIGNDRMKTNYHDKLAAIEKDRDSARKDWKKINNIKEMKEGYLSQV |
| | | VHEIAKLVIEYNAIVVFEDLNFGFKRGRFKVEKQVYQKLEKMLIEKLNY |
| | | LVFKDNEFDKTGGVLRAYQLTAPFETFKKMGKQTGIIYYVPAGFTSKIC |
| | | PVTGFVNQLYPKYESVSKSQEFFSKFDKICYNLDKGYFEFSFDYKNFGD |
| | | KAAKGKWTIASFGSRLINFRNSDKNHNWDTREVYPTKELEKLLKDYSI |
| | | EYGHGECIKAAICGESDKKFFAKLTSVLNTILQMRNSKTGTELDYLISPV |
| | | ADVNGNFFDSRQAPKNMPQDADANGAYHIGLKGLMLLGRIKNNQEGK |
| | | KLNLVIKNEEYFEFVQNRNN |
| SEQ | Porphyromonas | MKTQHFFEDFTSLYSLSKTIRFELKPIGKTLENIKKNGLIRRDEQRLDDY |
| ID | macacae | EKLKKVIDEYHEDFIANILSSFSFSEEILQSYIQNLSESEARAKIEKTMRD |
| NO: | (PmCas12a) | TLAKAFSEDERYKSIFKKELVKKDIPVWCPAYKSLCKKFDNFTTSLVPF |
| 39 | | HENRKNLYTSNEITASIPYRIVHVNLPKFIQNIEALCELQKKMGADLYLE |
| | | MMENLRNVWPSFVKTPDDLCNLKTYNHLMVQSSISEYNRFVGGYSTE |
| | | DGTKHQGINEWINIYRQRNKEMRLPGLVFLHKQILAKVDSSSFISDTLE |
| | | NDDQVFCVLRQFRKLFWNTVSSKEDDAASLKDLFCGLSGYDPEAIYVS |
| | | DAHLATISKNIFDRWNYISDAIRRKTEVLMPRKKESVERYAEKISKQIKK |
| | | RQSYSLAELDDLLAHYSEESLPAGFSLLSYFTSLGGQKYLVSDGEVILY |
| | | EEGSNIWDEVLIAFRDLQVILDKDFTEKKLGKDEEAVSVIKKALDSALR |
| | | LRKFFDLLSGTGAEIRRDSSFYALYTDRMDKLKGLLKMYDKVRNYLTK |
| | | KPYSIEKFKLHFDNPSLLSGWDKNKELNNLSVIFRQNGYYYLGIMTPKG |
| | | KNLFKTLPKLGAEEMFYEKMEYKQIAEPMLMLPKVFFPKKTKPAFAPD |
| | | QSVVDIYNKKTFKTGQKGFNKKDLYRLIDFYKEALTVHEWKLFNFSFS |
| | | PTEQYRNIGEFFDEVREQAYKVSMVNVPASYIDEAVENGKLYLFQIYN |
| | | KDFSPYSKGIPNLHTLYWKALFSEQNQSRVYKLCGGGELFYRKASLHM |
| | | QDTTVHPKGISIHKKNLNKKGETSLFNYDLVKDKRFTEDKFFFHVPISIN |
| | | YKNKKITNVNQMVRDYIAQNDDLQIIGIDRGERNLLYISRIDTRGNLLE |
| | | QFSLNVIESDKGDLRTDYQKILGDREQERLRRRQEWKSIESIKDLKDGY |
| | | MSQVVHKICNMVVEHKAIVVLENLNLSFMKGRKKVEKSVYEKFERML |
| | | VDKLNYLVVDKKNLSNEPGGLYAAYQLTNPLFSFEELHRYPQSGILFFV |
| | | DPWNTSLTDPSTGFVNLLGRINYTNVGDARKFFDRFNAIRYDGKGNILF |
| | | DLDLSRFDVRVETQRKLWTLTTFGSRIAKSKKSGKWMVERIENLSLCFL |
| | | ELFEQFNIGYRVEKDLKKAILSQDRKEFYVRLIYLFNLMMQIRNSDGEE |
| | | DYILSPALNEKNLQFDSRLIEAKDLPVDADANGAYNVARKGLMVVQRI |
| | | KRGDHESIHRIGRAQWLRYVQEGIVE |
| SEQ | Moraxella | MLFQDFTHLYPLSKTVRFELKPIDRTLEHIHAKNFLSQDETMADMHQK |
| ID | bovoculi | VKVILDDYHRDFIADMMGEVKLTKLAEFYDVYLKFRKNPKDDELQKQ |
| NO: | 237 | LKDLQAVLRKEIVKPIGNGGKYKAGYDRLFGAKLFKDGKELGDLAKF |
| 40 | (MbCas12a) | VIAQEGESSPKLAHLAHFEKFSTYFTGFHDNRKNMYSDEDKHTAIAYR |
| | | LIHENLPRFIDNLQILTTIKQKHSALYDQIINELTASGLDVSLASHLDGYH |
| | | KLLTQEGITAYNTLLGGISGEAGSPKIQGINELINSHHNQHCHKSERIAK |
| | | LRPLHKQILSDGMSVSFLPSKFADDSEMCQAVNEFYRHYADVFAKVQS |
| | | LFDGFDDHQKDGIYVEHKNLNELSKQAFGDFALLGRVLDGYYVDVVN |
| | | PEFNERFAKAKTDNAKAKLTKEKDKFIKGVHSLASLEQAIEHYTARHD |
| | | DESVQAGKLGQYFKHGLAGVDNPIQKIHNNHSTIKGFLERERPAGERA |
| | | LPKIKSGKNPEMTQLRQLKELLDNALNVAHFAKLLTTKTTLDNQDGNF |
| | | YGEFGVLYDELAKIPTLYNKVRDYLSQKPFSTEKYKLNFGNPTLLNGW |
| | | DLNKEKDNFGVILQKDGCYYLALLDKAHKKVFDNAPNTGKSIYQKMI |
| | | YKYLEVRKQFPKVFFSKEAIAINYHPSKELVEIKDKGRQRSDDERLKLY |
| | | RFILECLKIHPKYDKKFEGAIGDIQLFKKDKKGREVPISEKDLFDKINGIF |
| | | SSKPKLEMEDFFIGEFKRYNPSQDLVDQYNIYKKIDSNDNRKKENFYNN |
| | | HPKFKKDLVRYYYESMCKHEEWEESFEFSKKLQDIGCYVDVNELFTEI |
| | | ETRRLNYKISFCNINADYIDELVEQGQLYLFQIYNKDFSPKAHGKPNLH |
| | | TLYFKALFSEDNLADPIYKLNGEAQIFYRKASLDMNETTIHRAGEVLEN |
| | | KNPDNPKKRQFVYDIIKDKRYTQDKFMLHVPITMNFGVQGMTIKEFNK |
| | | KVNQSIQQYDEVNVIGIDRGERHLLYLTVINSKGEILEQCSLNDITTASA |
| | | NGTQMTTPYHKILDKREIERLNARVGWGEIETIKELKSGYLSHVVHQIS |
| | | QLMLKYNAIVVLEDLNFGFKRGRFKVEKQIYQNFENALIKKLNHLVLK |
| | | DKADDEIGSYKNALQLTNNFTDLKSIGKQTGFLFYVPAWNTSKIDPETG |
| | | FVDLLKPRYENIAQSQAFFGKFDKICYNADKDYFEFHIDYAKFTDKAK |
| | | NSRQIWTICSHGDKRYVYDKTANQNKGAAKGINVNDELKSLFARHHIN |
| | | EKQPNLVMDICQNNDKEFHKSLMYLLKTLLALRYSNASSDEDFILSPVA |
| | | NDEGVFFNSALADDTQPQNADANGAYHIALKGLWLLNELKNSDDLNK |
| | | VKLAIDNQTWLNFAQNR |
| SEQ | Moraxella | MGIHGVPAALFQDFTHLYPLSKTVRFELKPIGRTLEHIHAKNFLSQDET |
| ID | bovoculi | MADMYQKVKVILDDYHRDFIADMMGEVKLTKLAEFYDVYLKFRKNP |
| NO: | AAX08_00205 | KDDGLQKQLKDLQAVLRKESVKPIGSGGKYKTGYDRLFGAKLFKDGK |
| 41 | (Mb2Cas12a) | ELGDLAKFVIAQEGESSPKLAHLAHFEKFSTYFTGFHDNRKNMYSDED |
| | | KHTAIAYRLIHENLPRFIDNLQILTTIKQKHSALYDQIINELTASGLDVSL |
| | | ASHLDGYHKLLTQEGITAYNRIIGEVNGYTNKHNQICHKSERIAKLRPL |
| | | HKQILSDGMGVSFLPSKFADDSEMCQAVNEFYRHYTDVFAKVQSLFDG |
| | | FDDHQKDGIYVEHKNLNELSKQAFGDFALLGRVLDGYYVDVVNPEFN |
| | | ERFAKAKTDNAKAKLTKEKDKFIKGVHSLASLEQAIEHHTARHDDESV |
| | | QAGKLGQYFKHGLAGVDNPIQKIHNNHSTIKGFLERERPAGERALPKIK |
| | | SGKNPEMTQLRQLKELLDNALNVAHFAKLLTTKTTLDNQDGNFYGEF |
| | | GVLYDELAKIPTLYNKVRDYLSQKPFSTEKYKLNFGNPTLLNGWDLNK |
| | | EKDNFGVILQKDGCYYLALLDKAHKKVFDNAPNTGKNVYQKMVYKL |
| | | LPGPNKMLPKVFFAKSNLDYYNPSAELLDKYAKGTHKKGDNFNLKDC |
| | | HALIDFFKAGINKHPEWQHFGFKFSPTSSYRDLSDFYREVEPQGYQVKF |
| | | VDINADYIDELVEQGKLYLFQIYNKDFSPKAHGKPNLHTLYFKALFSED |
| | | NLADPIYKLNGEAQIFYRKASLDMNETTIHRAGEVLENKNPDNPKKRQ |
| | | FVYDIIKDKRYTQDKFMLHVPITMNFGVQGMTIKEFNKKVNQSIQQYD |
| | | EVNVIGIDRGERHLLYLTVINSKGEILEQRSLNDITTASANGTQVTTPYH |
| | | KILDKREIERLNARVGWGEIETIKELKSGYLSHVVHQINQLMLKYNAIV |
| | | VLEDLNFGFKRGRFKVEKQIYQNFENALIKKLNHLVLKDKADDEIGSY |
| | | KNALQLTNNFTDLKSIGKQTGFLFYVPAWNTSKIDPETGFVDLLKPRYE |
| | | NIAQSQAFFGKFDKICYNTDKGYFEFHIDYAKFTDKAKNSRQKWAICS |
| | | HGDKRYVYDKTANQNKGAAKGINVNDELKSLFARYHINDKQPNLVM |
| | | DICQNNDKEFHKSLMCLLKTLLALRYSNASSDEDFILSPVANDEGVFFN |
| | | SALADDTQPQNADANGAYHIALKGLWLLNELKNSDDLNKVKLAIDNQ |
| | | TWLNFAQNR |
| SEQ | Moraxella | MGIHGVPAALFQDFTHLYPLSKTVRFELKPIGKTLEHIHAKNFLNQDET |
| ID | bovoculi | MADMYQKVKAILDDYHRDFIADMMGEVKLTKLAEFYDVYLKFRKNP |
| NO: | AAX11_00205 | KDDGLQKQLKDLQAVLRKEIVKPIGNGGKYKAGYDRLFGAKLFKDGK |
| 42 | (Mb3Cas12a) | ELGDLAKFVIAQEGESSPKLAHLAHFEKFSTYFTGFHDNRKNMYSDED |
| | | KHTAIAYRLIHENLPRFIDNLQILATIKQKHSALYDQIINELTASGLDVSL |
| | | ASHLDGYHKLLTQEGITAYNTLLGGISGEAGSRKIQGINELINSHHNQH |
| | | CHKSERIAKLRPLHKQILSDGMGVSFLPSKFADDSEVCQAVNEFYRHY |
| | | ADVFAKVQSLFDGFDDYQKDGIYVEYKNLNELSKQAFGDFALLGRVL |
| | | DGYYVDVVNPEFNERFAKAKTDNAKAKLTKEKDKFIKGVHSLASLEQ |
| | | AIEHYTARHDDESVQAGKLGQYFKHGLAGVDNPIQKIHNNHSTIKGFL |
| | | ERERPAGERALPKIKSDKSPEIRQLKELLDNALNVAHFAKLLTTKTTLH |
| | | NQDGNFYGEFGALYDELAKIATLYNKVRDYLSQKPFSTEKYKLNFGNP |
| | | TLLNGWDLNKEKDNFGVILQKDGCYYLALLDKAHKKVFDNAPNTGKS |
| | | VYQKMIYKLLPGPNKMLPKVFFAKSNLDYYNPSAELLDKYAQGTHKK |
| | | GDNFNLKDCHALIDFFKAGINKHPEWQHFGFKFSPTSSYQDLSDFYREV |
| | | EPQGYQVKFVDINADYINELVEQGQLYLFQIYNKDFSPKAHGKPNLHT |
| | | LYFKALFSEDNLVNPIYKLNGEAEIFYRKASLDMNETTIHRAGEVLENK |
| | | NPDNPKKRQFVYDIIKDKRYTQDKFMLHVPITMNFGVQGMTIKEFNKK |
| | | VNQSIQQYDEVNVIGIDRGERHLLYLTVINSKGEILEQRSLNDITTASAN |
| | | GTQMTTPYHKILDKREIERLNARVGWGEIETIKELKSGYLSHVVHQISQ |
| | | LMLKYNAIVVLEDLNFGFKRGRFKVEKQIYQNFENALIKKLNHLVLKD |
| | | KADDEIGSYKNALQLTNNFTDLKSIGKQTGFLFYVPAWNTSKIDPETGF |
| | | VDLLKPRYENIAQSQAFFGKFDKICYNADRGYFEFHIDYAKFNDKAKN |
| | | SRQIWKICSHGDKRYVYDKTANQNKGATIGVNVNDELKSLFTRYHIND |
| | | KQPNLVMDICQNNDKEFHKSLMYLLKTLLALRYSNASSDEDFILSPVA |
| | | NDEGVFFNSALADDTQPQNADANGAYHIALKGLWLLNELKNSDDLNK |
| | | VKLAIDNQTWLNFAQNR |
| SEQ | Thiomicrospira | MGIHGVPAATKTFDSEFFNLYSLQKTVRFELKPVGETASFVEDFKNEGL |
| ID | sp. XS5 | KRVVSEDERRAVDYQKVKEIIDDYHRDFIEESLNYFPEQVSKDALEQAF |
| NO: | (TsCas12a) | HLYQKLKAAKVEEREKALKEWEALQKKLREKVVKCFSDSNKARFSRI |
| 43 | | DKKELIKEDLINWLVAQNREDDIPTVETFNNFTTYFTGFHENRKNIYSK |
| | | DDHATAISFRLIHENLPKFFDNVISFNKLKEGFPELKFDKVKEDLEVDYD |
| | | LKHAFEIEYFVNFVTQAGIDQYNYLLGGKTLEDGTKKQGMNEQINLFK |
| | | QQQTRDKARQIPKLIPLFKQILSERTESQSFIPKQFESDQELFDSLQKLHN |
| | | NCQDKFTVLQQAILGLAEADLKKVFIKTSDLNALSNTIFGNYSVFSDAL |
| | | NLYKESLKTKKAQEAFEKLPAHSIHDLIQYLEQFNSSLDAEKQQSTDTV |
| | | LNYFIKTDELYSRFIKSTSEAFTQVQPLFELEALSSKRRPPESEDEGAKG |
| | | QEGFEQIKRIKAYLDTLMEAVHFAKPLYLVKGRKMIEGLDKDQSFYEA |
| | | FEMAYQELESLIIPIYNKARSYLSRKPFKADKFKINFDNNTLLSGWDAN |
| | | KETANASILFKKDGLYYLGIMPKGKTFLFDYFVSSEDSEKLKQRRQKTA |
| | | EEALAQDGESYFEKIRYKLLPGASKMLPKVFFSNKNIGFYNPSDDILRIR |
| | | NTASHTKNGTPQKGHSKVEFNLNDCHKMIDFFKSSIQKHPEWGSFGFTF |
| | | SDTSDFEDMSAFYREVENQGYVISFDKIKETYIQSQVEQGNLYLFQIYN |
| | | KDFSPYSKGKPNLHTLYWKALFEEANLNNVVAKLNGEAEIFFRRHSIK |
| | | ASDKVVHPANQAIDNKNPHTEKTQSTFEYDLVKDKRYTQDKFFFHVPI |
| | | SLNFKAQGVSKFNDKVNGFLKGNPDVNIIGIDRGERHLLYFTVVNQKG |
| | | EILVQESLNTLMSDKGHVNDYQQKLDKKEQERDAARKSWTTVENIKE |
| | | LKEGYLSHVVHKLAHLIIKYNAIVCLEDLNFGFKRGRFKVEKQVYQKF |
| | | EKALIDKLNYLVFKEKELGEVGHYLTAYQLTAPFESFKKLGKQSGILFY |
| | | VPADYTSKIDPTTGFVNFLDLRYQSVEKAKQLLSDFNAIRFNSVQNYFE |
| | | FEIDYKKLTPKRKVGTQSKWVICTYGDVRYQNRRNQKGHWETEEVNV |
| | | TEKLKALFASDSKTTTVIDYANDDNLIDVILEQDKASFFKELLWLLKLT |
| | | MTLRHSKIKSEDDFILSPVKNEQGEFYDSRKAGEVWPKDADANGAYHI |
| | | ALKGLWNLQQINQWEKGKTLNLAIKNQDWFSFIQEKPYQE |
| SEQ | Butyrivibrio | MGIHGVPAAYYQNLTKKYPVSKTIRNELIPIGKTLENIRKNNILESDVKR |
| ID | sp. NC3005 | KQDYEHVKGIMDEYHKQLINEALDNYMLPSLNQAAEIYLKKHVDVED |
| NO: | (BsCas12a) | REEFKKTQDLLRREVTGRLKEHENYTKIGKKDILDLLEKLPSISEEDYN |
| 44 | | ALESFRNFYTYFTSYNKVRENLYSDEEKSSTVAYRLINENLPKFLDNIKS |
| | | YAFVKAAGVLADCIEEEEQDALFMVETFNMTLTQEGIDMYNYQIGKV |
| | | NSAINLYNQKNHKVEEFKKIPKMKVLYKQILSDREEVFIGEFKDDETLL |
| | | SSIGAYGNVLMTYLKSEKINIFFDALRESEGKNVYVKNDLSKTTMSNIV |
| | | FGSWSAFDELLNQEYDLANENKKKDDKYFEKRQKELKKNKSYTLEQM |
| | | SNLSKEDISPIENYIERISEDIEKICIYNGEFEKIVVNEHDSSRKLSKNIKAV |
| | | KVIKDYLDSIKELEHDIKLINGSGQELEKNLVVYVGQEEALEQLRPVDS |
| | | LYNLTRNYLTKKPFSTEKVKLNFNKSTLLNGWDKNKETDNLGILFFKD |
| | | GKYYLGIMNTTANKAFVNPPAAKTENVFKKVDYKLLPGSNKMLPKVF |
| | | FAKSNIGYYNPSTELYSNYKKGTHKKGPSFSIDDCHNLIDFFKESIKKHE |
| | | DWSKFGFEFSDTADYRDISEFYREVEKQGYKLTFTDIDESYINDLIEKNE |
| | | LYLFQIYNKDFSEYSKGKLNLHTLYFMMLFDQRNLDNVVYKLNGEAE |
| | | VFYRPASIAENELVIHKAGEGIKNKNPNRAKVKETSTFSYDIVKDKRYS |
| | | KYKFTLHIPITMNFGVDEVRRFNDVINNALRTDDNVNVIGIDRGERNLL |
| | | YVVVINSEGKILEQISLNSIINKEYDIETNYHALLDEREDDRNKARKDW |
| | | NTIENIKELKTGYLSQVVNVVAKLVLKYNAIICLEDLNFGFKRGRQKVE |
| | | KQVYQKFEKMLIEKLNYLVIDKSREQVSPEKMGGALNALQLTSKFKSF |
| | | AELGKQSGIIYYVPAYLTSKIDPTTGFVNLFYIKYENIEKAKQFFDGFDFI |
| | | RFNKKDDMFEFSFDYKSFTQKACGIRSKWIVYTNGERIIKYPNPEKNNL |
| | | FDEKVINVTDEIKGLFKQYRIPYENGEDIKEIIISKAEADFYKRLFRLLHQ |
| | | TLQMRNSTSDGTRDYIISPVKNDRGEFFCSEFSEGTMPKDADANGAYNI |
| | | ARKGLWVLEQIRQKDEGEKVNLSMTNAEWLKYAQLHLL |
| SEQ | AacCas12b | MAVKSIKVKLRLDDMPEIRAGLWKLHKEVNAGVRYYTEWLSLLRQEN |
| ID | | LYRRSPNGDGEQECDKTAEECKAELLERLRARQVENGHRGPAGSDDEL |
| NO: | | LQLARQLYELLVPQAIGAKGDAQQIARKFLSPLADKDAVGGLGIAKAG |
| 45 | | NKPRWVRMREAGEPGWEEEKEKAETRKSADRTADVLRALADFGLKPL |
| | | MRVYTDSEMSSVEWKPLRKGQAVRTWDRDMFQQAIERMMSWESWN |
| | | QRVGQEYAKLVEQKNRFEQKNFVGQEHLVHLVNQLQQDMKEASPGL |
| | | ESKEQTAHYVTGRALRGSDKVFEKWGKLAPDAPFDLYDAEIKNVQRR |
| | | NTRRFGSHDLFAKLAEPEYQALWREDASFLTRYAVYNSILRKLNHAKM |
| | | FATFTLPDATAHPIWTRFDKLGGNLHQYTFLFNEFGERRHAIRFHKLLK |
| | | VENGVAREVDDVTVPISMSEQLDNLLPRDPNEPIALYFRDYGAEQHFT |
| | | GEFGGAKIQCRRDQLAHMHRRRGARDVYLNVSVRVQSQSEARGERRP |
| | | PYAAVFRLVGDNHRAFVHFDKLSDYLAEHPDDGKLGSEGLLSGLRVM |
| | | SVDLGLRTSASISVFRVARKDELKPNSKGRVPFFFPIKGNDNLVAVHER |
| | | SQLLKLPGETESKDLRAIREERQRTLRQLRTQLAYLRLLVRCGSEDVGR |
| | | RERSWAKLIEQPVDAANHMTPDWREAFENELQKLKSLHGICSDKEWM |
| | | DAVYESVRRVWRHMGKQVRDWRKDVRSGERPKIRGYAKDVVGGNSI |
| | | EQIEYLERQYKFLKSWSFFGKVSGQVIRAEKGSRFAITLREHIDHAKED |
| | | RLKKLADRIIMEALGYVYALDERGKGKWVAKYPPCQLILLEELSEYQF |
| | | NNDRPPSENNQLMQWSHRGVFQELINQAQVHDLLVGTMYAAFSSRFD |
| | | ARTGAPGIRCRRVPARCTQEHNPEPFPWWLNKFVVEHTLDACPLRADD |
| | | LIPTGEGEIFVSPFSAEEGDFHQIHADLNAAQNLQQRLWSDFDISQIRLR |
| | | CDWGEVDGELVLIPRLTGKRTADSYSNKVFYTNTGVTYYERERGKKR |
| | | RKVFAQEKLSEEEAELLVEADEAREKSVVLMRDPSGIINRGNWTRQKE |
| | | FWSMVNQRIEGYLVKQIRSRVPLQDSACENTGDI |
| SEQ | Cas12 | MKKIDNFVGCYPVSKTLRFKAIPIGKTQENIEKKRLVEEDEVRAK |
| ID | Variant | DYKAVKKLIDRYHREFIEGVLDNVKLDGLEEYYMLFNKSDREES |
| NO: | | DNKKIEIMEERFRRVISKSFKNNEEYKKIFSKKIIEEILPNYIKDEEE |
| 46 | | KELVKGFKGFYTAFVGYAQNRENMYSDEKKSTAISYRIVNENMP |
| | | RFITNIKVFEKAKSILDVDKINEINEYILNNDYYVDDFFNIDFFNYV |
| | | LNQKGIDIYNAIIGGIVTGDGRKIQGLNECINLYNQENKKIRLPQF |
| | | KPLYKQILSESESMSFYIDEIESDDMLIDMLKESLQIDSTINNAIDD |
| | | LKVLFNNIFDYDLSGIFINNGLPITTISNDVYGQWSTISDGWNERY |
| | | DVLSNAKDKESEKYFEKRRKEYKKVKSFSISDLQELGGKDLSICK |
| | | KINEIISEMIDDYKSKIEEIQYLFDIKELEKPLVTDLNKIELIKNSLD |
| | | GLKRIERYVIPFLGTGKEQNRDEVFYGYFIKCIDAIKEIDGVYNKT |
| | | RNYLTKKPYSKDKFKLYFENPQLMGGWDRNKESDYRSTLLRKN |
| | | GKYYVAIIDKSSSNCMMNIEEDENDNYEKINYKLLPGPNKMLPK |
| | | VFFSKKNREYFAPSKEIERIYSTGTFKKDTNFVKKDCENLITFYKD |
| | | SLDRHEDWSKSFDFSFKESSAYRDISEFYRDVEKQGYRVSFDLLS |
| | | SNAVNTLVEEGKLYLFQLYNKDFSEKSHGIPNLHTMYFRSLFDD |
| | | NNKGNIRLNGGAEMFMRRASLNKQDVTVHKANQPIKNKNLLNP |
| | | KKTTTLPYDVYKDKRFTEDQYEVHIPITMNKVPNNPYKINHMVR |
| | | EQLVKDDNPYVIGIDRGERNLIYVVVVDGQGHIVEQLSLNEIINE |
| | | NNGISIRTDYHTLLDAKERERDESRKQWKQIENIKELKEGYISQV |
| | | VHKICELVEKYDAVIALEDLNSGFKNSRVKVEKQVYQKFEKMLI |
| | | TKLNYMVDKKKDYNKPGGVLNGYQLTTQFESFSKMGTQNGIMF |
| | | YIPAWLTSKMDPTTGFVDLLKPKYKNKADAQKFFSQFDSIRYDN |
| | | QEDAFVFKVNYTKFPRTDADYNKEWEIYTNGERIRVFRNPKKNN |
| | | EYDYETVNVSERMKELFDSYDLLYDKGELKETICEMEESKFFEEL |
| | | IKLFRLTLQMRNSISGRTDVDYLISPVKNSNGYFYNSNDYKKEGA |
| | | KYPKDADANGAYNIARKVLWAIEQFKMADEDKLDKTKISIKNQ |
| | | EWLEYAQTHCE |

Alternatively, the Type V CRISPR/Cas enzyme is a programmable Cas14 nuclease. A Cas14 protein of the present disclosure includes 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. A naturally occurring Cas14 protein functions as an endonuclease that catalyzes cleavage at a specific sequence in a target nucleic acid. A programmable Cas14 nuclease can be a Cas114a protein, a Cas114b protein, a Cas114c protein, a Cas114d protein, a Cas14e protein, a Cas14f protein, a Cas14g protein, a Cas14h protein, or a Cas14u protein. In some cases, a suitable Cas14 protein comprises an amino acid sequence having at least 8000, at least 85%, at least 90%, at least 95%, at least 98%, at least 99%, or 10000, amino acid sequence identity to any one of SEQ ID NO: 47-SEQ ID NO: 138.

TABLE 3
Cas14 Protein Sequences

| SEQ | |
|---|---|
| ID NO | Sequence |
| SEQ | MEVQKTVMKTLSLRILRPLYSQEIEKEIKEEKERRKQAGGTGELDGGFYKKLEKKHSE |
| ID | MFSFDRLNLLLNQLQREIAKVYNHAISELYIATIAQGNKSNKHYISSIVYNRAYGYFYN |
| NO: | AYIALGICSKVEANFRSNELLTQQSALPTAKSDNFPIVLHKQKGAEGEDGGFRISTEGS |
| 47 | DLIFEIPIPFYEYNGENRKEPYKWVKKGGQKPVLKLILSTFRRQRNKGWAKDEGTDAE |
| | IRKVTEGKYQVSQIEINRGKKLGEHQKWFANFSIEQPIYERKPNRSIVGGLDVGIRSPLV |
| | CAINNSFSRYSVDSNDVFKFSKQVFAFRRRLLSKNSLKRKGHGAAHKLEPITEMTEKN |
| | DKFRKKIIERWAKEVTNFFVKNQVGIVQIEDLSTMKDREDHFFNQYLRGFWPYYQMQ |
| | TLIENKLKEYGIEVKRVQAKYTSQLCSNPNCRYWNNYFNFEYRKVNKFPKFKCEKCN |
| | LEISADYNAARNLSTPDIEKFVAKATKGINLPEK |
| SEQ | MEEAKTVSKTLSLRILRPLYSAEIEKEIKEEKERRKQGGKSGELDSGFYKKLEKKHTQ |
| ID | MFGWDKLNLMLSQLQRQIARVFNQSISELYIETVIQGKKSNKHYTSKIVYNRAYSVFY |
| NO: | NAYLALGITSKVEANFRSTELLMQKSSLPTAKSDNFPILLHKQKGVEGEEGGFKISADG |
| 48 | NDLIFEIPIPFYEYDSANKKEPFKWIKKGGQKPTIKLILSTFRRQRNKGWAKDEGTDAEI |
| | RKVIEGKYQVSHIEINRGKKLGDHQKWFVNFTIEQPIYERKLDKNIIGGIDVGIKSPLVC |
| | AVNNSFARYSVDSNDVLKFSKQAFAFRRRLLSKNSLKRSGHGSKNKLDPITRMTEKN |
| | DRFRKKIIERWAKEVTNFFIKNQVGTVQIEDLSTMKDRQDNFFNQYLRGFWPYYQMQ |
| | NLIENKLKEYGIETKRIKARYTSQLCSNPSCRHWNSYFSFDHRKTNNFPKFKCEKCALE |
| | ISADYNAARNISTPDIEKFVAKATKGINLPDKNENVILE |
| SEQ | MAKNTITKTLKLRIVRPYNSAEVEKIVADEKNNREKIALEKNKDKVKEACSKHLKVA |
| ID | AYCTTQVERNACLFCKARKLDDKFYQKLRGQFPDAVFWQEISEIFRQLQKQAAEIYN |
| NO: | QSLIELYYEIFIKGKGIANASSVEHYLSDVCYTRAAELFKNAAIASGLRSKIKSNFRLKE |
| 49 | LKNMKSGLPTTKSDNFPIPLVKQKGGQYTGFEISNHNSDFIIKIPFGRWQVKKEIDKYR |
| | PWEKFDFEQVQKSPKPISLLLSTQRRKRNKGWSKDEGTEAEIKKVMNGDYQTSYIEVK |
| | RGSKIGEKSAWMLNLSIDVPKIDKGVDPSIIGGIDVGVKSPLVCAINNAFSRYSISDNDL |
| | FHFNKKMFARRRILLKKNRHKRAGHGAKNKLKPITILTEKSERFRKKLIERWACEIADF |
| | FIKNKVGTVQMENLESMKRKEDSYFNIRLRGFWPYAEMQNKIEFKLKQYGIEIRKVAP |
| | NNTSKTCSKCGHLNNYFNFEYRKKNKFPHFKCEKCNFKENADYNAALNISNPKLKST |
| | KEEP |
| SEQ | MERQKVPQIRKIVRVVPLRILRPKYSDVIENALKKFKEKGDDTNTNDFWRAIRDRDTE |
| ID | FFRKELNFSEDEINQLERDTLFRVGLDNRVLFSYFDFLQEKLMKDYNKIISKLFINRQSK |
| NO: | SSFENDLTDEEVEELIEKDVTPFYGAYIGKGIKSVIKSNLGGKFIKSVKIDRETKKVTKL |
| 50 | TAINIGLMGLPVAKSDTFPIKIIKTNPDYITFQKSTKENLQKIEDYETGIEYGDLLVQITIP |
| | WFKNENKDFSLIKTKEAIEYYKLNGVGKKDLLNINLVLTTYHIRKKKSWQIDGSSQSL |
| | VREMANGELEEKWKSFFDTFIKKYGDEGKSALVKRRVNKKSRAKGEKGRELNLDERI |
| | KRLYDSIKAKSFPSEINLIPENYKWKLHFSIEIPPMVNDIDSNLYGGIDFGEQNIATLCVK |
| | NIEKDDYDFLTIYGNDLLKHAQASYARRRIMRVQDEYKARGHGKSRKTKAQEDYSER |
| | MQKLRQKITERLVKQISDFFLWRNKFHMAVCSLRYEDLNTLYKGESVKAKRMRQFIN |
| | KQQLFNGIERKLKDYNSEIYVNSRYPHYTSRLCSKCGKLNLYFDFLKFRTKNIIIRKNP |
| | DGSEIKYMPFFICEFCGWKQAGDKNASANIADKDYQDKLNKEKEFCNIRKPKSKKEDI |
| | GEENEEERDYSRRFNRNSFIYNSLKKDNKLNQEKLFDEWKNQLKRKIDGRNKFEPKE |
| | YKDRFSYLFAYYQEIIKNESES |
| SEQ | MVPTELITKTLQLRVIRPLYFEEIEKELAELKEQKEKEFEETNSLLLESKKIDAKSLKKL |
| ID | KRKARSSAAVEFWKIAKEKYPDILTKPEMEFIFSEMQKMMARFYNKSMTNIFIEMNND |
| NO: | EKVNPLSLISKASTEANQVIKCSSISSGLNRKIAGSINKTKFKQVRDGLISLPTARTETFPI |
| 51 | SFYKSTANKDEIPISKINLPSEEEADLTITLPFPFFEIKKEKKGQKAYSYFNIIEKSGRSNN |
| | KIDLLLSTHRRQRRKGWKEEGGTSAEIRRLMEGEFDKEWEIYLGEAEKSEKAKNDLIK |
| | NMTRGKLSKDIKEQLEDIQVKYFSDNNVESWNDLSKEQKQELSKLRKKKVEELKDW |
| | KHVKEILKTRAKIGWVELKRGKRQRDRNKWFVNITITRPPFINKELDDTKFGGIDLGV |
| | KVPFVCAVHGSPARLIIKENEILQFNKMVSARNRQITKDSEQRKGRGKKNKFIKKEIFN |
| | ERNELFRKKIIERWANQIVKFFEDQKCATVQIENLESFDRTSYK |
| SEQ | MKSDTKDKKIIIHQTKTLSLRIVKPQSIPMEEFTDLVRYHQMIIFPVYNNGAIDLYKKLF |
| ID | KAKIQKGNEARAIKYFMNKIVYAPIANTVKNSYIALGYSTKMQSSFSGKRLWDLRFGE |
| NO: | ATPPTIKADFPLPFYNQSGFKVSSENGEFIIGIPFGQYTKKTVSDIEKKTSFAWDKFTLED |
| 52 | TTKKTLIELLLSTKTRKMNEGWKNNEGTEAEIKRVMDGTYQVTSLEILQRDDSWFVN |
| | FNIAYDSLKKQPDRDKIAGIHMGITRPLTAVIYNNKYRALSIYPNTVMHLTQKQLARIK |
| | EQRTNSKYATGGHGRNAKVTGTDTLSEAYRQRRKKIIEDWIASIVKFAINNEIGTIYLE |
| | DISNTNSFFAAREQKLIYLEDISNTNSFLSTYKYPISAISDTLQHKLEEKAIQVIRKKAYY |
| | VNQICSLCGHYNKGFTYQFRRKNKFPKMKCQGCLEATSTEFNAAANVANPDYEKLLI |
| | KHGLLQLKK |
| SEQ | MSTITRQVRLSPTPEQSRLLMAHCQQYISTVNVLVAAFDSEVLTGKVSTKDFRAALPS |
| ID | AVKNQALRDAQSVFKRSVELGCLPVLKKPHCQWNNQNWRVEGDQLILPICKDGKTQ |
| NO: | QERFRCAAVALEGKAGILRIKKKRGKWIADLTVTQEDAPESSGSAIMGVDLGIKVPAV |
| 53 | AHIGGKGTRFFGNGRSQRSMRRRFYARRKTLQKAKKLRAVRKSKGKEARWMKTINH |
| | QLSRQIVNHAHALGVGTIKIEALQGIRKGTTRKSRGAAARKNNRMTNTWSFSQLTLFI |
| | TYKAQRQGITVEQVDPAYTSQDCPACRARNGAQDRTYVCSECGWRGHRDTVGAINIS |
| | RRAGLSGHRRGATGA |
| SEQ | MIAQKTIKIKLNPTKEQIIKLNSIIEEYIKVSNFTAKKIAEIQESFTDSGLTQGTCSECGKE |
| ID | KTYRKYHLLKKDNKLFCITCYKRKYSQFTLQKVEFQNKTGLRNVAKLPKTYYTNAIR |
| NO: | FASDTFSGFDEIIKKKQNRLNSIQNRLNFWKELLYNPSNRNEIKIKVVKYAPKTDTREH |
| 54 | PHYYSEAEIKGRIKRLEKQLKKFKMPKYPEFTSETISLQRELYSWKNPDELKISSITDKN |
| | ESMNYYGKEYLKRYIDLINSQTPQILLEKENNSFYLCFPITKNIEMPKIDDTFEPVGIDW |
| | GITRNIAVVSILDSKTKKPKFVKFYSAGYILGKRKHYKSLRKHFGQKKRQDKINKLGT |
| | KEDRFIDSNIHKLAFLIVKEIRNHSNKPIILMENITDNREEAEKSMRQNILLHSVKSRLQ |
| | NYIAYKALWNNIPTNLVKPEHTSQICNRCGHQDRENRPKGSKLFKCVKCNYMSNADF |
| | NASINIARKFYIGEYEPFYKDNEKMKSGVNSISM |
| SEQ | LKLSEQENITTGVKFKLKLDKETSEGLNDYFDEYGKAINFAIKVIQKELAEDRFAGKVR |
| ID | LDENKKPLLNEDGKKIWDFPNEFCSCGKQVNRYVNGKSLCQECYKNKFTEYGIRKRM |
| NO: | YSAKGRKAEQDINIKNSTNKISKTHFNYAIREAFILDKSIKKQRKERFRRLREMKKKLQ |
| 55 | EFIEIRDGNKILCPKIEKQRVERYIHPSWINKEKKLEDFRGYSMSNVLGKIKILDRNIKRE |
| | EKSLKEKGQINFKARRLMLDKSVKFLNDNKISFTISKNLPKEYELDLPEKEKRLNWLK |
| | EKIKIIKNQKPKYAYLLRKDDNFYLQYTLETEFNLKEDYSGIVGIDRGVSHIAVYTFVH |
| | NNGKNERPLFLNSSEILRLKNLQKERDRFLRRKHNKKRKKSNMRNIEKKIQLILHNYS |
| | KQIVDFAKNKNAFIVFEKLEKPKKNRSKMSKKSQYKLSQFTFKKLSDLVDYKAKREGI |
| | KVLYISPEYTSKECSHCGEKVNTQRPFNGNSSLFKCNKCGVELNADYNASINIAKKGL |
| | NILNSTN |
| SEQ | MEESIITGVKFKLRIDKETTKKLNEYFDEYGKAINFAVKIIQKELADDRFAGKAKLDQN |
| ID | KNPILDENGKKIYEFPDEFCSCGKQVNKYVNNKPFCQECYKIRFTENGIRKRMYSAKG |
| NO: | RKAEHKINILNSTNKISKTHFNYAIREAFILDKSIKKQRKKRNERLRESKKRLQQFIDMR |
| 56 | DGKREICPTIKGQKVDRFIHPSWITKDKKLEDFRGYTLSIINSKIKILDRNIKREEKSLKE |
| | KGQIIFKAKRLMLDKSIRFVGDRKVLFTISKTLPKEYELDLPSKEKRLNWLKEKIEIIKN |
| | QKPKYAYLLRKNIESEKKPNYEYYLQYTLEIKPELKDFYDGAIGIDRGINHIAVCTFISN |
| | DGKVTPPKFFSSGEILRLKNLQKERDRFLLRKHNKNRKKGNMRVIENKINLILHRYSK |
| | QIVDMAKKLNASIVFEELGRIGKSRTKMKKSQRYKLSLFIFKKLSDLVDYKSRREGIRV |
| | TYVPPEYTSKECSHCGEKVNTQRPFNGNYSLFKCNKCGIQLNSDYNASINIAKKGLKIP |
| | NST |
| SEQ | LWTIVIGDFIEMPKQDLVTTGIKFKLDVDKETRKKLDDYFDEYGKAINFAVKIIQKNLK |
| ID | EDRFAGKIALGEDKKPLLDKDGKKIYNYPNESCSCGNQVRRYVNAKPFCVDCYKLKF |
| NO: | TENGIRKRMYSARGRKADSDINIKNSTNKISKTHFNYAIREGFILDKSLKKQRSKRIKKL |
| 57 | LELKRKLQEFIDIRQGQMVLCPKIKNQRVDKFIHPSWLKRDKKLEEFRGYSLSVVEGKI |
| | KIFNRNILREEDSLRQRGHVNFKANRIMLDKSVRFLDGGKVNFNLNKGLPKEYLLDLP |
| | KKENKLSWLNEKISLIKLQKPKYAYLLRREGSFFIQYTIENVPKTFSDYLGAIGIDRGIS |
| | HIAVCTFVSKNGVNKAPVFFSSGEILKLKSLQKQRDLFLRGKHNKIRKKSNMRNIDNKI |
| | NLILHKYSRNIVNLAKSEKAFIVFEKLEKIKKSRFKMSKSLQYKLSQFTFKKLSDLVEY |
| | KAKIEGIKVDYVPPEYTSKECSHCGEKVDTQRPFNGNSSLFKCNKCRVQLNADYNASI |
| | NIAKKSLNISN |
| SEQ | MSKTTISVKLKIIDLSSEKKEFLDNYFNEYAKATTFCQLRIRRLLRNTHWLGKKEKSSK |
| ID | KWIFESGICDLCGENKELVNEDRNSGEPAKICKRCYNGRYGNQMIRKLFVSTKKREVQ |
| NO: | ENMDIRRVAKLNNTHYHRIPEEAFDMIKAADTAEKRRKKNVEYDKKRQMEFIEMFND |
| 58 | EKKRAARPKKPNERETRYVHISKLESPSKGYTLNGIKRKIDGMGKKIERAEKGLSRKKI |
| | FGYQGNRIKLDSNWVRFDLAESEITIPSLFKEMKLRITGPTNVHSKSGQIYFAEWFERIN |
| | KQPNNYCYLIRKTSSNGKYEYYLQYTYEAEVEANKEYAGCLGVDIGCSKLAAAVYY |
| | DSKNKKAQKPIEIFTNPIKKIKMRREKLIKLLSRVKVRHRRRKLMQLSKTEPIIDYTCHK |
| | TARKIVEMANTAKAFISMENLETGIKQKQQARETKKQKFYRNMFLFRKLSKLIEYKAL |
| | LKGIKIVYVKPDYTSQTCSSCGADKEKTERPSQAIFRCLNPTCRYYQRDINADFNAAV |
| | NIAKKALNNTEVVTTLL |
| SEQ | MARAKNQPYQKLTTTTGIKFKLDLSEEEGKRFDEYFSEYAKAVNFCAKVIYQLRKNL |
| ID | KFAGKKELAAKEWKFEISNCDFCNKQKEIYYKNIANGQKVCKGCHRTNFSDNAIRKK |
| NO: | MIPVKGRKVESKFNIHNTTKKISGTHRHWAFEDAADIIESMDKQRKEKQKRLRREKRK |
| 59 | LSYFFELFGDPAKRYELPKVGKQRVPRYLHKIIDKDSLTKKRGYSLSYIKNKIKISERNI |
| | ERDEKSLRKASPIAFGARKIKMSKLDPKRAFDLENNVFKIPGKVIKGQYKFFGTNVAN |
| | EHGKKFYKDRISKILAGKPKYFYLLRKKVAESDGNPIFEYYVQWSIDTETPAITSYDNI |
| | LGIDAGITNLATTVLIPKNLSAEHCSHCGNNHVKPIFTKFFSGKELKAIKIKSRKQKYFL |
| | RGKHNKLVKIKRIRPIEQKVDGYCHVVSKQIVEMAKERNSCIALEKLEKPKKSKFRQR |
| | RREKYAVSMFVFKKLATFIKYKAAREGIEIIPVEPEGTSYTCSHCKNAQNNQRPYFKPN |
| | SKKSWTSMFKCGKCGIELNSDYNAAFNIAQKALNMTSA |
| SEQ | MDEKHFFCSYCNKELKISKNLINKISKGSIREDEAVSKAISIHNKKEHSLILGIKFKLFIE |
| ID | NKLDKKKLNEYFDNYSKAVTFAARIFDKIRSPYKFIGLKDKNTKKWTFPKAKCVFCLE |
| NO: | EKEVAYANEKDNSKICTECYLKEFGENGIRKKIYSTRGRKVEPKYNIFNSTKELSSTHY |
| 60 | NYAIRDAFQLLDALKKQRQKKLKSIFNQKLRLKEFEDIFSDPQKRIELSLKPHQREKRY |
| | IHLSKSGQESINRGYTLRFVRGKIKSLTRNIEREEKSLRKKTPIHFKGNRLMIFPAGIKFD |
| | FASNKVKISISKNLPNEFNFSGTNVKNEHGKSFFKSRIELIKTQKPKYAYVLRKIKREYS |
| | KLRNYEIEKIRLENPNADLCDFYLQYTIETESRNNEEINGIIGIDRGITNLACLVLLKKGD |
| | KKPSGVKFYKGNKILGMKIAYRKHLYLLKGKRNKLRKQRQIRAIEPKINLILHQISKDI |
| | VKIAKEKNFAIALEQLEKPKKARFAQRKKEKYKLALFTFKNLSTLIEYKSKREGIPVIY |
| | VPPEKTSQMCSHCAINGDEHVDTQRPYKKPNAQKPSYSLFKCNKCGIELNADYNAAF |
| | NIAQKGLKTLMLNHSH |
| SEQ | MLQTLLVKLDPSKEQYKMLYETMERFNEACNQIAETVFAIHSANKIEVQKTVYYPIRE |
| ID | KFGLSAQLTILAIRKVCEAYKRDKSIKPEFRLDGALVYDQRVLSWKGLDKVSLVTLQG |
| NO: | RQIIPIKFGDYQKARMDRIRGQADLILVKGVFYLCVVVEVSEESPYDPKGVLGVDLGIK |
| 61 | NLAVDSDGEVHSGEQTTNTRERLDSLKARLQSKGTKSAKRHLKKLSGRMAKFSKDV |
| | NHCISKKLVAKAKGTLMSIALEDLQGIRDRVTVRKAQRRNLHTWNFGLLRMFVDYK |
| | AKIAGVPLVFVDPRNTSRTCPSCGHVAKANRPTRDEFRCVSCGFAGAADHIAAMNIAF |
| | RAEVSQPIVTRFFVQSQAPSFRVG |
| SEQ | MDEEPDSAEPNLAPISVKLKLVKLDGEKLAALNDYFNEYAKAVNFCELKMQKIRKNL |
| ID | VNIRGTYLKEKKAWINQTGECCICKKIDELRCEDKNPDINGKICKKCYNGRYGNQMIR |
| NO: | KLFVSTNKRAVPKSLDIRKVARLHNTHYHRIPPEAADIIKAIETAERKRRNRILFDERRY |
| 62 | NELKDALENEEKRVARPKKPKEREVRYVPISKKDTPSKGYTMNALVRKVSGMAKKIE |
| | RAKRNLNKRKKIEYLGRRILLDKNWVRFDFDKSEISIPTMKEFFGEMRFEITGPSNVMS |
| | PNGREYFTKWFDRIKAQPDNYCYLLRKESEDETDFYLQYTWRPDAHPKKDYTGCLGI |
| | DIGGSKLASAVYFDADKNRAKQPIQIFSNPIGKWKTKRQKVIKVLSKAAVRHKTKKLE |
| | SLRNIEPRIDVHCHRIARKIVGMALAANAFISMENLEGGIREKQKAKETKKQKFSRNM |
| | FVFRKLSKLIEYKALMEGVKVVYIVPDYTSQLCSSCGTNNTKRPKQAIFMCQNTECRY |
| | FGKNINADFNAAINIAKKALNRKDIVRELS |
| SEQ | MEKNNSEQTSITTGIKFKLKLDKETKEKLNNYFDEYGKAINFAVRIIQMQLNDDRLAG |
| ID | KYKRDEKGKPILGEDGKKILEIPNDFCSCGNQVNHYVNGVSFCQECYKKRFSENGIRK |
| NO: | RMYSAKGRKAEQDINIKNSTNKISKTHFNYAIREAFNLDKSIKKQREKRFKKLKDMKR |
| 63 | KLQEFLEIRDGKRVICPKIEKQKVERYIHPSWINKEKKLEEFRGYSLSIVNSKIKSFDRNI |
| | QREEKSLKEKGQINFKAQRLMLDKSVKFLKDNKVSFTISKELPKTFELDLPKKEKKLN |
| | WLNEKLEIIKNQKPKYAYLLRKENNIFLQYTLDSIPEIHSEYSGAVGIDRGVSHIAVYTF |
| | LDKDGKNERPFFLSSSGILRLKNLQKERDKFLRKKHNKIRKKGNMRNIEQKINLILHEY |
| | SKQIVNFAKDKNAFIVFELLEKPKKSRERMSKKIQYKLSQFTFKKLSDLVDYKAKREGI |
| | KVIYVEPAYTSKDCSHCGERVNTQRPFNGNFSLFKCNKCGIVLNSDYNASLNIARKGL |
| | NISAN |
| SEQ | MAEEKFFFCEKCNKDIKIPKNYINKQGAEEKARAKHEHRVHALILGIKFKIYPKKEDIS |
| ID | KLNDYFDEYAKAVTFTAKIVDKLKAPFLFAGKRDKDTSKKKWVFPVDKCSFCKEKTE |
| NO: | INYRTKQGKNICNSCYLTEFGEQGLLEKIYATKGRKVSSSFNLFNSTKKLTGTHNNYV |
| 64 | VKESLQLLDALKKQRSKRLKKLSNTRRKLKQFEEMFEKEDKRFQLPLKEKQRELRFIH |
| | VSQKDRATEFKGYTMNKIKSKIKVLRRNIEREQRSLNRKSPVFFRGTRIRLSPSVQFDD |
| | KDNKIKLTLSKELPKEYSFSGLNVANEHGRKFFAEKLKLIKENKSKYAYLLRRQVNKN |
| | NKKPIYDYYLQYTVEFLPNIITNYNGILGIDRGINTLACIVLLENKKEKPSFVKFFSGKGI |
| | LNLKNKRRKQLYFLKGVHNKYRKQQKIRPIEPRIDQILHDISKQIIDLAKEKRVAISLEQ |
| | LEKPQKPKFRQSRKAKYKLSQFNFKTLSNYIDYKAKKEGIRVIYIAPEMTSQNCSRCA |
| | MKNDLHVNTQRPYKNTSSLFKCNKCGVELNADYNAAFNIAQKGLKILNS |
| SEQ | MISLKLKLLPDEEQKKLLDEMFWKWASICTRVGFGRADKEDLKPPKDAEGVWFSLTQ |
| ID | LNQANTDINDLREAMKHQKHRLEYEKNRLEAQRDDTQDALKNPDRREISTKRKDLFR |
| NO: | PKASVEKGFLKLKYHQERYWVRRLKEINKLIERKTKTLIKIEKGRIKFKATRITLHQGS |
| 65 | FKIRFGDKPAFLIKALSGKNQIDAPFVVVPEQPICGSVVNSKKYLDEITTNFLAYSVNA |
| | MLFGLSRSEEMLLKAKRPEKIKKKEEKLAKKQSAFENKKKELQKLLGRELTQQEEAII |
| | EETRNQFFQDFEVKITKQYSELLSKIANELKQKNDFLKVNKYPILLRKPLKKAKSKKIN |
| | NLSPSEWKYYLQFGVKPLLKQKSRRKSRNVLGIDRGLKHLLAVTVLEPDKKTFVWNK |
| | LYPNPITGWKWRRRKLLRSLKRLKRRIKSQKHETIHENQTRKKLKSLQGRIDDLLHNIS |
| | RKIVETAKEYDAVIVVEDLQSMRQHGRSKGNRLKTLNYALSLFDYANVMQLIKYKA |
| | GIEGIQIYDVKPAGTSQNCAYCLLAQRDSHEYKRSQENSKIGVCLNPNCQNHKKQIDA |
| | DLNAARVIASCYALKINDSQPFGTRKRFKKRTTN |
| SEQ | METLSLKLKLNPSKEQLLVLDKMFWKWASICTRLGLKKAEMSDLEPPKDAEGVWFS |
| ID | KTQLNQANTDVNDLRKAMQHQGKRIEYELDKVENRRNEIQEMLEKPDRRDISPNRKD |
| NO: | LFRPKAAVEKGYLKLKYHKLGYWSKELKTANKLIERKRKTLAKIDAGKMKFKPTRIS |
| 66 | LHTNSFRIKFGEEPKIALSTTSKHEKIELPLITSLQRPLKTSCAKKSKTYLDAAILNFLAY |
| | STNAALFGLSRSEEMLLKAKKPEKIEKRDRKLATKRESFDKKLKTLEKLLERKLSEKE |
| | KSVFKRKQTEFFDKFCITLDETYVEALHRIAEELVSKNKYLEIKKYPVLLRKPESRLRS |
| | KKLKNLKPEDWTYYIQFGFQPLLDTPKPIKTKTVLGIDRGVRHLLAVSIFDPRTKTFTF |
| | NRLYSNPIVDWKWRRRKLLRSIKRLKRRLKSEKHVHLHENQFKAKLRSLEGRIEDHFH |
| | NLSKEIVDLAKENNSVIVVENLGGMRQHGRGRGKWLKALNYALSHFDYAKVMQLIK |
| | YKAELAGVFVYDVAPAGTSINCAYCLLNDKDASNYTRGKVINGKKNTKIGECKTCKK |
| | EFDADLNAARVIALCYEKRLNDPQPFGTRKQFKPKKP |
| SEQ | MKALKLQLIPTRKQYKILDEMFWKWASLANRVSQKGESKETLAPKKDIQKIQFNATQ |
| ID | LNQIEKDIKDLRGAMKEQQKQKERLLLQIQERRSTISEMLNDDNNKERDPHRPLNFRP |
| NO: | KGWRKFHTSKHWVGELSKILRQEDRVKKTIERIVAGKISFKPKRIGIWSSNYKINFFKR |
| 67 | KISINPLNSKGFELTLMTEPTQDLIGKNGGKSVLNNKRYLDDSIKSLLMFALHSRFFGL |
| | NNTDTYLLGGKINPSLVKYYKKNQDMGEFGREIVEKFERKLKQEINEQQKKIIMSQIK |
| | EQYSNRDSAFNKDYLGLINEFSEVFNQRKSERAEYLLDSFEDKIKQIKQEIGESLNISDW |
| | DFLIDEAKKAYGYEEGFTEYVYSKRYLEILNKIVKAVLITDIYFDLRKYPILLRKPLDKI |
| | KKISNLKPDEWSYYIQFGYDSINPVQLMSTDKFLGIDRGLTHLLAYSVFDKEKKEFIIN |
| | QLEPNPIMGWKWKLRKVKRSLQHLERRIRAQKMVKLPENQMKKKLKSIEPKIEVHYH |
| | NISRKIVNLAKDYNASIVVESLEGGGLKQHGRKKNARNRSLNYALSLFDYGKIASLIK |
| | YKADLEGVPMYEVLPAYTSQQCAKCVLEKGSFVDPEIIGYVEDIGIKGSLLDSLFEGTE |
| | LSSIQVLKKIKNKIELSARDNHNKEINLILKYNFKGLVIVRGQDKEEIAEHPIKEINGKFA |
| | ILDFVYKRGKEKVGKKGNQKVRYTGNKKVGYCSKHGQVDADLNASRVIALCKYLDI |
| | NDPILFGEQRKSFK |
| SEQ | MVTRAIKLKLDPTKNQYKLLNEMFWKWASLANRFSQKGASKETLAPKDGTQKIQFN |
| ID | ATQLNQIKKDVDDLRGAMEKQGKQKERLLIQIQERLLTISEILRDDSKKEKDPHRPQNF |
| NO: | RPFGWRRFHTSAYWSSEASKLTRQVDRVRRTIERIKAGKINFKPKRIGLWSSTYKINFL |
| 68 | KKKINISPLKSKSFELDLITEPQQKIIGKEGGKSVANSKKYLDDSIKSLLIFAIKSRLFGLN |
| | NKDKPLFENIITPNLVRYHKKGQEQENFKKEVIKKFENKLKKEISQKQKEIIFSQIERQY |
| | ENRDATFSEDYLRAISEFSEIFNQRKKERAKELLNSFNEKIRQLKKEVNGNISEEDLKIL |
| | EVEAEKAYNYENGFIEWEYSEQFLGVLEKIARAVLISDNYFDLKKYPILIRKPTNKSKK |
| | ITNLKPEEWDYYIQFGYGLINSPMKIETKNFMGIDRGLTHLLAYSIFDRDSEKFTINQLE |
| | LNPIKGWKWKLRKVKRSLQHLERRMRAQKGVKLPENQMKKRLKSIEPKIESYYHNLS |
| | RKIVNLAKANNASIVVESLEGGGLKQHGRKKNSRHRALNYALSLFDYGKIASLIKYKS |
| | DLEGVPMYEVLPAYTSQQCAKCVLKKGSFVEPEIIGYIEEIGFKENLLTLLFEDTGLSSV |
| | QVLKKSKNKMTLSARDKEGKMVDLVLKYNFKGLVISQEKKKEEIVEFPIKEIDGKFAV |
| | LDSAYKRGKERISKKGNQKLVYTGNKKVGYCSVHGQVDADLNASRVIALCKYLGINE |
| | PIVFGEQRKSFK |
| SEQ | LDLITEPIQPHKSSSLRSKEFLEYQISDFLNFSLHSLFFGLASNEGPLVDFKIYDKIVIPKP |
| ID | EERFPKKESEEGKKLDSFDKRVEEYYSDKLEKKIERKLNTEEKNVIDREKTRIWGEVN |
| NO: | KLEEIRSIIDEINEIKKQKHISEKSKLLGEKWKKVNNIQETLLSQEYVSLISNLSDELTNK |
| 69 | KKELLAKKYSKFDDKIKKIKEDYGLEFDENTIKKEGEKAFLNPDKFSKYQFSSSYLKLI |
| | GEIARSLITYKGFLDLNKYPIIFRKPINKVKKIHNLEPDEWKYYIQFGYEQINNPKLETE |
| | NILGIDRGLTHILAYSVFEPRSSKFILNKLEPNPIEGWKWKLRKLRRSIQNLERRWRAQ |
| | DNVKLPENQMKKNLRSIEDKVENLYHNLSRKIVDLAKEKNACIVFEKLEGQGMKQHG |
| | RKKSDRLRGLNYKLSLFDYGKIAKLIKYKAEIEGIPIYRIDSAYTSQNCAKCVLESRRFA |
| | QPEEISCLDDFKEGDNLDKRILEGTGLVEAKIYKKLLKEKKEDFEIEEDIAMFDTKKVI |
| | KENKEKTVILDYVYTRRKEIIGTNHKKNIKGIAKYTGNTKIGYCMKHGQVDADLNAS |
| | RTIALCKNFDINNPEIWK |
| SEQ | MSDESLVSSEDKLAIKIKIVPNAEQAKMLDEMFKKWSSICNRISRGKEDIETLRPDEGK |
| ID | ELQFNSTQLNSATMDVSDLKKAMARQGERLEAEVSKLRGRYETIDASLRDPSRRHTN |
| NO: | PQKPSSFYPSDWDISGRLTPRFHTARHYSTELRKLKAKEDKMLKTINKIKNGKIVFKPK |
| 70 | RITLWPSSVNMAFKGSRLLLKPFANGFEMELPIVISPQKTADGKSQKASAEYMRNALL |
| | GLAGYSINQLLFGMNRSQKMLANAKKPEKVEKFLEQMKNKDANFDKKIKALEGKWL |
| | LDRKLKESEKSSIAVVRTKFFKSGKVELNEDYLKLLKHMANEILERDGFVNLNKYPILS |
| | RKPMKRYKQKNIDNLKPNMWKYYIQFGYEPIFERKASGKPKNIMGIDRGLTHLLAVA |
| | VFSPDQQKFLFNHLESNPIMHWKWKLRKIRRSIQHMERRIRAEKNKHIHEAQLKKRLG |
| | SIEEKTEQHYHIVSSKIINWAIEYEAAIVLESLSHMKQRGGKKSVRTRALNYALSLFDY |
| | EKVARLITYKARIRGIPVYDVLPGMTSKTCATCLLNGSQGAYVRGLETTKAAGKATK |
| | RKNMKIGKCMVCNSSENSMIDADLNAARVIAICKYKNLNDPQPAGSRKVFKRF |
| SEQ | MLALKLKIMPTEKQAEILDAMFWKWASICSRIAKMKKKVSVKENKKELSKKIPSNSDI |
| ID | WFSKTQLCQAEVDVGDHKKALKNFEKRQESLLDELKYKVKAINEVINDESKREIDPN |
| NO: | NPSKFRIKDSTKKGNLNSPKFFTLKKWQKILQENEKRIKKKESTIEKLKRGNIFFNPTKI |
| 71 | SLHEEEYSINFGSSKLLLNCFYKYNKKSGINSDQLENKFNEFQNGLNIICSPLQPIRGSSK |
| | RSFEFIRNSIINFLMYSLYAKLFGIPRSVKALMKSNKDENKLKLEEKLKKKKSSFNKTV |
| | KEFEKMIGRKLSDNESKILNDESKKFFEIIKSNNKYIPSEEYLKLLKDISEEIYNSNIDFKP |
| | YKYSILIRKPLSKFKSKKLYNLKPTDYKYYLQLSYEPFSKQLIATKTILGIDRGLKHLLA |
| | VSVFDPSQNKFVYNKLIKNPVFKWKKRYHDLKRSIRNRERRIRALTGVHIHENQLIKK |
| | LKSMKNKINVLYHNVSKNIVDLAKKYESTIVLERLENLKQHGRSKGKRYKKLNYVLS |
| | NFDYKKIESLISYKAKKEGVPVSNINPKYTSKTCAKCLLEVNQLSELKNEYNRDSKNS |
| | KIGICNIHGQIDADLNAARVIALCYSKNLNEPHFK |
| SEQ | VINLFGYKFALYPNKTQEELLNKHLGECGWLYNKAIEQNEYYKADSNIEEAQKKFELL |
| ID | PDKNSDEAKVLRGNISKDNYVYRTLVKKKKSEINVQIRKAVVLRPAETIRNLAKVKK |
| NO: | KGLSVGRLKFIPIREWDVLPFKQSDQIRLEENYLILEPYGRLKFKMHRPLLGKPKTFCIK |
| 72 | RTATDRWTISFSTEYDDSNMRKNDGGQVGIDVGLKTHLRLSNENPDEDPRYPNPKIW |
| | KRYDRRLTILQRRISKSKKLGKNRTRLRLRLSRLWEKIRNSRADLIQNETYEILSENKLI |
| | AIEDLNVKGMQEKKDKKGRKGRTRAQEKGLHRSISDAAFSEFRRVLEYKAKRFGSEV |
| | KPVSAIDSSKECHNCGNKKGMPLESRIYECPKCGLKIDRDLNSAKVILARATGVRPGS |
| | NARADTKISATAGASVQTEGTVSEDFRQQMETSDQKPMQGEGSKEPPMNPEHKSSGR |
| | GSKHVNIGCKNKVGLYNEDENSRSTEKQIMDENRSTTEDMVEIGALHSPVLTT |
| SEQ | MIASIDYEAVSQALIVFEFKAKGKDSQYQAIDEAIRSYRFIRNSCLRYWMDNKKVGKY |
| ID | DLNKYCKVLAKQYPFANKLNSQARQSAAECSWSAISRFYDNCKRKVSGKKGFPKFK |
| NO: | KHARSVEYKTSGWKLSENRKAITFTDKNGIGKLKLKGTYDLHFSQLEDMKRVRLVRR |
| 73 | ADGYYVQFCISVDVKVETEPTGKAIGLDVGIKYFLADSSGNTIENPQFYRKAEKKLNR |
| | ANRRKSKKYIRGVKPQSKNYHKARCRYARKHLRVSRQRKEYCKRVAYCVIHSNDVV |
| | AYEDLNVKGMVKNRHLAKSISDVAWSTFRHWLEYFAIKYGKLTIPVAPHNTSQNCSN |
| | CDKKVPKSLSTRTHICHHCGYSEDRDVNAAKNILKKALSTVGQTGSLKLGEIEPLLVL |
| | EQSCTRKFDL |
| SEQ | LAEENTLHLTLAMSLPLNDLPENRTRSELWRRQWLPQKKLSLLLGVNQSVRKAAADC |
| ID | LRWFEPYQELLWWEPTDPDGKKLLDKEGRPIKRTAGHMRVLRKLEEIAPFRGYQLGS |
| NO: | AVKNGLRHKVADLLLSYAKRKLDPQFTDKTSYPSIGDQFPIVWTGAFVCYEQSITGQL |
| 74 | YLYLPLFPRGSHQEDITNNYDPDRGPALQVFGEKEIARLSRSTSGLLLPLQFDKWGEAT |
| | FIRGENNPPTWKATHRRSDKKWLSEVLLREKDFQPKRVELLVRNGRIFVNVACEIPTK |
| | PLLEVENFMGVSFGLEHLVTVVVINRDGNVVHQRQEPARRYEKTYFARLERLRRRGG |
| | PFSQELETFHYRQVAQIVEEALRFKSVPAVEQVGNIPKGRYNPRLNLRLSYWPFGKLA |
| | DLTSYKAVKEGLPKPYSVYSATAKMLCSTCGAANKEGDQPISLKGPTVYCGNCGTRH |
| | NTGFNTALNLARRAQELFVKGVVAR |
| SEQ | MSQSLLKWHDMAGRDKDASRSLQKSAVEGVLLHLTASHRVALEMLEKSVSQTVAVT |
| ID | MEAAQQRLVIVLEDDPTKATSRKRVISADLQFTREEFGSLPNWAQKLASTCPEIATKY |
| NO: | ADKHINSIRIAWGVAKESTNGDAVEQKLQWQIRLLDVTMFLQQLVLQLADKALLEQI |
| 75 | PSSIRGGIGQEVAQQVTSHIQLLDSGTVLKAELPTISDRNSELARKQWEDAIQTVCTYA |
| | LPFSRERARILDPGKYAAEDPRGDRLINIDPMWARVLKGPTVKSLPLLFVSGSSIRIVKL |
| | TLPRKHAAGHKHTFTATYLVLPVSREWINSLPGTVQEKVQWWKKPDVLATQELLVG |
| | KGALKKSANTLVIPISAGKKRFFNHILPALQRGFPLQWQRIVGRSYRRPATHRKWFAQ |
| | LTIGYTNPSSLPEMALGIHFGMKDILWWALADKQGNILKDGSIPGNSILDFSLQEKGKI |
| | ERQQKAGKNVAGKKYGKSLLNATYRVVNGVLEFSKGISAEHASQPIGLGLETIRFVDK |
| | ASGSSPVNARHSNWNYGQLSGIFANKAGPAGFSVTEITLKKAQRDLSDAEQARVLAIE |
| | ATKRFASRIKRLATKRKDDTLFV |
| SEQ | VEPVEKERFYYRTYTFRLDGQPRTQNLTTQSGWGLLTKAVLDNTKHYWEIVHHARIA |
| ID | NQPIVFENPVIDEQGNPKLNKLGQPRFWKRPISDIVNQLRALFENQNPYQLGSSLIQGT |
| NO: | YWDVAENLASWYALNKEYLAGTATWGEPSFPEPHPLTEINQWMPLTFSSGKVVRLLK |
| 76 | NASGRYFIGLPILGENNPCYRMRTIEKLIPCDGKGRVTSGSLILFPLVGIYAQQHRRMTD |
| | ICESIRTEKGKLAWAQVSIDYVREVDKRRRMRRTRKSQGWIQGPWQEVFILRLVLAH |
| | KAPKLYKPRCFAGISLGPKTLASCVILDQDERVVEKQQWSGSELLSLIHQGEERLRSLR |
| | EQSKPTWNAAYRKQLKSLINTQVFTIVTFLRERGAAVRLESIARVRKSTPAPPVNFLLS |
| | HWAYRQITERLKDLAIRNGMPLTHSNGSYGVRFTCSQCGATNQGIKDPTKYKVDIESE |
| | TFLCSICSHREIAAVNTATNLAKQLLDE |
| SEQ | MNDTETSETLTSHRTVCAHLHVVGETGSLPRLVEAALAELITLNGRATQALLSLAKNG |
| ID | LVLRRDKEENLIAAELTLPCRKNKYADVAAKAGEPILATRINNKGKLVTKKWYGEGN |
| NO: | SYHIVRFTPETGMFTVRVFDRYAFDEELLHLHSEVVFGSDLPKGIKAKTDSLPANFLQA |
| 77 | VFTSFLELPFQGFPDIVVKPAMKQAAEQLLSYVQLEAGENQQAEYPDTNERDPELRLV |
| | EWQKSLHELSVRTEPFEFVRARDIDYYAETDRRGNRFVNITPEWTKFAESPFARRLPLK |
| | IPPEFCILLRRKTEGHAKIPNRIYLGLQIFDGVTPDSTLGVLATAEDGKLFWWHDHLDE |
| | FSNLEGKPEPKLKNKPQLLMVSLEYDREQRFEESVGGDRKICLVTLKETRNFRRGWN |
| | GRILGIHFQHNPVITWALMDHDAEVLEKGFIEGNAFLGKALDKQALNEYLQKGGKWV |
| | GDRSFGNKLKGITHTLASLIVRLAREKDAWIALEEISWVQKQSADSVANHEIVEQPHH |
| | SLTR |
| SEQ | MNDTETSETLTSHRTVCAHLHVVGETGSLPRLVEAALAELITLNGRATQALLSLAKNG |
| ID | LVLRRDKEENLIAAELTLPCRKNKYADVAAKAGEPILATRINNKGKLVTKKWYGEGN |
| NO: | SYHIVRFTPETGMFTVRVFDRYAFDEELLHLHSEVVFGSDLPKGIKAKTDSLPANFLQA |
| 78 | VFTSFLELPFQGFPDIVVKPAMKQAAEQLLSYVQLEAGENQQAEYPDTNERDPELRLV |
| | EWQKSLHELSVRTEPFEFVRARDIDYYAETDRRGNRFVNITPEWTKFAESPFARRLPLK |
| | IPPEFCILLRRKTEGHAKIPNRIYLGLQIFDGVTPDSTLGVLATAEDGKLFWWHDHLDE |
| | FSNLEGKPEPKLKNKPQLLMVSLEYDREQRFEESVGGDRKICLVTLKETRNFRRGRHG |
| | HTRTDRLPAGNTLWRADFATSAEVAAPKWNGRILGIHFQHNPVITWALMDHDAEVLE |
| | KGFIEGNAFLGKALDKQALNEYLQKGGKWVGDRSFGNKLKGITHTLASLIVRLAREK |
| | DAWIALEEISWVQKQSADSVANRRFSMWNYSRLATLIEWLGTDIATRDCGTAAPLAH |
| | KVSDYLTHFTCPECGACRKAGQKKEIADTVRAGDILTCRKCGFSGPIPDNFIAEFVAKK |
| | ALERMLKKKPV |
| SEQ | MAKRNFGEKSEALYRAVRFEVRPSKEELSILLAVSEVLRMLFNSALAERQQVFTEFIAS |
| ID | LYAELKSASVPEEISEIRKKLREAYKEHSISLFDQINALTARRVEDEAFASVTRNWQEE |
| NO: | TLDALDGAYKSFLSLRRKGDYDAHSPRSRDSGFFQKIPGRSGFKIGEGRIALSCGAGRK |
| 79 | LSFPIPDYQQGRLAETTKLKKFELYRDQPNLAKSGRFWISVVYELPKPEATTCQSEQVA |
| | FVALGASSIGVVSQRGEEVIALWRSDKHWVPKIEAVEERMKRRVKGSRGWLRLLNSG |
| | KRRMHMISSRQHVQDEREIVDYLVRNHGSHFVVTELVVRSKEGKLADSSKPERGGSL |
| | GLNWAAQNTGSLSRLVRQLEEKVKEHGGSVRKHKLTLTEAPPARGAENKLWMARKL |
| | RESFLKEV |
| SEQ | LAKNDEKELLYQSVKFEIYPDESKIRVLTRVSNILVLVWNSALGERRARFELYIAPLYE |
| ID | ELKKFPRKSAESNALRQKIREGYKEHIPTFFDQLKKLLTPMRKEDPALLGSVPRAYQEE |
| NO: | TLNTLNGSFVSFMTLRRNNDMDAKPPKGRAEDRFHEISGRSGFKIDGSEFVLSTKEQK |
| 80 | LRFPIPNYQLEKLKEAKQIKKFTLYQSRDRRFWISIAYEIELPDQRPFNPEEVIYIAFGAS |
| | SIGVISPEGEKVIDFWRPDKHWKPKIKEVENRMRSCKKGSRAWKKRAAARRKMYAM |
| | TQRQQKLNHREIVASLLRLGFHFVVTEYTVRSKPGKLADGSNPKRGGAPQGFNWSAQ |
| | NTGSFGEFILWLKQKVKEQGGTVQTFRLVLGQSERPEKRGRDNKIEMVRLLREKYLES |
| | QTIVV |
| SEQ | MAKGKKKEGKPLYRAVRFEIFPTSDQITLFLRVSKNLQQVWNEAWQERQSCYEQFFG |
| ID | SIYERIGQAKKRAQEAGFSEVWENEAKKGLNKKLRQQEISMQLVSEKESLLQELSIAF |
| NO: | QEHGVTLYDQINGLTARRIIGEFALIPRNWQEETLDSLDGSFKSFLALRKNGDPDAKPP |
| 81 | RQRVSENSFYKIPGRSGFKVSNGQIYLSFGKIGQTLTSVIPEFQLKRLETAIKLKKFELCR |
| | DERDMAKPGRFWISVAYEIPKPEKVPVVSKQITYLAIGASRLGVVSPKGEFCLNLPRSD |
| | YHWKPQINALQERLEGVVKGSRKWKKRMAACTRMFAKLGHQQKQHGQYEVVKKL |
| | LRHGVHFVVTELKVRSKPGALADASKSDRKGSPTGPNWSAQNTGNIARLIQKLTDKA |
| | SEHGGTVIKRNPPLLSLEERQLPDAQRKIFIAKKLREEFLADQK |
| SEQ | MAKREKKDDVVLRGTKMRIYPTDRQVTLMDMWRRRCISLWNLLLNLETAAYGAKN |
| ID | TRSKLGWRSIWARVVEENHAKALIVYQHGKCKKDGSFVLKRDGTVKHPPRERFPGDR |
| NO: | KILLGLFDALRHTLDKGAKCKCNVNQPYALTRAWLDETGHGARTADIIAWLKDFKGE |
| 82 | CDCTAISTAAKYCPAPPTAELLTKIKRAAPADDLPVDQAILLDLFGALRGGLKQKECD |
| | HTHARTVAYFEKHELAGRAEDILAWLIAHGGTCDCKIVEEAANHCPGPRLFIWEHELA |
| | MIMARLKAEPRTEWIGDLPSHAAQTVVKDLVKALQTMLKERAKAAAGDESARKTGF |
| | PKFKKQAYAAGSVYFPNTTMFFDVAAGRVQLPNGCGSMRCEIPRQLVAELLERNLKP |
| | GLVIGAQLGLLGGRIWRQGDRWYLSCQWERPQPTLLPKTGRTAGVKIAASIVFTTYD |
| | NRGQTKEYPMPPADKKLTAVHLVAGKQNSRALEAQKEKEKKLKARKERLRLGKLEK |
| | GHDPNALKPLKRPRVRRSKLFYKSAARLAACEAIERDRRDGFLHRVTNEIVHKFDAVS |
| | VQKMSVAPMMRRQKQKEKQIESKKNEAKKEDNGAAKKPRNLKPVRKLLRHVAMAR |
| | GRQFLEYKYNDLRGPGSVLIADRLEPEVQECSRCGTKNPQMKDGRRLLRCIGVLPDGT |
| | DCDAVLPRNRNAARNAEKRLRKHREAHNA |
| SEQ | MNEVLPIPAVGEDAADTIMRGSKMRIYPSVRQAATMDLWRRRCIQLWNLLLELEQAA |
| ID | YSGENRRTQIGWRSIWATVVEDSHAEAVRVAREGKKRKDGTFRKAPSGKEIPPLDPA |
| NO: | MLAKIQRQMNGAVDVDPKTGEVTPAQPRLFMWEHELQKIMARLKQAPRTHWIDDLP |
| 83 | SHAAQSVVKDLIKALQAMLRERKKRASGIGGRDTGFPKFKKNRYAAGSVYFANTQLR |
| | FEAKRGKAGDPDAVRGEFARVKLPNGVGWMECRMPRHINAAHAYAQATLMGGRIW |
| | RQGENWYLSCQWKMPKPAPLPRAGRTAAIKIAAAIPITTVDNRGQTREYAMPPIDRER |
| | IAAHAAAGRAQSRALEARKRRAKKREAYAKKRHAKKLERGIAAKPPGRARIKLSPGF |
| | YAAAAKLAKLEAEDANAREAWLHEITTQIVRNFDVIAVPRMEVAKLMKKPEPPEEKE |
| | EQVKAPWQGKRRSLKAARVMMRRTAMALIQTTLKYKAVDLRGPQAYEEIAPLDVTA |
| | AACSGCGVLKPEWKMARAKGREIMRCQEPLPGGKTCNTVLTYTRNSARVIGRELAVR |
| | LAERQKA |
| SEQ | MTTQKTYNFCFYDQRFFELSKEAGEVYSRSLEEFWKIYDETGVWLSKFDLQKHMRNK |
| ID | LERKLLHSDSFLGAMQQVHANLASWKQAKKVVPDACPPRKPKFLQAILFKKSQIKYK |
| NO: | NGFLRLTLGTEKEFLYLKWDINIPLPIYGSVTYSKTRGWKINLCLETEVEQKNLSENKY |
| 84 | LSIDLGVKRVATIFDGENTITLSGKKFMGLMHYRNKLNGKTQSRLSHKKKGSNNYKKI |
| | QRAKRKTTDRLLNIQKEMLHKYSSFIVNYAIRNDIGNIIIGDNSSTHDSPNMRGKTNQK |
| | ISQNPEQKLKNYIKYKFESISGRVDIVPEPYTSRKCPHCKNIKKSSPKGRTYKCKKCGFI |
| | FDRDGVGAINIYNENVSFGQIISPGRIRSLTEPIGMKFHNEIYFKSYVAA |
| SEQ | MSVRSFQARVECDKQTMEHLWRTHKVFNERLPEIIKILFKMKRGECGQNDKQKSLYK |
| ID | SISQSILEANAQNADYLLNSVSIKGWKPGTAKKYRNASFTWADDAAKLSSQGIHVYD |
| NO: | KKQVLGDLPGMMSQMVCRQSVEAISGHIELTKKWEKEHNEWLKEKEKWESEDEHK |
| 85 | KYLDLREKFEQFEQSIGGKITKRRGRWHLYLKWLSDNPDFAAWRGNKAVINPLSEKA |
| | QIRINKAKPNKKNSVERDEFFKANPEMKALDNLHGYYERNFVRRRKTKKNPDGFDHK |
| | PTFTLPHPTIHPRWFVFNKPKTNPEGYRKLILPKKAGDLGSLEMRLLTGEKNKGNYPD |
| | DWISVKFKADPRLSLIRPVKGRRVVRKGKEQGQTKETDSYEFFDKHLKKWRPAKLSG |
| | VKLIFPDKTPKAAYLYFTCDIPDEPLTETAKKIQWLETGDVTKKGKKRKKKVLPHGLV |
| | SCAVDLSMRRGTTGFATLCRYENGKIHILRSRNLWVGYKEGKGCHPYRWTEGPDLGH |
| | IAKHKREIRILRSKRGKPVKGEESHIDLQKHIDYMGEDRFKKAARTIVNFALNTENAAS |
| | KNGFYPRADVLLLENLEGLIPDAEKERGINRALAGWNRRHLVERVIEMAKDAGFKRR |
| | VFEIPPYGTSQVCSKCGALGRRYSIIRENNRREIRFGYVEKLFACPNCGYCANADHNAS |
| | VNLNRRFLIEDSFKSYYDWKRLSEKKQKEEIETIESKLMDKLCAMHKISRGSISK |
| SEQ | MHLWRTHCVFNQRLPALLKRLFAMRRGEVGGNEAQRQVYQRVAQFVLARDAKDSV |
| ID | DLLNAVSLRKRSANSAFKKKATISCNGQAREVTGEEVFAEAVALASKGVFAYDKDD |
| NO: | MRAGLPDSLFQPLTRDAVACMRSHEELVATWKKEYREWRDRKSEWEAEPEHALYLN |
| 86 | LRPKFEEGEAARGGRFRKRAERDHAYLDWLEANPQLAAWRRKAPPAVVPIDEAGKR |
| | RIARAKAWKQASVRAEEFWKRNPELHALHKIHVQYLREFVRPRRTRRNKRREGFKQR |
| | PTFTMPDPVRHPRWCLFNAPQTSPQGYRLLRLPQSRRTVGSVELRLLTGPSDGAGFPD |
| | AWVNVRFKADPRLAQLRPVKVPRTVTRGKNKGAKVEADGFRYYDDQLLIERDAQVS |
| | GVKLLFRDIRMAPFADKPIEDRLLSATPYLVFAVEIKDEARTERAKAIRFDETSELTKSG |
| | KKRKTLPAGLVSVAVDLDTRGVGFLTRAVIGVPEIQQTHHGVRLLQSRYVAVGQVEA |
| | RASGEAEWSPGPDLAHIARHKREIRRLRQLRGKPVKGERSHVRLQAHIDRMGEDRFK |
| | KAARKIVNEALRGSNPAAGDPYTRADVLLYESLETLLPDAERERGINRALLRWNRAK |
| | LIEHLKRMCDDAGIRHFPVSPFGTSQVCSKCGALGRRYSLARENGRAVIRFGWVERLF |
| | ACPNPECPGRRPDRPDRPFTCNSDHNASVNLHRVFALGDQAVAAFRALAPRDSPARTL |
| | AVKRVEDTLRPQLMRVHKLADAGVDSPF |
| SEQ | MATLVYRYGVRAHGSARQQDAVVSDPAMLEQLRLGHELRNALVGVQHRYEDGKRA |
| ID | VWSGFASVAAADHRVTTGETAVAELEKQARAEHSADRTAATRQGTAESLKAARAAV |
| NO: | KQARADRKAAMAAVAEQAKPKIQALGDDRDAEIKDLYRRFCQDGVLLPRCGRCAGD |
| 87 | LRSDGDCTDCGAAHEPRKLYWATYNAIREDHQTAVKLVEAKRKAGQPARLRFRRWT |
| | GDGTLTVQLQRMHGPACRCVTCAEKLTRRARKTDPQAPAVAADPAYPPTDPPRDPAL |
| | LASGQGKWRNVLQLGTWIPPGEWSAMSRAERRRVGRSHIGWQLGGGRQLTLPVQLH |
| | RQMPADADVAMAQLTRVRVGGRHRMSVALTAKLPDPPQVQGLPPVALHLGWRQRP |
| | DGSLRVATWACPQPLDLPPAVADVVVSHGGRWGEVIMPARWLADAEVPPRLLGRRD |
| | KAMEPVLEALADWLEAHTEACTARMTPALVRRWRSQGRLAGLTNRWRGQPPTGSA |
| | EILTYLEAWRIQDKLLWERESHLRRRLAARRDDAWRRVASWLARHAGVLVVDDADI |
| | AELRRRDDPADTDPTMPASAAQAARARAALAAPGRLRHLATITATRDGLGVHTVASA |
| | GLTRLHRKCGHQAQPDPRYAASAVVTCPGCGNGYDQDYNAAMLMLDRQQQP |
| SEQ | MSRVELHRAYKFRLYPTPAQVAELAEWERQLRRLYNLAHSQRLAAMQRHVRPKSPG |
| ID | VLKSECLSCGAVAVAEIGTDGKAKKTVKHAVGCSVLECRSCGGSPDAEGRTAHTAAC |
| NO: | SFVDYYRQGREMTQLLEEDDQLARVVCSARQETLRDLEKAWQRWHKMPGFGKPHF |
| 88 | KKRIDSCRIYFSTPKSWAVDLGYLSFTGVASSVGRIKIRQDRVWPGDAKFSSCHVVRD |
| | VDEWYAVFPLTFTKEIEKPKGGAVGINRGAVHAIADSTGRVVDSPKFYARSLGVIRHR |
| | ARLLDRKVPFGRAVKPSPTKYHGLPKADIDAAAARVNASPGRLVYEARARGSIAAAE |
| | AHLAALVLPAPRQTSQLPSEGRNRERARRFLALAHQRVRRQREWFLHNESAHYAQSY |
| | TKIAIEDWSTKEMTSSEPRDAEEMKRVTRARNRSILDVGWYELGRQIAYKSEATGAEF |
| | AKVDPGLRETETHVPEAIVRERDVDVSGMLRGEAGISGTCSRCGGLLRASASGHADA |
| | ECEVCLHVEVGDVNAAVNVLKRAMFPGAAPPSKEKAKVTIGIKGRKKKRAA |
| SEQ | MSRVELHRAYKFRLYPTPVQVAELSEWERQLRRLYNLGHEQRLLTLTRHLRPKSPGV |
| ID | LKGECLSCDSTQVQEVGADGRPKTTVRHAEQCPTLACRSCGALRDAEGRTAHTVACA |
| NO: | FVDYYRQGREMTELLAADDQLARVVCSARQEVLRDLDKAWQRWRKMPGFGKPRFK |
| 89 | RRTDSCRIYFSTPKAWKLEGGHLSFTGAATTVGAIKMRQDRNWPASVQFSSCHVVRD |
| | VDEWYAVFPLTFVAEVARPKGGAVGINRGAVHAIADSTGRVVDSPRYYARALGVIRH |
| | RARLFDRKVPSGHAVKPSPTKYRGLSAIEVDRVARATGFTPGRVVTEALNRGGVAYA |
| | ECALAAIAVLGHGPERPLTSDGRNREKARKFLALAHQRVRRQREWFLHNESAHYART |
| | YSKIAIEDWSTKEMTASEPQGEETRRVTRSRNRSILDVGWYELGRQLAYKTEATGAEF |
| | AQVDPGLKETETNVPKAIADARDVDVSGMLRGEAGISGTCSKCGGLLRAPASGHADA |
| | ECEICLNVEVGDVNAAVNVLKRAMFPGDAPPASGEKPKVSIGIKGRQKKKKAA |
| SEQ | MEAIATGMSPERRVELGILPGSVELKRAYKFRLYPMKVQQAELSEWERQLRRLYNLA |
| ID | HEQRLAALLRYRDWDFQKGACPSCRVAVPGVHTAACDHVDYFRQAREMTQLLEVD |
| NO: | AQLSRVICCARQEVLRDLDKAWQRWRKKLGGRPRFKRRTDSCRIYLSTPKHWEIAGR |
| 90 | YLRLSGLASSVGEIRIEQDRAFPEGALLSSCSIVRDVDEWYACLPLTFTQPIERAPHRSV |
| | GLNRGWHALADSDGRVVDSPKFFERALATVQKRSRDLARKVSGSRNAHKARIKLA |
| | KAHQRVRRQRAAFLHQESAYYSKGFDLVALEDMSVRKMTATAGEAPEMGRGAQRD |
| | LNRGILDVGWYELARQIDYKRLAHGGELLRVDPGQTTPLACVTEEQPARGISSACAVC |
| | GIPLARPASGNARMRCTACGSSQVGDVNAAENVLTRALSSAPSGPKSPKASIKIKGRQ |
| | KRLGTPANRAGEASGGDPPVRGPVEGGTLAYVVEPVSESQSDT |
| SEQ | MTVRTYKYRAYPTPEQAEALTSWLRFASQLYNAALEHRKNAWGRHDAHGRGFRFW |
| ID | DGDAAPRKKSDPPGRWVYRGGGGAHISKNDQGKLLTEFRREHAELLPPGMPALVQH |
| NO: | EVLARLERSMAAFFQRATKGQKAGYPRWRSEHRYDSLTFGLTSPSKERFDPETGESLG |
| 91 | RGKTVGAGTYHNGDLRLTGLGELRILEHRRIPMGAIPKSVIVRRSGKRWFVSIAMEMP |
| | SVEPAASGRPAVGLDMGVVTWGTAFTADTSAAAALVADLRRMATDPSDCRRLEELE |
| | REAAQLSEVLAHCRARGLDPARPRRCPKELTKLYRRSLHRLGELDRACARIRRRLQAA |
| | HDIAEPVPDEAGSAVLIEGSNAGMRHARRVARTQRRVARRTRAGHAHSNRRKKAVQ |
| | AYARAKERERSARGDHRHKVSRALVRQFEEISVEALDIKQLTVAPEHNPDPQPDLPAH |
| | VQRRRNRGELDAAWGAFFAALDYKAADAGGRVARKPAPHTTQECARCGTLVPKPIS |
| | LRVHRCPACGYTAPRTVNSARNVLQRPLEEPGRAGPSGANGRGVPHAVA |
| SEQ | MNCRYRYRIYPTPGQRQSLARLFGCVRVVWNDALFLCRQSEKLPKNSELQKLCITQA |
| ID | KKTEARGWLGQVSAIPLQQSVADLGVAFKNFFQSRSGKRKGKKVNPPRVKRRNNRQ |
| NO: | GARFTRGGFKVKTSKVYLARIGDIKIKWSRPLPSEPSSVTVIKDCAGQYFLSFVVEVKP |
| 92 | EIKPPKNPSIGIDLGLKTFASCSNGEKIDSPDYSRLYRKLKRCQRRLAKRQRGSKRRER |
| | MRVKVAKLNAQIRDKRKDFLHKLSTKVVNENQVIALEDLNVGGMLKNRKLSRAISQ |
| | AGWYEFRSLCEGKAEKHNRDFRVISRWEPTSQVCSECGYRWGKIDLSVRSIVCINCGV |
| | EHDRDDNASVNIEQAGLKVGVGHTHDSKRTGSACKTSNGAVCVEPSTHREYVQLTLF |
| | DW |
| SEQ | MKSRWTFRCYPTPEQEQHLARTFGCVRFVWNWALRARTDAFRAGERIGYPATDKAL |
| ID | TLLKQQPETVWLNEVSSVCLQQALRDLQVAFSNFFDKRAAHPSFKRKEARQSANYTE |
| NO: | RGFSFDHERRILKLAKIGAIKVKWSRKAIPHPSSIRLIRTASGKYFVSLVVETQPAPMPE |
| 93 | TGESVGVDFGVARLATLSNGERISNPKHGAKWQRRLAFYQKRLARATKGSKRRMRIK |
| | RHVARIHEKIGNSRSDTLHKLSTDLVTRFDLICVEDLNLRGMVKNHSLARSLHDASIGS |
| | AIRMIEEKAERYGKNVVKIDRWFPSSKTCSDCGHIVEQLPLNVREWTCPECGTTHDRD |
| | ANAAANILAVGQTVSAHGGTVRRSRAKASERKSQRSANRQGVNRA |
| SEQ | KEPLNIGKTAKAVFKEIDPTSLNRAANYDASIELNCKECKFKPFKNVKRYEFNFYNNW |
| ID | YRCNPNSCLQSTYKAQVRKVEIGYEKLKNEILTQMQYYPWFGRLYQNFFHDERDKM |
| NO: | TSLDEIQVIGVQNKVFFNTVEKAWREIIKKRFKDNKETMETIPELKHAAGHGKRKLSN |
| 94 | KSLLRRRFAFVQKSFKFVDNSDVSYRSFSNNIACVLPSRIGVDLGGVISRNPKREYIPQE |
| | ISFNAFWKQHEGLKKGRNIEIQSVQYKGETVKRIEADTGEDKAWGKNRQRRFTSLILK |
| | LVPKQGGKKVWKYPEKRNEGNYEYFPIPIEFILDSGETSIRFGGDEGEAGKQKHLVIPF |
| | NDSKATPLASQQTLLENSRFNAEVKSCIGLAIYANYFYGYARNYVISSIYHKNSKNGQ |
| | AITAIYLESIAHNYVKAIERQLQNLLLNLRDFSFMESHKKELKKYFGGDLEGTGGAQK |
| | RREKEEKIEKEIEQSYLPRLIRLSLTKMVTKQVEM |
| SEQ | ELIVNENKDPLNIGKTAKAVFKEIDPTSINRAANYDASIELACKECKFKPFNNTKRHDF |
| ID | SFYSNWHRCSPNSCLQSTYRAKIRKTEIGYEKLKNEILNQMQYYPWFGRLYQNFFNDQ |
| NO: | RDKMTSLDEIQVTGVQNKIFFNTVEKAWREIIKKRFRDNKETMRTIPDLKNKSGHGSR |
| 95 | KLSNKSLLRRRFAFAQKSFKLVDNSDVSYRAFSNNVACVLPSKIGVDIGGIINKDLKRE |
| | YIPQEITFNVFWKQHDGLKKGRNIEIHSVQYKGEIVKRIEADTGEDKAWGKNRQRRFT |
| | SLILKITPKQGGKKIWKFPEKKNASDYEYFPIPIEFILDNGDASIKFGGEEGEVGKQKHL |
| | LIPFNDSKATPLSSKQMLLETSRFNAEVKSTIGLALYANYFVSYARNYVIKSTYHKNSK |
| | KGQIVTEIYLESISQNFVRAIQRQLQSLMLNLKDWGFMQTHKKELKKYFGSDLEGSKG |
| | GQKRREKEEKIEKEIEASYLPRLIRLSLTKSVTKAEEM |
| SEQ | PEEKTSKLKPNSINLAANYDANEKFNCKECKFHPFKNKKRYEFNFYNNLHGCKSCTKS |
| ID | TNNPAVKRIEIGYQKLKFEIKNQMEAYPWFGRLRINFYSDEKRKMSELNEMQVTGVK |
| NO: | NKIFFDAIECAWREILKKRFRESKETLITIPKLKNKAGHGARKHRNKKLLIRRRAFMKK |
| 96 | NFHFLDNDSISYRSFANNIACVLPSKVGVDIGGIISPDVGKDIKPVDISLNLMWASKEGI |
| | KSGRKVEIYSTQYDGNMVKKIEAETGEDKSWGKNRKRRQTSLLLSIPKPSKQVQEFDF |
| | KEWPRYKDIEKKVQWRGFPIKIIFDSNHNSIEFGTYQGGKQKVLPIPFNDSKTTPLGSK |
| | MNKLEKLRFNSKIKSRLGSAIAANKFLEAARTYCVDSLYHEVSSANAIGKGKIFIEYYL |
| | EILSQNYIEAAQKQLQRFIESIEQWFVADPFQGRLKQYFKDDLKRAKCFLCANREVQT |
| | TCYAAVKLHKSCAEKVKDKNKELAIKERNNKEDAVIKEVEASNYPRVIRLKLTKTITN |
| | KAM |
| SEQ | SESENKIIEQYYAFLYSFRDKYEKPEFKNRGDIKRKLQNKWEDFLKEQNLKNDKKLSN |
| ID | YIFSNRNFRRSYDREEENEEGIDEKKSKPKRINCFEKEKNLKDQYDKDAINASANKDG |
| NO: | AQKWGCFECIFFPMYKIESGDPNKRIIINKTRFKLFDFYLNLKGCKSCLRSTYHPYRSN |
| 97 | VYIESNYDKLKREIGNFLQQKNIFQRMRKAKVSEGKYLTNLDEYRLSCVAMHFKNRW |
| | LFFDSIQKVLRETIKQRLKQMRESYDEQAKTKRSKGHGRAKYEDQVRMIRRRAYSAQ |
| | AHKLLDNGYITLFDYDDKEINKVCLTAINQEGFDIGGYLNSDIDNVMPPIEISFHLKWK |
| | YNEPILNIESPFSKAKISDYLRKIREDLNLERGKEGKARSKKNVRRKVLASKGEDGYKK |
| | IFTDFFSKWKEELEGNAMERVLSQSSGDIQWSKKKRIHYTTLVLNINLLDKKGVGNLK |
| | YYEIAEKTKILSFDKNENKFWPITIQVLLDGYEIGTEYDEIKQLNEKTSKQFTIYDPNTKI |
| | IKIPFTDSKAVPLGMLGINIATLKTVKKTERDIKVSKIFKGGLNSKIVSKIGKGIYAGYFP |
| | TVDKEILEEVEEDTLDNEFSSKSQRNIFLKSIIKNYDKMLKEQLFDFYSFLVRNDLGVRF |
| | LTDRELQNIEDESFNLEKRFFETDRDRIARWFDNTNTDDGKEKFKKLANEIVDSYKPR |
| | LIRLPVVRVIKRIQPVKQREM |
| SEQ | KYSTRDFSELNEIQVTACKQDEFFKVIQNAWREIIKKRFLENRENFIEKKIFKNKKGRG |
| ID | KRQESDKTIQRNRASVMKNFQLIENEKIILRAPSGHVACVFPVKVGLDIGGFKTDDLEK |
| NO: | NIFPPRTITINVFWKNRDRQRKGRKLEVWGIKARTKLIEKVHKWDKLEEVKKKRLKSL |
| 98 | EQKQEKSLDNWSEVNNDSFYKVQIDELQEKIDKSLKGRTMNKILDNKAKESKEAEGL |
| | YIEWEKDFEGEMLRRIEASTGGEEKWGKRRQRRHTSLLLDIKNNSRGSKEIINFYSYAK |
| | QGKKEKKIEFFPFPLTITLDAEEESPLNIKSIPIEDKNATSKYFSIPFTETRATPLSILGDRV |
| | QKFKTKNISGAIKRNLGSSISSCKIVQNAETSAKSILSLPNVKEDNNMEIFINTMSKNYF |
| | RAMMKQMESFIFEMEPKTLIDPYKEKAIKWFEVAASSRAKRKLKKLSKADIKKSELLL |
| | SNTEEFEKEKQEKLEALEKEIEEFYLPRIVRLQLTKTILETPVM |
| SEQ | KKLQLLGHKILLKEYDPNAVNAAANFETSTAELCGQCKMKPFKNKRRFQYTFGKNY |
| ID | HGCLSCIQNVYYAKKRIVQIAKEELKHQLTDSIASIPYKYTSLFSNTNSIDELYILKQER |
| NO: | AAFFSNTNSIDELYITGIENNIAFKVISAIWDEIIKKRRQRYAESLTDTGTVKANRGHGG |
| 99 | TAYKSNTRQEKIRALQKQTLHMVTNPYISLARYKNNYIVATLPRTIGMHIGAIKDRDP |
| | QKKLSDYAINFNVFWSDDRQLIELSTVQYTGDMVRKIEAETGENNKWGENMKRTKTS |
| | LLLEILTKKTTDELTFKDWAFSTKKEIDSVTKKTYQGFPIGIIFEGNESSVKFGSQNYFPL |
| | PFDAKITPPTAEGFRLDWLRKGSFSSQMKTSYGLAIYSNKVTNAIPAYVIKNMFYKIAR |
| | AENGKQIKAKFLKKYLDIAGNNYVPFIIMQHYRVLDTFEEMPISQPKVIRLSLTKTQHII |
| | IKKDKTDSKM |
| SEQ | NTSNLINLGKKAINISANYDANLEVGCKNCKFLSSNGNFPRQTNVKEGCHSCEKSTYE |
| ID | PSIYLVKIGERKAKYDVLDSLKKFTFQSLKYQSKKSMKSRNKKPKELKEFVIFANKNK |
| NO: | AFDVIQKSYNHLILQIKKEINRMNSKKRKKNHKRRLFRDREKQLNKLRLIESSNLFLPR |
| 100 | ENKGNNHVFTYVAIHSVGRDIGVIGSYDEKLNFETELTYQLYFNDDKRLLYAYKPKQ |
| | NKIIKIKEKLWNLRKEKEPLDLEYEKPLNKSITFSIKNDNLFKVSKDLMLRRAKFNIQG |
| | KEKLSKEERKINRDLIKIKGLVNSMSYGRFDELKKEKNIWSPHIYREVRQKEIKPCLIKN |
| | GDRIEIFEQLKKKMERLRRFREKRQKKISKDLIFAERIAYNFHTKSIKNTSNKINIDQEA |
| | KRGKASYMRKRIGYETFKNKYCEQCLSKGNVYRNVQKGCSCFENPFDWIKKGDENL |
| | LPKKNEDLRVKGAFRDEALEKQIVKIAFNIAKGYEDFYDNLGESTEKDLKLKFKVGTT |
| | INEQESLKL |
| SEQ | TSNPIKLGKKAINISANYDSNLQIGCKNCKFLSYNGNFPRQTNVKEGCHSCEKSTYEPP |
| ID | VYTVRIGERRSKYDVLDSLKKFIFLSLKYRQSKKMKTRSKGIRGLEEFVISANLKKAM |
| NO: | DVIQKSYRHLILNIKNEIVRMNGKKRNKNHKRLLFRDREKQLNKLRLIEGSSFFKPPTV |
| 101 | KGDNSIFTCVAIHNIGRDIGIAGDYFDKLEPKIELTYQLYYEYNPKKESEINKRLLYAYK |
| | PKQNKIIEIKEKLWNLRKEKSPLDLEYEKPLTKSITFLVKRDGVFRISKDLMLRKAKFII |
| | QGKEKLSKEERKINRDLIKIKSNIISLTYGRFDELKKDKTIWSPHIFRDVKQGKITPCIER |
| | KGDRMDIFQQLRKKSERLRENRKKRQKKISKDLIFAERIAYNFHTKSIKNTSNLINIKHE |
| | AKRGKASYMRKRIGNETFRIKYCEQCFPKNNVYKNVQKGCSCFEDPFEYIKKGNEDLI |
| | PNKNQDLKAKGAFRDDALEKQIIKVAFNIAKGYEDFYENLKKTTEKDIRLKFKVGTIIS |
| | EEM |
| SEQ | NNSINLSKKAINISANYDANLQVRCKNCKFLSSNGNFPRQTDVKEGCHSCEKSTYEPP |
| ID | VYDVKIGEIKAKYEVLDSLKKFTFQSLKYQLSKSMKFRSKKIKELKEFVIFAKESKALN |
| NO: | VINRSYKHLILNIKNDINRMNSKKRIKNHKGRLFLDRQKQLSKLKLIEGSSFFVPAKNV |
| 102 | GNKSVFTCVAIHSIGRDIGIAGLYDSFTKPVNEITYQIFFSGERRLLYAYKPKQLKILSIK |
| | ENLWSLKNEKKPLDLLYEKPLGKNLNFNVKGGDLFRVSKDLMIRNAKFNVHGRQRLS |
| | DEERLINRNFIKIKGEVVSLSYGRFEELKKDRKLWSPHIFKDVRQNKIKPCLVMQGQRI |
| | DIFEQLKRKLELLKKIRKSRQKKLSKDLIFGERIAYNFHTKSIKNTSNKINIDSDAKRGR |
| | ASYMRKRIGNETFKLKYCDVCFPKANVYRRVQNGCSCSENPYNYIKKGDKDLLPKKD |
| | EGLAIKGAFRDEKLNKQIIKVAFNIAKGYEDFYDDLKKRTEKDVDLKFKIGTTVLDQK |
| | PMEIFDGIVITWL |
| SEQ | LLTTVVETNNLAKKAINVAANFDANIDRQYYRCTPNLCRFIAQSPRETKEKDAGCSSC |
| ID | TQSTYDPKVYVIKIGKLLAKYEILKSLKRFLFMNRYFKQKKTERAQQKQKIGTELNEM |
| NO: | SIFAKATNAMEVIKRATKHCTYDIIPETKSLQMLKRRRHRVKVRSLLKILKERRMKIKK |
| 103 | IPNTFIEIPKQAKKNKSDYYVAAALKSCGIDVGLCGAYEKNAEVEAEYTYQLYYEYKG |
| | NSSTKRILYCYNNPQKNIREFWEAFYIQGSKSHVNTPGTIRLKMEKFLSPITIESEALDF |
| | RVWNSDLKIRNGQYGFIKKRSLGKEAREIKKGMGDIKRKIGNLTYGKSPSELKSIHVY |
| | RTERENPKKPRAARKKEDNFMEIFEMQRKKDYEVNKKRRKEATDAAKIMDFAEEPIR |
| | HYHTNNLKAVRRIDMNEQVERKKTSVFLKRIMQNGYRGNYCRKCIKAPEGSNRDEN |
| | VLEKNEGCLDCIGSEFIWKKSSKEKKGLWHTNRLLRRIRLQCFTTAKAYENFYNDLFE |
| | KKESSLDIIKLKVSITTKSM |
| SEQ | ASTMNLAKQAINFAANYDSNLEIGCKGCKFMSTWSKKSNPKFYPRQNNQANKCHSCT |
| ID | YSTGEPEVPIIEIGERAAKYKIFTALKKFVFMSVAYKERRRQRFKSKKPKELKELAICSN |
| NO: | REKAMEVIQKSVVHCYGDVKQEIPRIRKIKVLKNHKGRLFYKQKRSKIKIAKLEKGSFF |
| 104 | KTFIPKVHNNGCHSCHEASLNKPILVTTALNTIGADIGLINDYSTIAPTETDISWQVYYE |
| | FIPNGDSEAVKKRLLYFYKPKGALIKSIRDKYFKKGHENAVNTGFFKYQGKIVKGPIKF |
| | VNNELDFARKPDLKSMKIKRAGFAIPSAKRLSKEDREINRESIKIKNKIYSLSYGRKKTL |
| | SDKDIIKHLYRPVRQKGVKPLEYRKAPDGFLEFFYSLKRKERRLRKQKEKRQKDMSEII |
| | DAADEFAWHRHTGSIKKTTNHINFKSEVKRGKVPIMKKRIANDSFNTRHCGKCVKQG |
| | NAINKYYIEKQKNCFDCNSIEFKWEKAALEKKGAFKLNKRLQYIVKACFNVAKAYES |
| | FYEDFRKGEEESLDLKFKIGTTTTLKQYPQNKARAM |
| SEQ | HSHNLMLTKLGKQAINFAANYDANLEIGCKNCKFLSYSPKQANPKKYPRQTDVHEDG |
| ID | NIACHSCMQSTKEPPVYIVPIGERKSKYEILTSLNKFTFLALKYKEKKRQAFRAKKPKE |
| NO: | LQELAIAFNKEKAIKVIDKSIQHLILNIKPEIARIQRQKRLKNRKGKLLYLHKRYAIKMG |
| 105 | LIKNGKYFKVGSPKKDGKKLLVLCALNTIGRDIGIIGNIEENNRSETEITYQLYFDCLDA |
| | NPNELRIKEIEYNRLKSYERKIKRLVYAYKPKQTKILEIRSKFFSKGHENKVNTGSFNFE |
| | NPLNKSISIKVKNSAFDFKIGAPFIMLRNGKFHIPTKKRLSKEEREINRTLSKIKGRVFRL |
| | TYGRNISEQGSKSLHIYRKERQHPKLSLEIRKQPDSFIDEFEKLRLKQNFISKLKKQRQK |
| | KLADLLQFADRIAYNYHTSSLEKTSNFINYKPEVKRGRTSYIKKRIGNEGFEKLYCETCI |
| | KSNDKENAYAVEKEELCFVCKAKPFTWKKTNKDKLGIFKYPSRIKDFIRAAFTVAKSY |
| | NDFYENLKKKDLKNEIFLKFKIGLILSHEKKNHISIAKSVAEDERISGKSIKNILNKSIKL |
| | EKNCYSCFFHKEDM |
| SEQ | SLERVIDKRNLAKKAINIAANFDANINKGFYRCETNQCMFIAQKPRKTNNTGCSSCLQS |
| ID | TYDPVIYVVKVGEMLAKYEILKSLKRFVFMNRSFKQKKTEKAKQKERIGGELNEMSIF |
| NO: | ANAALAMGVIKRAIRHCHVDIRPEINRLSELKKTKHRVAAKSLVKIVKQRKTKWKGIP |
| 106 | NSFIQIPQKARNKDADFYVASALKSGGIDIGLCGTYDKKPHADPRWTYQLYFDTEDES |
| | EKRLLYCYNDPQAKIRDFWKTFYERGNPSMVNSPGTIEFRMEGFFEKMTPISIESKDFD |
| | FRVWNKDLLIRRGLYEIKKRKNLNRKAREIKKAMGSVKRVLANMTYGKSPTDKKSIP |
| | VYRVEREKPKKPRAVRKEENELADKLENYRREDFLIRNRRKREATEIAKIIDAAEPPIR |
| | HYHTNHLRAVKRIDLSKPVARKNTSVFLKRIMQNGYRGNYCKKCIKGNIDPNKDECR |
| | LEDIKKCICCEGTQNIWAKKEKLYTGRINVLNKRIKQMKLECFNVAKAYENFYDNLA |
| | ALKEGDLKVLKLKVSIPALNPEASDPEEDM |
| SEQ | NASINLGKRAINLSANYDSNLVIGCKNCKFLSFNGNFPRQTNVREGCHSCDKSTYAPE |
| ID | VYIVKIGERKAKYDVLDSLKKFTFQSLKYQIKKSMRERSKKPKELLEFVIFANKDKAF |
| NO: | NVIQKSYEHLILNIKQEINRMNGKKRIKNHKKRLFKDREKQLNKLRLIGSSSLFFPREN |
| 107 | KGDKDLFTYVAIHSVGRDIGVAGSYESHIEPISDLTYQLFINNEKRLLYAYKPKQNKIIE |
| | LKENLWNLKKEKKPLDLEFTKPLEKSITFSVKNDKLFKVSKDLMLRQAKFNIQGKEKL |
| | SKEERQINRDFSKIKSNVISLSYGRFEELKKEKNIWSPHIYREVKQKEIKPCIVRKGDRIE |
| | LFEQLKRKMDKLKKFRKERQKKISKDLNFAERIAYNFHTKSIKNTSNKINIDQEAKRG |
| | KASYMRKRIGNESFRKKYCEQCFSVGNVYHNVQNGCSCFDNPIELIKKGDEGLIPKGK |
| | EDRKYKGALRDDNLQMQIIRVAFNIAKGYEDFYNNLKEKTEKDLKLKFKIGTTISTQE |
| | SNNKEM |
| SEQ | SNLIKLGKQAINFAANYDANLEVGCKNCKFLSSTNKYPRQTNVHLDNKMACRSCNQS |
| ID | TMEPAIYIVRIGEKKAKYDIYNSLTKFNFQSLKYKAKRSQRFKPKQPKELQELSIAVRK |
| NO: | EKALDIIQKSIDHLIQDIRPEIPRIKQQKRYKNHVGKLFYLQKRRKNKLNLIGKGSFFKV |
| 108 | FSPKEKKNELLVICALTNIGRDIGLIGNYNTIINPLFEVTYQLYYDYIPKKNNKNVQRRL |
| | LYAYKSKNEKILKLKEAFFKRGHENAVNLGSFSYEKPLEKSLTLKIKNDKDDFQVSPS |
| | LRIRTGRFFVPSKRNLSRQEREINRRLVKIKSKIKNMTYGKFETARDKQSVHIFRLERQ |
| | KEKLPLQFRKDEKEFMEEFQKLKRRTNSLKKLRKSRQKKLADLLQLSEKVVYNNHTG |
| | TLKKTSNFLNFSSSVKRGKTAYIKELLGQEGFETLYCSNCINKGQKTRYNIETKEKCFS |
| | CKDVPFVWKKKSTDKDRKGAFLFPAKLKDVIKATFTVAKAYEDFYDNLKSIDEKKPY |
| | IKFKIGLILAHVRHEHKARAKEEAGQKNIYNKPIKIDKNCKECFFFKEEAM |
| SEQ | NTTRKKFRKRTGFPQSDNIKLAYCSAIVRAANLDADIQKKHNQCNPNLCVGIKSNEQS |
| ID | RKYEHSDRQALLCYACNQSTGAPKVDYIQIGEIGAKYKILQMVNAYDFLSLAYNLTK |
| NO: | LRNGKSRGHQRMSQLDEVVIVADYEKATEVIKRSINHLLDDIRGQLSKLKKRTQNEHI |
| 109 | TEHKQSKIRRKLRKLSRLLKRRRWKWGTIPNPYLKNWVFTKKDPELVTVALLHKLGR |
| | DIGLVNRSKRRSKQKLLPKVGFQLYYKWESPSLNNIKKSKAKKLPKRLLIPYKNVKLF |
| | DNKQKLENAIKSLLESYQKTIKVEFDQFFQNRTEEIIAEEQQTLERGLLKQLEKKKNEF |
| | ASQKKALKEEKKKIKEPRKAKLLMEESRSLGFLMANVSYALFNTTIEDLYKKSNVVSG |
| | CIPQEPVVVFPADIQNKGSLAKILFAPKDGFRIKFSGQHLTIRTAKFKIRGKEIKILTKTK |
| | REILKNIEKLRRVWYREQHYKLKLFGKEVSAKPRFLDKRKTSIERRDPNKLADQTDDR |
| | QAELRNKEYELRHKQHKMAERLDNIDTNAQNLQTLSFWVGEADKPPKLDEKDARGF |
| | GVRTCISAWKWFMEDLLKKQEEDPLLKLKLSIM |
| SEQ | PKKPKFQKRTGFPQPDNLRKEYCLAIVRAANLDADFEKKCTKCEGIKTNKKGNIVKGR |
| ID | TYNSADKDNLLCYACNISTGAPAVDYVFVGALEAKYKILQMVKAYDFHSLAYNLAK |
| NO: | LWKGRGRGHQRMGGLNEVVIVSNNEKALDVIEKSLNHFHDEIRGELSRLKAKFQNEH |
| 110 | LHVHKESKLRRKLRKISRLLKRRRWKWDVIPNSYLRNFTFTKTRPDFISVALLHRVGR |
| | DIGLVTKTKIPKPTDLLPQFGFQIYYTWDEPKLNKLKKSRLRSEPKRLLVPYKKIELYK |
| | NKSVLEEAIRHLAEVYTEDLTICFKDFFETQKRKFVSKEKESLKRELLKELTKLKKDFS |
| | ERKTALKRDRKEIKEPKKAKLLMEESRSLGFLAANTSYALFNLIAADLYTKSKKACST |
| | KLPRQLSTILPLEIKEHKSTTSLAIKPEEGFKIRFSNTHLSIRTPKFKMKGADIKALTKRK |
| | REILKNATKLEKSWYGLKHYKLKLYGKEVAAKPRFLDKRNPSIDRRDPKELMEQIEN |
| | RRNEVKDLEYEIRKGQHQMAKRLDNVDTNAQNLQTKSFWVGEADKPPELDSMEAK |
| | KLGLRTCISAWKWFMKDLVLLQEKSPNLKLKLSLTEM |
| SEQ | KFSKRQEGFLIPDNIDLYKCLAIVRSANLDADVQGHKSCYGVKKNGTYRVKQNGKKG |
| ID | VKEKGRKYVFDLIAFKGNIEKIPHEAIEEKDQGRVIVLGKFNYKLILNIEKNHNDRASL |
| NO: | EIKNKIKKLVQISSLETGEFLSDLLSGKIGIDEVYGIIEPDVFSGKELVCKACQQSTYAPL |
| 111 | VEYMPVGELDAKYKILSAIKGYDFLSLAYNLSRNRANKKRGHQKLGGGELSEVVISA |
| | NYDKALNVIKRSINHYHVEIKPEISKLKKKMQNEPLKVMKQARIRRELHQLSRKVKRL |
| | KWKWGMIPNPELQNIIFEKKEKDFVSYALLHTLGRDIGLFKDTSMLQVPNISDYGFQIY |
| | YSWEDPKLNSIKKIKDLPKRLLIPYKRLDFYIDTILVAKVIKNLIELYRKSYVYETFGEE |
| | YGYAKKAEDILFDWDSINLSEGIEQKIQKIKDEFSDLLYEARESKRQNFVESFENILGLY |
| | DKNFASDRNSYQEKIQSMIIKKQQENIEQKLKREFKEVIERGFEGMDQNKKYYKVLSP |
| | NIKGGLLYTDTNNLGFFRSHLAFMLLSKISDDLYRKNNLVSKGGNKGILDQTPETMLT |
| | LEFGKSNLPNISIKRKFFNIKYNSSWIGIRKPKFSIKGAVIREITKKVRDEQRLIKSLEGV |
| | WHKSTHFKRWGKPRFNLPRHPDREKNNDDNLMESITSRREQIQLLLREKQKQQEKMA |
| | GRLDKIDKEIQNLQTANFQIKQIDKKPALTEKSEGKQSVRNALSAWKWFMEDLIKYQ |
| | KRTPILQLKLAKM |
| SEQ | KFSKRQEGFVIPENIGLYKCLAIVRSANLDADVQGHVSCYGVKKNGTYVLKQNGKKSI |
| ID | REKGRKYASDLVAFKGDIEKIPFEVIEEKKKEQSIVLGKFNYKLVLDVMKGEKDRASL |
| NO: | TMKNKSKKLVQVSSLGTDEFLLTLLNEKFGIEEIYGIIEPEVFSGKKLVCKACQQSTYA |
| 112 | PLVEYMPVGELDSKYKILSAIKGYDFLSLAYNLARHRSNKKRGHQKLGGGELSEVVIS |
| | ANNAKALNVIKRSLNHYYSEIKPEISKLRKKMQNEPLKVGKQARMRRELHQLSRKVK |
| | RLKWKWGKIPNLELQNITFKESDRDFISYALLHTLGRDIGMFNKTEIKMPSNILGYGFQ |
| | IYYDWEEPKLNTIKKSKNTPKRILIPYKKLDFYNDSILVARAIKELVGLFQESYEWEIFG |
| | NEYNYAKEAEVELIKLDEESINGNVEKKLQRIKENFSNLLEKAREKKRQNFIESFESIAR |
| | LYDESFTADRNEYQREIQSFIIEKQKQSIEKKLKNEFKKIVEKKFNEQEQGKKHYRVLN |
| | PTIINEFLPKDKNNLGFLRSKIAFILLSKISDDLYKKSNAVSKGGEKGIIKQQPETILDLEF |
| | SKSKLPSINIKKKLFNIKYTSSWLGIRKPKFNIKGAKIREITRRVRDVQRTLKSAESSWY |
| | ASTHFRRWGFPRFNQPRHPDKEKKSDDRLIESITLLREQIQILLREKQKGQKEMAGRLD |
| | DVDKKIQNLQTANFQIKQTGDKPALTEKSAGKQSFRNALSAWKWFMENLLKYQNKT |
| | PDLKLKIARTVM |
| SEQ | KWIEPNNIDFNKCLAITRSANLDADVQGHKMCYGIKTNGTYKAIGKINKKHNTGIIEK |
| ID | RRTYVYDLIVTKEKNEKIVKKTDFMAIDEEIEFDEKKEKLLKKYIKAEVLGTGELIRKD |
| NO: | LNDGEKFDDLCSIEEPQAFRRSELVCKACNQSTYASDIRYIPIGEIEAKYKILKAIKGYD |
| 113 | FLSLKYNLGRLRDSKKRGHQKMGQGELKEFVICANKEKALDVIKRSLNHYLNEVKDE |
| | ISRLNKKMQNEPLKVNDQARWRRELNQISRRLKRLKWKWGEIPNPELKNLIFKSSRPE |
| | FVSYALIHTLGRDIGLINETELKPNNIQEYGFQIYYKWEDPELNHIKKVKNIPKRFIIPYK |
| | NLDLFGKYTILSRAIEGILKLYSSSFQYKSFKDPNLFAKEGEKKITNEDFELGYDEKIKKI |
| | KDDFKSYKKALLEKKKNTLEDSLNSILSVYEQSLLTEQINNVKKWKEGLLKSKESIHK |
| | QKKIENIEDIISRIEELKNVEGWIRTKERDIVNKEETNLKREIKKELKDSYYEEVRKDFS |
| | DLKKGEESEKKPFREEPKPIVIKDYIKFDVLPGENSALGFFLSHLSFNLFDSIQYELFEKS |
| | RLSSSKHPQIPETILDL |
| SEQ | FRKFVKRSGAPQPDNLNKYKCIAIVRAANLDADIMSNESSNCVMCKGIKMNKRKTAK |
| ID | GAAKTTELGRVYAGQSGNLLCTACTKSTMGPLVDYVPIGRIRAKYTILRAVKEYDFLS |
| NO: | LAYNLARTRVSKKGGRQKMHSLSELVIAAEYEIAWNIIKSSVIHYHQETKEEISGLRKK |
| 114 | LQAEHIHKNKEARIRREMHQISRRIKRLKWKWHMIPNSELHNFLFKQQDPSFVAVALL |
| | HTLGRDIGMINKPKGSAKREFIPEYGFQIYYKWMNPKLNDINKQKYRKMPKRSLIPYK |
| | NLNVFGDRELIENAMHKLLKLYDENLEVKGSKFFKTRVVAISSKESEKLKRDLLWKG |
| | ELAKIKKDFNADKNKMQELFKEVKEPKKANALMKQSRNMGFLLQNISYGALGLLAN |
| | RMYEASAKQSKGDATKQPSIVIPLEMEFGNAFPKLLLRSGKFAMNVSSPWLTIRKPKF |
| | VIKGNKIKNITKLMKDEKAKLKRLETSYHRATHFRPTLRGSIDWDSPYFSSPKQPNTHR |
| | RSPDRLSADITEYRGRLKSVEAELREGQRAMAKKLDSVDMTASNLQTSNFQLEKGED |
| | PRLTEIDEKGRSIRNCISSWKKFMEDLMKAQEANPVIKIKIALKDESSVLSEDSM |
| SEQ | KFHPENLNKSYCLAIVRAANLDADIQGHINCIGIKSNKSDRNYENKLESLQNVELLCKA |
| ID | CTKSTYKPNINSVPVGEKKAKYSILSEIKKYDFNSLVYNLKKYRKGKSRGHQKLNELR |
| NO: | ELVITSEYKKALDVINKSVNHYLVNIKNKMSKLKKILQNEHIHVGTLARIRRERNRISR |
| 115 | KLDHYRKKWKFVPNKILKNYVFKNQSPDFVSVALLHKLGRDIGLITKTAILQKSFPEY |
| | SLQLYYKYDTPKLNYLKKSKFKSLPKRILISYKYPKFDINSNYIEESIDKLLKLYEESPIY |
| | KNNSKIIEFFKKSEDNLIKSENDSLKRGIMKEFEKVTKNFSSKKKKLKEELKLKNEDKN |
| | SKMLAKVSRPIGFLKAYLSYMLFNIISNRIFEFSRKSSGRIPQLPSCIINLGNQFENFKNEL |
| | QDSNIGSKKNYKYFCNLLLKSSGFNISYEEEHLSIKTPNFFINGRKLKEITSEKKKIRKEN |
| | EQLIKQWKKLTFFKPSNLNGKKTSDKIRFKSPNNPDIERKSEDNIVENIAKVKYKLEDL |
| | LSEQRKEFNKLAKKHDGVDVEAQCLQTKSFWIDSNSPIKKSLEKKNEKVSVKKKMKA |
| | IRSCISAWKWFMADLIEAQKETPMIKLKLALM |
| SEQ | TTLVPSHLAGIEVMDETTSRNEDMIQKETSRSNEDENYLGVKNKCGINVHKSGRGSSK |
| ID | HEPNMPPEKSGEGQMPKQDSTEMQQRFDESVTGETQVSAGATASIKTDARANSGPRV |
| NO: | GTARALIVKASNLDRDIKLGCKPCEYIRSELPMGKKNGCNHCEKSSDIASVPKVESGFR |
| 116 | KAKYELVRRFESFAADSISRHLGKEQARTRGKRGKKDKKEQMGKVNLDEIAILKNES |
| | LIEYTENQILDARSNRIKEWLRSLRLRLRTRNKGLKKSKSIRRQLITLRRDYRKWIKPNP |
| | YRPDEDPNENSLRLHTKLGVDIGVQGGDNKRMNSDDYETSFSITWRDTATRKICFTKP |
| | KGLLPRHMKFKLRGYPELILYNEELRIQDSQKFPLVDWERIPIFKLRGVSLGKKKVKAL |
| | NRITEAPRLVVAKRIQVNIESKKKKVLTRYVYNDKSINGRLVKAEDSNKDPLLEFKKQ |
| | AEEINSDAKYYENQEIAKNYLWGCEGLHKNLLEEQTKNPYLAFKYGFLNIV |
| SEQ | LDFKRTCSQELVLLPEIEGLKLSGTQGVTSLAKKLINKAANVDRDESYGCHHCIHTRTS |
| ID | LSKPVKKDCNSCNQSTNHPAVPITLKGYKIAFYELWHRFTSWAVDSISKALHRNKVM |
| NO: | GKVNLDEYAVVDNSHIVCYAVRKCYEKRQRSVRLHKRAYRCRAKHYNKSQPKVGRI |
| 117 | YKKSKRRNARNLKKEAKRYFQPNEITNGSSDALFYKIGVDLGIAKGTPETEVKVDVSI |
| | CFQVYYGDARRVLRVRKMDELQSFHLDYTGKLKLKGIGNKDTFTIAKRNESLKWGST |
| | KYEVSRAHKKFKPFGKKGSVKRKCNDYFRSIASWSCEAASQRAQSNLKNAFPYQKAL |
| | VKCYKNLDYKGVKKNDMWYRLCSNRIFRYSRIAEDIAQYQSDKGKAKFEFVILAQSV |
| | AEYDISAIM |
| SEQ | VFLTDDKRKTALRKIRSAFRKTAEIALVRAQEADSLDRQAKKLTIETVSFGAPGAKNA |
| ID | FIGSLQGYNWNSHRANVPSSGSAKDVFRITELGLGIPQSAHEASIGKSFELVGNVVRYT |
| NO: | ANLLSKGYKKGAVNKGAKQQREIKGKEQLSFDLISNGPISGDKLINGQKDALAWWLI |
| 118 | DKMGFHIGLAMEPLSSPNTYGITLQAFWKRHTAPRRYSRGVIRQWQLPFGRQLAPLIH |
| | NFFRKKGASIPIVLTNASKKLAGKGVLLEQTALVDPKKWWQVKEQVTGPLSNIWERS |
| | VPLVLYTATFTHKHGAAHKRPLTLKVIRISSGSVFLLPLSKVTPGKLVRAWMPDINILR |
| | DGRPDEAAYKGPDLIRARERSFPLAYTCVTQIADEWQKRALESNRDSITPLEAKLVTG |
| | SDLLQIHSTVQQAVEQGIGGRISSPIQELLAKDALQLVLQQLFMTVDLLRIQWQLKQEV |
| | ADGNTSEKAVGWAIRISNIHKDAYKTAIEPCTSALKQAWNPLSGFEERTFQLDASIVRK |
| | RSTAKTPDDELVIVLRQQAAEMTVAVTQSVSKELMELAVRHSATLHLLVGEVASKQL |
| | SRSADKDRGAMDHWKLLSQSM |
| SEQ | EDLLQKALNTATNVAAIERHSCISCLFTESEIDVKYKTPDKIGQNTAGCQSCTFRVGYS |
| ID | GNSHTLPMGNRIALDKLRETIQRYAWHSLLFNVPPAPTSKRVRAISELRVAAGRERLFT |
| NO: | VITFVQTNILSKLQKRYAANWTPKSQERLSRLREEGQHILSLLESGSWQQKEVVREDQ |
| 119 | DLIVCSALTKPGLSIGAFCRPKYLKPAKHALVLRLIFVEQWPGQIWGQSKRTRRMRRR |
| | KDVERVYDISVQAWALKGKETRISECIDTMRRHQQAYIGVLPFLILSGSTVRGKGDCPI |
| | LKEITRMRYCPNNEGLIPLGIFYRGSANKLLRVVKGSSFTLPMWQNIETLPHPEPFSPEG |
| | WTATGALYEKNLAYWSALNEAVDWYTGQILSSGLQYPNQNEFLARLQNVIDSIPRKW |
| | FRPQGLKNLKPNGQEDIVPNEFVIPQNAIRAHHVIEWYHKTNDLVAKTLLGWGSQTTL |
| | NQTRPQGDLRFTYTRYYFREKEVPEV |
| SEQ | VPKKKLMRELAKKAVFEAIFNDPIPGSFGCKRCTLIDGARVTDAIEKKQGAKRCAGCE |
| ID | PCTFHTLYDSVKHALPAATGCDRTAIDTGLWEILTALRSYNWMSFRRNAVSDASQKQ |
| NO: | VWSIEELAIWADKERALRVILSALTHTIGKLKNGFSRDGVWKGGKQLYENLAQKDLA |
| 120 | KGLFANGEIFGKELVEADHDMLAWTIVPNHQFHIGLIRGNWKPAAVEASTAFDARWL |
| | TNGAPLRDTRTHGHRGRRFNRTEKLTVLCIKRDGGVSEEFRQERDYELSVMLLQPKN |
| | KLKPEPKGELNSFEDLHDHWWFLKGDEATALVGLTSDPTVGDFIQLGLYIRNPIKAHG |
| | ETKRRLLICFEPPIKLPLRRAFPSEAFKTWEPTINVFRNGRRDTEAYYDIDRARVFEFPE |
| | TRVSLEHLSKQWEVLRLEPDRENTDPYEAQQNEGAELQVYSLLQEAAQKMAPKVVID |
| | PFGQFPLELFSTFVAQLFNAPLSDTKAKIGKPLDSGFVVESHLHLLEEDFAYRDFVRVT |
| | FMGTEPTFRVIHYSNGEGYWKKTVLKGKNNIRTALIPEGAKAAVDAYKNKRCPLTLE |
| | AAILNEEKDRRLVLGNKALSLLAQTARGNLTILEALAAEVLRPLSGTEGVVHLHACVT |
| | RHSTLTESTETDNM |
| SEQ | VEKLFSERLKRAMWLKNEAGRAPPAETLTLKHKRVSGGHEKVKEELQRVLRSLSGTN |
| ID | QAAWNLGLSGGREPKSSDALKGEKSRVVLETVVFHSGHNRVLYDVIEREDQVHQRSS |
| NO: | IMHMRRKGSNLLRLWGRSGKVRRKMREEVAEIKPVWHKDSRWLAIVEEGRQSVVGI |
| 121 | SSAGLAVFAVQESQCTTAEPKPLEYVVSIWFRGSKALNPQDRYLEFKKLKTTEALRGQ |
| | QYDPIPFSLKRGAGCSLAIRGEGIKFGSRGPIKQFFGSDRSRPSHADYDGKRRLSLFSKY |
| | AGDLADLTEEQWNRTVSAFAEDEVRRATLANIQDFLSISHEKYAERLKKRIESIEEPVS |
| | ASKLEAYLSAIFETFVQQREALASNFLMRLVESVALLISLEEKSPRVEFRVARYLAESK |
| | EGFNRKAM |
| SEQ | VVITQSELYKERLLRVMEIKNDRGRKEPRESQGLVLRFTQVTGGQEKVKQKLWLIFEG |
| ID | FSGTNQASWNFGQPAGGRKPNSGDALKGPKSRVTYETVVFHFGLRLLSAVIERHNLK |
| NO: | QQRQTMAYMKRRAAARKKWARSGKKCSRMRNEVEKIKPKWHKDPRWFDIVKEGEP |
| 122 | SIVGISSAGFAIYIVEEPNFPRQDPLEIEYAISIWFRRDRSQYLTFKKIQKAEKLKELQYN |
| | PIPFRLKQEKTSLVFESGDIKFGSRGSIEHFRDEARGKPPKADMDNNRRLTMFSVFSGN |
| | LTNLTEEQYARPVSGLLAPDEKRMPTLLKKLQDFFTPIHEKYGERIKQRLANSEASKRP |
| | FKKLEEYLPAIYLEFRARREGLASNWVLVLINSVRTLVRIKSEDPYIEFKVSQYLLEKE |
| | DNKAL |
| SEQ | KQDALFEERLKKAIFIKRQADPLQREELSLLPPNRKIVTGGHESAKDTLKQILRAINGTN |
| ID | QASWNPGTPSGKRDSKSADALAGPKSRVKLETVVFHVGHRLLKKVVEYQGHQKQQH |
| NO: | GLKAFMRTCAAMRKKWKRSGKVVGELREQLANIQPKWHYDSRPLNLCFEGKPSVVG |
| 123 | LRSAGIALYTIQKSVVPVKEPKPIEYAVSIWFRGPKAMDREDRCLEFKKLKIATELRKL |
| | QFEPIVSTLTQGIKGFSLYIQGNSVKFGSRGPIKYFSNESVRQRPPKADPDGNKRLALFS |
| | KFSGDLSDLTEEQWNRPILAFEGIIRRATLGNIQDYLTVGHEQFAISLEQLLSEKESVLQ |
| | MSIEQQRLKKNLGKKAENEWVESFGAEQARKKAQGIREYISGFFQEYCSQREQWAEN |
| | WVQQLNKSVRLFLTIQDSTPFIEFRVARYLPKGEKKKGKAM |
| SEQ | ANHAERHKRLRKEANRAANRNRPLVADCDTGDPLVGICRLLRRGDKMQPNKTGCRS |
| ID | CEQVEPELRDAILVSGPGRLDNYKYELFQRGRAMAVHRLLKRVPKLNRPKKAAGNDE |
| NO: | KKAENKKSEIQKEKQKQRRMMPAVSMKQVSVADFKHVIENTVRHLFGDRRDREIAE |
| 124 | CAALRAASKYFLKSRRVRPRKLPKLANPDHGKELKGLRLREKRAKLKKEKEKQAELA |
| | RSNQKGAVLHVATLKKDAPPMPYEKTQGRNDYTTFVISAAIKVGATRGTKPLLTPQP |
| | REWQCSLYWRDGQRWIRGGLLGLQAGIVLGPKLNRELLEAVLQRPIECRMSGCGNPL |
| | QVRGAAVDFFMTTNPFYVSGAAYAQKKFKPFGTKRASEDGAAAKAREKLMTQLAK |
| | VLDKVVTQAAHSPLDGIWETRPEAKLRAMIMALEHEWIFLRPGPCHNAAEEVIKCDC |
| | TGGHAILWALIDEARGALEHKEFYAVTRAHTHDCEKQKLGGRLAGFLDLLIAQDVPL |
| | DDAPAARKIKTLLEATPPAPCYKAATSIATCDCEGKFDKLWAIIDATRAGHGTEDLWA |
| | RTLAYPQNVNCKCKAGKDLTHRLADFLGLLIKRDGPFRERPPHKVTGDRKLVFSGDK |
| | KCKGHQYVILAKAHNEEVVRAWISRWGLKSRTNKAGYAATELNLLLNWLSICRRRW |
| | MDMLTVQRDTPYIRMKTGRLVVDDKKERKAM |
| SEQ | AKQREALRVALERGIVRASNRTYTLVTNCTKGGPLPEQCRMIERGKARAMKWEPKLV |
| ID | GCGSCAAATVDLPAIEEYAQPGRLDVAKYKLTTQILAMATRRMMVRAAKLSRRKGQ |
| NO: | WPAKVQEEKEEPPEPKKMLKAVEMRPVAIVDFNRVIQTTIEHLWAERANADEAELKA |
| 125 | LKAAAAYFGPSLKIRARGPPKAAIGRELKKAHRKKAYAERKKARRKRAELARSQARG |
| | AAAHAAIRERDIPPMAYERTQGRNDVTTIPIAAAIKIAATRGARPLPAPKPMKWQCSL |
| | YWNEGQRWIRGGMLTAQAYAHAANIHRPMRCEMWGVGNPLKVRAFEGRVADPDG |
| | AKGRKAEFRLQTNAFYVSGAAYRNKKFKPFGTDRGGIGSARKKRERLMAQLAKILDK |
| | VVSQAAHSPLDDIWHTRPAQKLRAMIKQLEHEWMFLRPQAPTVEGTKPDVDVAGNM |
| | QRQIKALMAPDLPPIEKGSPAKRFTGDKRKKGERAVRVAEAHSDEVVTAWISRWGIQ |
| | TRRNEGSYAAQELELLLNWLQICRRRWLDMTAAQRVSPYIRMKSGRMITDAADEGV |
| | APIPLVENM |
| SEQ | KSISGRSIKHMACLKDMLKSEITEIEEKQKKESLRKWDYYSKFSDEILFRRNLNVSANH |
| ID | DANACYGCNPCAFLKEVYGFRIERRNNERIISYRRGLAGCKSCVQSTGYPPIEFVRRKF |
| NO: | GADKAMEIVREVLHRRNWGALARNIGREKEADPILGELNELLLVDARPYFGNKSAAN |
| 126 | ETNLAFNVITRAAKKFRDEGMYDIHKQLDIHSEEGKVPKGRKSRLIRIERKHKAIHGLD |
| | PGETWRYPHCGKGEKYGVWLNRSRLIHIKGNEYRCLTAFGTTGRRMSLDVACSVLGH |
| | PLVKKKRKKGKKTVDGTELWQIKKATETLPEDPIDCTFYLYAAKPTKDPFILKVGSLK |
| | APRWKKLHKDFFEYSDTEKTQGQEKGKRVVRRGKVPRILSLRPDAKFKVSIWDDPYN |
| | GKNKEGTLLRMELSGLDGAKKPLILKRYGEPNTKPKNFVFWRPHITPHPLTFTPKHDF |
| | GDPNKKTKRRRVFNREYYGHLNDLAKMEPNAKFFEDREVSNKKNPKAKNIRIQAKES |
| | LPNIVAKNGRWAAFDPNDSLWKLYLHWRGRRKTIKGGISQEFQEFKERLDLYKKHED |
| | ESEWKEKEKLWENHEKEWKKTLEIHGSIAEVSQRCVMQSMMGPLDGLVQKKDYVHI |
| | GQSSLKAADDAWTFSANRYKKATGPKWGKISVSNLLYDANQANAELISQSISKYLSK |
| | QKDNQGCEGRKMKFLIKIIEPLRENFVKHTRWLHEMTQKDCEVRAQFSRVSM |
| SEQ | FPSDVGADALKHVRMLQPRLTDEVRKVALTRAPSDRPALARFAAVAQDGLAFVRHL |
| ID | NVSANHDSNCTFPRDPRDPRRGPCEPNPCAFLREVWGFRIVARGNERALSYRRGLAGC |
| NO: | KSCVQSTGFPSVPFHRIGADDCMRKLHEILKARNWRLLARNIGREREADPLLTELSEYL |
| 127 | LVDARTYPDGAAPNSGRLAENVIKRAAKKFRDEGMRDIHAQLRVHSREGKVPKGRL |
| | QRLRRIERKHRAIHALDPGPSWEAEGSARAEVQGVAVYRSQLLRVGHHTQQIEPVGIV |
| | ARTLFGVGRTDLDVAVSVLGAPLTKRKKGSKTLESTEDFRIAKARETRAEDKIEVAFV |
| | LYPTASLLRDEIPKDAFPAMRIDRFLLKVGSVQADREILLQDDYYRFGDAEVKAGKNK |
| | GRTVTRPVKVPRLQALRPDAKFRVNVWADPFGAGDSPGTLLRLEVSGVTRRSQPLRL |
| | LRYGQPSTQPANFLCWRPHRVPDPMTFTPRQKFGERRKNRRTRRPRVFERLYQVHIKH |
| | LAHLEPNRKWFEEARVSAQKWAKARAIRRKGAEDIPVVAPPAKRRWAALQPNAELW |
| | DLYAHDREARKRFRGGRAAEGEEFKPRLNLYLAHEPEAEWESKRDRWERYEKKWTA |
| | VLEEHSRMCAVADRTLPQFLSDPLGARMDDKDYAFVGKSALAVAEAFVEEGTVERA |
| | QGNCSITAKKKFASNASRKRLSVANLLDVSDKADRALVFQAVRQYVQRQAENGGVE |
| | GRRMAFLRKLLAPLRQNFVCHTRWLHM |
| SEQ | AARKKKRGKIGITVKAKEKSPPAAGPFMARKLVNVAANVDGVEVHLCVECEADAHG |
| ID | SASARLLGGCRSCTGSIGAEGRLMGSVDVDRERVIAEPVHTETERLGPDVKAFEAGTA |
| NO: | ESKYAIQRGLEYWGVDLISRNRARTVRKMEEADRPESSTMEKTSWDEIAIKTYSQAYH |
| 128 | ASENHLFWERQRRVRQHALALFRRARERNRGESPLQSTQRPAPLVLAALHAEAAAIS |
| | GRARAEYVLRGPSANVRAAAADIDAKPLGHYKTPSPKVARGFPVKRDLLRARHRIVG |
| | LSRAYFKPSDVVRGTSDAIAHVAGRNIGVAGGKPKEIEKTFTLPFVAYWEDVDRVVH |
| | CSSFKADGPWVRDQRIKIRGVSSAVGTFSLYGLDVAWSKPTSFYIRCSDIRKKFHPKGF |
| | GPMKHWRQWAKELDRLTEQRASCVVRALQDDEELLQTMERGQRYYDVFSCAATHA |
| | TRGEADPSGGCSRCELVSCGVAHKVTKKAKGDTGIEAVAVAGCSLCESKLVGPSKPR |
| | VHRQMAALRQSHALNYLRRLQREWEALEAVQAPTPYLRFKYARHLEVRSM |
| SEQ | AAKKKKQRGKIGISVKPKEGSAPPADGPFMARKLVNVAANVDGVEVNLCIECEADAH |
| ID | GSAPARLLGGCKSCTGSIGAEGRLMGSVDVDRADAIAKPVNTETEKLGPDVQAFEAG |
| NO: | TAETKYALQRGLEYWGVDLISRNRSRTVRRTEEGQPESATMEKTSWDEIAIKSYTRAY |
| 129 | HASENHLFWERQRRVRQHALALFKRAKERNRGDSTLPREPGHGLVAIAALACEAYAV |
| | GGRNLAETVVRGPTFGTARAVRDVEIASLGRYKTPSPKVAHGSPVKRDFLRARHRIVG |
| | LARAYYRPSDVVRGTSDAIAHVAGRNIGVAGGKPRAVEAVFTLPFVAYWEDVDRVV |
| | HCSSFQVSAPWNRDQRMKIAGVTTAAGTFSLHGGELKWAKPTSFYIRCSDTRRKFRP |
| | KGFGPMKRWRQWAKDLDRLVEQRASCVVRALQDDAALLETMERGQRYYDVFACA |
| | VTHATRGEADRLAGCSRCALTPCQEAHRVTTKPRGDAGVEQVQTSDCSLCEGKLVGP |
| | SKPRLHRTLTLLRQEHGLNYLRRLQREWESLEAVQVPTPYLRFKYARHLEVRSM |
| SEQ | TDSQSESVPEVVYALTGGEVPGRVPPDGGSAEGARNAPTGLRKQRGKIKISAKPSKPG |
| ID | SPASSLARTLVNEAANVDGVQSSGCATCRMRANGSAPRALPIGCVACASSIGRAPQEE |
| NO: | TVCALPTTQGPDVRLLEGGHALRKYDIQRALEYWGVDLIGRNLDRQAGRGMEPAEG |
| 130 | ATATMKRVSMDELAVLDFGKSYYASEQHLFAARQRRVRQHAKALKIRAKHANRSGS |
| | VKRALDRSRKQVTALAREFFKPSDVVRGDSDALAHVVGRNLGVSRHPAREIPQTFTLP |
| | LCAYWEDVDRVISCSSLLAGEPFARDQEIRIEGVSSALGSLRLYRGAIEWHKPTSLYIR |
| | CSDTRRKFRPRGGLKKRWRQWAKDLDRLVEQRACCIVRSLQADVELLQTMERAQRF |
| | YDVHDCAATHVGPVAVRCSPCAGKQFDWDRYRLLAALRQEHALNYLRRLQREWES |
| | LEAQQVKMPYLRFKYARKLEVSGPLIGLEVRREPSMGTAIAEM |
| SEQ | AGTAGRRHGSLGARRSINIAGVTDRHGRWGCESCVYTRDQAGNRARCAPCDQSTYA |
| ID | PDVQEVTIGQRQAKYTIFLTLQSFSWTNTMRNNKRAAAGRSKRTTGKRIGQLAEIKIT |
| NO: | GVGLAHAHNVIQRSLQHNITKMWRAEKGKSKRVARLKKAKQLTKRRAYFRRRMSR |
| 131 | QSRGNGFFRTGKGGIHAVAPVKIGLDVGMIASGSSEPADEQTVTLDAIWKGRKKKIRL |
| | IGAKGELAVAACRFREQQTKGDKCIPLILQDGEVRWNQNNWQCHPKKLVPLCGLEVS |
| | RKFVSQADRLAQNKVASPLAARFDKTSVKGTLVESDFAAVLVNVTSIYQQCHAMLLR |
| | SQEPTPSLRVQRTITSM |
| SEQ | GVRFSPAQSQVFFRTVIPQSVEARFAINMAAIHDAAGAFGCSVCRFEDRTPRNAKAVH |
| ID | GCSPCTRSTNRPDVFVLPVGAIKAKYDVFMRLLGFNWTHLNRRQAKRVTVRDRIGQL |
| NO: | DELAISMLTGKAKAVLKKSICHNVDKSFKAMRGSLKKLHRKASKTGKSQLRAKLSDL |
| 132 | RERTNTTQEGSHVEGDSDVALNKIGLDVGLVGKPDYPSEESVEVVVCLYFVGKVLILD |
| | AQGRIRDMRAKQYDGFKIPIIQRGQLTVLSVKDLGKWSLVRQDYVLAGDLRFEPKISK |
| | DRKYAECVKRIALITLQASLGFKERIPYYVTKQVEIKNASHIAFVTEAIQNCAENFREM |
| | TEYLMKYQEKSPDLKVLLTQLM |
| SEQ | RAVVGKVFLEQARRALNLATNFGTNHRTGCNGCYVTPGKLSIPQDGEKNAAGCTSCL |
| ID | MKATASYVSYPKPLGEKVAKYSTLDALKGFPWYSLRLNLRPNYRGKPINGVQEVAPV |
| NO: | SKFRLAEEVIQAVQRYHFTELEQSFPGGRRRLRELRAFYTKEYRRAPEQRQHVVNGDR |
| 133 | NIVVVTVLHELGFSVGMFNEVELLPKTPIECAVNVFIRGNRVLLEVRKPQFDKERLLVE |
| | SLWKKDSRRHTAKWTPPNNEGRIFTAEGWKDFQLPLLLGSTSRSLRAIEKEGFVQLAP |
| | GRDPDYNNTIDEQHSGRPFLPLYLYLQGTISQEYCVFAGTWVIPFQDGISPYSTKDTFQ |
| | PDLKRKAYSLLLDAVKHRLGNKVASGLQYGRFPAIEELKRLVRMHGATRKIPRGEKD |
| | LLKKGDPDTPEWWLLEQYPEFWRLCDAAAKRVSQNVGLLLSLKKQPLWQRRWLESR |
| | TRNEPLDNLPLSMALTLHLTNEEAL |
| SEQ | AAVYSKFYIENHFKMGIPETLSRIRGPSIIQGFSVNENYINIAGVGDRDFIFGCKKCKYT |
| ID | RGKPSSKKINKCHPCKRSTYPEPVIDVRGSISEFKYKIYNKLKQEPNQSIKQNTKGRMN |
| NO: | PSDHTSSNDGIIINGIDNRIAYNVIFSSYKHLMEKQINLLRDTTKRKARQIKKYNNSGKK |
| 134 | KHSLRSQTKGNLKNRYHMLGMFKKGSLTITNEGDFITAVRKVGLDISLYKNESLNKQE |
| | VETELCLNIKWGRTKSYTVSGYIPLPINIDWKLYLFEKETGLTLRLFGNKYKIQSKKFLI |
| | AQLFKPKRPPCADPVVKKAQKWSALNAHVQQMAGLFSDSHLLKRELKNRMHKQLD |
| | FKSLWVGTEDYIKWFEELSRSYVEGAEKSLEFFRQDYFCFNYTKQTTM |
| SEQ | PQQQRDLMLMAANYDQDYGNGCGPCTVVASAAYRPDPQAQHGCKRHLRTLGASAV |
| ID | THVGLGDRTATITALHRLRGPAALAARARAAQAASAPMTPDTDAPDDRRRLEAIDAD |
| NO: | DVVLVGAHRALWSAVRRWADDRRAALRRRLHSEREWLLKDQIRWAELYTLIEASGT |
| 135 | PPQGRWRNTLGALRGQSRWRRVLAPTMRATCAETHAELWDALAELVPEMAKDRRG |
| | LLRPPVEADALWRAPMIVEGWRGGHSVVVDAVAPPLDLPQPCAWTAVRLSGDPRQR |
| | WGLHLAVPPLGQVQPPDPLKATLAVSMRHRGGVRVRTLQAMAVDADAPMQRHLQV |
| | PLTLQRGGGLQWGIHSRGVRRREARSMASWEGPPIWTGLQLVNRWKGQGSALLAPD |
| | RPPDTPPYAPDAAVAPAQPDTKRARRTLKEACTVCRCAPGHMRQLQVTLTGDGTWR |
| | RFRLRAPQGAKRKAEVLKVATQHDERIANYTAWYLKRPEHAAGCDTCDGDSRLDGA |
| | CRGCRPLLVGDQCFRRYLDKIEADRDDGLAQIKPKAQEAVAAMAAKRDARAQKVAA |
| | RAAKLSEATGQRTAATRDASHEARAQKELEAVATEGTTVRHDAAAVSAFGSWVARK |
| | GDEYRHQVGVLANRLEHGLRLQELMAPDSVVADQQRASGHARVGYRYVLTAM |
| SEQ | AVAHPVGRGNAGSPGARGPEELPRQLVNRASNVTRPATYGCAPCRHVRLSIPKPVLTG |
| ID | CRACEQTTHPAPKRAVRGGADAAKYDLAAFFAGWAADLEGRNRRRQVHAPLDPQP |
| NO: | DPNHEPAVTLQKIDLAEVSIEEFQRVLARSVKHRHDGRASREREKARAYAQVAKKRR |
| 136 | NSHAHGARTRRAVRRQTRAVRRAHRMGANSGEILVASGAEDPVPEAIDHAAQLRRRI |
| | RACARDLEGLRHLSRRYLKTLEKPCRRPRAPDLGRARCHALVESLQAAERELEELRRC |
| | DSPDTAMRRLDAVLAAAASTDATFATGWTVVGMDLGVAPRGSAAPEVSPMEMAISV |
| | FWRKGSRRVIVSKPIAGMPIRRHELIRLEGLGTLRLDGNHYTGAGVTKGRGLSEGTEP |
| | DFREKSPSTLGFTLSDYRHESRWRPYGAKQGKTARQFFAAMSRELRALVEHQVLAPM |
| | GPPLLEAHERRFETLLKGQDNKSIHAGGGGRYVWRGPPDSKKRPAADGDWFRFGRG |
| | HADHRGWANKRHELAANYLQSAFRLWSTLAEAQEPTPYARYKYTRVTM |
| SEQ | WDFLTLQVYERHTSPEVCVAGNSTKCASGTRKSDHTHGVGVKLGAQEINVSANDDR |
| ID | DHEVGCNICVISRVSLDIKGWRYGCESCVQSTPEWRSIVRFDRNHKEAKGECLSRFEY |
| NO: | WGAQSIARSLKRNKLMGGVNLDELAIVQNENVVKTSLKHLFDKRKDRIQANLKAVK |
| 137 | VRMRERRKSGRQRKALRRQCRKLKRYLRSYDPSDIKEGNSCSAFTKLGLDIGISPNKPP |
| | KIEPKVEVVFSLFYQGACDKIVTVSSPESPLPRSWKIKIDGIRALYVKSTKVKFGGRTFR |
| | AGQRNNRRKVRPPNVKKGKRKGSRSQFFNKFAVGLDAVSQQLPIASVQGLWGRAET |
| | KKAQTICLKQLESNKPLKESQRCLFLADNWVVRVCGFLRALSQRQGPTPYIRYRYRCN |
| | M |
| SEQ | ARNVGQRNASRQSKRESAKARSRRVTGGHASVTQGVALINAAANADRDHTTGCEPC |
| ID | TWERVNLPLQEVIHGCDSCTKSSPFWRDIKVVNKGYREAKEEIMRIASGISADHLSRAL |
| NO: | SHNKVMGRLNLDEVCILDFRTVLDTSLKHLTDSRSNGIKEHIRAVHRKIRMRRKSGKT |
| 138 | ARALRKQYFALRRQWKAGHKPNSIREGNSLTALRAVGFDVGVSEGTEPMPAPQTEVV |
| | LSVFYKGSATRILRISSPHPIAKRSWKVKIAGIKALKLIRREHDFSFGRETYNASQRAEK |
| | RKFSPHAARKDFFNSFAVQLDRLAQQLCVSSVENLWVTEPQQKLLTLAKDTAPYGIRE |
| | GARFADTRARLAWNWVFRVCGFTRALHQEQEPTPYCRFTWRSKM |

In some embodiments, the Type V CRISPR/Cas enzyme is a CasΦ nuclease. A CasΦ polypeptide can function as an endonuclease that catalyzes cleavage at a specific sequence in a target nucleic acid. A programmable CasΦ nuclease of the present disclosure may have a single active site in a RuvC domain that is capable of catalyzing pre-crRNA processing and nicking or cleaving of nucleic acids. This compact catalytic site may render the programmable CasΦ nuclease especially advantageous for genome engineering and new functionalities for genome manipulation.

TABLE 4 provides amino acid sequences of illustrative CasΦ polypeptides that can be used in compositions and methods of the disclosure.

TABLE 4
CasΦ Amino Acid Sequences

| | SEQ ID | |
|---|---|---|
| Name | NO | Amino Acid Sequence |
| CasΦ.l | SEQ ID | MADTPTLFTQFLRHHLPGQRFRKDILKQAGRILANKGEDATIA |
| | NO: 139 | FLRGKSEESPPDFQPPVKCPIIACSRPLTEWPIYQASVAIQGYV |
| | | YGQSLAEFEASDPGCSKDGLLGWFDKTGVCTDYFSVQGLNLI |
| | | FQNARKRYIGVQTKVTNRNEKRHKKLKRINAKRIAEGLPELT |
| | | SDEPESALDETGHLIDPPGLNTNIYCYQQVSPKPLALSEVNQLP |
| | | TAYAGYSTSGDDPIQPMVTKDRLSISKGQPGYIPEHQRALLSQ |
| | | KKHRRMRGYGLKARALLVIVRIQDDWAVIDLRSLLRNAYWR |
| | | RIVQTKEPSTITKLLKLVTGDPVLDATRMVATFTYKPGIVQVR |
| | | SAKCLKNKQGSKLFSERYLNETVSVTSIDLGSNNLVAVATYR |
| | | LVNGNTPELLQRFTLPSHLVKDFERYKQAHDTLEDSIQKTAV |
| | | ASLPQGQQTEIRMWSMYGFREAQERVCQELGLADGSIPWNV |
| | | MTATSTILTDLFLARGGDPKKCMFTSEPKKKKNSKQVLYKIR |
| | | DRAWAKMYRTLLSKETREAWNKALWGLKRGSPDYARLSKR |
| | | KEELARRCVNYTISTAEKRAQCGRTIVALEDLNIGFFHGRGKQ |
| | | EPGWVGLFTRKKENRWLMQALHKAFLELAHHRGYHVIEVNP |
| | | AYTSQTCPVCRHCDPDNRDQHNREAFHCIGCGFRGNADLDV |
| | | ATHNIAMVAITGESLKRARGSVASKTPQPLAAE |
| CasΦ.2 | SEQ ID | MPKPAVESEFSKVLKKHFPGERFRSSYMKRGGKILAAQGEEA |
| | NO: 140 | VVAYLQGKSEEEPPNFQPPAKCHVVTKSRDFAEWPIMKASEA |
| | | IQRYIYALSTTERAACKPGKSSESHAAWFAATGVSNHGYSHV |
| | | QGLNLIFDHTLGRYDGVLKKVQLRNEKARARLESINASRADE |
| | | GLPEIKAEEEEVATNETGHLLQPPGINPSFYVYQTISPQAYRPR |
| | | DEIVLPPEYAGYVRDPNAPIPLGVVRNRCDIQKGCPGYIPEWQ |
| | | REAGTAISPKTGKAVTVPGLSPKKNKRMRRYWRSEKEKAQD |
| | | ALLVTVRIGTDWVVIDVRGLLRNARWRTIAPKDISLNALLDLF |
| | | TGDPVIDVRRNIVTFTYTLDACGTYARKWTLKGKQTKATLD |
| | | KLTATQTVALVAIDLGQTNPISAGISRVTQENGALQCEPLDRF |
| | | TLPDDLLKDISAYRIAWDRNEEELRARSVEALPEAQQAEVRA |
| | | LDGVSKETARTQLCADFGLDPKRLPWDKMSSNTTFISEALLS |
| | | NSVSRDQVFFTPAPKKGAKKKAPVEVMRKDRTWARAYKPRL |
| | | SVEAQKLKNEALWALKRTSPEYLKLSRRKEELCRRSINYVIEK |
| | | TRRRTQCQIVIPVIEDLNVRFFHGSGKRLPGWDNFFTAKKENR |
| | | WFIQGLHKAFSDLRTHRSFYVFEVRPERTSITCPKCGHCEVGN |
| | | RDGEAFQCLSCGKTCNADLDVATHNLTQVALTGKTMPKREE |
| | | PRDAQGTAPARKTKKASKSKAPPAEREDQTPAQEPSQTS |
| CasΦ.3 | SEQ ID | MYILEMADLKSEPSLLAKLLRDRFPGKYWLPKYWKLAEKKR |
| | NO: 141 | LTGGEEAACEYMADKQLDSPPPNFRPPARCVILAKSRPFEDW |
| | | PVHRVASKAQSFVIGLSEQGFAALRAAPPSTADARRDWLRSH |
| | | GASEDDLMALEAQLLETIMGNAISLHGGVLKKIDNANVKAA |
| | | KRLSGRNEARLNKGLQELPPEQEGSAYGADGLLVNPPGLNLN |
| | | IYCRKSCCPKPVKNTARFVGHYPGYLRDSDSILISGTMDRLTII |
| | | EGMPGHIPAWQREQGLVKPGGRRRRLSGSESNMRQKVDPST |
| | | GPRRSTRSGTVNRSNQRTGRNGDPLLVEIRMKEDWVLLDAR |
| | | GLLRNLRWRESKRGLSCDHEDLSLSGLLALFSGDPVIDPVRNE |
| | | VVFLYGEGIIPVRSTKPVGTRQSKKLLERQASMGPLTLISCDL |
| | | GQTNLIAGRASAISLTHGSLGVRSSVRIELDPEIIKSFERLRKDA |
| | | DRLETEILTAAKETLSDEQRGEVNSHEKDSPQTAKASLCRELG |
| | | LHPPSLPWGQMGPSTTFIADMLISHGRDDDAFLSHGEFPTLEK |
| | | RKKFDKRFCLESRPLLSSETRKALNESLWEVKRTSSEYARLSQ |
| | | RKKEMARRAVNFVVEISRRKTGLSNVIVNIEDLNVRIFHGGG |
| | | KQAPGWDGFFRPKSENRWFIQAIHKAFSDLAAHHGIPVIESDP |
| | | QRTSMTCPECGHCDSKNRNGVRFLCKGCGASMDADFDAACR |
| | | NLERVALTGKPMPKPSTSCERLLSATTGKVCSDHSLSHDAIEK |
| | | AS |
| CasΦ.4 | SEQ ID | MEKEITELTKIRREFPNKKFSSTDMKKAGKLLKAEGPDAVRD |
| | NO: 142 | FLNSCQEIIGDFKPPVKTNIVSISRPFEEWPVSMVGRAIQEYYFS |
| | | LTKEELESVHPGTSSEDHKSFFNITGLSNYNYTSVQGLNLIFKN |
| | | AKAIYDGTLVKANNKNKKLEKKFNEINHKRSLEGLPIITPDFE |
| | | EPFDENGHLNNPPGINRNIYGYQGCAAKVFVPSKHKMVSLPK |
| | | EYEGYNRDPNLSLAGFRNRLEIPEGEPGHVPWFQRMDIPEGQI |
| | | GHVNKIQRFNFVHGKNSGKVKFSDKTGRVKRYHHSKYKDAT |
| | | KPYKFLEESKKVSALDSILAIITIGDDWVVFDIRGLYRNVFYRE |
| | | LAQKGLTAVQLLDLFTGDPVIDPKKGVVTFSYKEGVVPVFSQ |
| | | KIVPRFKSRDTLEKLTSQGPVALLSVDLGQNEPVAARVCSLK |
| | | NINDKITLDNSCRISFLDDYKKQIKDYRDSLDELEIKIRLEAINS |
| | | LETNQQVEIRDLDVFSADRAKANTVDMFDIDPNLISWDSMSD |
| | | ARVSTQISDLYLKNGGDESRVYFEINNKRIKRSDYNISQLVRP |
| | | KLSDSTRKNLNDSIWKLKRTSEEYLKLSKRKLELSRAVVNYTI |
| | | RQSKLLSGINDIVIILEDLDVKKKFNGRGIRDIGWDNFFSSRKE |
| | | NRWFIPAFHKAFSELSSNRGLCVIEVNPAWTSATCPDCGFCSK |
| | | ENRDGINFTCRKCGVSYHADIDVATLNIARVAVLGKPMSGPA |
| | | DRERLGDTKKPRVARSRKTMKRKDISNSTVEAMVTA |
| CasΦ.5 | SEQ ID | MDMLDTETNYATETPAQQQDYSPKPPKKAQRAPKGFSKKAR |
| | NO: 143 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEKI |
| | | LWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADIR |
| | | GALRNAQWRKLLKPDATYQSLFNLFTGDPVVNTRTNHLTMA |
| | | YREGVVNIVKSRSFKGRQTREHLLTLLGQGKTVAGVSFDLGQ |
| | | KHAAGLLAAHFGLGEDGNPVFTPIQACFLPQRYLDSLTNYRN |
| | | RYDALTLDMRRQSLLALTPAQQQEFADAQRDPGGQAKRACC |
| | | LKLNLNPDEIRWDLVSGISTMISDLYIERGGDPRDVHQQVETK |
| | | PKGKRKSEIRILKIRDGKWAYDFRPKIADETRKAQREQLWKL |
| | | QKASSEFERLSRYKINIARAIANWALQWGRELSGCDIVIPVLE |
| | | DLNVGSKFFDGKGKWLLGWDNRFTPKKENRWFIKVLHKAV |
| | | AELAPHRGVPVYEVMPHRTSMTCPACHYCHPTNREGDRFEC |
| | | QSCHVVKNTDRDVAPYNILRVAVEGKTLDRWQAEKKPQAEP |
| | | DRPMILIDNQES |
| CasΦ.6 | SEQ ID | MDMLDTETNYATETPAQQQDYSPKPPKKAQRAPKGFSKKAR |
| | NO: 144 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEKI |
| | | LWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADIR |
| | | GALRNAQWRKLLKPDATYQSLFNLFTGDPVVNTRTNHLTMA |
| | | YREGVVDIVKSRSFKGRQTREHLLTLLGQGKTVAGVSFDLGQ |
| | | KHAAGLLAAHFGLGEDGNPVFTPIQACFLPQRYLDSLTNYRN |
| | | RYDALTLDMRRQSLLALTPAQQQEFADAQRDPGGQAKRACC |
| | | LKLNLNPDEIRWDLVSGISTMISDLYIERGGDPRDVHQQVETK |
| | | PKGKRKSEIRILKIRDGKWAYDFRPKIADETRKAQREQLWKL |
| | | QKASSEFERLSRYKINIARAIANWALQWGRELSGCDIVIPVLE |
| | | DLNVGSKFFDGKGKWLLGWDNRFTPKKENRWFIKVLHKAV |
| | | AELAPHKGVPVYEVMPHRTSMTCPACHYCHPTNREGDRFEC |
| | | QSCHVVKNTDRDVAPYNILRVAVEGKTLDRWQAEKKPQAEP |
| | | DRPMILIDNQES |
| CasΦ.7 | SEQ ID | MSSLPTPLELLKQKHADLFKGLQFSSKDNKMAGKVLKKDGE |
| | NO: 145 | EAALAFLSERGVSRGELPNFRPPAKTLVVAQSRPFEEFPIYRVS |
| | | EAIQLYVYSLSVKELETVPSGSSTKKEHQRFFQDSSVPDFGYT |
| | | SVQGLNKIFGLARGIYLGVITRGENQLQKAKSKHEALNKKRR |
| | | ASGEAETEFDPTPYEYMTPERKLAKPPGVNHSIMCYVDISVDE |
| | | FDFRNPDGIVLPSEYAGYCREINTAIEKGTVDRLGHLKGGPGY |
| | | IPGHQRKESTTEGPKINFRKGRIRRSYTALYAKRDSRRVRQGK |
| | | LALPSYRHHMMRLNSNAESAILAVIFFGKDWVVFDLRGLLRN |
| | | VRWRNLFVDGSTPSTLLGMFGDPVIDPKRGVVAFCYKEQIVP |
| | | VVSKSITKMVKAPELLNKLYLKSEDPLVLVAIDLGQTNPVGV |
| | | GVYRVMNASLDYEVVTRFALESELLREIESYRQRTNAFEAQIR |
| | | AETFDAMTSEEQEEITRVRAFSASKAKENVCHRFGMPVDAVD |
| | | WATMGSNTIHIAKWVMRHGDPSLVEVLEYRKDNEIKLDKNG |
| | | VPKKVKLTDKRIANLTSIRLRFSQETSKHYNDTMWELRRKHP |
| | | VYQKLSKSKADFSRRVVNSIIRRVNHLVPRARIVFIIEDLKNLG |
| | | KVFHGSGKRELGWDSYFEPKSENRWFIQVLHKAFSETGKHK |
| | | GYYIIECWPNWTSCTCPKCSCCDSENRHGEVFRCLACGYTCN |
| | | TDFGTAPDNLVKIATTGKGLPGPKKRCKGSSKGKNPKIARSSE |
| | | TGVSVTESGAPKVKKSSPTQTSQSSSQSAP |
| CasΦ.8 | SEQ ID | MNKIEKEKTPLAKLMNENFAGLRFPFAIIKQAGKKLLKEGEL |
| | NO: 146 | KTIEYMTGKGSIEPLPNFKPPVKCLIVAKRRDLKYFPICKASCE |
| | | IQSYVYSLNYKDFMDYFSTPMTSQKQHEEFFKKSGLNIEYQN |
| | | VAGLNLIFNNVKNTYNGVILKVKNRNEKLKKKAIKNNYEFEE |
| | | IKTFNDDGCLINKPGINNVIYCFQSISPKILKNITHLPKEYNDYD |
| | | CSVDRNIIQKYVSRLDIPESQPGHVPEWQRKLPEFNNTNNPRR |
| | | RRKWYSNGRNISKGYSVDQVNQAKIEDSLLAQIKIGEDWIILD |
| | | IRGLLRDLNRRELISYKNKLTIKDVLGFFSDYPIIDIKKNLVTFC |
| | | YKEGVIQVVSQKSIGNKKSKQLLEKLIENKPIALVSIDLGQTNP |
| | | VSVKISKLNKINNKISIESFTYRFLNEEILKEIEKYRKDYDKLEL |
| | | KLINEA |
| CasΦ.9 | SEQ ID | MDMLDTETNYATETPSQQQDYSPKPPKKDRRAPKGFSKKAR |
| | NO: 147 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEKI |
| | | LWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADIR |
| | | GALRNAQWRKLLKPDATYQSLFNLFTGDPVVNTRTNHLTMA |
| | | YREGVVDIVKSRSFKGRQTREHLLTLLGQGKTVAGVSFDLGQ |
| | | KHAAGLLAAHFGLGEDGNPVFTPIQACFLPQRYLDSLTNYRN |
| | | RYDALTLDMRRQSLLALTPAQQQEFADAQRDPGGQAKRACC |
| | | LKLNLNPDEIRWDLVSGISTMISDLYIERGGDPRDVHQQVETK |
| | | PKGKRKSEIRILKIRDGKWAYDFRPKIADETRKAQREQLWKL |
| | | QKASSEFERLSRYKINIARAIANWALQWGRELSGCDIVIPVLE |
| | | DLNVGSKFFDGKGKWLLGWDNRFTPKKENRWFIKVLHKAV |
| | | AELAPHRGVPVYEVMPHRTSMTCPACHYCHPTNREGDRFEC |
| | | QSCHVVKNTDRDVAPYNILRVAVEGKTLDRWQAEKKPQAEP |
| | | DRPMILIDNQES |
| CasΦ.10 | SEQ ID | MDMLDTETNYATETPSQQQDYSPKPPKKDRRAPKGFSKKAR |
| | NO: 148 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEKI |
| | | LWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADIR |
| | | GALRNAQWRKLLKPDATYQSLFNLFTGDPVVNTRTNHLTMA |
| | | YREGVVNIVKSRSFKGRQTREHLLTLLGQGKTVAGVSFDLGQ |
| | | KHAAGLLAAHFGLGEDGNPVFTPIQACFLPQRYLDSLTNYRN |
| | | RYDALTLDMRRQSLLALTPAQQQEFADAQRDPGGQAKRACC |
| | | LKLNLNPDEIRWDLVSGISTMISDLYIERGGDPRDVHQQVETK |
| | | PKGKRKSEIRILKIRDGKWAYDFRPKIADETRKAQREQLWKL |
| | | QKASSEFERLSRYKINIARAIANWALQWGRELSGCDIVIPVLE |
| | | DLNVGSKFFDGKGKWLLGWDNRFTPKKENRWFIKVLHKAV |
| | | AELAPHRGVPVYEVMPHRTSMTCPACHYCHPTNREGDRFEC |
| | | QSCHVVKNTDRDVAPYNILRVAVEGKTLDRWQAEKKPQAEP |
| | | DRPMILIDNQES |
| CasΦ.11 | SEQ ID | MSNKTTPPSPLSLLLRAHFPGLKFESQDYKIAGKKLRDGGPEA |
| | NO: 149 | VISYLTGKGQAKLKDVKPPAKAFVIAQSRPFIEWDLVRVSRQI |
| | | QEKIFGIPATKGRPKQDGLSETAFNEAVASLEVDGKSKLNEET |
| | | RAAFYEVLGLDAPSLHAQAQNALIKSAISIREGVLKKVENRNE |
| | | KNLSKTKRRKEAGEEATFVEEKAHDERGYLIHPPGVNQTIPG |
| | | YQAVVIKSCPSDFIGLPSGCLAKESAEALTDYLPHDRMTIPKG |
| | | QPGYVPEWQHPLLNRRKNRRRRDWYSASLNKPKATCSKRSG |
| | | TPNRKNSRTDQIQSGRFKGAIPVLMRFQDEWVIIDIRGLLRNA |
| | | RYRKLLKEKSTIPDLLSLFTGDPSIDMRQGVCTFIYKAGQACS |
| | | AKMVKTKNAPEILSELTKSGPVVLVSIDLGQTNPIAAKVSRVT |
| | | QLSDGQLSHETLLRELLSNDSSDGKEIARYRVASDRLRDKLA |
| | | NLAVERLSPEHKSEILRAKNDTPALCKARVCAALGLNPEMIA |
| | | WDKMTPYTEFLATAYLEKGGDRKVATLKPKNRPEMLRRDIK |
| | | FKGTEGVRIEVSPEAAEAYREAQWDLQRTSPEYLRLSTWKQE |
| | | LTKRILNQLRHKAAKSSQCEVVVMAFEDLNIKMMHGNGKW |
| | | ADGGWDAFFIKKRENRWFMQAFHKSLTELGAHKGVPTIEVT |
| | | PHRTSITCTKCGHCDKANRDGERFACQKCGFVAHADLEIATD |
| | | NIERVALTGKPMPKPESERSGDAKKSVGARKAAFKPEEDAEA |
| | | AE |
| CasΦ.12 | SEQ ID | MIKPTVSQFLTPGFKLIRNHSRTAGLKLKNEGEEACKKFVREN |
| | NO: 150 | EIPKDECPNFQGGPAIANIIAKSREFTEWEIYQSSLAIQEVIFTLP |
| | | KDKLPEPILKEEWRAQWLSEHGLDTVPYKEAAGLNLIIKNAV |
| | | NTYKGVQVKVDNKNKNNLAKINRKNEIAKLNGEQEISFEEIK |
| | | AFDDKGYLLQKPSPNKSIYCYQSVSPKPFITSKYHNVNLPEEYI |
| | | GYYRKSNEPIVSPYQFDRLRIPIGEPGYVPKWQYTFLSKKENK |
| | | RRKLSKRIKNVSPILGIICIKKDWCVFDMRGLLRTNHWKKYH |
| | | KPTDSINDLFDYFTGDPVIDTKANVVRFRYKMENGIVNYKPV |
| | | REKKGKELLENICDQNGSCKLATVDVGQNNPVAIGLFELKKV |
| | | NGELTKTLISRHPTPIDFCNKITAYRERYDKLESSIKLDAIKQLT |
| | | SEQKIEVDNYNNNFTPQNTKQIVCSKLNINPNDLPWDKMISGT |
| | | HFISEKAQVSNKSEIYFTSTDKGKTKDVMKSDYKWFQDYKPK |
| | | LSKEVRDALSDIEWRLRRESLEFNKLSKSREQDARQLANWISS |
| | | MCDVIGIENLVKKNNFFGGSGKREPGWDNFYKPKKENRWWI |
| | | NAIHKALTELSQNKGKRVILLPAMRTSITCPKCKYCDSKNRN |
| | | GEKFNCLKCGIELNADIDVATENLATVAITAQSMPKPTCERSG |
| | | DAKKPVRARKAKAPEFHDKLAPSYTVVLREAV |
| CasΦ.13 | SEQ ID | MRQPAEKTAFQVFRQEVIGTQKLSGGDAKTAGRLYKQGKME |
| | NO: 151 | AAREWLLKGARDDVPPNFQPPAKCLVVAVSHPFEEWDISKTN |
| | | HDVQAYIYAQPLQAEGHLNGLSEKWEDTSADQHKLWFEKTG |
| | | VPDRGLPVQAINKIAKAAVNRAFGVVRKVENRNEKRRSRDN |
| | | RIAEHNRENGLTEVVREAPEVATNADGFLLHPPGIDPSILSYAS |
| | | VSPVPYNSSKHSFVRLPEEYQAYNVEPDAPIPQFVVEDRFAIPP |
| | | GQPGYVPEWQRLKCSTNKHRRMRQWSNQDYKPKAGRRAKP |
| | | LEFQAHLTRERAKGALLVVMRIKEDWVVFDVRGLLRNVEWR |
| | | KVLSEEAREKLTLKGLLDLFTGDPVIDTKRGIVTFLYKAEITKI |
| | | LSKRTVKTKNARDLLLRLTEPGEDGLRREVGLVAVDLGQTHP |
| | | IAAAIYRIGRTSAGALESTVLHRQGLREDQKEKLKEYRKRHT |
| | | ALDSRLRKEAFETLSVEQQKEIVTVSGSGAQITKDKVCNYLG |
| | | VDPSTLPWEKMGSYTHFISDDFLRRGGDPNIVHFDRQPKKGK |
| | | VSKKSQRIKRSDSQWVGRMRPRLSQETAKARMEADWAAQN |
| | | ENEEYKRLARSKQELARWCVNTLLQNTRCITQCDEIVVVIED |
| | | LNVKSLHGKGAREPGWDNFFTPKTENRWFIQILHKTFSELPK |
| | | HRGEHVIEGCPLRTSITCPACSYCDKNSRNGEKFVCVACGATF |
| | | HADFEVATYNLVRLATTGMPMPKSLERQGGGEKAGGARKA |
| | | RKKAKQVEKIVVQANANVTMNGASLHSP |
| CasΦ.14 | SEQ ID | MSSLPTPLELLKQKHADLFKGLQFSSKDNKMAGKVLKKDGE |
| | NO: 152 | EAALAFLSERGVSRGELPNFRPPAKTLVVAQSRPFEEFPIYRVS |
| | | EAIQLYVYSLSVKELETVPSGSSTKKEHQRFFQDSSVPDFGYT |
| | | SVQGLNKIFGLARGIYLGVITRGENQLQKAKSKHEALNKKRR |
| | | ASGEAETEFDPTPYEYMTPERKLAKPPGVNHSIMCYVDISVDE |
| | | FDFRNPDGIVLPSEYAGYCREINTAIEKGTVDRLGHLKGGPGY |
| | | IPGHQRKESTTEGPKINFRKGRIRRSYTALYAKRDSRRVRQGK |
| | | LALPSYRHHMMRLNSNAESAILAVIFFGKDWVVFDLRGLLRN |
| | | VRWRNLFVDGSTPSTLLGMFGDPVIDPKRGVVAFCYKEQIVP |
| | | VVSKSITKMVKAPELLNKLYLKSEDPLVLVAIDLGQTNPVGV |
| | | GVYRVMNASLDYEVVTRFALESELLREIESYRQRTNAFEAQIR |
| | | AETFDAMTSEEQEEITRVRAFSASKAKENVCHRFGMPVDAVD |
| | | WATMGSNTIHIAKWVMRHGDPSLVEVLEYRKDNEIKLDKNG |
| | | VPKKVKLTDKRIANLTSIRLRFSQETSKHYNDTMWELRRKHP |
| | | VYQKLSKSKADFSRRVVNSIIRRVNHLVPRARIVFIIEDLKNLG |
| | | KVFHGSGKRELGWDSYFEPKSENRWFIQVLHKAFSETGKHK |
| | | GYYIIECWPNWTSCTCPKCSCCDSENRHGEVFRCLACGYTCN |
| | | TDFGTAPDNLVKIATTGKGLPGPKKRCKGSSKGKNPKIARSSE |
| | | TGVSVTESGAPKVKKSSPTQTSQSSSQSAP |
| CasΦ.15 | SEQ ID | MIKPTVSQFLTPGFKLIRNHSRTAGLKLKNEGEEACKKFVREN |
| | NO: 153 | EIPKDECPNFQGGPAIANIIAKSREFTEWEIYQSSLAIQEVIFTLP |
| | | KDKLPEPILKEEWRAQWLSEHGLDTVPYKEAAGLNLIIKNAV |
| | | NTYKGVQVKVDNKNKNNLAKINRKNEIAKLNGEQEISFEEIK |
| | | AFDDKGYLLQKPSPNKSIYCYQSVSPKPFITSKYHNVNLPEEYI |
| | | GYYRKSNEPIVSPYQFDRLRIPIGEPGYVPKWQYTFLSKKENK |
| | | RRKLSKRIKNVSPILGIICIKKDWCVFDMRGLLRTNHWKKYH |
| | | KPTDSINDLFDYFTGDPVIDTKANVVRFRYKMENGIVNYKPV |
| | | REKKGKELLENICDQNGSCKLATVDVGQNNPVAIGLFELKKV |
| | | NGELTKTLISRHPTPIDFCNKITAYRERYDKLESSIKLDAIKQLT |
| | | SEQKIEVDNYNNNFTPQNTKQIVCSKLNINPNDLPWDKMISGT |
| | | HFISEKAQVSNKSEIYFTSTDKGKTKDVMKSDYKWFQDYKPK |
| | | LSKEVRDALSDIEWRLRRESLEFNKLSKSREQDARQLANWISS |
| | | MCDVIGIENLVKKNNFFGGSGKREPGWDNFYKPKKENRWWI |
| | | NAIHKALTELSQNKGKRVILLPAMRTSITCPKCKYCDSKNRN |
| | | GEKFNCLKCGIELNADIDVATENLATVAITAQSMPKPTCERSG |
| | | DAKKPVRARKAKAPEFHDKLAPSYTVVLREAV |
| CasΦ.16 | SEQ ID | MSNKTTPPSPLSLLLRAHFPGLKFESQDYKIAGKKLRDGGPEA |
| | NO: 154 | VISYLTGKGQAKLKDVKPPAKAFVIAQSRPFIEWDLVRVSRQI |
| | | QEKIFGIPATKGRPKQDGLSETAFNEAVASLEVDGKSKLNEET |
| | | RAAFYEVLGLDAPSLHAQAQNALIKSAISIREGVLKKVENRNE |
| | | KNLSKTKRRKEAGEEATFVEEKAHDERGYLIHPPGVNQTIPG |
| | | YQAVVIKSCPSDFIGLPSGCLAKESAEALTDYLPHDRMTIPKG |
| | | QPGYVPEWQHPLLNRRKNRRRRDWYSASLNKPKATCSKRSG |
| | | TPNRKNSRTDQIQSGRFKGAIPVLMRFQDEWVIIDIRGLLRNA |
| | | RYRKLLKEKSTIPDLLSLFTGDPSIDMRQGVCTFIYKAGQACS |
| | | AKMVKTKNAPEILSELTKSGPVVLVSIDLGQTNPIAAKVSRVT |
| | | QLSDGQLSHETLLRELLSNDSSDGKEIARYRVASDRLRDKLA |
| | | NLAVERLSPEHKSEILRAKNDTPALCKARVCAALGLNPEMIA |
| | | WDKMTPYTEFLATAYLEKGGDRKVATLKPKNRPEMLRRDIK |
| | | FKGTEGVRIEVSPEAAEAYREAQWDLQRTSPEYLRLSTWKQE |
| | | LTKRILNQLRHKAAKSSQCEVVVMAFEDLNIKMMHGNGKW |
| | | ADGGWDAFFIKKRENRWFMQAFHKSLTELGAHKGVPTIEVT |
| | | PHRTSITCTKCGHCDKANRDGERFACQKCGFVAHADLEIATD |
| | | NIERVALTGKPMPKPESERSGDAKKSVGARKAAFKPEEDAEA |
| | | AE |
| CasΦ.17 | SEQ ID | MYSLEMADLKSEPSLLAKLLRDRFPGKYWLPKYWKLAEKKR |
| | NO: 155 | LTGGEEAACEYMADKQLDSPPPNFRPPARCVILAKSRPFEDW |
| | | PVHRVASKAQSFVIGLSEQGFAALRAAPPSTADARRDWLRSH |
| | | GASEDDLMALEAQLLETIMGNAISLHGGVLKKIDNANVKAA |
| | | KRLSGRNEARLNKGLQELPPEQEGSAYGADGLLVNPPGLNLN |
| | | IYCRKSCCPKPVKNTARFVGHYPGYLRDSDSILISGTMDRLTII |
| | | EGMPGHIPAWQREQGLVKPGGRRRRLSGSESNMRQKVDPST |
| | | GPRRSTRSGTVNRSNQRTGRNGDPLLVEIRMKEDWVLLDAR |
| | | GLLRNLRWRESKRGLSCDHEDLSLSGLLALFSGDPVIDPVRNE |
| | | VVFLYGEGIIPVRSTKPVGTRQSKKLLERQASMGPLTLISCDL |
| | | GQTNLIAGRASAISLTHGSLGVRSSVRIELDPEIIKSFERLRKDA |
| | | DRLETEILTAAKETLSDEQRGEVNSHEKDSPQTAKASLCRELG |
| | | LHPPSLPWGQMGPSTTFIADMLISHGRDDDAFLSHGEFPTLEK |
| | | RKKFDKRFCLESRPLLSSETRKALNESLWEVKRTSSEYARLSQ |
| | | RKKEMARRAVNFVVEISRRKTGLSNVIVNIEDLNVRIFHGGG |
| | | KQAPGWDGFFRPKSENRWFIQAIHKAFSDLAAHHGIPVIESDP |
| | | QRTSMTCPECGHCDSKNRNGVRFLCKGCGASMDADFDAACR |
| | | NLERVALTGKPMPKPSTSCERLLSATTGKVCSDHSLSHDAIEK |
| | | AS |
| CasΦ.18 | SEQ ID | MEKEITELTKIRREFPNKKFSSTDMKKAGKLLKAEGPDAVRD |
| | NO: 156 | FLNSCQEIIGDFKPPVKTNIVSISRPFEEWPVSMVGRAIQEYYFS |
| | | LTKEELESVHPGTSSEDHKSFFNITGLSNYNYTSVQGLNLIFKN |
| | | AKAIYDGTLVKANNKNKKLEKKFNEINHKRSLEGLPIITPDFE |
| | | EPFDENGHLNNPPGINRNIYGYQGCAAKVFVPSKHKMVSLPK |
| | | EYEGYNRDPNLSLAGFRNRLEIPEGEPGHVPWFQRMDIPEGQI |
| | | GHVNKIQRFNFVHGKNSGKVKFSDKTGRVKRYHHSKYKDAT |
| | | KPYKFLEESKKVSALDSILAIITIGDDWVVFDIRGLYRNVFYRE |
| | | LAQKGLTAVQLLDLFTGDPVIDPKKGVVTFSYKEGVVPVFSQ |
| | | KIVPRFKSRDTLEKLTSQGPVALLSVDLGQNEPVAARVCSLK |
| | | NINDKITLDNSCRISFLDDYKKQIKDYRDSLDELEIKIRLEAINS |
| | | LETNQQVEIRDLDVFSADRAKANTVDMFDIDPNLISWDSMSD |
| | | ARVSTQISDLYLKNGGDESRVYFEINNKRIKRSDYNISQLVRP |
| | | KLSDSTRKNLNDSIWKLKRTSEEYLKLSKRKLELSRAVVNYTI |
| | | RQSKLLSGINDIVIILEDLDVKKKFNGRGIRDIGWDNFFSSRKE |
| | | NRWFIPAFHKTFSELSSNRGLCVIEVNPAWTSATCPDCGFCSK |
| | | ENRDGINFTCRKCGVSYHADIDVATLNIARVAVLGKPMSGPA |
| | | DRERLGDTKKPRVARSRKTMKRKDISNSTVEAMVTA |
| CasΦ.19 | SEQ ID | MLVRTSTLVQDNKNSRSASRAFLKKPKMPKNKHIKEPTELAK |
| | NO: 157 | LIRELFPGQRFTRAINTQAGKILKHKGRDEVVEFLKNKGIDKE |
| | | QFMDFRPPTKARIVATSGAIEEFSYLRVSMAIQECCFGKYKFP |
| | | KEKVNGKLVLETVGLTKEELDDFLPKKYYENKKSRDRFFLKT |
| | | GICDYGYTYAQGLNEIFRNTRAIYEGVFTKVNNRNEKRREKK |
| | | DKYNEERRSKGLSEEPYDEDESATDESGHLINPPGVNLNIWTC |
| | | EGFCKGPYVTKLSGTPGYEVILPKVFDGYNRDPNEIISCGITDR |
| | | FAIPEGEPGHIPWHQRLEIPEGQPGYVPGHQRFADTGQNNSGK |
| | | ANPNKKGRMRKYYGHGTKYTQPGEYQEVFRKGHREGNKRR |
| | | YWEEDFRSEAHDCILYVIHIGDDWVVCDLRGPLRDAYRRGLV |
| | | PKEGITTQELCNLFSGDPVIDPKHGVVTFCYKNGLVRAQKTIS |
| | | AGKKSRELLGALTSQGPIALIGVDLGQTEPVGARAFIVNQARG |
| | | SLSLPTLKGSFLLTAENSSSWNVFKGEIKAYREAIDDLAIRLKK |
| | | EAVATLSVEQQTEIESYEAFSAEDAKQLACEKFGVDSSFILWE |
| | | DMTPYHTGPATYYFAKQFLKKNGGNKSLIEYIPYQKKKSKKT |
| | | PKAVLRSDYNIACCVRPKLLPETRKALNEAIRIVQKNSDEYQR |
| | | LSKRKLEFCRRVVNYLVRKAKKLTGLERVIIAIEDLKSLEKFF |
| | | TGSGKRDNGWSNFFRPKKENRWFIPAFHKAFSELAPNRGFYV |
| | | IECNPARTSITDPDCGYCDGDNRDGIKFECKKCGAKHHTDLD |
| | | VAPLNIAIVAVTGRPMPKTVSNKSKRERSGGEKSVGASRKRN |
| | | HRKSKANQEMLDATSSAAE |
| CasΦ.20 | SEQ ID | MPKIKKPTEISLLRKEVFPDLHFAKDRMRAASLVLKNEGREA |
| | NO: 158 | AIEYLRVNHEDKPPNFMPPAKTPYVALSRPLEQWPIAQASIAI |
| | | QKYIFGLTKDEFSATKKLLYGDKSTPNTESRKRWFEVTGVPN |
| | | FGYMSAQGLNAIFSGALARYEGVVQKVENRNKKRFEKLSEK |
| | | NQLLIEEGQPVKDYVPDTAYHTPETLQKLAENNHVRVEDLGD |
| | | MIDRLVHPPGIHRSIYGYQQVPPFAYDPDNPKGIILPKAYAGY |
| | | TRKPHDIIEAMPNRLNIPEGQAGYIPEHQRDKLKKGGRVKRLR |
| | | TTRVRVDATETVRAKAEALNAEKARLRGKEAILAVFQIEEDW |
| | | ALIDMRGLLRNVYMRKLIAAGELTPTTLLGYFTETLTLDPRRT |
| | | EATFCYHLRSEGALHAEYVRHGKNTRELLLDLTKDNEKIALV |
| | | TIDLGQRNPLAAAIFRVGRDASGDLTENSLEPVSRMLLPQAYL |
| | | DQIKAYRDAYDSFRQNIWDTALASLTPEQQRQILAYEAYTPD |
| | | DSKENVLRLLLGGNVMPDDLPWEDMTKNTHYISDRYLADGG |
| | | DPSKVWFVPGPRKRKKNAPPLKKPPKPRELVKRSDHNISHLSE |
| | | FRPQLLKETRDAFEKAKIDTERGHVGYQKLSTRKDQLCKEIL |
| | | NWLEAEAVRLTRCKTMVLGLEDLNGPFFNQGKGKVRGWVS |
| | | FFRQKQENRWIVNGFRKNALARAHDKGKYILELWPSWTSQT |
| | | CPKCKHVHADNRHGDDFVCLQCGARLHADAEVATWNLAVV |
| | | AIQGHSLPGPVREKSNDRKKSGSARKSKKANESGKVVGAWA |
| | | AQATPKRATSKKETGTARNPVYNPLETQASCPAP |
| CasΦ.21 | SEQ ID | MTPSPQIARLVETPLAAALKAHHPGKKFRSDYLKKAGKILKD |
| | NO: 159 | QGVEAAMAHLDGKDQAEPPNFKPPAKCRIVARSREFSEWPIV |
| | | KASVEIQKYIYGLTLEERKACDPGKSSASHKAWFAKTGVNTF |
| | | GYSSVQGFNLIFGHTLGRYDGVLVKTENLNKKRAEKNERFRA |
| | | KALAEGRAEPVCPPLVTATNDTGQDVTLEDGRVVRPGQLLQP |
| | | PGINPNIYAYQQVSPKAYVPGIIELPEEFQGYSRDPNAVILPLV |
| | | PRDRLSIPKGQPGYVPEPHREGLTGRKDRRMRRYYETERGTK |
| | | LKRPPLTAKGRADKANEALLVVVRIDSDWVVMDVRGLLRNA |
| | | RWRRLVSKEGITLNGLLDLFTGDPVLNPKDCSVSRDTGDPVN |
| | | DPRHGVVTFCYKLGVVDVCSKDRPIKGFRTKEVLERLTSSGT |
| | | VGMVSIDLGQTNPVAAAVSRVTKGLQAETLETFTLPDDLLGK |
| | | VRAYRAKTDRMEEGFRRNALRKLTAEQQAEITRYNDATEQQ |
| | | AKALVCSTYGIGPEEVPWERMTSNTTYISDHILDHGGDPDTVF |
| | | FMATKRGQNKPTLHKRKDKAWGQKFRPAISVETRLARQAAE |
| | | WELRRASLEFQKLSVWKTELCRQAVNYVMERTKKRTQCDVI |
| | | IPVIEDLPVPLFHGSGKRDPGWANFFVHKRENRWFIDGLHKAF |
| | | SELGKHRGIYVFEVCPQRTSITCPKCGHCDPDNRDGEKFVCLS |
| | | CQATLNADLDVATTNLVRVALTGKVMPRSERSGDAQTPGPA |
| | | RKARTGKIKGSKPTSAPQGATQTDAKAHLSQTGV |
| CasΦ.22 | SEQ ID | MTPSPQIARLVETPLAAALKAHHPGKKFRSDYLKKAGKILKD |
| | NO: 160 | QGVEAAMAHLDGKDQAEPPNFKPPAKCRIVARSREFSEWPIV |
| | | KASVEIQKYIYGLTLEERKACDPGKSSASHKAWFAKTGVNTF |
| | | GYSSVQGFNLIFGHTLGRYDGVLVKTENLNKKRAEKNERFRA |
| | | KALAEGRAEPVCPPLVTATNDTGQDVTLEDGRVVRPGQLLQP |
| | | PGINPNIYAYQQVSPKAYVPGIIELPEEFQGYSRDPNAVILPLV |
| | | PRDRLSIPKGQPGYVPEPHREGLTGRKDRRMRRYYETERGTK |
| | | LKRPPLTAKGRADKANEALLVVVRIDSDWVVMDVRGLLRNA |
| | | RWRRLVSKEGITLNGLLDLFTGDPVLNPKDCSVSRDTGDPVN |
| | | DPRHGVVTFCYKLGVVDVCSKDRPIKGFRTKEVLERLTSSGT |
| | | VGMVSIDLGQTNPVAAAVSRVTKGLQAETLETFTLPDDLLGK |
| | | VRAYRAKTDRMEEGFRRNALRKLTAEQQAEITRYNDATEQQ |
| | | AKALVCSTYGIGPEEVPWERMTSNTTYISDHILDHGGDPDTVF |
| | | FMATKRGQNKPTLHKRKDKAWGQKFRPAISVETRLARQAAE |
| | | WELRRASLEFQKLSVWKTELCRQAVNYVMERTKKRTQCDVI |
| | | IPVIEDLPVPLFHGSGKRDPGWANFFVHKRENRWFIDGLHKAF |
| | | SELGKHRGIYVFEVCPQRTSITCPKCGHCDPDNRDGEKFVCLS |
| | | CQATLHADLDVATTNLVRVALTGKVMPRSERSGDAQTPGPA |
| | | RKARTGKIKGSKPTSAPQGATQTDAKAHLSQTGV |
| CasΦ.23 | SEQ ID | MKTEKPKTALTLLREEVFPGKKYRLDVLKEAGKKLSTKGRE |
| | NO: 161 | ATIEFLTGKDEERPQNFQPPAKTSIVAQSRPFDQWPIVQVSLA |
| | | VQKYIYGLTQSEFEANKKALYGETGKAISTESRRAWFEATGV |
| | | DNFGFTAAQGINPIFSQAVARYEGVIKKVENRNEKKLKKLTK |
| | | KNLLRLESGEEIEDFEPEATFNEEGRLLQPPGANPNIYCYQQIS |
| | | PRIYDPSDPKGVILPQIYAGYDRKPEDIISAGVPNRLAIPEGQPG |
| | | YIPEHQRAGLKTQGRIRCRASVEAKARAAILAVVHLGEDWVV |
| | | LDLRGLLRNVYWRKLASPGTLTLKGLLDFFTGGPVLDARRGI |
| | | ATFSYTLKSAAAVHAENTYKGKGTREVLLKLTENNSVALVT |
| | | VDLGQRNPLAAMIARVSRTSQGDLTYPESVEPLTRLFLPDPFL |
| | | EEVRKYRSSYDALRLSIREAAIASLTPEQQAEIRYIEKFSAGDA |
| | | KKNVAEVFGIDPTQLPWDAMTPRTTYISDLFLRMGGDRSRVF |
| | | FEVPPKKAKKAPKKPPKKPAGPRIVKRTDGMIARLREIRPRLS |
| | | AETNKAFQEARWEGERSNVAFQKLSVRRKQFARTVVNHLVQ |
| | | TAQKMSRCDTVVLGIEDLNVPFFHGRGKYQPGWEGFFRQKK |
| | | ENRWLINDMHKALSERGPHRGGYVLELTPFWTSLRCPKCGH |
| | | TDSANRDGDDFVCVKCGAKLHSDLEVATANLALVAITGQSIP |
| | | RPPREQSSGKKSTGTARMKKTSGETQGKGSKACVSEALNKIE |
| | | QGTARDPVYNPLNSQVSCPAP |
| CasΦ.24 | SEQ ID | VYNPDMKKPNNIRRIREEHFEGLCFGKDVLTKAGKIYEKDGE |
| | NO: 162 | EAAIDFLMGKDEEDPPNFKPPAKTTIVAQSRPFDQWPIYQVSQ |
| | | AVQERVFAYTEEEFNASKEALFSGDISSKSRDFWFKTNNISDQ |
| | | GIGAQGLNTILSHAFSRYSGVIKKVENRNKKRLKKLSKKNQL |
| | | KIEEGLEILEFKPDSAFNENGLLAQPPGINPNIYGYQAVTPFVF |
| | | DPDNPGDVILPKQYEGYSRKPDDIIEKGPSRLDIPKGQPGYVPE |
| | | HQRKNLKKKGRVRLYRRTPPKTKALASILAVLQIGKDWVLFD |
| | | MRGLLRSVYMREAATPGQISAKDLLDTFTGCPVLNTRTGEFT |
| | | FCYKLRSEGALHARKIYTKGETRTLLTSLTSENNTIALVTVDL |
| | | GQRNPAAIMISRLSRKEELSEKDIQPVSRRLLPDRYLNELKRY |
| | | RDAYDAFRQEVRDEAFTSLCPEHQEQVQQYEALTPEKAKNL |
| | | VLKHFFGTHDPDLPWDDMTSNTHYIANLYLERGGDPSKVFFT |
| | | RPLKKDSKSKKPRKPTKRTDASISRLPEIRPKMPEDARKAFEK |
| | | AKWEIYTGHEKFPKLAKRVNQLCREIANWIEKEAKRLTLCDT |
| | | VVVGIEDLSLPPKRGKGKFQETWQGFFRQKFENRWVIDTLKK |
| | | AIQNRAHDKGKYVLGLAPYWTSQRCPACGFIHKSNRNGDHF |
| | | KCLKCEALFHADSEVATWNLALVAVLGKGITNPDSKKPSGQ |
| | | KKTGTTRKKQIKGKNKGKETVNVPPTTQEVEDIIAFFEKDDET |
| | | VRNPVYKPTGT |
| CasΦ.25 | SEQ ID | MKKPNNIRRIREEHFEGLCFGKDVLTKAGKIYEKDGEEAAIDF |
| | NO: 163 | LMGKDEEDPPNFKPPAKTTIVAQSRPFDQWPIYQVSQAVQER |
| | | VFAYTEEEFNASKEALFSGDISSKSRDFWFKTNNISDQGIGAQ |
| | | GLNTILSHAFSRYSGVIKKVENRNKKRLKKLSKKNQLKIEEGL |
| | | EILEFKPDSAFNENGLLAQPPGINPNIYGYQAVTPFVFDPDNPG |
| | | DVILPKQYEGYSRKPDDIIEKGPSRLDIPKGQPGYVPEHQRKN |
| | | LKKKGRVRLYRRTPPKTKALASILAVLQIGKDWVLFDMRGLL |
| | | RSVYMREAATPGQISAKDLLDTFTGCPVLNTRTGEFTFCYKL |
| | | RSEGALHARKIYTKGETRTLLTSLTSENNTIALVTVDLGQRNP |
| | | AAIMISRLSRKEELSEKDIQPVSRRLLPDRYLNELKRYRDAYD |
| | | AFRQEVRDEAFTSLCPEHQEQVQQYEALTPEKAKNLVLKHFF |
| | | GTHDPDLPWDDMTSNTHYIANLYLERGGDPSKVFFTRPLKKD |
| | | SKSKKPRKPTKRTDASISRLPEIRPKMPEDARKAFEKAKWEIY |
| | | TGHEKFPKLAKRVNQLCREIANWIEKEAKRLTLCDTVVVGIE |
| | | DLSLPPKRGKGKFQETWQGFFRQKFENRWVIDTLKKAIQNRA |
| | | HDKGKYVLGLAPYWTSQRCPACGFIHKSNRNGDHFKCLKCE |
| | | ALFHADSEVATWNLALVAVLGKGITNPDSKKPSGQKKTGTT |
| | | RKKQIKGKNKGKETVNVPPTTQEVEDIIAFFEKDDETVRNPVY |
| | | KPTGT |
| CasΦ.26 | SEQ ID | VIKTHFPAGRFRKDHQKTAGKKLKHEGEEACVEYLRNKVSD |
| | NO: 164 | YPPNFKPPAKGTIVAQSRPFSEWPIVRASEAIQKYVYGLTVAE |
| | | LDVFSPGTSKPSHAEWFAKTGVENYGYRQVQGLNTIFQNTVN |
| | | RFKGVLKKVENRNKKSLKRQEGANRRRVEEGLPEVPVTVES |
| | | ATDDEGRLLQPPGVNPSIYGYQGVAPRVCTDLQGFSGMSVDF |
| | | AGYRRDPDAVLVESLPEGRLSIPKGERGYVPEWQRDPERNKF |
| | | PLREGSRRQRKWYSNACHKPKPGRTSKYDPEALKKASAKDA |
| | | LLVSISIGEDWAIIDVRGLLRDARRRGFTPEEGLSLNSLLGLFT |
| | | EYPVFDVQRGLITFTYKLGQVDVHSRKTVPTFRSRALLESLVA |
| | | KEEIALVSVDLGQTNPASMKVSRVRAQEGALVAEPVHRMFLS |
| | | DVLLGELSSYRKRMDAFEDAIRAQAFETMTPEQQAEITRVCD |
| | | VSVEVARRRVCEKYSISPQDVPWGEMTGHSTFIVDAVLRKGG |
| | | DESLVYFKNKEGETLKFRDLRISRMEGVRPRLTKDTRDALNK |
| | | AVLDLKRAHPTFAKLAKQKLELARRCVNFIEREAKRYTQCER |
| | | VVFVIEDLNVGFFHGKGKRDRGWDAFFTAKKENRWVIQALH |
| | | KAFSDLGLHRGSYVIEVTPQRTSMTCPRCGHCDKGNRNGEKF |
| | | VCLQCGATLHADLEVATDNIERVALTGKAMPKPPVRERSGD |
| | | VQKAGTARKARKPLKPKQKTEPSVQEGSSDDGVDKSPGDAS |
| | | RNPVYNPSDTLSI |
| CasΦ.27 | SEQ ID | MAKAKTLAALLRELLPGQHLAPHHRWVANKLLMTSGDAAA |
| | NO: 165 | FVIGKSVSDPVRGSFRKDVITKAGRIFKKDGPDAAAAFLDGK |
| | | WEDRPPNFQPPAKAAIVAISRSFDEWPIVKVSCAIQQYLYALP |
| | | VQEFESSVPEARAQAHAAWFQDTGVDDCNFKSTQGLNAIFN |
| | | HGKRTYEGVLKKAQNRNDKKNLRLERINAKRAEAGQAPLVA |
| | | GPDESPTDDAGCLLHPPGINANIYCYQQVSPRPYEQSCGIQLPP |
| | | EYAGYNRLSNVAIPPMPNRLDIPQGQPGYVPEHHRHGIKKFG |
| | | RVRKRYGVVPGRNRDADGKRTRQVLTEAGAAAKARDSVLA |
| | | VIRIGDDWTVVDLRGLLRNAQWRKLVPDGGITVQGLLDLFTG |
| | | DPVIDPRRGVVTFIYKADSVGIHSEKVCRGKQSKNLLERLCA |
| | | MPEKSSTRLDCARQAVALVSVDLGQRNPVAARFSRVSLAEG |
| | | QLQAQLVSAQFLDDAMVAMIRSYREEYDRFESLVREQAKAA |
| | | LSPEQLSEIVRHEADSAESVKSCVCAKFGIDPAGLSWDKMTSG |
| | | TWRIADHVQAAGGDVEWFFFKTCGKGKEIKTVRRSDFNVAK |
| | | QFRLRLSPETRKDWNDAIWELKRGNPAYVSFSKRKSEFARRV |
| | | VNDLVHRARRAVRCDEVVFAIEDLNISFFHGKGQRQMGWDA |
| | | FFEVKQENRWFIQALHKAFVERATHKGGYVLEVAPARTSTTC |
| | | PECRHCDPESRRGEQFCCIKCRHTCHADLEVATFNIEQVALTG |
| | | VSLPKRLSSTLL |
| CasΦ.28 | SEQ ID | MSKEKTPPSAYAILKAKHFPDLDFEKKHKMMAGRMFKNGAS |
| | NO: 166 | EQEVVQYLQGKGSESLMDVKPPAKSPILAQSRPFDEWEMVRT |
| | | SRLIQETIFGIPKRGSIPKRDGLSETQFNELVASLEVGGKPMLN |
| | | KQTRAIFYGLLGIKPPTFHAMAQNILIDLAINIRKGVLKKVDNL |
| | | NEKNRKKVKRIRDAGEQDVMVPAEVTAHDDRGYLNHPPGV |
| | | NPTIPGYQGVVIPFPEGFEGLPSGMTPVDWSHVLVDYLPHDRL |
| | | SIPKGSPGYIPEWQRPLLNRHKGRRHRSWYANSLNKPRKSRT |
| | | EEAKDRQNAGKRTALIEAERLKGVLPVLMRFKEDWLIIDARG |
| | | LLRNARYRGVLPEGSTLGNLIDLFSDSPRVDTRRGICTFLYRK |
| | | GRAYSTKPVKRKESKETLLKLTEKSTIALVSIDLGQTNPLTAK |
| | | LSKVRQVDGCLVAEPVLRKLIDNASEDGKEIARYRVAHDLLR |
| | | ARILEDAIDLLGIYKDEVVRARSDTPDLCKERVCRFLGLDSQA |
| | | IDWDRMTPYTDFIAQAFVAKGGDPKVVTIKPNGKPKMFRKD |
| | | RSIKNMKGIRLDISKEASSAYREAQWAIQRESPDFQRLAVWQS |
| | | QLTKRIVNQLVAWAKKCTQCDTVVLAFEDLNIGMMHGSGK |
| | | WANGGWNALFLHKQENRWFMQAFHKALTELSAHKGIPTIEV |
| | | LPHRTSITCTQCGHCHPGNRDGERFKCLKCEFLANTDLEIATD |
| | | NIERVALTGLPMPKGERSSAKRKPGGTRKTKKSKHSGNSPLA |
| | | AE |
| CasΦ.29 | SEQ ID | MEKAGPTSPLSVLIHKNFEGCRFQIDHLKIAGRKLAREGEAAA |
| | NO: 167 | IEYLLDKKCEGLPPNFQPPAKGNVIAQSRPFTEWAPYRASVAI |
| | | QKYIYSLSVDERKVCDPGSSSDSHEKWFKQTGVQNYGYTHV |
| | | QGLNLIFKHALARYDGVLKKVDNRNEKNRKKAERVNSFRRE |
| | | EGLPEEVFEEEKATDETGHLLQPPGVNHSIYCYQSVRPKPFNP |
| | | RKPGGISLPEAYSGYSLKPQDELPIGSLDRLSIPPGQPGYVPEW |
| | | QRSQLTTQKHRRKRSWYSAQKWKPRTGRTSTFDPDRLNCAR |
| | | AQGAILAVVRIHEDWVVFDVRGLLRNALWRELAGKGLTVRD |
| | | LLDFFTGDPVVDTKRGVVTFTYKLGKVDVHSLRTVRGKRSK |
| | | KVLEDLTLSSDVGLVTIDLGQTNVLAADYSKVTRSENGELLA |
| | | VPLSKSFLPKHLLHEVTAYRTSYDQMEEGFRRKALLTLTEDQ |
| | | QVEVTLVRDFSVESSKTKLLQLGVDVTSLPWEKMSSNTTYIS |
| | | DQLLQQGADPASLFFDGERDGKPCRHKKKDRTWAYLVRPKV |
| | | SPETRKALNEALWALKNTSPEFESLSKRKIQFSRRCMNYLLNE |
| | | AKRISGCGQVVFVIEDLNVRVHHGRGKRAIGWDNFFKPKREN |
| | | RWFMQALHKAASELAIHRGMHIIEACPARSSITCPKCGHCDPE |
| | | NRCSSDREKFLCVKCGAAFHADLEVATFNLRKVALTGTALPK |
| | | SIDHSRDGLIPKGARNRKLKEPQANDEKACA |
| CasΦ.30 | SEQ ID | MKEQSPLSSVLKSNFPGKKFLSADIRVAGRKLAQLGEAAAVE |
| | NO: 168 | YLSPRQRDSVPNFRPPAFCTVVAKSRPFEEWPIYKASVLLQEQ |
| | | IYGMTGQEFEERCGSIPTSLSGLRQWASSVGLGAAMEGLHVQ |
| | | GMNLMVKNAINRYKGVLVKVENRNKKLVEANEAKNSSREE |
| | | RGLPPLRPPELGSAFGPDGRLVNPPGIDKSIRLYQGVSPVPVVK |
| | | TTGRPTVHRLDIPAGEKGHVPLWQREAGLVKEGPRRRRMWY |
| | | SNSNLKRSRKDRSAEASEARKADSVVVRVSVKEDWVDIDVR |
| | | GLLRNVAWRGIERAGESTEDLLSLFSGDPVVDPSRDSVVFLY |
| | | KEGVVDVLSKKVVGAGKSRKQLEKMVSEGPVALVSCDLGQT |
| | | NYVAARVSVLDESLSPVRSFRVDPREFPSADGSQGVVGSLDRI |
| | | RADSDRLEAKLLSEAEASLPEPVRAEIEFLRSERPSAVAGRLCL |
| | | KLGIDPRSIPWEKMGSTTSFISEALSAKGSPLALHDGAPIKDSR |
| | | FAHAARGRLSPESRKALNEALWERKSSSREYGVISRRKSEASR |
| | | RMANAVLSESRRLTGLAVVAVNLEDLNMVSKFFHGRGKRAP |
| | | GWAGFFTPKMENRWFIRSIHKAMCDLSKHRGITVIESRPERTS |
| | | ISCPECGHCDPENRSGERFSCKSCGVSLHADFEVATRNLERVA |
| | | LTGKPMPRRENLHSPEGATASRKTRKKPREATASTFLDLRSVL |
| | | SSAENEGSGPAARAG |
| CasΦ.31 | SEQ ID | MLPPSNKIGKSMSLKEFINKRNFKSSIIKQAGKILKKEGEEAVK |
| | NO: 169 | KYLDDNYVEGYKKRDFPITAKCNIVASNRKIEDFDISKFSSFIQ |
| | | NYVFNLNKDNFEEFSKIKYNRKSFDELYKKIANEIGLEKPNYE |
| | | NIQGEIAVIRNAINIYNGVLKKVENRNKKIQEKNQSKDPPKLL |
| | | SAFDDNGFLAERPGINETIYGYQSVRLRHLDVEKDKDIIVQLP |
| | | DIYQKYNKKSTDKISVKKRLNKYNVDEYGKLISKRRKERINK |
| | | DDAILCVSNFGDDWIIFDARGLLRQTYRYKLKKKGLCIKDLL |
| | | NLFTGDPIINPTKTDLKEALSLSFKDGIINNRTLKVKNYKKCPE |
| | | LISELIRDKGKVAMISIDLGQTNPISYRLSKFTANNVAYIENGVI |
| | | SEDDIVKMKKWREKSDKLENLIKEEAIASLSDDEQREVRLYE |
| | | NDIADNTKKKILEKFNIREEDLDFSKMSNNTYFIRDCLKNKNI |
| | | DESEFTFEKNGKKLDPTDACFAREYKNKLSELTRKKINEKIWE |
| | | IKKNSKEYHKISIYKKETIRYIVNKLIKQSKEKSECDDIIVNIEK |
| | | LQIGGNFFGGRGKRDPGWNNFFLPKEENRWFINACHKAFSEL |
| | | APHKGIIVIESDPAYTSQTCPKCENCDKENRNGEKFKCKKCNY |
| | | EANADIDVATENLEKIAKNGRRLIKNFDQLGERLPGAEMPGG |
| | | ARKRKPSKSLPKNGRGAGVGSEPELINQSPSQVIA |
| CasΦ.32 | SEQ ID | VPDKKETPLVALCKKSFPGLRFKKHDSRQAGRILKSKGEGAA |
| | NO: 170 | VAFLEGKGGTTQPNFKPPVKCNIVAMSRPLEEWPIYKASVVIQ |
| | | KYVYAQSYEEFKATDPGKSEAGLRAWLKATRVDTDGYFNV |
| | | QGLNLIFQNARATYEGVLKKVENRNSKKVAKIEQRNEHRAER |
| | | GLPLLTLDEPETALDETGHLRHRPGINCSVFGYQHMKLKPYV |
| | | PGSIPGVTGYSRDPSTPIAACGVDRLEIPEGQPGYVPPWDREN |
| | | LSVKKHRRKRASWARSRGGAIDDNMLLAVVRVADDWALLD |
| | | LRGLLRNTQYRKLLDRSVPVTIESLLNLVTNDPTLSVVKKPGK |
| | | PVRYTATLIYKQGVVPVVKAKVVKGSYVSKMLDDTTETFSL |
| | | VGVDLGVNNLIAANALRIRPGKCVERLQAFTLPEQTVEDFFRF |
| | | RKAYDKHQENLRLAAVRSLTAEQQAEVLALDTFGPEQAKMQ |
| | | VCGHLGLSVDEVPWDKVNSRSSILSDLAKERGVDDTLYMFPF |
| | | FKGKGKKRKTEIRKRWDVNWAQHFRPQLTSETRKALNEAK |
| | | WEAERNSSKYHQLSIRKKELSRHCVNYVIRTAEKRAQCGKVI |
| | | VAVEDLHHSFRRGGKGSRKSGWGGFFAAKQEGRWLMDALF |
| | | GAFCDLAVHRGYRVIKVDPYNTSRTCPECGHCDKANRDRVN |
| | | REAFICVCCGYRGNADIDVAAYNIAMVAITGVSLRKAARASV |
| | | ASTPLESLAAE |
| CasΦ.33 | SEQ ID | MSKTKELNDYQEALARRLPGVRHQKSVRRAARLVYDRQGE |
| | NO: 171 | DAMVAFLDGKEVDEPYTLQPPAKCHILAVSRPIEEWPIARVT |
| | | MAVQEHVYALPVHEVEKSRPETTEGSRSAWFKNSGVSNHGV |
| | | THAQTLNAILKNAYNVYNGVIKKVENRNAKKRDSLAAKNKS |
| | | RERKGLPHFKADPPELATDEQGYLLQPPSPNSSVYLVQQHLR |
| | | TPQIDLPSGYTGPVVDPRSPIPSLIPIDRLAIPPGQPGYVPLHDR |
| | | EKLTSNKHRRMKLPKSLRAQGALPVCFRVFDDWAVVDGRGL |
| | | LRHAQYRRLAPKNVSIAELLELYTGDPVIDIKRNLMTFRFAEA |
| | | VVEVTARKIVEKYHNKYLLKLTEPKGKPVREIGLVSIDLNVQ |
| | | RLIALAIYRVHQTGESQLALSPCLHREILPAKGLGDFDKYKSK |
| | | FNQLTEEILTAAVQTLTSAQQEEYQRYVEESSHEAKADLCLK |
| | | YSITPHELAWDKMTSSTQYISRWLRDHGWNASDFTQITKGRK |
| | | KVERLWSDSRWAQELKPKLSNETRRKLEDAKHDLQRANPEW |
| | | QRLAKRKQEYSRHLANTVLSMAREYTACETVVIAIENLPMKG |
| | | GFVDGNGSRESGWDNFFTHKKENRWMIKDIHKALSDLAPNR |
| | | GVHVLEVNPQYTSQTCPECGHRDKANRDPIQRERFCCTHCGA |
| | | QRHADLEVATHNIAMVATTGKSLTGKSLAPQRLQEAAE |
| CasΦ.41 | SEQ ID | VLLSDRIQYTDPSAPIPAMTVVDRRKIKKGEPGYVPPFMRKNL |
| | NO: 172 | STNKHRRMRLSRGQKEACALPVGLRLPDGKDGWDFIIFDGRA |
| | | LLRACRRLRLEVTSMDDVLDKFTGDPRIQLSPAGETIVTCMLK |
| | | PQHTGVIQQKLITGKMKDRLVQLTAEAPIAMLTVDLGEHNLV |
| | | ACGAYTVGQRRGKLQSERLEAFLLPEKVLADFEGYRRDSDEH |
| | | SETLRHEALKALSKRQQREVLDMLRTGADQARESLCYKYGL |
| | | DLQALPWDKMSSNSTFIAQHLMSLGFGESATHVRYRPKRKAS |
| | | ERTILKYDSRFAAEEKIKLTDETRRAWNEAIWECQRASQEFRC |
| | | LSVRKLQLARAAVNWTLTQAKQRSRCPRVVVVVEDLNVRF |
| | | MHGGGKRQEGWAGFFKARSEKRWFIQALHKAYTELPTNRGI |
| | | HVMEVNPARTSITCTKCGYCDPENRYGEDFHCRNPKCKVRG |
| | | GHVANADLDIATENLARVALSGPMPKAPKLK |
| CasΦ.34 | SEQ ID | MTPSFGYQMIIVTPIHHASGAWATLRLLFLNPKTSGVMLGMT |
| | NO: 173 | KTKSAFALMREEVFPGLLFKSADLKMAGRKFAKEGREAAIEY |
| | | LRGKDEERPANFKPPAKGDIIAQSRPFDQWPIVQVSQAIQKYIF |
| | | GLTKAEFDATKTLLYGEGNHPTTESRRRWFEATGVPDFGFTS |
| | | AQGLNAIFSSALARYEGVIQKVENRNEKRLKKLSEKNQRLVE |
| | | EGHAVEAYVPETAFHTLESLKALSEKSLVPLDDLMDKIDRLA |
| | | QPPGINPCLYGYQQVAPYIYDPENPRGVVLPDLYLGYCRKPD |
| | | DPITACPNRLDIPKGQPGYIPEHQRGQLKKHGRVRRFRYTNPQ |
| | | AKARAKAQTAILAVLRIDEDWVVMDLRGLLRNVYFREVAAP |
| | | GELTARTLLDTFTGCPVLNLRSNVVTFCYDIESKGALHAEYV |
| | | RKGWATRNKLLDLTKDGQSVALLSVDLGQRHPVAVMISRLK |
| | | RDDKGDLSEKSIQVVSRTFADQYVDKLKRYRVQYDALRKEIY |
| | | DAALVSLPPEQQAEIRAYEAFAPGDAKANVLSVMFQGEVSPD |
| | | ELPWDKMNTNTHYISDLYLRRGGDPSRVFFVPQPSTPKKNAK |
| | | KPPAPRKPVKRTDENVSHMPEFRPHLSNETREAFQKAKWTM |
| | | ERGNVRYAQLSRFLNQIVREANNWLVSEAKKLTQCQTVVWA |
| | | IEDLHVPFFHGKGKYHETWDGFFRQKKEDRWFVNVFHKAISE |
| | | RAPNKGEYVMEVAPYRTSQRCPVCGFVDADNRHGDHFKCLR |
| | | CGVELHADLEVATWNIALVAVQGHGIAGPPREQSCGGETAG |
| | | TARKGKNIKKNKGLADAVTVEAQDSEGGSKKDAGTARNPVY |
| | | IPSESQVNCPAP |
| CasΦ.35 | SEQ ID | MKPKTPKPPKTPVAALIDKHFPGKRFRASYLKSVGKKLKNQG |
| | NO: 174 | EDVAVRFLTGKDEERPPNFQPPAKSNIVAQSRPIEEWPIHKVS |
| | | VAVQEYVYGLTVAEKEACSDAGESSSSHAAWFAKTGVENFG |
| | | YTSVQGLNKIFPPTFNRFDGVIKKVENRNEKKRQKATRINEAK |
| | | RNKGQSEDPPEAEVKATDDAGYLLQPPGINHSVYGYQSITLCP |
| | | YTAEKFPTIKLPEEYAGYHSNPDAPIPAGVPDRLAIPEGQPGH |
| | | VPEEHRAGLSTKKHRRVRQWYAMANWKPKPKRTSKPDYDR |
| | | LAKARAQGALLIVIRIDEDWVVVDARGLLRNVRWRSLGKREI |
| | | TPNELLDLFTGDPVLDLKRGVVTFTYAEGVVNVCSRSTTKGK |
| | | QTKVLLDAMTAPRDGKKRQIGMVAVDLGQTNPIAAEYSRVG |
| | | KNAAGTLEATPLSRSTLPDELLREIALYRKAHDRLEAQLREEA |
| | | VLKLTAEQQAENARYVETSEEGAKLALANLGVDTSTLPWDA |
| | | MTGWSTCISDHLINHGGDTSAVFFQTIRKGTKKLETIKRKDSS |
| | | WADIVRPRLTKETREALNDFLWELKRSHEGYEKLSKRLEELA |
| | | RRAVNHVVQEVKWLTQCQDIVIVIEDLNVRNFHGGGKRGGG |
| | | WSNFFTVKKENRWFMQALHKAFSDLAAHRGIPVLEVYPART |
| | | SITCLGCGHCDPENRDGEAFVCQQCGATFHADLEVATRNIAR |
| | | VALTGEAMPKAPAREQPGGAKKRGTSRRRKLTEVAVKSAEP |
| | | TIHQAKNQQLNGTSRDPVYKGSELPAL |
| CasΦ.43 | SEQ ID | MSEITDLLKANFKGKTFKSADMRMAGRILKKSGAQAVIKYLS |
| | NO: 175 | DKGAVDPPDFRPPAKCNIIAQSRPFDEWPICKASMAIQQHIYG |
| | | LTKNEFDESSPGTSSASHEQWFAKTGVDTHGFTHVQGLNLIF |
| | | QHAKKRYEGVIKKVENYNEKERKKFEGINERRSKEGMPLLEP |
| | | RLRTAFGDDGKFAEKPGVNPSIYLYQQTSPRPYDKTKHPYVH |
| | | APFELKEITTIPTQDDRLKIPFGAPGHVPEKHRSQLSMAKHKR |
| | | RRAWYALSQNKPRPPKDGSKGRRSVRDLADLKAASLADAIPL |
| | | VSRVGFDWVVIDGRGLLRNLRWRKLAHEGMTVEEMLGFFSG |
| | | DPVIDPRRNVATFIYKAEHATVKSRKPIGGAKRAREELLKATA |
| | | SSDGVIRQVGLISVDLGQTNPVAYEISRMHQANGELVAEHLE |
| | | YGLLNDEQVNSIQRYRAAWDSMNESFRQKAIESLSMEAQDEI |
| | | MQASTGAAKRTREAVLTMFGPNATLPWSRMSSNTTCISDALI |
| | | EVGKEEETNFVTSNGPRKRTDAQWAAYLRPRVNPETRALLN |
| | | QAVWDLMKRSDEYERLSKRKLEMARQCVNFVVARAEKLTQ |
| | | CNNIGIVLENLVVRNFHGSGRRESGWEGFFEPKRENRWFMQV |
| | | LHKAFSDLAQHRGVMVFEVHPAYSSQTCPACRYVDPKNRSS |
| | | EDRERFKCLKCGRSFNADREVATFNIREIARTGVGLPKPDCER |
| | | SRGVQTTGTARNPGRSLKSNKNPSEPKRVLQSKTRKKITSTET |
| | | QNEPLATDLKT |
| CasΦ.44 | SEQ ID | MTPKTESPLSALCKKHFPGKRFRTNYLKDAGKILKKHGEDAV |
| | NO: 176 | VAFLSDKQEDEPANFCPPAKVHILAQSRPFEDWPINLASKAIQ |
| | | TYVYGLTADERKTCEPGTSKESHDRWFKETGVDHHGFTSVQ |
| | | GLNLIFKHTLNRYDGVIKKVETRNEKRRSSVVRINEKKAAEG |
| | | LPLIAAEAEETAFGEDGRLLQPPGVNHSIYCFQQVSPQPYSSK |
| | | KHPQVVLPHAVQGVDPDAPIPVGRPNRLDIPKGQPGYVPEWQ |
| | | RPHLSMKCT<RVRMWYARANWRRKPGRRSVLNEARLKEASA |
| | | KGALPIVLVIGDDWLVMDARGLLRSVFWRRVAKPGLSLSELL |
| | | NVTPTGLFSGDPVIDPKRGLVTFTSKLGVVAVHSRKPTRGKKS |
| | | KDLLLKMTKPTDDGMPRHVGMVAIDLGQTNPVAAEYSRVV |
| | | QSDAGTLKQEPVSRGVLPDDLLKDVARYRRAYDLTEESIRQE |
| | | AIALLSEGHRAEVTKLDQTTANETKRLLVDRGVSESLPWEKM |
| | | SSNTTYISDCLVALGKTDDVFFVPKAKKGKKETGIAVKRKDH |
| | | GWSKLLRPRTSPEARKALNENQWAVKRASPEYERLSRRKLEL |
| | | GRRCVNHIIQETKRWTQCEDIVVVLEDLNVGFFHGSGKRPDG |
| | | WDNFFVSKRENRWFIQVLHKAFGDLATHRGTHVIEVHPARTS |
| | | ITCIKCGHCDAGNRDGESFVCLASACGDRRHADLEVATRNVA |
| | | RVAITGERMPPSEQARDVQKAGGARKRKPSARNVKSSYPAV |
| | | EPAPASP |
| CasΦ.36 | SEQ ID | MSDNKMKKLSKEEKPLTPLQILIRKYIDKSQYPSGFKTTIIKQA |
| | NO: 177 | GVRIKSVKSEQDEINLANWIISKYDPTYIKRDFNPSAKCQIIATS |
| | | RSVADFDIVKMSNKVQEIFFASSHLDKNVFDIGKSKSDHDSW |
| | | FERNNVDRGIYTYSNVQGMNLIFSNTKNTYLGVAVKAQNKFS |
| | | SKMKRIQDINNFRITNHQSPLPIPDEIKIYDDAGFLLNPPGVNP |
| | | NIFGYQSCLLKPLENKEIISKTSFPEYSRLPADMIEVNYKISNRL |
| | | KFSNDQKGFIQFKDKLNLFKINSQELFSKRRRLSGQPILLVASF |
| | | GDDWVVLDGRGLLRQVYYRGIAKPGSITISELLGFFTGDPIVD |
| | | PIRGVVSLGFKPGVLSQETLKTTSARIFAEKLPNLVLNNNVGL |
| | | MSIDLGQTNPVSYRLSEITSNMSVEHICSDFLSQDQISSIEKAKT |
| | | SLDNLEEEIAIKAVDHLSDEDKINFANFSKLNLPEDTRQSLFEK |
| | | YPELIGSKLDFGSMGSGTSYIADELIKFENKDAFYPSGKKKFD |
| | | LSFSRDLRKKLSDETRKSYNDALFLEKRTNDKYLKNAKRRKQ |
| | | IVRTVANSLVSKIEELGLTPVINIENLAMSGGFFDGRGKREKG |
| | | WDNFFKVKKENRWVMKDFHKAFSELSPHHGVIVIESPPYCTS |
| | | VTCTKCNFCDKKNRNGHKFTCQRCGLDANADLDIATENLEK |
| | | VAISGKRMPGSERSSDERKVAVARKAKSPKGKAIKGVKCTIT |
| | | DEPALLSANSQDCSQSTS |
| CasΦ.37 | SEQ ID | MALSLAEVRERHFKGLRFRSSYLKRAGKILKKEGEAACVAYL |
| | NO: 178 | TGKDEESPPNFKPPAKCDVVAQSRPFEEWPIVQASVAVQSYV |
| | | YGLTKEAFEAFNPGTTKQSHEACLAATGIDTCGYSNVQGLNL |
| | | IFRQAKNRYEGVITKVENRNKKAKKKLTRKNEWRQKNGHSE |
| | | LPEAPEELTFNDEGRLLQPPGINPSLYTYQQISPTPWSPKDSSIL |
| | | PPQYAGYERDPNAPIPFGVAKDRLTIASGCPGYIPEWMRTAGE |
| | | KTNPRTQKKFMHPGLSTRKNKRMRLPRSVRSAPLGALLVTIH |
| | | LGEDWLVLDVRGLLRNARWRGVAPKDISTQGLLNLFTGDPVI |
| | | DTRRGVVTFTYKPETVGIHSRTWLYKGKQTKEVLEKLTQDQT |
| | | VALVAIDLGQTNPVSAAASRVSRSGENLSIETVDRFFLPDELIK |
| | | ELRLYRMAHDRLEERIREESTLALTEAQQAEVRALEHVVRDD |
| | | AKNKVCAAFNLDAASLPWDQMTSNTTYLSEAILAQGVSRDQ |
| | | VFFTPNPKKGSKEPVEVMRKDRAWVYAFKAKLSEETRKAKN |
| | | EALWALKRASPDYARLSKRREELCRRSVNMVINRAKKRTQC |
| | | QVVIPVLEDLNIGFFHGSGKRLPGWDNFFVAKKENRWLMNG |
| | | LHKSFSDLAVHRGFYVFEVMPHRTSITCPACGHCDSENRDGE |
| | | AFVCLSCKRTYHADLDVATHNLTQVAGTGLPMPEREHPGGT |
| | | KKPGGSRKPESPQTHAPILHRTDYSESADRLGS |
| CasΦ.45 | SEQ ID | QAVIKYLSDKGAVDPPDFRPPAKCNIIAQSRPFDEWPICKASM |
| | NO: 179 | AIQQHIYGLTKNEFDESSPGTSSASHEQWFAKTGVDTHGFTH |
| | | VQGLNLIFQHAKKRYEGVIKKVENYNEKERKKFEGINERRSK |
| | | EGMPLLEPRLRTAFGDDGKFAEKPGVNPSIYLYQQTSPRPYD |
| | | KTKHPYVHAPFELKEITTIPTQDDRLKIPFGAPGHVPEKHRSQL |
| | | SMAKHKRRRAWYALSQNKPRPPKDGSKGRRSVRDLADLKA |
| | | ASLADAIPLVSRVGFDWVVIDGRGLLRNLRWRKLAHEGMTV |
| | | EEMLGFFSGDPVIDPRRNVATFIYKAEHATVKSRKPIGGAKRA |
| | | REELLKATASSDGVIRQVGLISVDLGQTNPVAYEISRMHQAN |
| | | GELVAEHLEYGLLNDEQVNSIQRYRAAWDSMNESFRQKAIES |
| | | LSMEAQDEIMQASTGAAKRTREAVLTMFGPNATLPWSRMSS |
| | | NTTCISDALIEVGKEEETNFVTSNGPRKRTDAQWAAYLRPRV |
| | | NPETRALLNQAVWDLMKRSDEYERLSKRKLEMARQCVNFV |
| | | VARAEKLTQCNNIGIVLENLVVRNFHGSGRRESGWEGFFEPK |
| | | RENRWFMQVLHKAFSDLAQHRGVMVFEVHPAYSSQTCPACR |
| | | YVDPKNRSSEDRERFKCLKCGRSFNADREVATFNIREIARTGV |
| | | GLPKPDCERSRDVQTPGTARKSGRSLKSQDNLSEPKRVLQSK |
| | | TRKKITSTETQNEPLATDLKT |
| CasΦ.38 | SEQ ID | MIKEQSELSKLIEKYYPGKKFYSNDLKQAGKHLKKSEHLTAK |
| | NO: 180 | ESEELTVEFLKSCKEKLYDFRPPAKALIISTSRPFEEWPIYKASE |
| | | SIQKYIYSLTKEELEKYNISTDKTSQENFFKESLIDNYGFANVS |
| | | GLNLIFQHTKAIYDGVLKKVNNRNNKILKKYKRKIEEGIEIDSP |
| | | ELEKAIDESGHFINPPGINKNIYCYQQVSPTIFNSFKETKIICPFN |
| | | YKRNPNDIIQKGVIDRLAIPFGEPGYIPDHQRDKVNKHKKRIR |
| | | KYYKNNENKNKDAILAKINIGEDWVLFDLRGLLRNAYWRKL |
| | | IPKQGITPQQLLDMFSGDPVIDPIKNNITFIYKESIIPIHSESIIKTK |
| | | KSKELLEKLTKDEQIALVSIDLGQTNPVAARFSRLSSDLKPEH |
| | | VSSSFLPDELKNEICRYREKSDLLEIEIKNKAIKMLSQEQQDEI |
| | | KLVNDISSEELKNSVCKKYNIDNSKIPWDKMNGFTTFIADEFI |
| | | NNGGDKSLVYFTAKDKKSKKEKLVKLSDKKIANSFKPKISKE |
| | | TREILNKITWDEKISSNEYKKLSKRKLEFARRATNYLINQAKK |
| | | ATRLNNVVLVVEDLNSKFFHGSGKREDGWDNFFIPKKENRW |
| | | FIQALHKSLTDVSIHRGINVIEVRPERTSITCPKCGCCDKENRK |
| | | GEDFKCIKCDSVYHADLEVATFNIEKVAITGESMPKPDCERLG |
| | | GEESIG |
| CasΦ.39 | SEQ ID | VAFLDGKEVDEPYTLQPPAKCHILAVSRPIEEWPIARVTMAVQ |
| | NO: 181 | EHVYALPVHEVEKSRPETTEGSRSAWFKNSGVSNHGVTHAQ |
| | | TLNAILKNAYNVYNGVIKKVENRNAKKRDSLAAKNKSRERK |
| | | GLPHFKADPPELATDEQGYLLQPPSPNSSVYLVQQHLRTPQID |
| | | LPSGYTGPVVDPRSPIPSLIPIDRLAIPPGQPGYVPLHDREKLTS |
| | | NKHRRMKLPKSLRAQGALPVCFRVFDDWAVVDGRGLLRHA |
| | | QYRRLAPKNVSIAELLELYTGDPVIDIKRNLMTFRFAEAVVEV |
| | | TARKIVEKYHNKYLLKLTEPKGKPVREIGLVSIDLNVQRLIAL |
| | | AIYRVHQTGESQLALSPCLHREILPAKGLGDFDKYKSKFNQLT |
| | | EEILTAAVQTLTSAQQEEYQRYVEESSHEAKADLCLKYSITPH |
| | | ELAWDKMTSSTQYISRWLRDHGWNASDFTQITKGRKKVERL |
| | | WSDSRWAQELKPKLSNETRRKLEDAKHDLQRANPEWQRLA |
| | | KRKQEYSRHLANTVLSMAREYTACETVVIAIENLPMKGGFVD |
| | | GNGSRESGWDNFFTHKKENRWMIKDIHKALSDLAPNRGVHV |
| | | LEVNPQYTSQTCPECGHRDKANRDPIQRERFCCTHCGAQRHA |
| | | DLEVATHNIAMVATTGKSLTGKSLAPQRLQ |
| CasΦ.42 | SEQ ID | LEIPEGEPGHVPWFQRMDIPEGQIGHVNKIQRFNFVHGKNSGK |
| | NO: 182 | VKFSDKTGRVKRYHHSKYKDATKPYKFLEESKKVSALDSILA |
| | | IITIGDDWVVFDIRGLYRNVFYRELAQKGLTAVQLLDLFTGDP |
| | | VIDPKKGIITFSYKEGVVPVFSQKIVSRFKSRDTLEKLTSQGPV |
| | | ALLSVDLGQNEPVAARVCSLKNINDKIALDNSCRIPFLDDYKK |
| | | QIKDYRDSLDELEIKIRLEAINSLDVNQQVEIRDLDVFSADRAK |
| | | ASTVDMFDIDPNLISWDSMSDARFSTQISDLYLKNGGDESRV |
| | | YFEINNKRIKRSDYNISQLVRPKLSDSTRKNLNDSIWKLKRTSE |
| | | EYLKLSKRKLELSRAVVNYTIRQSKLLSGINDIVIILEDLDVKK |
| | | KFNGRGIRDIGWDNFFSSRKENRWFIPAFHKSFSELSSNRGLC |
| | | VIEVNPAWTSATCPDCGFCSKENRDGINFTCRKCGVSYHADI |
| | | DVATLNIARVAVLGKPMSGPADRERLGGTKKPRVARSRKDM |
| | | KRKDISNGTVEVMVTA |
| CasΦ.46 | SEQ ID | IPSFGYLDRLKIAKGQPGYIPEWQRETINPSKKVRRYWATNHE |
| | NO: 183 | KIRNAIPLVVFIGDDWVIIDGRGLLRDARRRKLADKNTTIEQL |
| | | LEMVSNDPVIDSTRGIATLSYVEGVVPVRSFIPIGEKKGREYLE |
| | | KSTQKESVTLLSVDIGQINPVSCGVYKVSNGCSKIDFLDKFFL |
| | | DKKHLDAIQKYRTLQDSLEASIVNEALDEIDPSFKKEYQNINS |
| | | QTSNDVKKSLCTEYNIDPEAISWQDITAHSTLISDYLIDNNITN |
| | | DVYRTVNKAKYKTNDFGWYKKFSAKLSKEAREALNEKIWEL |
| | | KIASSKYKKLSVRKKEIARTIANDCVKRAETYGDNVVVAMES |
| | | LTKNNKVMSGRGKRDPGWHNLGQAKVENRWFIQAISSAFED |
| | | KATHHGTPVLKVNPAYTSQTCPSCGHCSKDNRSSKDRTIFVC |
| | | KSCGEKFNADLDVATYNIAHVAFSGKKLSPPSEKSSATKKPRS |
| | | ARKSKKSRKS |
| CasΦ.47 | SEQ ID | SPIEKLLNGLLVKITFGNDWIICDARGLLDNVQKGIIHKSYFTN |
| | NO: 184 | KSSLVDLIDLFTCNPIVNYKNNVVTFCYKEGVVDVKSFTPIKS |
| | | GPKTQENLIKKLKYSRFQNEKDACVLGVGVDVGVTNPFAING |
| | | FKMPVDESSEWVMLNEPLFTIETSQAFREEIMAYQQRTDEMN |
| | | DQFNQQSIDLLPPEYKVEFDNLPEDINEVAKYNLLHTLNIPNN |
| | | FLWDKMSNTTQFISDYLIQIGRGTETEKTITTKKGKEKILTIRD |
| | | VNWFNTFKPKISEETGKARTEIKRDLQKNSDQFQKLAKSREQ |
| | | SCRTWVNNVTEEAKIKSGCPLIIFVIEALVKDNRVFSGKGHRA |
| | | IGWHNFGKQKNERRWWVQAIHKAFQEQGVNHGYPVILCPPQ |
| | | YTSQTCPKCNHVDRDNRSGEKFKCLKYGWIGNADLDVGAYN |
| | | IARVAITGKALSKPLEQKKIKKAKNKT |
| CasΦ.48 | SEQ ID | LLDNVQKGIIHKSYFTNKSSLVDLIDLFTCNPIVNYKNNVVTF |
| | NO: 185 | CYKEGVVDVKSFTPIKSGPKTQENLIKKLKYSRFQNEKDACV |
| | | LGVGVDVGVTNPFAINGFKMPVDESSEWVMLNEPLFTIETSQ |
| | | AFREEIMAYQQRTDEMNDQFNQQSIDLLPPEYKVEFDNLPEDI |
| | | NEVAKYNLLHTLNIPNNFLWDKMSNTTQFISDYLIQIGRGTET |
| | | EKTITTKKGKEKILTIRDVNWFNTFKPKISEETGKARTEIKRDL |
| | | QKNSDQFQKLAKSREQSCRTWVNNVTEEAKIKSGCPLIIFVIE |
| | | ALVKDNRVFSGKGHRAIGWHNFGKQKNERRWWVQAIHKAF |
| | | QEQGVNHGYPVILCPPQYTSQTCPKCNHVDRDNRSGEKFKCL |
| | | KYGWIGNADLDVGAYNIARVAITGKALSKPLEQKKIKKAKN |
| | | KT |
| CasΦ.49 | SEQ ID | MIKPTVSQFLTPGFKLIRNHSRTAGLKLKNEGEEACKKFVREN |
| | NO: 186 | EIPKDECPNFQGGPAIANIIAKSREFTEWEIYQSSLAIQEVIFTLP |
| | | KDKLPEPILKEEWRAQWLSEHGLDTVPYKEAAGLNLIIKNAV |
| | | NTYKGVQVKVDNKNKNNLAKINRKNEIAKLNGEQEISFEEIK |
| | | AFDDKGYLLQKPSPNKSIYCYQSVSPKPFITSKYHNVNLPEEYI |
| | | GYYRKSNEPIVSPYQFDRLRIPIGEPGYVPKWQYTFLSKKENK |
| | | RRKLSKRIKNVSPILGIICIKKDWCVFDMRGLLRTNHWKKYH |
| | | KPTDSINDLFDYFTGDPVIDTKANVVRFRYKMENGIVNYKPV |
| | | REKKGKELLENICDQNGSCKLATVDVGQNNPVAIGLFELKKV |
| | | NGELTKTLISRHPTPIDFCNKITAYRERYDKLESSIKLDAIKQLT |
| | | SEQKIEVDNYNNNFTPQNTKQIVCSKLNINPNDLPWDKMISGT |
| | | HFISEKAQVSNKSEIYFTSTDKGKTKDVMKSDYKWFQDYKPK |
| | | LSKEVRDALSDIEWRLRRESLEFNKLSKSREQDARQLANWISS |
| | | MCDVIGIENLVKKNNFFGGSGKREPGWDNFYKPKKENRWWI |
| | | NAIHKALTELSQNKGKRVILLPAMRTSITCPKCKYCDSKNRN |
| | | GEKFNCLKCGIELNADIDVATENLATVAITAQSMPKPTCERSG |
| | | DAKKPVRARKAKAPEFHDKLAPSYTVVLREAVKRPAATKK |
| | | AGQAKKKKEF |
| | | (Bold sequence is Nuclear Localization Signal) |

In some embodiments, any of the programmable CasΦ nuclease of the present disclosure (e.g., any one of SEQ ID NO: 139-SEQ ID NO: 186 or fragments or variants thereof) may include a nuclear localization signal (NLS). In some cases, said NLS may have a sequence of KRPAATKKAGQAKKKKEF (SEQ ID NO: 187).

A CasΦ polypeptide or a variant thereof can comprise at least 70%, at least 75%, at least 80%, at least 85%, at least 90%, at least 92%, at least 95%, at least 97%, at least 99%, or 100% sequence identity with any one of SEQ ID NO: 139-SEQ ID NO: 186.

In some embodiments, the CasΦ nuclease comprises more than 200 amino acids, more than 300 amino acids, more than 400 amino acids. In some embodiments, the CasΦ nuclease comprises less than 1500 amino acids, less than 1000 amino acids or less than 900 amino acids. In some embodiments, the CasΦ nuclease comprises between 200 and 1500 amino acids, between 300 and 1000 amino acids, or between 400 and 900 amino acids. In preferred embodiments, the CasΦ nuclease comprises between 400 and 900 amino acids.

A programmable CasΦ nuclease of the present disclosure may have a single active site in a RuvC domain that is capable of catalyzing pre-crRNA processing and nicking or cleaving of nucleic acids. This compact catalytic site may render the programmable CasΦ nuclease especially advantageous for genome engineering and new functionalities for genome manipulation.

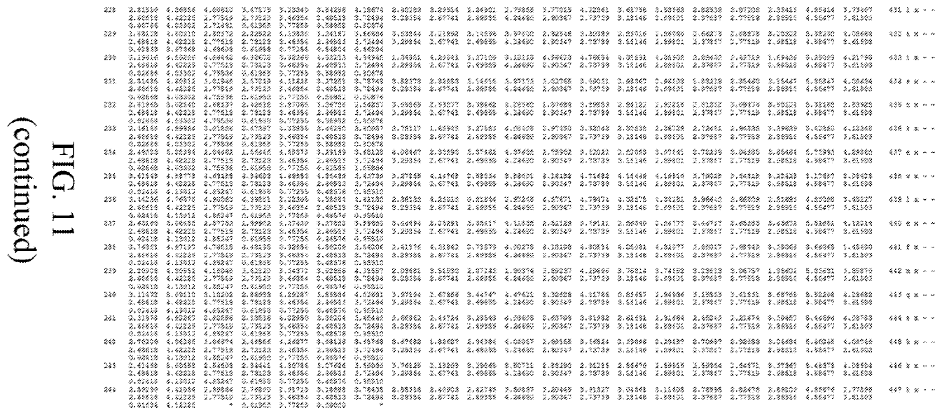

In some embodiments, the RuvC domain is a RuvC-like domain. Various RuvC-like domains are known in the art and are easily identified using online tools such as InterPro (https://www.ebi.ac.uk/interpro/). For example, a RuvC-like domain may be a domain which shares homology with a region of TnpB proteins of the IS605 and other related families of transposons, as described in review articles such as Shmakov et al. (Nature Reviews Microbiology volume 15, pages 169-182(2017)) and Koonin E. V. and Makarova K. S. (2019, Phil. Trans. R. Soc., B 374:20180087). In some embodiments, the RuvC-like domain shares homology with the transposase IS605, OrfB, C-terminal. A transposase IS605, OrfB, C-terminal is easily identified by the skilled person using bioinformatics tools, such as PFAM (Finn et al. (Nucleic Acids Res. 2014 Jan. 1; 42(Database issue): D222-D230); El-Gebali et al. (2019) Nucleic Acids Res. doi:10.1093/nar/gky995). PFAM is a database of protein families in which each entry is composed of a seed alignment which forms the basis to build a profile hidden Markov model (HMM) using the HMMER software (hmmer.org). It is readily accessible via pfam.xfam.org, maintained by EMBL-EBI, which easily allows an amino acid sequence to be analyzed against the current release of PFAM (e.g. version 33.1 from May 2020), but local builds can also be implemented using publicly- and freely-available database files and tools. A transposase IS605, OrfB, C-terminal is easily identified by the skilled person using the HMM PF07282. PF07282 is reproduced for reference in FIG. 11 (accession number PF07282.12). The skilled person would also be able to identify a RuvC domain, for example with the HMM PF18516, using the PFAM tool. PF18516 is reproduced for reference in FIG. 12 (accession number PF18516.2). In some embodiments, the programmable CasΦ nuclease comprises a RuvC-like domain which matches PFAM family PF07282 but does not match PFAM family PF18516, as assessed using the PFAM tool (e.g. using PFAM version 33.1, and the HMM accession numbers PF07282.12 and PF18516.2). PFAM searches should ideally be performed using an E-value cut-off set at 1.0.

### Detector Nucleic Acids

Described herein are reagents comprising a single stranded detector nucleic acid comprising a detection moiety, wherein the detector nucleic acid is capable of being cleaved by the activated programmable nuclease, thereby generating a first detectable signal. As used herein, a detector nucleic acid is used interchangeably with reporter or reporter molecule. As described herein, nucleic acid sequences comprising DNA may be detected using a DNA-activated programmable RNA nuclease, a DNA-activated programmable DNA nuclease, or a combination thereof, and other reagents disclosed herein. The DNA-activated programmable RNA nuclease may be activated and cleave the detector RNA upon binding of an engineered guide nucleic acid to a target DNA. In some cases, the detector nucleic acid is a single-stranded nucleic acid sequence comprising ribonucleotides. Additionally, detection by a DNA-activated programmable RNA nuclease, which can cleave RNA reporters, allows for multiplexing with a DNA-activated programmable DNA nuclease that can cleave DNA reporters (e.g., Type V CRISPR enzyme). In some cases, the detector nucleic acid is a single-stranded nucleic acid sequence comprising deoxyribonucleotides.

The detector nucleic acid can be a single-stranded nucleic acid sequence comprising at least one deoxyribonucleotide and at least one ribonucleotide. In some cases, the detector nucleic acid is a single-stranded nucleic acid comprising at least one ribonucleotide residue at an internal position that functions as a cleavage site. In some cases, the detector nucleic acid comprises at least 2, 3, 4, 5, 6, 7, 8, 9, or 10 ribonucleotide residues at an internal position. In some cases, the detector nucleic acid comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some cases, the detector nucleic acid has only ribonucleotide residues. In some cases, the detector nucleic acid has only deoxyribonucleotide residues. In some cases, the detector nucleic acid comprises nucleotides resistant to cleavage by the programmable nuclease described herein. In some cases, the detector nucleic acid comprises synthetic nucleotides. In some cases, the detector nucleic acid comprises at least one ribonucleotide residue and at least one non-ribonucleotide residue. In some cases, the detector nucleic acid is 5-20, 5-15, 5-10, 7-20, 7-15, or 7-10 nucleotides in length. In some cases, the detector nucleic acid is from 3 to 20, from 4 to 10, from 5 to 10, or from 5 to 8 nucleotides in length. In some cases, the detector nucleic acid comprises at least one uracil ribonucleotide. In some cases, the detector nucleic acid comprises at least two uracil ribonucleotides. Sometimes the detector nucleic acid has only uracil ribonucleotides. In some cases, the detector nucleic acid comprises at least one adenine ribonucleotide. In some cases, the detector nucleic acid comprises at least two adenine ribonucleotide. In some cases, the detector nucleic acid has only adenine ribonucleotides. In some cases, the detector nucleic acid comprises at least one cytosine ribonucleotide. In some cases, the detector nucleic acid comprises at least two cytosine ribonucleotide. In some cases, the detector nucleic acid comprises at least one guanine ribonucleotide. In some cases, the detector nucleic acid comprises at least two guanine ribonucleotide. A detector nucleic acid can comprise only unmodified ribonucleotides, only unmodified deoxyribonucleotides, or a combination thereof. In some cases, the detector nucleic acid is from 5 to 12 nucleotides in length. In some cases, the detector nucleic acid is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the detector nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a detector nucleic acid can be 5, 8, or 10 nucleotides in length. For cleavage by a programmable nuclease comprising Cas12, a detector nucleic acid can be 10 nucleotides in length.

The single stranded detector nucleic acid comprises a detection moiety capable of generating a first detectable signal. Sometimes the detector nucleic acid comprises a protein capable of generating a signal. A signal can be a calorimetric, potentiometric, amperometric, optical (e.g., fluorescent, colorometric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the detector nucleic acid. Sometimes the detection moiety is at the 3′ terminus of the detector nucleic acid. In some cases, the detection moiety is at the 5′ terminus of the detector nucleic acid. In some cases, the quenching moiety is at the 3′ terminus of the detector nucleic acid. In some cases, the single-stranded detector nucleic acid is at least one population of the single-stranded nucleic acid capable of generating a first detectable signal. In some cases, the single-stranded detector nucleic acid is a population of the single stranded nucleic acid capable of generating a first detectable signal. Optionally, there are more than one population of single-stranded detector nucleic acid. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded detector nucleic acids capable of generating a detectable signal. In some cases there are from 2 to 50, from 3 to 40, from 4 to 30, from 5 to 20, or from 6 to 10 different populations of single-stranded detector nucleic acids capable of generating a detectable signal.

TABLE 5
Exemplary Single Stranded Detector Nucleic Acid

| 5′ Detection | | |
|---|---|---|
| Moiety* | Sequence (SEQ ID NO:) | 3′ Quencher* |
| /56-FAM/ | rUrUrUrUrU (SEQ ID NO: 1) | /3IABkFQ/ |
| /5IRD700/ | rUrUrUrUrU (SEQ ID NO: 1) | /3IRQC1N/ |
| /5TYE665/ | rUrUrUrUrU (SEQ ID NO: 1) | /3IAbRQSp/ |
| /5Alex594N/ | rUrUrUrUrU (SEQ ID NO: 1) | /3IAbRQSp/ |
| /5ATTO633N/ | rUrUrUrUrU (SEQ ID NO: 1) | /3IAbRQSp/ |
| /56-FAM/ | rUrUrUrUrUrUrUrU (SEQ ID NO: 2) | /3IABkFQ/ |
| /5IRD700/ | rUrUrUrUrUrUrUrU (SEQ ID NO: 2) | /3IRQC1N/ |
| /5TYE665/ | rUrUrUrUrUrUrUrU (SEQ ID NO: 2) | /3IAbRQSp/ |
| /5Alex594N/ | rUrUrUrUrUrUrUrU (SEQ ID NO: 2) | /3IAbRQSp/ |
| /5ATTO633N/ | rUrUrUrUrUrUrUrU (SEQ ID NO: 2) | /3IAbRQSp/ |
| /56-FAM/ | rUrUrUrUrUrUrUrUrUrU (SEQ ID NO: 3) | /3IABkFQ/ |
| /5IRD700/ | rUrUrUrUrUrUrUrUrUrU (SEQ ID NO: 3) | /3IRQC1N/ |
| /5TYE665/ | rUrUrUrUrUrUrUrUrUrU (SEQ ID NO: 3) | /3IAbRQSp/ |
| /5Alex594N/ | rUrUrUrUrUrUrUrUrUrU (SEQ ID NO: 3) | /3IAbRQSp/ |
| /5ATTO633N/ | rUrUrUrUrUrUrUrUrUrU (SEQ ID NO: 3) | /3IAbRQSp/ |
| /56-FAM/ | TTTTrUrUTTTT (SEQ ID NO: 4) | /3IABkFQ/ |
| /5IRD700/ | TTTTrUrUTTTT (SEQ ID NO: 4) | /3IRQC1N/ |
| /5TYE665/ | TTTTrUrUTTTT (SEQ ID NO: 4) | /3IAbRQSp/ |
| /5Alex594N/ | TTTTrUrUTTTT (SEQ ID NO: 4) | /3IAbRQSp/ |
| /5ATTO633N/ | TTTTrUrUTTTT(SEQ ID NO: 4) | /3IAbRQSp/ |
| /56-FAM/ | TTrUrUTT (SEQ ID NO: 5) | /3IABkFQ/ |
| /5IRD700/ | TTrUrUTT (SEQ ID NO: 5) | /3IRQC1N/ |
| /5TYE665/ | TTrUrUTT (SEQ ID NO: 5) | /3IAbRQSp/ |
| /5Alex594N/ | TTrUrUTT (SEQ ID NO: 5) | /3IAbRQSp/ |
| /5ATTO633N/ | TTrUrUTT (SEQ ID NO: 5) | /3IAbRQSp/ |
| /56-FAM/ | TArArUGC (SEQ ID NO: 6) | /3IABkFQ/ |
| /5IRD700/ | TArArUGC (SEQ ID NO: 6) | /3IRQC1N/ |
| /5TYE665/ | TArArUGC (SEQ ID NO: 6) | /3IAbRQSp/ |
| /5Alex594N/ | TArArUGC (SEQ ID NO: 6) | /3IAbRQSp/ |
| /5ATTO633N/ | TArArUGC (SEQ ID NO: 6) | /3IAbRQSp/ |
| /56-FAM/ | TArUrGGC (SEQ ID NO: 7) | /3IABkFQ/ |
| /5IRD700/ | TArUrGGC (SEQ ID NO: 7) | /3IRQC1N/ |
| /5TYE665/ | TArUrGGC (SEQ ID NO: 7) | /3IAbRQSp/ |
| /5Alex594N/ | TArUrGGC (SEQ ID NO: 7) | /3IAbRQSp/ |
| /5ATTO633N/ | TArUrGGC (SEQ ID NO: 7) | /3IAbRQSp/ |
| /56-FAM/ | rUrUrUrUrU (SEQ ID NO: 8) | /3IABkFQ/ |
| /5IRD700/ | rUrUrUrUrU (SEQ ID NO: 8) | /3IRQC1N/ |
| /5TYE665/ | rUrUrUrUrU (SEQ ID NO: 8) | /3IAbRQSp/ |
| /5Alex594N/ | rUrUrUrUrU (SEQ ID NO: 8) | /3IAbRQSp/ |
| /5ATTO633N/ | rUrUrUrUrU (SEQ ID NO: 8) | /3IAbRQSp/ |
| /56-FAM/ | TTATTATT (SEQ ID NO: 9) | /3IABkFQ/ |
| /56-FAM/ | TTATTATT (SEQ ID NO: 9) | /3IABkFQ/ |
| /5IRD700/ | TTATTATT (SEQ ID NO: 9) | /3IRQC1N/ |
| /5TYE665/ | TTATTATT (SEQ ID NO: 9) | /3IAbRQSp/ |
| /5Alex594N/ | TTATTATT (SEQ ID NO: 9) | /3IAbRQSp/ |
| /5ATTO633N/ | TTATTATT (SEQ ID NO: 9) | /3IAbRQSp/ |
| /56-FAM/ | TTTTTT (SEQ ID NO: 10) | /3IABkFQ/ |
| /56-FAM/ | TTTTTTTT (SEQ ID NO: 11) | /3IABkFQ/ |
| /56-FAM/ | TTTTTTTTTT (SEQ ID NO: 12) | /3IABkFQ/ |
| /56-FAM/ | TTTTTTTTTTTT (SEQ ID NO: 13) | /3IABkFQ/ |
| /56-FAM/ | TTTTTTTTTTTTTT (SEQ ID NO: 14) | /3IABkFQ/ |
| /56-FAM/ | AAAAAA (SEQ ID NO: 15) | /3IABkFQ/ |
| /56-FAM/ | CCCCCC (SEQ ID NO: 16) | /3IABkFQ/ |
| /56-FAM/ | GGGGGG (SEQ ID NO: 17) | /3IABkFQ/ |
| /56-FAM/ | TTATTATT (SEQ ID NO: 9) | /3IABkFQ/ |

/56-FAM/: 5′ 6-Fluorescein (Integrated DNA Technologies)
/3IABkFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester) (Integrated DNA Technologies)
/5ATTO633N/: 5′ ATTO TM 633 (NHS Ester) (Integrated DNA Technologies)
/3IRQC1N/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

A detection moiety can be an infrared fluorophore. A detection moiety can be a fluorophore that emits fluorescence in the range of from 500 nm and 720 nm. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the detection moiety emits fluorescence in the range from 450 nm to 750 nm, from 500 nm to 650 nm, or from 550 to 650 nm. A detection moiety can be a fluorophore that emits a fluorescence in the same range as 6-Fluorescein, IRDye 700, TYE 665, Alex Fluor, or ATTO TM 633 (NHS Ester). A detection moiety can be fluorescein amidite, 6-Fluorescein, IRDye 700, TYE 665, Alex Fluor 594, or ATTO TM 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Any of the detection moieties described herein can be from any commercially available source, can be an alternative with a similar function, a generic, or a non-tradename of the detection moieties listed.

A detection moiety can be chosen for use based on the type of sample to be tested. For example, a detection moiety that is an infrared fluorophore is used with a urine sample. As another example, SEQ ID NO: 1 with a fluorophore that emits a fluorescence around 520 nm is used for testing in non-urine samples, and SEQ ID NO: 8 with a fluorophore that emits a fluorescence around 700 nm is used for testing in urine samples.

A quenching moiety can be chosen based on its ability to quench the detection moiety. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety that emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the quenching moiety quenches a detection moiety that emits fluorescence in the range from 450 nm to 750 nm, from 500 nm to 650 nm, or from 550 to 650 nm. A quenching moiety can quench fluorescein amidite, 6-Fluorescein, IRDye 700, TYE 665, Alex Fluor 594, or ATTO TM 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety can be Iowa Black RQ (Integrated DNA Technologies), Iowa Black FQ (Integrated DNA Technologies) or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of the detection moiety indicates that cleavage by the programmable nuclease has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively or in combination, the detection moiety comprises a polypeptide. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety can be any moiety capable of generating a calorimetric, potentiometric, amperometric, optical (e.g., fluorescent, colorometric, etc.), or piezo-electric signal. A detector nucleic acid, sometimes, is protein-nucleic acid that is capable of generating a calorimetric, potentiometric, amperometric, optical (e.g., fluorescent, colorometric, etc.), or piezo-electric signal upon cleavage of the nucleic acid. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorometric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid.

Often, the protein-nucleic acid is an enzyme-nucleic acid. The enzyme may be sterically hindered when present as in the enzyme-nucleic acid, but then functional upon cleavage from the nucleic acid. Often, the enzyme is an enzyme that produces a reaction with a substrate. An enzyme can be invertase. Often, the substrate of invertase is sucrose and DNS reagent. In some cases, it is preferred that the nucleic acid (e.g., DNA) and invertase are conjugated using a heterobifunctiona linker via sulfo-SMCC chemistry.

Sometimes the protein-nucleic acid is a substrate-nucleic acid. Often the substrate is a substrate that produces a reaction with an enzyme.

A protein-nucleic acid may be attached to a solid support. The solid support, for example, is a surface. A surface can be an electrode. Sometimes the solid support is a bead. Often the bead is a magnetic bead. Upon cleavage, the protein is liberated from the solid and interacts with other mixtures. For example, the protein is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

Often, the signal is a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorometric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system is capable of detecting more than one type of target nucleic acid, wherein the system comprises more than one type of engineered guide nucleic acid and more than one type of detector nucleic acid. In some cases, the detectable signal is generated directly by the cleavage event. Alternatively or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 μM, 1 μM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 μM, 1 aM to 200 μM, 1 aM to 100 μM, 1 aM to 10 μM, 1 aM to 1 μM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 μM, 10 aM to 200 μM, 10 aM to 100 μM, 10 aM to 10 μM, 10 aM to 1 μM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 μM, 100 aM to 200 μM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, from 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases the threshold of detection is in a range of from 2 aM to 100 pM, from 20 aM to 50 pM, from 50 aM to 20 pM, from 200 aM to 5 pM, or from 500 aM to 2 pM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single stranded target nucleic acid is detected in a sample is in a range of from 2 aM to 100 pM, from 20 aM to 50 pM, from 50 aM to 20 pM, from 200 aM to 5 pM, or from 500 aM to 2 pM. In some cases, the minimum concentration at which a single stranded target nucleic acid can be detected in a sample is in a range of from 1 aM to 100 pM. In some cases, the minimum concentration at which a single stranded target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some embodiments, the target nucleic acid is present in the cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in the cleavage reaction at a concentration of from 20 nM to 50 μM, from 50 nM to 20 μM, or from 200 nM to 5 μM.

In some cases, the methods, compositions, reagents, enzymes, and kits described herein may be used to detect a target single-stranded nucleic acid in a sample where the sample is contacted with the reagents for a predetermined length of time sufficient for the trans cleavage to occur or cleavage reaction to reach completion. In some cases, the devices, systems, fluidic devices, kits, and methods described herein detect a target single-stranded nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes. In some cases, the sample is contacted with the reagents for from 5 minutes to 120 minutes, from 5 minutes to 100 minutes, from 10 minutes to 90 minutes, from 15 minutes to 45 minutes, or from 20 minutes to 35 minutes. In some cases, the devices, systems, fluidic devices, kits, and methods described herein can detect a target nucleic acid in a sample in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some cases, the devices, systems, fluidic devices, kits, and methods described herein can detect a target nucleic acid in a sample in from 5 minutes to 10 hours, from 10 minutes to 8 hours, from 15 minutes to 6 hours, from 20 minutes to 5 hours, from 30 minutes to 2 hours, or from 45 minutes to 1 hour.

When an engineered guide nucleic acid binds to a target nucleic acid, the programmable nuclease's trans cleavage activity can be initiated, and detector nucleic acids can be cleaved, resulting in the detection of fluorescence. Some methods as described herein can a method of assaying for a target nucleic acid in a sample comprises contacting the sample to a complex comprising an engineered guide nucleic acid comprising a segment that is reverse complementary to a segment of the target nucleic acid and a programmable nuclease that exhibits sequence independent cleavage upon forming a complex comprising the segment of the engineered guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The cleaving of the detector nucleic acid using the programmable nuclease may cleave with an efficiency of 50% as measured by a change in a signal that is calorimetric, potentiometric, amperometric, optical (e.g., fluorescent, colorometric, etc.), or piezo-electric, as non-limiting examples. Some methods as described herein can be a method of detecting a target nucleic acid in a sample comprising contacting the sample comprising the target nucleic acid with an engineered guide nucleic acid targeting a target nucleic acid segment, a programmable nuclease capable of being activated when complexed with the engineered guide nucleic acid and the target nucleic acid segment, a single stranded detector nucleic acid comprising a detection moiety, wherein the detector nucleic acid is capable of being cleaved by the activated programmable nuclease, thereby generating a first detectable signal, cleaving the single stranded detector nucleic acid using the programmable nuclease that cleaves as measured by a change in color, and measuring the first detectable signal on the support medium. The cleaving of the single stranded detector nucleic acid using the programmable nuclease may cleave with an efficiency of 50% as measured by a change in color. In some cases, the cleavage efficiency is at least 40%, 50%, 60%, 70%, 80%, 90%, or 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with an engineered guide nucleic acid targeting a target nucleic acid segment, a programmable nuclease capable of being activated when complexed with the engineered guide nucleic acid and the target nucleic acid segment, and a single stranded detector nucleic acid comprising a detection moiety, wherein the detector nucleic acid is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample. In some embodiments, the first detectable signal can be detectable within from 1 to 120, from 5 to 100, from 10 to 90, from 15 to 80, from 20 to 60, or from 30 to 45 minutes of contacting the sample.

In some cases, the methods, reagents, enzymes, and kits described herein detect a target single-stranded nucleic acid with a programmable nuclease and a single-stranded detector nucleic acid in a sample where the sample is contacted with the reagents for a predetermined length of time sufficient for trans cleavage of the single stranded detector nucleic acid. In a preferred embodiment, a Cas13a programmable nuclease us used to detect the presence of a single-stranded DNA target nucleic acid. For example, a programmable nuclease is LbuCas13a that detects a target nucleic acid and a single stranded detector nucleic acid comprises two adjacent uracil nucleotides with a green detectable moiety that is detected upon cleavage. As another example, a programmable nuclease is LbaCas13a that detects a target nucleic acid and a single-stranded detector nucleic acid comprises two adjacent adenine nucleotides with a red detectable moiety that is detected upon cleavage.

### Buffers

The reagents described herein can also include buffers, which are compatible with the methods, compositions, reagents, enzymes, and kits disclosed herein. These buffers are compatible with the other reagents, samples, and support mediums as described herein for detection of an ailment, such as a disease, cancer, or genetic disorder, or genetic information, such as for phenotyping, genotyping, or determining ancestry. As described herein, nucleic acid sequences comprising DNA may be detected using a DNA-activated programmable RNA nuclease and other reagents disclosed herein. Additionally, detection by a DNA-activated programmable RNA nuclease, which can cleave RNA reporters, allows for multiplexing with other programmable nucleases, such as a DNA-activated programmable DNA nuclease that can cleave DNA reporters (e.g., Type V CRISPR enzyme). The methods described herein can also include the use of buffers, which are compatible with the methods disclosed herein. For example, a buffer comprises 20 mM HEPES pH 6.8, 50 mM KCl, 5 mM MgCl₂, and 5% glycerol. In some instances the buffer comprises from 0 to 100, 0 to 75, 0 to 50, 0 to 25, 0 to 20, 0 to 10, 0 to 5, 5 to 10,5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM HEPES pH 6.8. The buffer can comprise to 0 to 500, 0 to 400, 0 to 300, 0 to 250, 0 to 200,0 to 150,0 to 100,0 to 75,0 to 50,0 to 25,0 to 20,0 to 10,0 to 5,5 to 10,5 to 15,5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer comprises 0 to 100,0 to 75,0 to 50,0 to 25,0 to 20,0 to 10,0 to 5,5 to 10,5 to 15,5 to 20,5 to 25, to 30,5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. The buffer can comprise 0 to 25, 0 to 20, 0 to 10, 0 to 5, 5 to 10, 5 to 15, 5 to 20, 5 to 25, 5 to 30% glycerol. The buffer can comprise 0 to 30, 2 to 25, or 10 to 20% glycerol.

As another example, a buffer comprises 100 mM Imidazole pH 7.5; 250 mM KCl, 25 mM MgCl₂, 50 ug/mL BSA, 0.05% Igepal Ca-630, and 25% Glycerol. In some instances the buffer comprises 0 to 500, 0 to 400, 0 to 300, 0 to 250, 0 to 200, 0 to 150, 0 to 100, 0 to 75, 0 to 50,0 to 25,0 to 20,0 to 10,0 to 5,5 to 10,5 to 15,5 to 20,5 to 25, to 30,5 to 40,5 to 50,5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM Imidazole pH 7.5. In some instances the buffer comprises 100 to 250, 100 to 200, or 150 to 200 mM Imdazole pH 7.5. The buffer can comprise 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. The buffer, in some instances, comprises 0 to 100, 0 to 75, 0 to 50, 0 to 25, 0 to 20, 0 to 10, 0 to 5, 5 to 50, 5 to 75,5 to 100, 10 to 20, 10 to 50, 10 to 75, 10 to 100,25 to 50,25 to 7525 to 100, 50 to 75, or 50 to 100 ug/mL BSA. In some instances, the buffer comprises 0 to 1, 0 to 0.5, 0 to 0.25, 0 to 0.01, 0 to 0.05, 0 to 0.025, 0 to 0.01, 0.01 to 0.025, 0.01 to 0.05, 0.01 to 0.1, 0.01 to 0.25, 0.01, to 0.5, 0.01 to 1, 0.025 to 0.05, 0.025 to 0.1, 0.025, to 0.5, 0.025 to 1, 0.05 to 0.1, 0.05 to 0.25, 0.05 to 0.5, 0.05 to 0.75, 0.05 to 1, 0.1 to 0.25, 0.1 to 0.5, or 0.1 to 1% Igepal Ca-630. The buffer can comprise 0 to 25, 0 to 20, 0 to 10, 0 to 5, 5 to 10, 5 to 15, 5 to 20, 5 to 25, 5 to 30% glycerol. The buffer can comprise 0 to 30, 2 to 25, or 10 to 20% glycerol.

### Stability

Present in this disclosure are stable compositions of the reagents and the programmable nuclease system for use in the methods as discussed herein. The reagents and programmable nuclease system described herein may be stable in various storage conditions including refrigerated, ambient, and accelerated conditions. Disclosed herein are stable reagents. The stability may be measured for the reagents and programmable nuclease system themselves or the reagents and programmable nuclease system present on the support medium.

In some instances, stable as used herein refers to a reagents having about 5% w/w or less total impurities at the end of a given storage period. Stability may be assessed by HPLC or any other known testing method. The stable reagents may have about 10% w/w, about 5% w/w, about 4% w/w, about 30% w/w, about 2% w/w, about 10% w/w, or about 0.50% w/w total impurities at the end of a given storage period. The stable reagents may have from 0.5% w/w to 10% w/w, from 1% w/w to 8% w/w, from 2% w/w to 7% w/w, or from 3% w/w to 5% w/w total impurities at the end of a given storage period.

In some embodiments, stable as used herein refers to a reagents and programmable nuclease system having about 10% r less loss of detection activity at the end of a given storage period and at a given storage condition. Detection activity can be assessed by known positive sample using a known method. Alternatively or combination, detection activity can be assessed by the sensitivity, accuracy, or specificity. In some embodiments, the stable reagents has about 10%, about 9%, about 8%, about 7%, about 6%, about 5%, about 4%, about 3%, about 2%, about 1%, or about 0.5% loss of detection activity at the end of a given storage period. In some embodiments, the stable reagents has from 0.5% to 10%, from 1% to 8%, from 2% to 7%, or from 3% to 5% loss of detection activity at the end of a given storage period.

In some embodiments, the stable composition has zero loss of detection activity at the end of a given storage period and at a given storage condition. The given storage condition may comprise humidity of equal to or less than 10%, 20%, 30%, 40%, 50%, 60%, 70%, 80%, 90%, or 100% relative humidity. The controlled storage environment may comprise humidity from 0% to 50% relative humidity, from 0% to 40% relative humidity, from 0% to 30% relative humidity, from 0% to 20% relative humidity, or from 0% to 10% relative humidity. The controlled storage environment may comprise humidity from 10% to 80%, from 10% to 70%, from 10% to 60%, from 20% to 50%, from 20% to 40%, or from 20% to 30% relative humidity. The controlled storage environment may comprise temperatures of about −100° C., about −80° C., about −20° C., about 4° C., about 25° C. (room temperature), or about 40° C. The controlled storage environment may comprise temperatures from −80° C. to 25° C., or from −100° C. to 40° C. The controlled storage environment may comprise temperatures from −20° C. to 40° C., from −20° C. to 4° C., or from 4° C. to 40° C. The controlled storage environment may protect the system or kit from light or from mechanical damage. The controlled storage environment may be sterile or aseptic or maintain the sterility of the light conduit. The controlled storage environment may be aseptic or sterile.

### Multiplexing

The methods and systems disclosed herein can be carried out for multiplexed detection. These methods of multiplexing are, for example, consistent with fluidic devices disclosed herein for detection of a target nucleic acid sequence within the sample, wherein the fluidic device may comprise multiple pumps, valves, reservoirs, and chambers for sample preparation, amplification of a target nucleic acid sequence within the sample, mixing with a programmable nuclease, and detection of a detectable signal arising from cleavage of detector nucleic acids by the programmable nuclease within the fluidic system itself.

Methods consistent with the present disclosure include a multiplexing method of assaying for a target nucleic acid in a sample. A multiplexing method comprises contacting the sample to a complex comprising an engineered guide nucleic acid comprising a segment that is reverse complementary to a segment of the target nucleic acid (e.g., DNA) and a programmable nuclease (e.g., a DNA-activated programmable RNA nuclease, such as Cas13) that exhibits sequence independent cleavage upon forming a complex comprising the segment of the engineered guide nucleic acid binding to the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, multiplexing method of assaying for a target nucleic acid in a sample, for example, comprises: a) contacting the sample to a complex comprising an engineered guide nucleic acid comprising a segment that is reverse complementary to a segment of the target nucleic acid and a programmable nuclease that exhibits sequence independent cleavage upon forming a complex comprising the segment of the engineered guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Multiplexing can be either spatial multiplexing wherein multiple different target nucleic acids at the same time, but the reactions are spatially separated. Often, the multiple target nucleic acids are detected using the same programmable nuclease, but different engineered guide nucleic acids. The multiple target nucleic acids sometimes are detected using the different programmable nucleases. For example, a DNA-activated programmable RNA nuclease and a DNA-activated programmable DNA nuclease can both be used in a single assay to directly detect DNA targets encoding different sequences. The activated DNA-activated programmable RNA nuclease cleaves an RNA reporter, generating a first detectable signal and the activated DNA-activated programmable DNA nuclease cleaves a DNA reporter, generating a second detectable signal. In some embodiments, the first and second detectable signals are different, and those allow simultaneous detection of more than one target DNA sequences using two programmable nucleases. In some embodiments, the DNA-activated programmable DNA nuclease and the DNA-activated programmable RNA nuclease are complexed to an engineered guide nucleic acid that hybridizes to the same target DNA. The activated DNA-activated programmable RNA nuclease cleaves an RNA reporter, generating a first detectable signal and the activated a DNA-activated programmable DNA nuclease cleaves a DNA reporter, generating a second detectable signal. The first detectable signal and the second detectable signal can be the same, thus, allowing for signal amplification by cleavage of reporters by two different programmable nucleases that are activated by the same target DNA.

Sometimes, multiplexing can be single reaction multiplexing wherein multiple different target nucleic acids are detected in a single reaction volume. Often, at least two different programmable nucleases are used in single reaction multiplexing. For example, multiplexing can be enabled by immobilization of multiple categories of detector nucleic acids within a fluidic system, to enable detection of multiple target nucleic acids within a single fluidic system. Multiplexing allows for detection of multiple target nucleic acids in one kit or system. In some cases, the multiple target nucleic acids comprise different target nucleic acids to a virus, a bacterium, or a pathogen responsible for one disease. In some cases, the multiple target nucleic acids comprise different target nucleic acids associated with a cancer or genetic disorder. Multiplexing for one disease, cancer, or genetic disorder increases at least one of sensitivity, specificity, or accuracy of the assay to detect the presence of the disease in the sample. In some cases, the multiple target nucleic acids comprise target nucleic acids directed to different viruses, bacteria, or pathogens responsible for more than one disease. In some cases, multiplexing allows for discrimination between multiple target nucleic acids, such as target nucleic acids that comprise different genotypes of the same bacteria or pathogen responsible for a disease, for example, for a wild-type genotype of a bacteria or pathogen and for genotype of a bacteria or pathogen comprising a mutation, such as a single nucleotide polymorphism (SNP) that can confer resistance to a treatment, such as antibiotic treatment. For example, multiplexing comprises method of assaying comprising a single assay for a microorganism species using a first programmable nuclease and an antibiotic resistance pattern in a microorganism using a second programmable nuclease. Sometimes, multiplexing allows for discrimination between multiple target nucleic acids of different HPV strains, for example, HPV16 and HPV18. In some cases, the multiple target nucleic acids comprise target nucleic acids directed to different cancers or genetic disorders. Often, multiplexing allows for discrimination between multiple target nucleic acids, such as target nucleic acids that comprise different genotypes, for example, for a wild-type genotype and for SNP genotype. Multiplexing for multiple diseases, cancers, or genetic disorders provides the capability to test a panel of diseases from a single sample. For example, multiplexing for multiple diseases can be valuable in a broad panel testing of a new patient or in epidemiological surveys. Often multiplexing is used for identifying bacterial pathogens in sepsis or other diseases associated with multiple pathogens.

Furthermore, signals from multiplexing can be quantified. For example, a method of quantification for a disease panel comprises assaying for a plurality of unique target nucleic acids in a plurality of aliquots from a sample, assaying for a control nucleic acid control in a second aliquot of the sample, and quantifying a plurality of signals of the plurality of unique target nucleic acids by measuring signals produced by cleavage of detector nucleic acids compared to the signal produced in the second aliquot. Often the plurality of unique target nucleic acids are from a plurality of bacterial pathogens in the sample. Sometimes the quantification of a signal of the plurality correlates with a concentration of a unique target nucleic acid of the plurality for the unique target nucleic acid of the plurality that produced the signal of the plurality. The disease panel can be for any communicable disease, such as sepsis.

In some instances, multiplexed detection detects at least 2 different target nucleic acids in a single reaction. In some instances, multiplexed detection detects at least 3 different target nucleic acids in a single reaction. In some instances, multiplexed detection detects at least 4 different target nucleic acids in a single reaction. In some instances, multiplexed detection detects at least 5 different target nucleic acids in a single reaction. In some cases, multiplexed detection detects at least 6, 7, 8, 9, or 10 different target nucleic acids in a single reaction. In some instances, the multiplexed kits detect at least 2 different target nucleic acids in a single kit. In some instances, the multiplexed kits detect at least 3 different target nucleic acids in a single kit. In some instances, the multiplexed kits detect at least 4 different target nucleic acids in a single kit. In some instances, the multiplexed kits detect at least 5 different target nucleic acids in a single kit. In some instances, the multiplexed kits detect at least 6, 7, 8, 9, or 10 different target nucleic acids in a single kit. In some instances, the multiplexed kits detect from 2 to 10, from 3 to 9, from 4 to 8, or from 5 to 7 different target nucleic acids in a single kit.

### Support Medium

A number of support mediums are consistent with the compositions and methods disclosed herein. These support mediums are, for example, consistent with fluidic devices disclosed herein for detection of a target nucleic acid within the sample, wherein the fluidic device may comprise multiple pumps, valves, reservoirs, and chambers for sample preparation, amplification of a target nucleic acid within the sample, mixing with a programmable nuclease, and detection of a detectable signal arising from cleavage of detector nucleic acids by the programmable nuclease within the fluidic system itself. These support mediums are compatible with the samples, reagents, and fluidic devices described herein for detection of an ailment, such as a viral infection. A support medium described herein can provide a way to present the results from the activity between the reagents and the sample. The support medium provides a medium to present the detectable signal in a detectable format. Optionally, the support medium concentrates the detectable signal to a detection spot in a detection region to increase the sensitivity, specificity, or accuracy of the assay. The support mediums can present the results of the assay and indicate the presence or absence of the disease of interest targeted by the target nucleic acid. The result on the support medium can be read by eye or using a machine. The support medium helps to stabilize the detectable signal generated by the cleaved detector molecule on the surface of the support medium. In some instances, the support medium is a lateral flow assay strip. In some instances, the support medium is a PCR plate. The PCR plate can have 96 wells or 384 wells. The PCR plate can have a subset number of wells of a 96 well plate or a 384 well plate. A subset number of wells of a 96 well PCR plate is, for example, 1, 2, 3,4, 5,6,7, 8,9, 10, 11, 12, 13, 14, 15, 16, 17, 18, 19, 20, 21, 22, 23, 24, 25, 30, 35, 40, 45, 50, 55, 60, 65, 70, 75, 80, 85, 90, or 95 wells. For example, a PCR subset plate can have 4 wells wherein a well is the size of a well from a 96 well PCR plate (e.g., a 4 well PCR subset plate wherein the wells are the size of a well from a 96 well PCR plate). A subset number of wells of a 384 well PCR plate is, for example, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, or 380 wells. For example, a PCR subset plate can have 20 wells wherein a well is the size of a well from a 384 well PCR plate (e.g., a 20 well PCR subset plate wherein the wells are the size of a well from a 384 well PCR plate). The PCR plate or PCR subset plate can be paired with a fluorescent light reader, a visible light reader, or other imaging device. Often, the imaging device is a digital camera, such a digital camera on a mobile device. The mobile device may have a software program or a mobile application that can capture an image of the PCR plate or PCR subset plate, identify the assay being performed, detect the individual wells and the sample therein, provide image properties of the individuals wells comprising the assayed sample, analyze the image properties of the contents of the individual wells, and provide a result.

The support medium has at least one specialized zone or region to present the detectable signal. The regions comprise at least one of a sample pad region, a nucleic acid amplification region, a conjugate pad region, a detection region, and a collection pad region. In some instances, the regions are overlapping completely, overlapping partially, or in series and in contact only at the edges of the regions, where the regions are in fluid communication with its adjacent regions. In some instances, the support medium has a sample pad located upstream of the other regions; a conjugate pad region having a means for specifically labeling the detector moiety; a detection region located downstream from sample pad; and at least one matrix which defines a flow path in fluid connection with the sample pad. In some instances, the support medium has an extended base layer on top of which the various zones or regions are placed. The extended base layer may provide a mechanical support for the zones.

Described herein are sample pads that provide an area to apply the sample to the support medium. The sample may be applied to the support medium by a dropper or a pipette on top of the sample pad, by pouring or dispensing the sample on top of the sample pad region, or by dipping the sample pad into a reagent chamber holding the sample. The sample can be applied to the sample pad prior to reaction with the reagents when the reagents are placed on the support medium or be reacted with the reagents prior to application on the sample pad. The sample pad region can transfer the reacted reagents and sample into the other zones of the support medium. Transfer of the reacted reagents and sample may be by capillary action, diffusion, convection or active transport aided by a pump. In some cases, the support medium is integrated with or overlayed by microfluidic channels to facilitate the fluid transport.

The dropper or the pipette may dispense a predetermined volume. In some cases, the predetermined volume may range from about 1 μl to about 1000 μl, about 1 μl to about 500 μl, about 1 μl to about 100 μl, or about 1 μl to about 50 μl. In some cases, the predetermined volume may be at least 1 μl, 2 μl, 3 μl, 4 μl, 5 μl, 6 μl, 7 μl, 8 μl, 9 μl, 10 μl, 25 μl, 50 μl, 75 μl, 100 μl, 250 μl, 500 μl, 750 μl, or 1000 μl. The predetermined volume may be no more than 5 μl, 10 μl, 25 μl, 50 μl, 75 μl, 100 μl, 250 μl, 500 μl, 750 μl, or 1000 μl. The dropper or the pipette may be disposable or be single-use.

Optionally, a buffer or a fluid may also be applied to the sample pad to help drive the movement of the sample along the support medium. In some cases, the volume of the buffer or the fluid may range from about 1 μl to about 1000 μl, about 1 μl to about 500 μl, about 1 μl to about 100 μl, or about 1 μl to about 50 μl. In some cases, the volume of the buffer or the fluid may be at least 1 μl, 2 μl, 3 μl, 4 μl, 5 μl, 6 μl, 7 μl, 8 μl, 9 μl, 10 μl, 25 μl, 50 μl, 75 μl, 100 μl, 250 μl, 500 μl, 750 μl, or 1000 μl. The volume of the buffer or the fluid may be no more than than 5 μl, 10 μl, 25 μl, 50 μl, 75 μl, 100 μl, 250 μl, 500 μl, 750 μl, or 1000 μl. In some cases, the buffer or fluid may have a ratio of the sample to the buffer or fluid of at least 1:1, 1:2, 1:3, 1:4, 1:5, 1:6, 1:7, 1:8, 1:9, or 1:10.

The sample pad can be made from various materials that transfer most of the applied reacted reagents and samples to the subsequent regions. The sample pad may comprise cellulose fiber filters, woven meshes, porous plastic membranes, glass fiber filters, aluminum oxide coated membranes, nitrocellulose, paper, polyester filter, or polymer-based matrices. The material for the sample pad region may be hydrophilic and have low non-specific binding. The material for the sample pad may range from about 50 μm to about 1000 μm, about 50 μm to about 750 μm, about 50 μm to about 500 μm, or about 100 μm to about 500 μm.

The sample pad can be treated with chemicals to improve the presentation of the reaction results on the support medium. The sample pad can be treated to enhance extraction of nucleic acid in the sample, to control the transport of the reacted reagents and sample or the conjugate to other regions of the support medium, or to enhance the binding of the cleaved detection moiety to the conjugate binding molecule on the surface of the conjugate or to the capture molecule in the detection region. The chemicals may comprise detergents, surfactants, buffers, salts, viscosity enhancers, or polypeptides. In some instances, the chemical comprises bovine serum albumin.

Described herein are conjugate pads that provide a region on the support medium comprising conjugates coated on its surface by conjugate binding molecules that can bind to the detector moiety from the cleaved detector molecule or to the control molecule. The conjugate pad can be made from various materials that facilitate binding of the conjugate binding molecule to the detection moiety from cleaved detector molecule and transfer of most of the conjugate-bound detection moiety to the subsequent regions. The conjugate pad may comprise the same material as the sample pad or other zones or a different material than the sample pad. The conjugate pad may comprise glass fiber filters, porous plastic membranes, aluminum oxide coated membranes, paper, cellulose fiber filters, woven meshes, polyester filter, or polymer-based matrices. The material for the conjugate pad region may be hydrophilic, have low non-specific binding, or have consistent fluid flow properties across the conjugate pad. In some cases, the material for the conjugate pad may range from about 50 μm to about 1000 μm, about 50 μm to about 750 μm, about 50 μm to about 500 μm, or about 100 μm to about 500 μm.

Further described herein are conjugates that are placed on the conjugate pad and immobilized to the conjugate pad until the sample is applied to the support medium. The conjugates may comprise a nanoparticle, a gold nanoparticle, a latex nanoparticle, a quantum dot, a chemiluminescent nanoparticle, a carbon nanoparticle, a selenium nanoparticle, a fluorescent nanoparticle, a liposome, or a dendrimer. The surface of the conjugate may be coated by a conjugate binding molecule that binds to the detection moiety from the cleaved detector molecule.

### Detection Methods

Disclosed herein are methods of assaying for a target nucleic acid as described herein wherein a signal is detected. In some embodiments, the methods disclosed herein are methods of assaying for a target deoxyribonucleic acid as described herein using a DNA-activated programmable RNA nuclease wherein a signal is detected. For example, a method of assaying for a target nucleic acid in a sample comprises contacting the sample to a complex comprising an engineered guide nucleic acid comprising a segment that is reverse complementary to a segment of the target nucleic acid and a programmable nuclease that exhibits sequence independent cleavage upon forming a complex comprising the segment of the engineered guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, a method of assaying for a target nucleic acid in a sample, for example, comprises: a) contacting the sample to a complex comprising an engineered guide nucleic acid comprising a segment that is reverse complementary to a segment of the target nucleic acid and a DNA-activated programmable RNA nuclease that exhibits sequence independent cleavage upon forming a complex comprising the segment of the engineered guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid. As described herein, nucleic acid sequences comprising DNA may be detected using a DNA-activated programmable RNA nuclease and other reagents disclosed herein.

Present in this disclosure are methods of assaying for a target nucleic acid as described herein. In some embodiments, the method is a method of assaying for a target deoxyribonucleic acid using a DNA-activated programmable RNA nuclease, wherein assaying comprises detecting cleavage of an RNA reporter. For example, a method of assaying for a target nucleic acid in a sample comprises contacting the sample to a complex comprising an engineered guide nucleic acid comprising a segment that is reverse complementary to a segment of the target nucleic acid and a programmable nuclease (e.g., a DNA-activated programmable RNA nuclease) that exhibits sequence independent cleavage upon forming a complex comprising the segment of the engineered guide nucleic acid binding to the segment of the target nucleic acid (e.g. target deoxyribonucleic acid); and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, a method of assaying for a target nucleic acid in a sample, for example, comprises: a) contacting the sample to a complex comprising an engineered guide nucleic acid comprising a segment that is reverse complementary to a segment of the target nucleic acid and a programmable nuclease that exhibits sequence independent cleavage upon forming a complex comprising the segment of the engineered guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

A number of detection devices and methods are consistent with methods disclosed herein. For example, any device that can measure or detect a calorimetric, potentiometric, amperometric, optical (e.g., fluorescent, colorometric, etc.), or piezo-electric signal. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorometric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid. Sometimes, the detector nucleic acid is protein-nucleic acid. Often, the protein-nucleic acid is an enzyme-nucleic acid.

The results from the detection region from a completed assay can be detected and analyzed in various ways. For example, by a glucometer. In some cases, the positive control spot and the detection spot in the detection region is visible by eye, and the results can be read by the user. In some cases, the positive control spot and the detection spot in the detection region is visualized by an imaging device or other device depending on the type of signal. Often, the imaging device is a digital camera, such a digital camera on a mobile device. The mobile device may have a software program or a mobile application that can capture an image of the support medium, identify the assay being performed, detect the detection region and the detection spot, provide image properties of the detection spot, analyze the image properties of the detection spot, and provide a result. Alternatively or in combination, the imaging device can capture fluorescence, ultraviolet (UV), infrared (IR), or visible wavelength signals. The imaging device may have an excitation source to provide the excitation energy and captures the emitted signals. In some cases, the excitation source can be a camera flash and optionally a filter. In some cases, the imaging device is used together with an imaging box that is placed over the support medium to create a dark room to improve imaging. The imaging box can be a cardboard box that the imaging device can fit into before imaging. In some instances, the imaging box has optical lenses, mirrors, filters, or other optical elements to aid in generating a more focused excitation signal or to capture a more focused emission signal. Often, the imaging box and the imaging device are small, handheld, and portable to facilitate the transport and use of the assay in remote or low resource settings.

The assay described herein can be visualized and analyzed by a mobile application (app) or a software program. Using the graphic user interface (GUI) of the app or program, an individual can take an image of the support medium, including the detection region, barcode, reference color scale, and fiduciary markers on the housing, using a camera on a mobile device. The program or app reads the barcode or identifiable label for the test type, locate the fiduciary marker to orient the sample, and read the detectable signals, compare against the reference color grid, and determine the presence or absence of the target nucleic acid, which indicates the presence of the gene, virus, or the agent responsible for the disease, cancer, or genetic disorder. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, clean up of an environment. The methods for detection of a target nucleic acid described herein further can comprises reagents protease treatment of the sample. The sample can be treated with protease, such as Protease K, before amplification or before assaying for a detectable signal. Often, a protease treatment is for no more than 15 minutes. Sometimes, the protease treatment is for no more than 1, 5, 10, 15, 20, 25, 30, or more minutes, or any value from 1 to 30 minutes. Sometimes, the protease treatment is from 1 to 30, from 5 to 25, from 10 to 20, or from 10 to 15 minutes. The kit or system for detection of a target nucleic acid described herein further comprises reagents for nucleic acid amplification of target nucleic acids in the sample. Isothermal nucleic acid amplification allows the use of the kit or system in remote regions or low resource settings without specialized equipment for amplification. Often, the reagents for nucleic acid amplification comprise a recombinase, a oligonucleotide primer, a single-stranded DNA binding (SSB) protein, and a polymerase. Sometimes, nucleic acid amplification of the sample improves at least one of sensitivity, specificity, or accuracy of the assay in detecting the target nucleic acid. In some cases, the nucleic acid amplification is performed in a nucleic acid amplification region on the support medium. Alternatively or in combination, the nucleic acid amplification is performed in a reagent chamber, and the resulting sample is applied to the support medium. Sometimes, the nucleic acid amplification is isothermal nucleic acid amplification. In some cases, the nucleic acid amplification is transcription mediated amplification (TMA). Nucleic acid amplification is helicase dependent amplification (HDA) or circular helicase dependent amplification (cHDA) in other cases. In additional cases, nucleic acid amplification is strand displacement amplification (SDA). In some cases, nucleic acid amplification is by recombinase polymerase amplification (RPA). In some cases, nucleic acid amplification is by at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value from 1 to 60 minutes. Sometimes, the nucleic acid amplification is performed for from 1 to 60, from 5 to 55, from 10 to 50, from 15 to 45, from 20 to 40, or from 25 to 35 minutes. Sometimes, the nucleic acid amplification reaction is performed at a temperature of around 20-45° C. In some cases, the nucleic acid amplification reaction is performed at a temperature no greater than 20° C., 25° C., 30° C., 35° C., 37° C., 40° C., 45° C., or any value from 20° C. to 45° C. In some cases, the nucleic acid amplification reaction is performed at a temperature of at least 20° C., 25° C., 30° C., 35° C., 37° C., 40° C., or 45° C., or any value from 20° C. to 45° C. In some cases, the nucleic acid amplification reaction is performed at a temperature of from 20° C. to 45° C., from 25° C. to 40° C., from 30° C. to 40° C., or from 35° C. to 40° C.

Sometimes, the total time for the performing the method described herein is no greater than 3 hours, 2 hours, 1 hour, 50 minutes, 40 minutes, 30 minutes, 20 minutes, or any value from 3 hours to 20 minutes. Often, a method of nucleic acid detection from a raw sample comprises protease treating the sample for no more than 15 minutes, amplifying (can also be referred to as pre-amplifying) the sample for no more than 15 minutes, subjecting the sample to a programmable nuclease-mediated detection, and assaying nuclease mediated detection. The total time for performing this method, sometimes, is no greater than 3 hours, 2 hours, 1 hour, 50 minutes, 40 minutes, 30 minutes, 20 minutes, or any value from 3 hours to 20 minutes. Often, the protease treatment is Protease K. Often the amplifying is thermal cycling amplification. Sometimes the amplifying is isothermal amplification.

### Detection/Visualization Devices

A number of detection or visualization devices and methods are consistent with the methods, compositions, reagents, enzymes, and kits disclosed herein. As described herein, a target nucleic acid comprising DNA may be detected using a DNA-activated programmable RNA nuclease and other reagents disclosed herein. A DNA-activated programmable RNA nuclease may also be multiplexed as described herein. Sometimes, the signal generated for detection is a calorimetric, potentiometric, amperometric, optical (e.g., fluorescent, colorometric, etc.), or piezo-electric signal. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorometric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid. Sometimes, the detector nucleic acid is protein-nucleic acid. Often, the protein-nucleic acid is an enzyme-nucleic acid. The detection/visualization can be analyzed using various methods, as further described below. The results from the detection region from a completed assay can be visualized and analyzed in various ways. In some cases, the positive control spot and the detection spot in the detection region is visible by eye, and the results can be read by the user. In some cases, the positive control spot and the detection spot in the detection region is visualized by an imaging device. Often, the imaging device is a digital camera, such a digital camera on a mobile device. The mobile device may have a software program or a mobile application that can capture an image of the support medium, identify the assay being performed, detect the detection region and the detection spot, provide image properties of the detection spot, analyze the image properties of the detection spot, and provide a result. Alternatively or in combination, the imaging device can capture fluorescence, ultraviolet (UV), infrared (IR), or visible wavelength signals. The imaging device may have an excitation source to provide the excitation energy and captures the emitted signals. In some cases, the excitation source can be a camera flash and optionally a filter. In some cases, the imaging device is used together with an imaging box that is placed over the support medium to create a dark room to improve imaging. The imaging box can be a cardboard box that the imaging device can fit into before imaging. In some instances, the imaging box has optical lenses, mirrors, filters, or other optical elements to aid in generating a more focused excitation signal or to capture a more focused emission signal. Often, the imaging box and the imaging device are small, handheld, and portable to facilitate the transport and use of the assay in remote or low resource settings.

The assay described herein can be visualized and analyzed by a mobile application (app) or a software program. Using the graphic user interface (GUI) of the app or program, an individual can take an image of the support medium, including the detection region, barcode, reference color scale, and fiduciary markers on the housing, using a camera on a mobile device. The program or app reads the barcode or identifiable label for the test type, locate the fiduciary marker to orient the sample, and read the detectable signals, compare against the reference color grid, and determine the presence or absence of the target nucleic acid, which indicates the presence of the gene, virus, or the agent responsible for the disease, cancer, or genetic disorder. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, clean up of an environment.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein the terms “individual,” “subject,” and “patient” are used interchangeably and include any member of the animal kingdom, including humans.

As used herein the term “antibody” refers to, but not limited to, a monoclonal antibody, a synthetic antibody, a polyclonal antibody, a multispecific antibody (including a bi-specific antibody), a human antibody, a humanized antibody, a chimeric antibody, a single-chain Fvs (scFv) (including bi-specific scFvs), a single chain antibody, a Fab fragment, a F(ab′) fragment, a disulfide-linked Fvs (sdFv), or an epitope-binding fragment thereof. In some cases, the antibody is an immunoglobulin molecule or an immunologically active portion of an immunoglobulin molecule. In some instances, an antibody is animal in origin including birds and mammals. Alternately, an antibody is human or a humanized monoclonal antibody.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A composition comprising: a) a DNA-activated programmable RNA nuclease; and b) an engineered guide nucleic acid comprising a first segment that is reverse complementary to a segment of a target deoxyribonucleic acid, wherein the engineered guide nucleic acid comprises a second segment that binds to the DNA-activated programmable RNA nuclease to form a complex. 2. The composition of embodiment 1, further comprising a detector nucleic acid. 3. The composition of embodiment 2, wherein the detector nucleic acid comprises an RNA sequence. 4. The composition of embodiment 3, wherein the detector nucleic acid is an RNA reporter. 5. The composition of any one of embodiments 1-4, wherein the composition further comprises the target deoxyribonucleic acid. 6. The composition of any one of embodiments 1-5, wherein the target deoxyribonucleic acid is an amplicon of a nucleic acid. 7. The composition of embodiment 6, wherein the nucleic acid is a deoxyribonucleic acid or a ribonucleic acid. 8. The composition of any one of embodiments 1-7, wherein the DNA-activated programmable RNA nuclease comprises a HEPN domain. 9. The composition of any one of embodiments 1-8, wherein the DNA-activated programmable RNA nuclease comprises two HEPN domains. 10. The composition of any one of embodiments 1-9, wherein the DNA-activated programmable RNA nuclease is a Type VI CRISPR/Cas enzyme. 11. The composition of any one of embodiments 1-10, wherein the DNA-activated programmable RNA nuclease is a Cas13 protein. 12. The composition of any one of embodiments 1-11, wherein the Cas13 protein comprises a Cas13a polypeptide, a Cas13b polypeptide, a Cas13c polypeptide, a Cas13c polypeptide, a Cas13d polypeptide, or a Cas13e polypeptide. 13. The composition of any one of embodiments 11-12, wherein the Cas13 protein is a Cas13a polypeptide. 14. The composition of embodiment 13, wherein the Cas13a polypeptide is LbuCas13a or LwaCas13a. 15. The composition of any one of embodiments 1-14, wherein the DNA-activated programmable RNA nuclease has at least 80%, at least 85%, at least 90%, at least 92%, at least 95%, at least 97%, or at least 99% sequence identity to any one of SEQ ID NO: 18-SEQ ID NO: 35. 16. The composition of any one of embodiments 1-15, wherein the DNA-activated programmable RNA nuclease is selected from any one of SEQ ID NO: 18-SEQ ID NO: 35. 17. The composition of any one of embodiments 1-16, wherein the composition has a pH from pH 6.8 to pH 8.2. 18. The composition of any one of embodiments 1-17, wherein the target deoxyribonucleic acid lacks a guanine at the 3′ end. 19. The composition of any one of embodiments 1-18, wherein the terminal 3′ nucleotide in the segment of the target deoxyribonucleic acid is A, C or T. 20. The composition of any one of embodiments 1-19, wherein the target deoxyribonucleic acid is a single-stranded deoxyribonucleic acid. 21. The composition of any one of embodiments 1-20, wherein the target deoxyribonucleic acid is single stranded deoxyribonucleic acid oligonucleotides. 22. The composition of any one of embodiments 1-21, wherein the target deoxyribonucleic acid is genomic single stranded deoxyribonucleic acids. 23. The composition of any one of embodiments 1-22, wherein the target deoxyribonucleic acid has a length of from 18 to 100 nucleotides. 24. The composition of any one of embodiments 1-23, wherein the target deoxyribonucleic acid has a length of from 18 to 30 nucleotides. 25. The composition of any one of embodiments 1-24, wherein the target deoxyribonucleic acid has a length of 20 nucleotides. 26. The composition of any one of embodiments 1-26, wherein the composition is present within a support medium. 27. A lateral flow device comprising the composition of any one of embodiments 1-26. 28. A device configured for fluorescence detection comprising the composition of any one of embodiments 1-26. 29. The composition of any one of embodiments 1-26, further comprising a second engineered guide nucleic acid comprising a first segment that is reverse complementary to a segment of a second target deoxyribonucleic acid; and a DNA-activated programmable DNA nuclease, wherein the second engineered guide nucleic acid comprises a second segment that binds to the DNA-activated programmable DNA nuclease to form a complex. 30. The composition of embodiment 29, further comprising a DNA reporter. 31. The composition of any one of embodiments 29-30, wherein the DNA-activated programmable DNA nuclease comprises a RuvC catalytic domain. 32. The composition of any one of embodiments 29-31, wherein the DNA-activated programmable DNA nuclease comprises a type V CRISPR/Cas enzyme. 33. The composition of embodiment 32, wherein the type V CRISPR/Cas enzyme is a Cas12 protein. 34. The composition of embodiment 33, wherein the Cas12 protein comprises a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, and a C2c9 polypeptide. 35. The composition of any one of embodiments 33-34, wherein the Cas12 protein has at least 80%, at least 85%, at least 90%, at least 92%, at least 95%, at least 97%, or at least 99% sequence identity to any one of SEQ ID NO: 36-SEQ ID NO: 46. 36. The composition of any one of embodiments 33-35, wherein the Cas12 protein is selected from SEQ ID NO: 36-SEQ ID NO: 46. 37. The composition of embodiment 32, wherein the type V CRIPSR/Cas enzyme is a Cas14 protein. 38. The composition of embodiment 37, wherein the Cas14 protein comprises a Cas14a polypeptide, a Cas14b polypeptide, a Cas14c polypeptide, a Cas14d polypeptide, a Cas14e polypeptide, a Cas14f polypeptide, a Cas14g polypeptide, a Cas14h polypeptide, a Cas14i polypeptide, a Cas14j polypeptide, or a Cas14k polypeptide. 39. The composition of any one of embodiments 37-38, wherein the Cas14 protein has at least 80%, at least 85%, at least 90%, at least 92%, at least 95%, at least 97%, or at least 99% sequence identity to any one of SEQ ID NO: 47-SEQ ID NO: 138. 40. The composition of any one of embodiments 37-39, wherein the Cas14 protein is selected from SEQ ID NO: 47-SEQ ID NO: 138. 41. The composition of embodiment 32, wherein the type V CRIPSR/Cas enzyme is a CasΦ protein. 42. The composition of embodiment 41, wherein the CasΦ protein has at least 80%, at least 85%, at least 90%, at least 92%, at least 95%, at least 97%, or at least 99% sequence identity to any one of SEQ ID NO: 139-SEQ ID NO: 186. 43. The composition of any one of embodiments 41-42, wherein the CasΦ protein is selected from SEQ ID NO: 139-SEQ ID NO: 186. 44. A method of assaying for a target deoxyribonucleic acid in a sample, the method comprising: contacting the sample to a complex comprising: a DNA-activated programmable RNA nuclease; and an engineered guide nucleic acid comprising a first segment that is reverse complementary to a segment of a target deoxyribonucleic acid and a second segment that binds to the DNA-activated programmable RNA nuclease; and assaying for a signal produced by cleavage of at least some RNA reporters of a plurality of RNA reporters by the DNA-activated programmable RNA nuclease upon hybridization of the first segment of the engineered guide nucleic acid to the segment of the target deoxyribonucleic acid. 45. A method of assaying for a target ribonucleic acid in a sample, the method comprising: amplifying the target ribonucleic acid in a sample to produce a target deoxyribonucleic acid; contacting the target deoxyribonucleic acid to a complex comprising: a DNA-activated programmable RNA nuclease; and an engineered guide nucleic acid comprising a first segment that is reverse complementary to a segment of a target deoxyribonucleic acid and a second segment that binds to the DNA-activated programmable RNA nuclease; and assaying for a signal produced by cleavage of at least some RNA reporters of a plurality of RNA reporters by the DNA-activated programmable RNA nuclease upon hybridization of the first segment of the engineered guide nucleic acid to the segment of the target deoxyribonucleic acid. 46. The method of any one of embodiments 44-45, wherein the DNA-activated programmable RNA nuclease comprises a HEPN domain. 47. The method of any one of embodiments 44-46, wherein the DNA-activated programmable RNA nuclease comprises two HEPN domains. 48. The method of any one of embodiments 44-47, wherein the DNA-activated programmable RNA nuclease is a Type VI CRISPR/Cas enzyme. 49. The method of any one of embodiments 44-48, wherein the DNA-activated programmable RNA nuclease is a Cas13 protein. 50. The method of any embodiment 49, wherein the Cas13 protein comprises a Cas13a polypeptide, a Cas13b polypeptide, a Cas13c polypeptide, a Cas13c polypeptide, a Cas13d polypeptide, or a Cas13e polypeptide. 51. The method of any one of embodiments 49-50, wherein the Cas13 protein is a Cas13a polypeptide. 52. The method of embodiment 51, wherein the Cas13a polypeptide is LbuCas13a or LwaCas13a. 53. The method of any one of embodiments 44-52, wherein the DNA-activated programmable RNA nuclease has at least 80%, at least 85%, at least 90%, at least 92%, at least 95%, at least 97%, or at least 99% sequence identity to any one of SEQ ID NO: 18-SEQ ID NO: 35. 54. The method of any one of embodiments 44-53, wherein the DNA-activated programmable RNA nuclease is selected from any one of SEQ ID NO: 18-SEQ ID NO: 35. 55. The method of any one of embodiments 44-54, wherein cleavage of the at least some RNA reporters of the plurality of reporters occurs from pH 6.8 to pH 8.2. 56. The method of any one of embodiments 44-55, wherein the target deoxyribonucleic acid lacks a guanine at the 3′ end. 57. The method of any one of embodiments 44-56, wherein the terminal 3′ nucleotide in the segment of the target deoxyribonucleic acid is A, C or T. 58. The method of any one of embodiments 44-57, wherein the target deoxyribonucleic acid is a single-stranded deoxyribonucleic acid. 59. The method of any one of embodiments 44-58, wherein the target deoxyribonucleic acid is an amplicon of a ribonucleic acid. 60. The method of any one of embodiments 44-59, wherein the target deoxyribonucleic acid or the ribonucleic acid is from an organism. 61. The method of embodiment 60, wherein the organism is a virus, bacteria, plant, or animal. 62. The method of any one of embodiments 44-61, wherein the target deoxyribonucleic acid is produced by a nucleic acid amplification method. 63. The method of any one of embodiments 44-62, wherein the amplifying comprises isothermal amplification. 64. The method of any one of embodiments 44-62, wherein the amplifying comprises thermal amplification. 65. The method of any one of embodiments 44-64, wherein the amplifying comprises recombinase polymerase amplification (RPA), transcription mediated amplification (TMA), strand displacement amplification (SDA), helicase dependent amplification (HDA), loop mediated amplification (LAMP), rolling circle amplification (RCA), single primer isothermal amplification (SPIA), ligase chain reaction (LCR), simple method amplifying RNA targets (SMART), or improved multiple displacement amplification (IMDA), or nucleic acid sequence-based amplification (NASBA). 66. The method of any one of embodiments 44-65, wherein the amplifying is loop mediated amplification (LAMP). 67. The method of any one of embodiments 44-66, wherein the signal is fluorescence, luminescence, colorimetric, electrochemical, enzymatic, calorimetric, optical, amperometric, or potentiometric. 68. The method of any one of embodiments 44-67, further comprising contacting the sample to a second engineered guide nucleic acid comprising a first segment that is reverse complementary to a segment of a second target deoxyribonucleic acid; and a DNA-activated programmable DNA nuclease, wherein the second engineered guide nucleic acid comprises a second segment that binds to the DNA-activated programmable DNA nuclease to form a complex. 69. The method of embodiment 68, further comprising assaying for a signal produced by cleavage of at least some DNA reporters of a plurality of DNA reporters. 70. The composition of any one of embodiments 1-43 or the method of any one of embodiments 44-69, wherein the engineered guide nucleic acid comprises a crRNA. 71. The composition of any one of embodiments 1-43 or the method of any one of embodiments 44-70, wherein the engineered guide nucleic acid comprises a crRNA and a tracrRNA. 72. The method of any one of embodiments 44-71, wherein the signal is present prior to cleavage of the at least some RNA reporters. 73. The method of any one of embodiments 44-71, wherein the signal is absent prior to cleavage of the at least some RNA reporters. 74. The method of any one of embodiments 44-73, wherein the sample comprises blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. 75. The method of any one of embodiments 44-74, wherein the method is carried out on a support medium. 76. The method of any one of embodiments 44-75, wherein the method is carried out on a lateral flow assay device. 77. The method of any one of embodiments 44-76, wherein the method is carried out on a device configured for fluorescence detection. 78. The method of any one of embodiments 68-77, wherein the DNA-activated programmable DNA nuclease comprises a RuvC catalytic domain. 79. The method of any one of embodiments 68-78, wherein the DNA-activated programmable DNA nuclease comprises a type V CRISPR/Cas enzyme. 80. The method of embodiment 79, wherein the type V CRISPR/Cas enzyme is a Cas12 protein. 81. The method of embodiment 80, wherein the Cas12 protein comprises a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, and a C2c9 polypeptide. 82. The method of any one of embodiments 80-81, wherein the Cas12 protein has at least 80%, at least 85%, at least 90%, at least 92%, at least 95%, at least 97%, or at least 99% sequence identity to any one of SEQ ID NO: 36-SEQ ID NO: 46. 83. The method of any one of embodiments 80-82, wherein the Cas12 protein is selected from SEQ ID NO: 36-SEQ ID NO: 46. 84. The method of embodiment 79, wherein the type V CRIPSR/Cas enzyme is a Cas14 protein. 85. The method of embodiment 84, wherein the Cas14 protein comprises a Cas14a polypeptide, a Cas14b polypeptide, a Cas14c polypeptide, a Cas14d polypeptide, a Cas14e polypeptide, a Cas14f polypeptide, a Cas14g polypeptide, a Cas14h polypeptide, a Cas14i polypeptide, a Cas14j polypeptide, or a Cas14k polypeptide. 86. The method of any one of embodiments 84-85, wherein the Cas14 protein has at least 80%, at least 85%, at least 90%, at least 92%, at least 95%, at least 97%, or at least 99% sequence identity to any one of SEQ ID NO: 47-SEQ ID NO: 138. 87. The method of any one of embodiments 84-86, wherein the Cas14 protein is selected from SEQ ID NO: 47-SEQ ID NO: 138. 88. The method of embodiment 79, wherein the type V CRIPSR/Cas enzyme is a CasΦ protein. 89. The method of embodiment 88, wherein the CasΦ protein has at least 80%, at least 85%, at least 90%, at least 92%, at least 95%, at least 97%, or at least 99% sequence identity to any one of SEQ ID NO: 139-SEQ ID NO: 186. 90. The method of any one of embodiments 88-89, wherein the CasΦ protein is selected from SEQ ID NO: 139-SEQ ID NO: 186. 91. The composition of any one of embodiments 1-43 or 70-71, wherein the target deoxyribonucleic acid is a reverse transcribed ribonucleic acid. 92. The method of any one of embodiments 44-90, wherein the amplifying the target ribonucleic acid in a sample to produce a target deoxyribonucleic acid comprises reverse transcribing the target ribonucleic acid in the sample to produce the target deoxyribonucleic acid. 93. The composition of any one of embodiments 1-43, 70-71, or 91, wherein the composition further comprises a reagent for reverse transcription. 94. The composition of any one of embodiments 1-43, 70-71, 91, or 93, wherein the composition further comprises a reagent for amplification. 95. The composition of any one of embodiments 1-43, 70-71, 91, or 93-94, wherein the composition further comprises a reagent for in vitro transcription. 96. The method of any one of embodiments 44-90 or 92, wherein the method comprises contacting the target deoxyribonucleic acid or the target ribonucleic acid with a reagent for reverse transcription. 97. The method of any one of embodiments 44-90, 92, or 96, wherein the method comprises contacting the target deoxyribonucleic acid or the target ribonucleic acid with a reagent for amplification. 98. The method of any one of embodiments 44-90, 92, or 96-97, wherein the method comprises contacting the target deoxyribonucleic acid or the target ribonucleic acid with a reagent for in vitro transcription. 99. The composition or method of any one of embodiments 93-98, wherein the reagent for reverse transcription comprises a reverse transcriptase, an oligonucleotide primer, dNTPs, or any combination thereof. 100. The composition or method of any one of embodiments 93-99, wherein the reagent for amplification comprises a primer, a polymerase, dNTPs, or any combination thereof 101. The composition or method of any one of embodiments 93-100, wherein the reagent for in vitro transcription comprise an RNA polymerase, NTPs, a primer, or any combination thereof 102. A method of assaying for a target deoxyribonucleic acid in a sample, the method comprising: contacting the sample to the composition of any one of embodiments 1-43; and assaying for a signal produced by cleavage of at least some RNA reporters of a plurality of RNA reporters by the DNA-activated programmable RNA nuclease upon hybridization of the first segment of the engineered guide nucleic acid to the segment of the target deoxyribonucleic acid. 103. A method of assaying for a target ribonucleic acid in a sample, the method comprising: amplifying the target ribonucleic acid in a sample to produce a target deoxyribonucleic acid; contacting the target deoxyribonucleic acid to the composition of any one of embodiments 1-43; and assaying for a signal produced by cleavage of at least some RNA reporters of a plurality of RNA reporters by the DNA-activated programmable RNA nuclease upon hybridization of the first segment of the engineered guide nucleic acid to the segment of the target deoxyribonucleic acid. 104. The use of a composition according to any one of embodiments 1-26, 29-43, 70, 71, 90, or 93-95 in a method of assaying for a target deoxyribonucleic acid in a sample. 105. The use of a DNA-activated programmable RNA nuclease in a method of assaying for a target deoxyribonucleic acid in a sample according to any one of embodiments 44, 46-90, or 96-102. 106. The use of a DNA-activated programmable RNA nuclease in a method of assaying for a target ribonucleic acid in a sample according to any one of embodiments 45-90, 96-101, or 103.

### EXAMPLES

The following examples are illustrative and non-limiting to the scope of the devices, systems, fluidic devices, kits, and methods described herein.

### Example 1

### Cas13a Detection of DNA

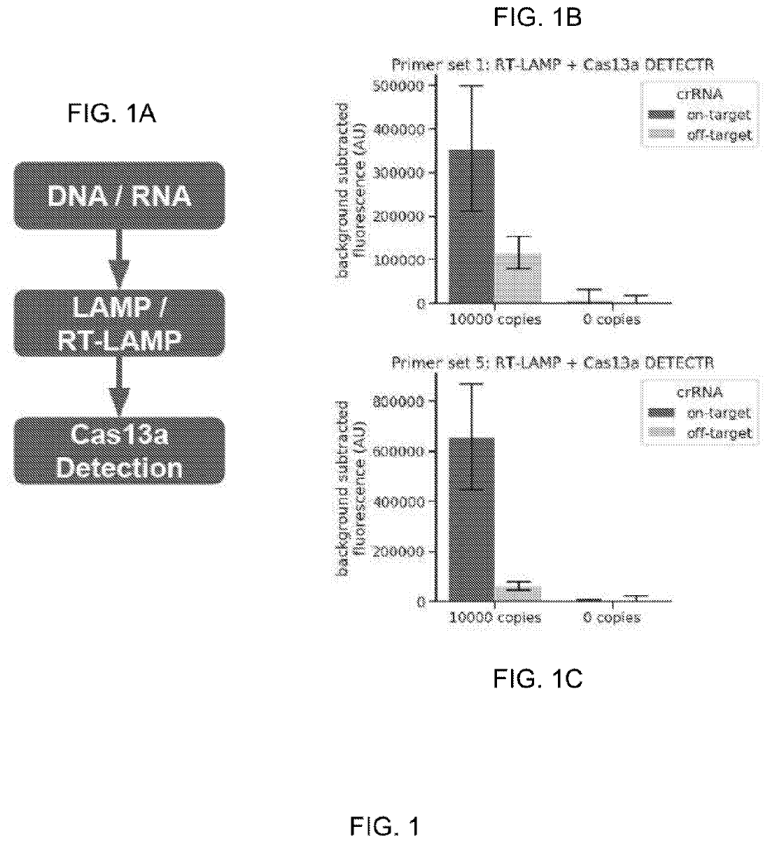

This example describes Cas13a detection of target DNA. Cas13a was used to detect a target RT-LAMP DNA amplicon from Influenza A RNA. FIG. 1A shows a schematic The RT-LAMP reaction was performed at 55° C. for 30 minutes with a starting RNA concentration of 10,000 viral genome copies or 0 viral genome copies, as a control. Two different primer sets showed the same results (FIG. 1B and FIG. 1C). After completion of the RT-LAMP reaction, 1 pL of amplicon was added to a 20 μL Cas13a detection reaction. On-target and off-target crRNAs were used to show specific detection by Cas13a at 37° C. of the RT-LAMP DNA amplicon.

FIG. 1A shows a schematic of the workflow including providing DNA/RNA, LAMP/RT-LAMP, and Cas13a detection. FIG. 1B shows Cas13a specific detection of target RT-LAMP DNA amplicon with a first primer set as measured by background subtracted fluorescence on the y-axis. On-target crRNA results are shown by the darker bars and off-target crRNA control results are shown in lighter bars. A starting RNA concentration of 10,000 viral genome copies is shown in the left two bars and 0 viral genome copies (negative control) is shown in the right two bars. FIG. 1C shows Cas13a specific detection of target RT-LAMP DNA amplicon with a second primer set as measured by background subtracted fluorescence on the y-axis. On-target crRNA results are shown by the darker bars and off-target crRNA control results are shown in lighter bars. A starting RNA concentration of 10,000 viral genome copies is shown in the left two bars and 0 viral genome copies (negative control) is shown in the right two bars.

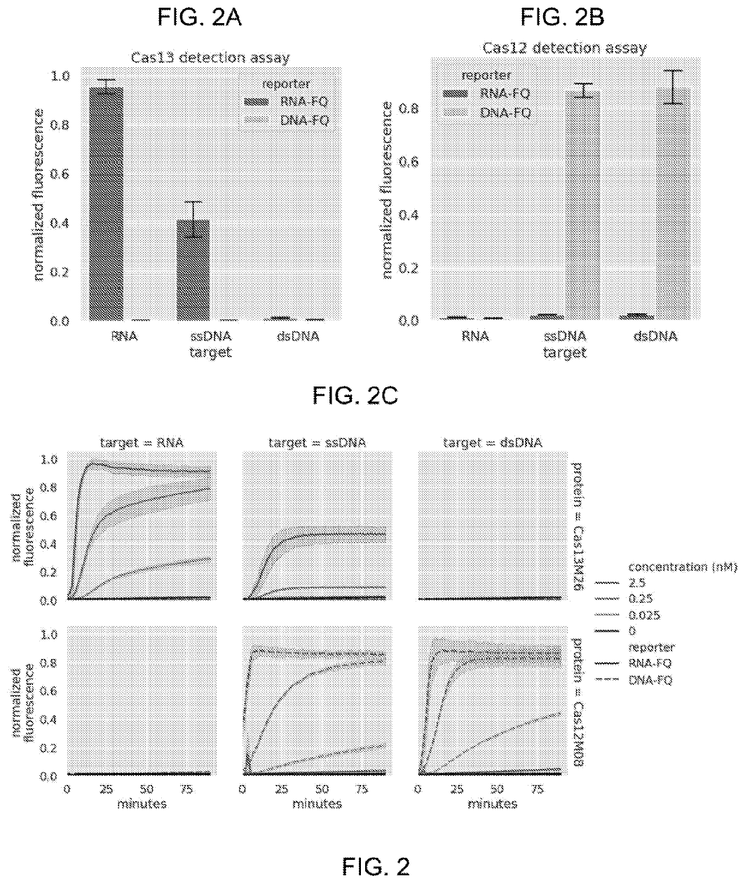

Cas13a recognized target ssDNA and target RNA. FIG. 2A shows a Cas13 detection assay using 2.5 nM RNA, single-stranded DNA (ssDNA), or double-stranded (dsDNA) as target nucleic acids, where detection was measured by fluorescence for each of the target nucleic acids tested. The reaction was performed at 37° C. for 20 minutes with both RNA-FQ (RNA-fluorescence quenched reporter) and DNA-FQ reporter substrates. Results showed that Cas13 initiates trans-cleavage activity for RNA-FQ for both target RNA and target ssDNA. Data was normalized to max fluorescence signal for each reporter substrate. FIG. 2B shows Cas12 detection assay using 2.5 nM RNA, ssDNA, and dsDNA as target nucleic acids, where detection was measured by fluorescence for each of the target nucleic acids tested. Reactions were performed at 37° C. for 20 minutes with both RNA-FQ and DNA-FQ reporter substrates. Results supported the previously established preference for Cas12 for either target ssDNA or target dsDNA and specificity for DNA-FQ. Data was normalized to max fluorescence signal for each reporter substrate. FIG. 2C shows the performance of Cas13 and Cas12 on target RNA, target ssDNA, and target dsDNA at various concentrations, where detection was measured by fluorescence for each of the target nucleic acids tested. Reactions were performed at 37° C. for 90 minutes with both RNA-FQ and DNA-FQ reporter substrates. Data was normalized to max fluorescence signal for each reporter substrate. Results indicated picomolar sensitivity of Cas13 for target ssDNA.

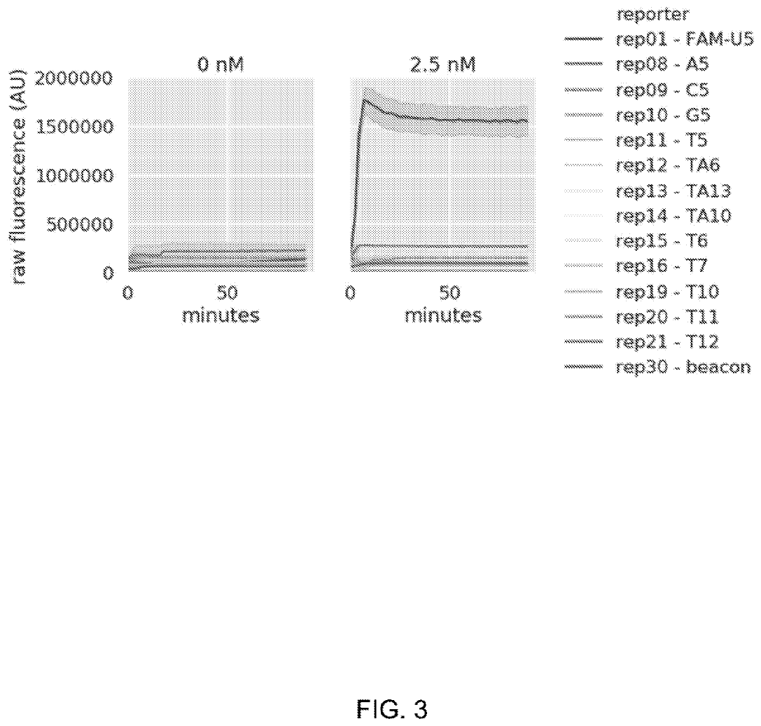

Cas13a trans-cleavage activity was found to be specific for RNA reporters when targeting target ssDNA. FIG. 3 shows an Lbu-Cas13a (SEQ ID NO: 19) detection assay using 2.5 nM target ssDNA with 170 nM of various reporter substrates, wherein detection was measured by fluorescence for each of the reporter substrates tested. A single RNA-FQ reporter substrate (rep01-FAM-U5) was tested and 13 DNA-FQ reporter substrates were tested. TABLE 6 below shows the sequence of each of the reporters tested.

TABLE 6
Reporter Sequences

| Reporter | |
|---|---|
| ID | Sequence |
| rep01 | /56-FAM/rUrUrUrUrU (SEQ ID NO: l)/ |
| | 3IABkFQ/ |
| rep08 | /56-FAM/AAAAA (SEQ ID NO: 194)/3IABkFQ/ |
| rep09 | /56-FAM/CCCCC (SEQ ID NO: 195)/3IABkFQ/ |
| rep10 | /56-FAM/GGGGG (SEQ ID NO: 196)/3IABkFQ/ |
| rep11 | /56-FAM/TTTTT (SEQ ID NO: 197)/3IABkFQ/ |
| rep12 | /56-FAM/TTATTA (SEQ ID NO: 198)/3IABkFQ/ |
| rep13 | /56-FAM/TTATTATT (SEQ ID NO: 9)/3IABkFQ/ |
| rep14 | /56-FAM/ATTATTATTA (SEQ ID NO: 199)/ |
| | 3IABkFQ/ |
| rep15 | /56-FAM/TTTTTT (SEQ ID NO: 10)/3IABkFQ/ |
| rep16 | /56-FAM/TTTTTTT (SEQ ID NO: 200)/ |
| | 3IABkFQ/ |
| rep17 | /56-FAM/TTTTTTTTTT (SEQ ID NO: 12)/ |
| | 3IABkFQ/ |
| rep18 | /56-FAM/TTTTTTTTTTT (SEQ ID NO: 201)/ |
| | 3IABkFQ/ |
| rep19 | /56-FAM/TTTTTTTTTTTT (SEQ ID NO: 13)/ |
| | 3IABkFQ/ |
| rep30 | /FAM/CCGGCAGCCATAACGCCGTGAATACGTTCTGCCG |
| | G (SEQ ID NO: 202)/BHQl/ |

Results indicated that Cas13 trans-cleavage was specific for RNA reporters, even when activated by target ssDNA.

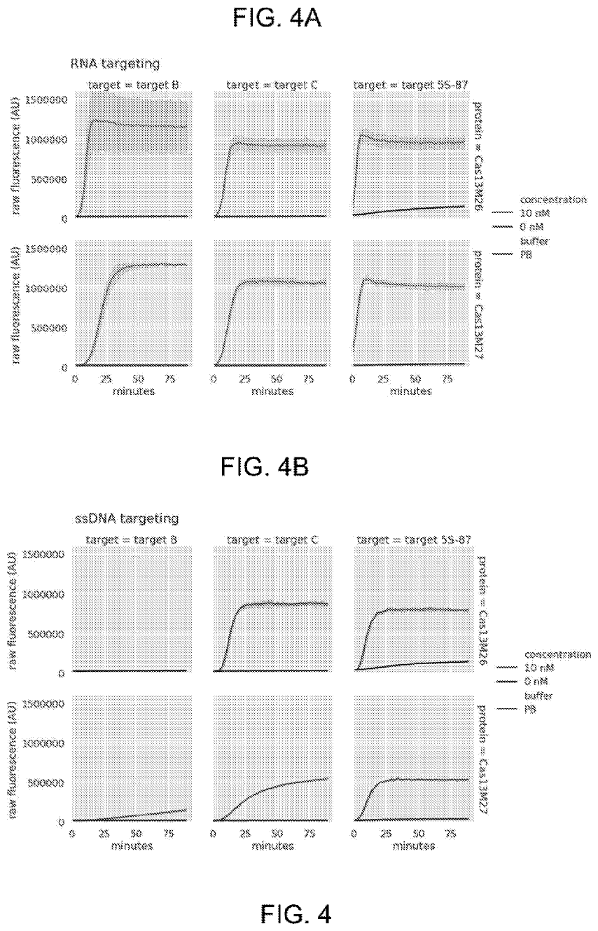

Multiple Cas13 family members detected target ssDNA. FIG. 4A shows the results of Cas13 detection assays for Lbu-Cas13a (SEQ ID NO: 19) and Lwa-Cas13a (SEQ ID NO: 25) using 10 nM of target RNA or no target RNA (shown as 0 nM), where detection was measured by fluorescence resulting from cleavage of reporters over time. Three target RNAs encoding different sequences were evaluated with corresponding gRNAs. Results showed similar detection of all three target nucleic acids for both Cas13 family members. FIG. 4B shows the results of Cas13 detection assays for Lbu-Cas13a (SEQ ID NO: 19) and Lwa-Cas13a (SEQ ID NO: 25) using 10 nM of target ssDNA or no target ssDNA (shown as 0 nM), where detection was measured by fluorescence resulting from cleavage of reporters over time. Three target DNA and their corresponding gRNAs, with the same sequence as the target RNAs, were evaluated. Results showed Cas13 family preferences in target ssDNA recognition, with Lbu-Cas13a (SEQ ID NO: 19) exhibiting faster detection for some target nucleic acids and Lwa-Cas13a (SEQ ID NO: 25) exhibiting faster detection for other targets

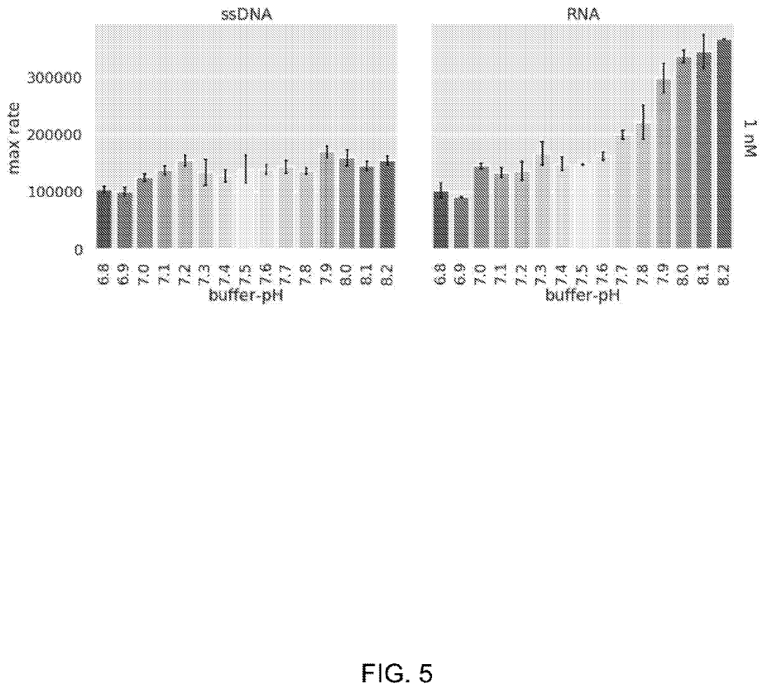

Cas13 detection of target ssDNA was robust at multiple pH values. FIG. 5 shows Lbu-Cas13a (SEQ ID NO: 19) detection assay using 1 nM target RNA (at left) or target ssDNA (at right) in buffers with various pH values ranging from 6.8 to 8.2. Reactions were performed at 37° C. for 20 minutes with RNA-FQ reporter substrates. Results indicated enhanced Cas13 RNA detection at buffers with a higher pH (7.9 to 8.2), whereas Cas13 ssDNA detection was consistent across pH conditions (6.8 to 8.2).

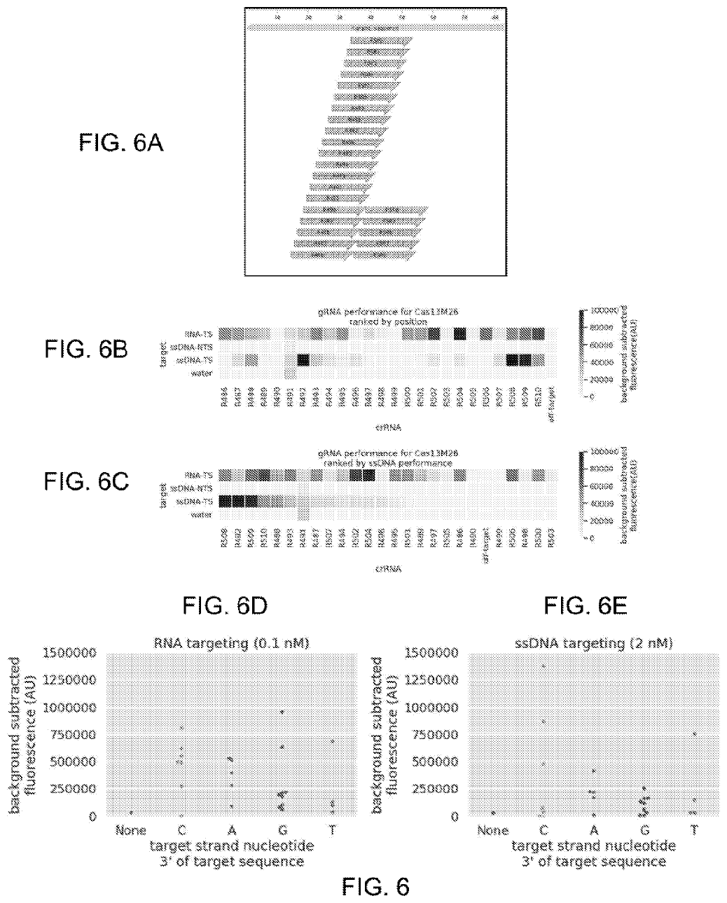

Cas13 preferences for target ssDNA were found to be distinct from preferences for target RNA. FIG. 6A shows guide RNAs (gRNAs) tiled along a target sequence at 1 nucleotide intervals. FIG. 6B shows Cas13M26 detection assays using 0.1 nM RNA or 2 nM target ssDNA with gRNAs tiled at 1 nucleotide intervals and an off-target gRNA. Guide RNAs were ranked by their position along the sequence of the target nucleic acid. FIG. 6C shows data from FIG. 6B ranked by performance of target ssDNA. Results showed that gRNA performance on target ssDNA did not correlate with the performance of the same gRNAs on RNA. FIG. 6D shows performance of gRNAs for each nucleotide on a 3′ end of a target RNA. Results indicated that there are high performing gRNAs on target RNAs regardless of target nucleotide identity at this position. FIG. 6E shows performance of gRNAs for each nucleotide on a 3′ end of a target ssDNA. Results indicated that a G in the target at this position performed worse than other gRNAs.

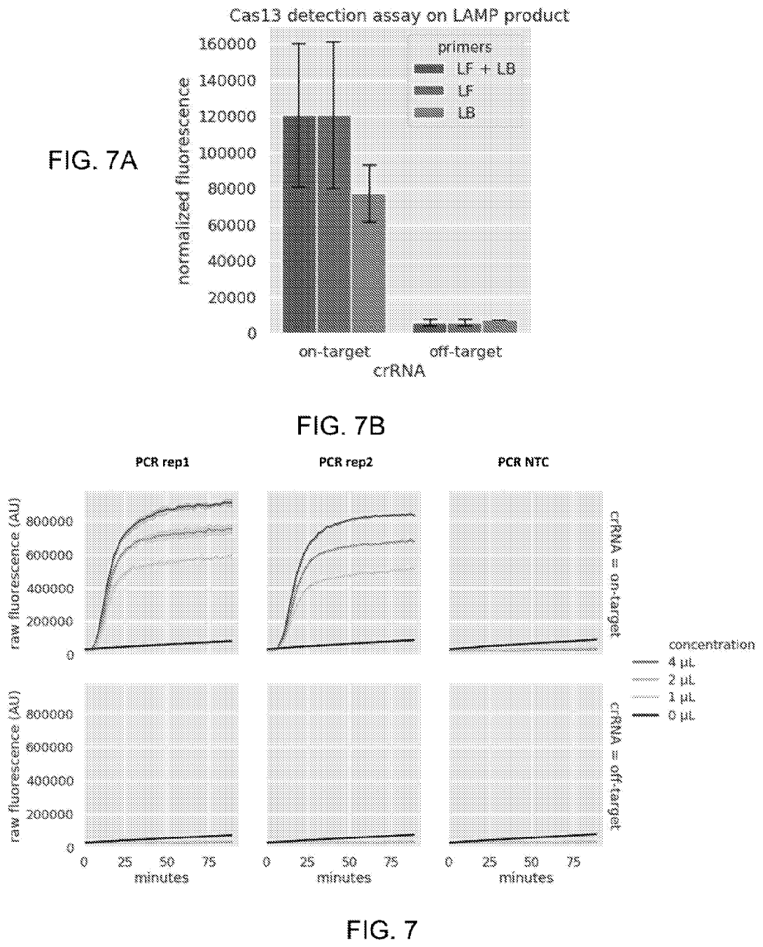

Cas13a detected target DNA generated by nucleic acid amplification methods (PCR, LAMP). FIG. 7A shows Lbu-Cas13a (SEQ ID NO: 19) detection assays using 1 μL of target DNA amplicon from various LAMP isothermal nucleic acid amplification reactions. LAMP conditions tested included 6-primer with both loop-forward (LF) and loop-reverse (LB), asymmetric LAMP with LF only, and asymmetric LAMP with LB only. All tested LAMP reactions generated an Lbu-Cas13a (SEQ ID NO: 19) compatible target DNA. FIG. 7B shows Cas13M26 detection assays using various amounts of PCR reaction as a target DNA. Results indicated that PCR generated enough target ssDNA to enable Cas13 detection.

### Example 2

### Detection of Influenza Using a DNA-Activated Programmable RNA Nuclease

This example describes detection of an influenza viral infection in a sample using a DNA-activated programmable RNA nuclease, such as Cas13a. A fluid sample, for example saliva, is obtained from an individual who may be at risk for influenza. The RNA in the fluid sample is reverse transcribed into cDNA using a reverse transcriptase enzyme. The reverse transcribed cDNA from the fluid sample is combined with a DNA-activated programmable RNA nuclease, a guide RNA comprising a sequence that is reverse complementary to a target cDNA sequence found in the influenza genome, and an RNA reporter.

If influenza is present in the fluid sample, the guide RNA binds to the reverse transcribed target cDNA and the DNA-activated programmable RNA nuclease is activated. The activated DNA-activated programmable RNA nuclease cleaves the RNA reporter. Upon cleavage of the RNA reporter, a detectable signal is generated, indicating that the sample is positive for influenza.

### Example 3

### Detection of Dengue Using a DNA-Activated Programmable RNA Nuclease

This example describes detection of a dengue viral infection in a sample using a DNA-activated programmable nuclease, such as Cas13a. A fluid sample, for example saliva, is obtained from an individual who may be at risk for dengue. The RNA in the fluid sample is reverse transcribed into cDNA using a reverse transcriptase enzyme. The reverse transcribed cDNA from the fluid sample is combined with a DNA-activated programmable RNA nuclease, a guide RNA comprising a sequence that is reverse complementary to a target cDNA sequence found in the dengue genome, and an RNA reporter.

If dengue is present in the fluid sample, the guide RNA binds to the reverse transcribed target cDNA and the DNA-activated programmable RNA nuclease is activated. The activated DNA-activated programmable RNA nuclease cleaves the RNA reporter. Upon cleavage of the detector RNA, a detectable signal is generated, indicating that the sample is positive for dengue.

### Example 4

### Detection of Multiple Infectious Species Using a DNA-Activated Programmable RNA Nuclease

This example describes detection of multiple infectious species in a sample using a DNA-activated programmable RNA nuclease, such as Cas13a. A fluid sample, for example saliva, is obtained from an individual who may be at risk for sepsis. The fluid sample is combined with a Cas13 programmable nuclease, multiple guide RNAs comprising sequences that are reverse complementary to target DNA sequence found in the genomes of bacterial and viral species associated with sepsis, and an RNA reporter.

If sepsis is present in the fluid sample, the guide RNAs binds to one or more of the target DNAs and the DNA-activated programmable RNA nuclease is activated. The activated DNA-activated programmable RNA nuclease cleaves the RNA reporter. Upon cleavage of the RNA reporter, a detectable signal is generated indicating that the sample is positive for sepsis.

### Example 5

### Detection of Streptococcus pyogenes Using a DNA-Activated Programmable RNA Nuclease

This example describes detection of a strep bacterial infection in a sample using a DNA-activated programmable RNA nuclease, such as Cas13a. A fluid sample, for example saliva, is obtained from an individual who may be at risk for strep. The fluid sample is combined with a DNA-activated programmable RNA nuclease, a guide RNA comprising a sequence that is reverse complementary to a target DNA sequence found in the Streptococcus pyogenes genome, and an RNA reporter.

If strep is present in the fluid sample, the guide RNA binds to the target DNA and the DNA-activated programmable RNA nuclease is activated. The activated DNA-activated programmable RNA nuclease cleaves the RNA reporter. Upon cleavage of the RNA reporter, a detectable signal is generated, indicating that the sample is positive for strep.

### Example 6

### Detection of Malaria Using a DNA-Activated Programmable RNA Programmable Nuclease

This example describes detection of a malaria parasitic infection in a sample using a DNA-activated programmable RNA nuclease, such as Cas13a. A fluid sample, for example saliva, is obtained from an individual who may be at risk for malaria. The fluid sample is combined with DNA-activated programmable RNA nuclease, a guide RNA comprising a sequence that is reverse complementary to a target DNA sequence found in the Plasmodium falciparum: genome, and an RNA reporter.

If malaria is present in the fluid sample, the guide RNA binds to the target DNA and the Cas13 programmable nuclease is activated. The activated DNA-activated programmable RNA nuclease cleaves RNA reporter. Upon cleavage of the RNA reporter, a detectable signal is produced indicating that the sample is positive for malaria.

### Example 7

### Detection of a Viral Infection Using a DNA-Activated Programmable RNA

This example describes detection of a viral infection in a sample using a DNA-activated programmable RNA nuclease, such as Cas13a. A fluid sample, for example saliva, is obtained from an individual who may be at risk for the viral infection. The fluid sample is combined with a DNA-activated programmable RNA nuclease, a guide RNA comprising a sequence that is reverse complementary to a target DNA sequence found in the viral genome, and an RNA reporter.

If the virus is present in the fluid sample, the guide RNA binds to the target DNA and the DNA-activated programmable RNA nuclease is activated. The activated DNA-activated programmable RNA nuclease cleaves the RNA reporter. Upon cleavage of the RNA reporter, a detectable signal is produced indicating that the sample is positive for the viral infection.

### Example 8

### Detection of a Cancer-Associated Mutation Using a DNA-Activated Programmable RNA

This example describes detection of a cancer-associated mutation in a sample using a DNA-activated programmable RNA nuclease, such as Cas13a. For example, the cancer-associated mutation is a mutation in BRCA1 or BRCA2. A fluid sample, for example saliva, is obtained from an individual who may be at risk for breast or ovarian cancer. The fluid sample is combined with a DNA-activated programmable RNA nuclease, a guide RNA comprising a sequence that is reverse complementary to a cancer-associated mutant target DNA sequence, and an RNA reporter.

If a target DNA comprising the cancer-associated mutation is present in the fluid sample, the guide RNA binds to the target DNA and the DNA-activated programmable RNA nuclease is activated. The activated DNA-activated programmable RNA nuclease cleaves the RNA reporter. Upon cleavage of the RNA reporter, a detectable signal is produced indicating that the sample is positive for the cancer-associated mutation.

### Example 9

### Detection of a Nucleotide Insertion Using a DNA-Activated Programmable RNA

This example describes detection of a nucleotide insertion in a sample using a DNA-activated programmable RNA nuclease, such as Cas13a. A fluid sample, for example saliva, is obtained from an individual, for example an individual who may be at risk for a disease associated with a nucleotide insertion such as Huntington's disease. The fluid sample is combined with a DNA-activated programmable RNA nuclease, a guide RNA comprising a sequence that is reverse complementary to a DNA sequence encoding the nucleotide insertion, for example a polyQ tract in the huntingtin gene (e.g., reverse complementary to a sequence comprising CAG repeats), and an RNA reporter.

If a target DNA comprising the nucleotide insertion is present in the fluid sample, the guide RNA binds to the target DNA and the DNA-activated programmable RNA nuclease is activated. The activated DNA-activated programmable RNA nuclease cleaves the RNA reporter. Upon cleavage of the RNA reporter, a detectable signal is produced indicating that the sample is positive for the nucleotide insertion.

### Example 10

### Detection of a Single Nucleotide Polymorphism Using a DNA-Activated Programmable RNA Nuclease

This example describes detection of a single nucleotide polymorphism in a sample using a DNA-activated programmable RNA nuclease, such as a Cas13a. A fluid sample, for example saliva, is obtained from an individual, for example an individual who may be at risk for a disease associated with a single nucleotide polymorphism such as sickle-cell anemia. The fluid sample is combined with a DNA-activated programmable RNA nuclease, a guide RNA comprising a sequence that is reverse complementary to a DNA sequence encoding the single nucleotide polymorphism, for example a single nucleotide polymorphism associated with sickle-cell anemia, and an RNA reporter.

If a target DNA comprising the single nucleotide polymorphism is present in the fluid sample, the guide RNA binds to the target DNA and the DNA-activated programmable RNA nuclease is activated. The activated programmable nuclease cleaves the RNA reporter. Upon cleavage of the RNA reporter, a detectable signal is produced indicating that the sample is positive for the single nucleotide polymorphism.

### Example 11

### Effects of gRNA Sequence on ssDNA Detection Using a Cas13 DNA-Activated Programmable RNA Nuclease

This example describes the effects of gRNA sequence on detection of ssDNA oligonucleotides of equal concentrations using an LbuCas13a DNA-activated programmable RNA nuclease of SEQ ID NO: 19. Assays were run using either 2 nM ssDNA oligonucleotides targeted by various crRNAs or no target (shown as 0 μM). Reactions were carried out at 37° C. for 90 minutes with 170 nM of an RNA-FQ reporter substrate (/5-6FAM/rUrUrUrUrU (SEQ ID NO: 1)/3IABkFQ/). Sequences of the guides used in the assay are shown below in TABLE 7.

TABLE 7
Guide Sequences

| Guide | Sequence |
|---|---|
| R1463 | GCCACCCCAAAAAUGAAGGGGACUAAAACAccgaacgaaccacc |
| | agcaga SEQ ID NO: 188 |
| R1464 | GCCACCCCAAAAAUGAAGGGGACUAAAACAcgaacgaaccaccag |
| | cagaa SEQ ID NO: 189 |
| R1465 | GCCACCCCAAAAAUGAAGGGGACUAAAACAgaacgaaccaccagc |
| | agaag SEQ ID NO: 190 |
| R1488 | GCCACCCCAAAAAUGAAGGGGACUAAAACAaaacagaggugaggc |
| | gguca SEQ ID NO: 191 |
| R1490 | GCCACCCCAAAAAUGAAGGGGACUAAAACAacagaggugaggcgg |
| | ucagu SEQ ID NO: 192 |
| R1491 | GCCACCCCAAAAAUGAAGGGGACUAAAACAcagaggugaggcggu |
| | cagua SEQ ID NO: 193 |

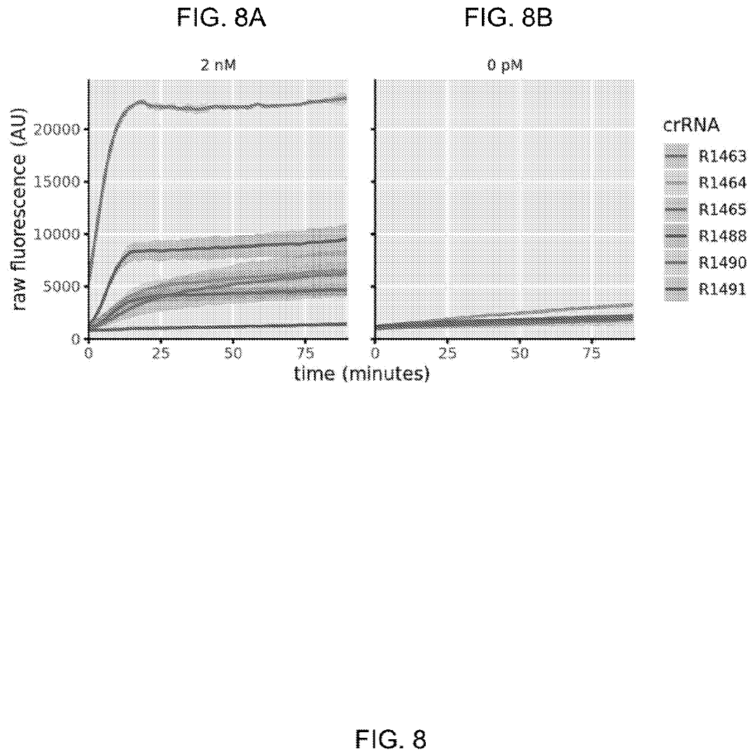

Results are shown in FIG. 8. FIG. 8A shows results from assays in which ssDNA oligonucleotides were present at 2 nM. FIG. 8B shows results from assays in which no target (shown as 0 μM) was. As shown in FIG. 8A, a detection assay in which the guide corresponding to R1490 was used resulted in rapid high levels of fluorescence, indicative of trans cleavage of the RNA-FQ reporter substrate by the activated DNA-activated programmable RNA nuclease upon hybridization of R1490 to the target ssDNA oligonucleotide. As shown in FIG. 8A, guides that worked best were R1490 and R1491 followed by similar levels of activity observed with R1464, R1465, and R1463.

### Example 12

### Detection of M13mp18 ssDNA Using a Cas13 DNA-Activated Programmable RNA Nuclease

This example describes detection of ssDNA genome from the bacteriophage M13mp18 using an LbuCas13a DNA-activated programmable RNA nuclease of (SEQ ID NO: 19). Assays were run using either 2 nM of ssDNA from the M13mp18 bacteriophage or no target (shown as 0 pM). Reactions were carried out at 37° C. for 90 minutes with 170 nM of an RNA-FQ reporter substrate (/5-6FAM/rUrUrUrUrU (SEQ ID NO: 1)/3IABkFQ/). Sequences of the guides used in the assay are shown below in TABLE 8.

TABLE 8
Guide Sequences

| Guide | Sequence |
|---|---|
| R1490 | GCCACCCCAAAAAUGAAGGGGACUAAAACAacagaggugaggcg |
| | gucagu SEQ ID NO: 192 |
| R1488 | GCCACCCCAAAAAUGAAGGGGACUAAAACAaaacagaggugagg |
| | cgguca SEQ ID NO: 191 |
| R1491 | GCCACCCCAAAAAUGAAGGGGACUAAAACAcagaggugaggcgg |
| | ucagua SEQ ID NO: 193 |

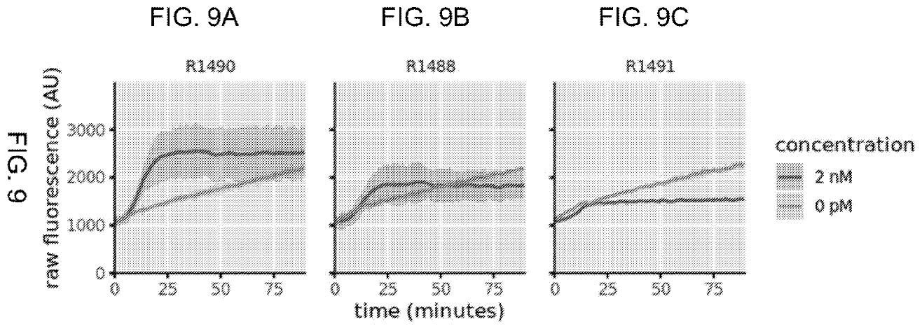

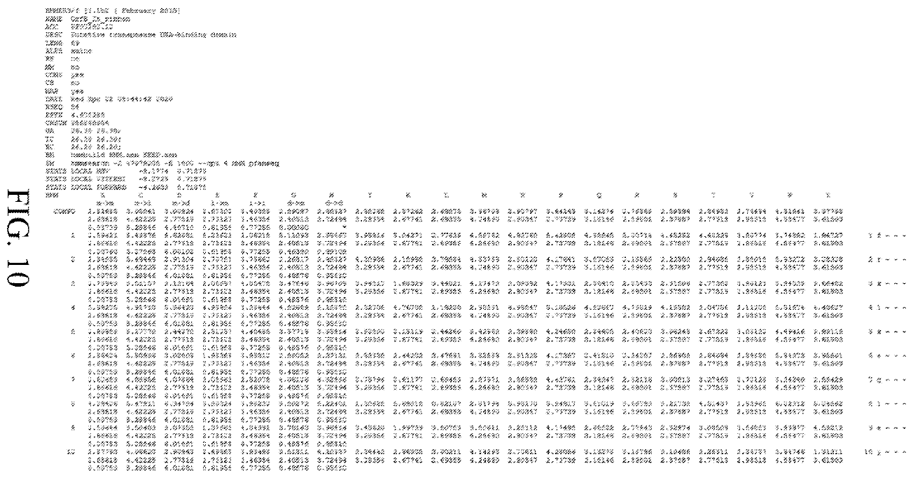

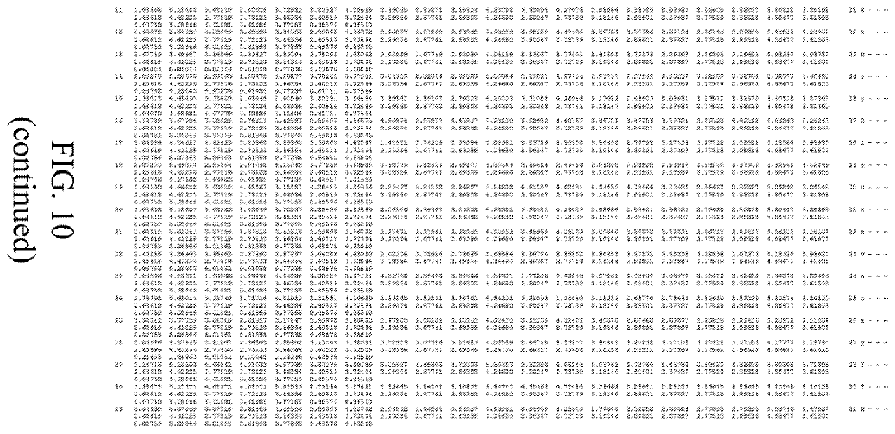

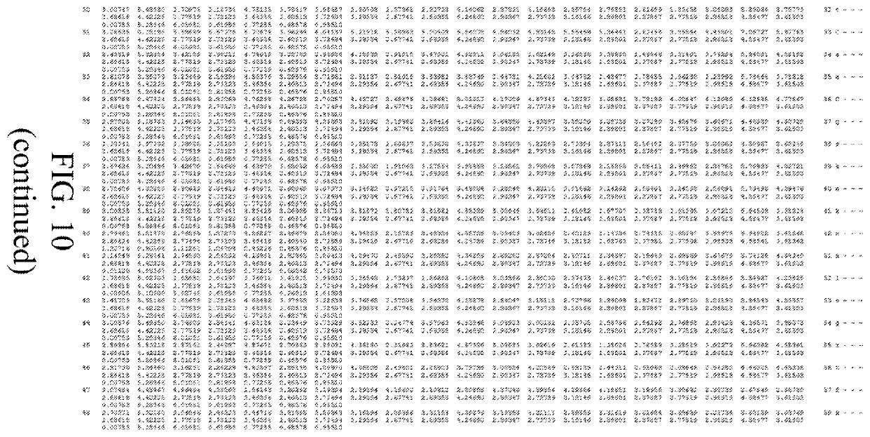

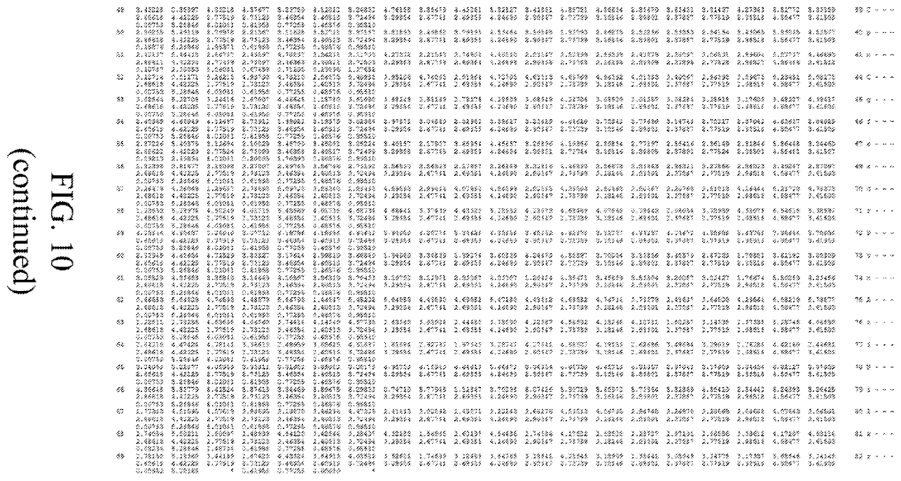

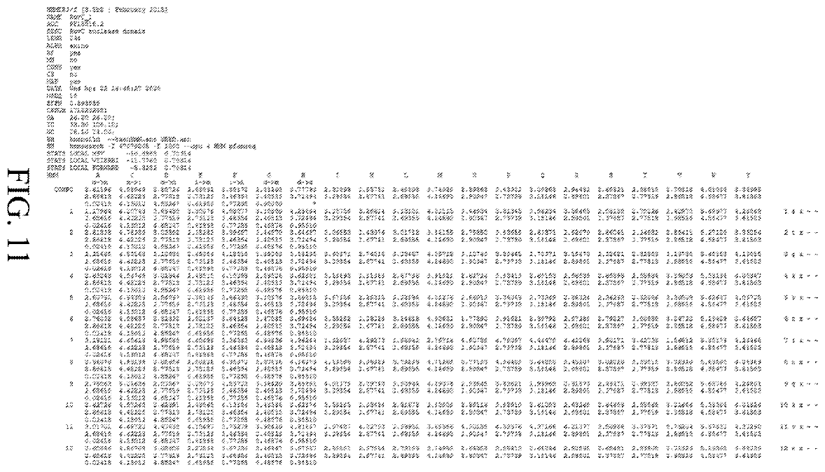

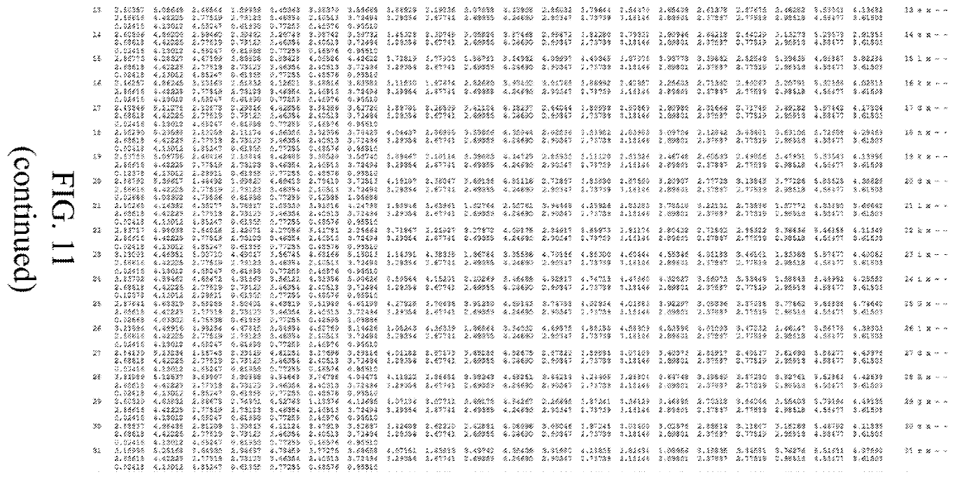

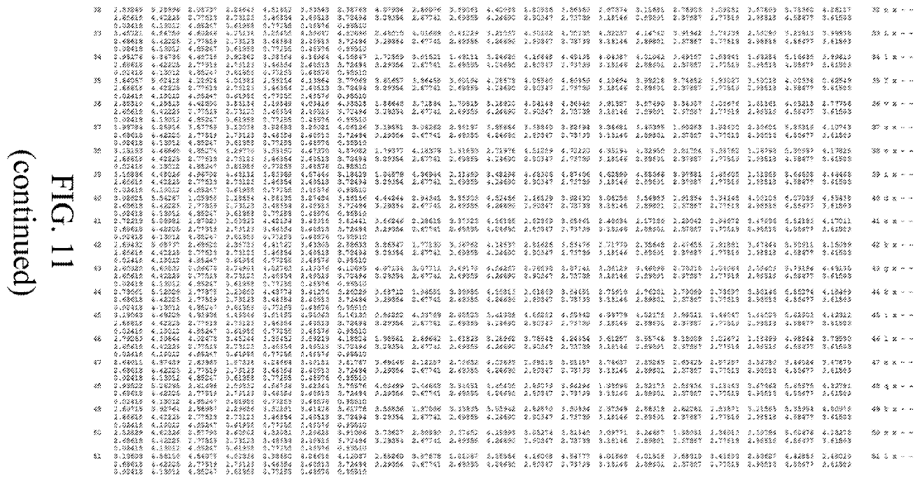

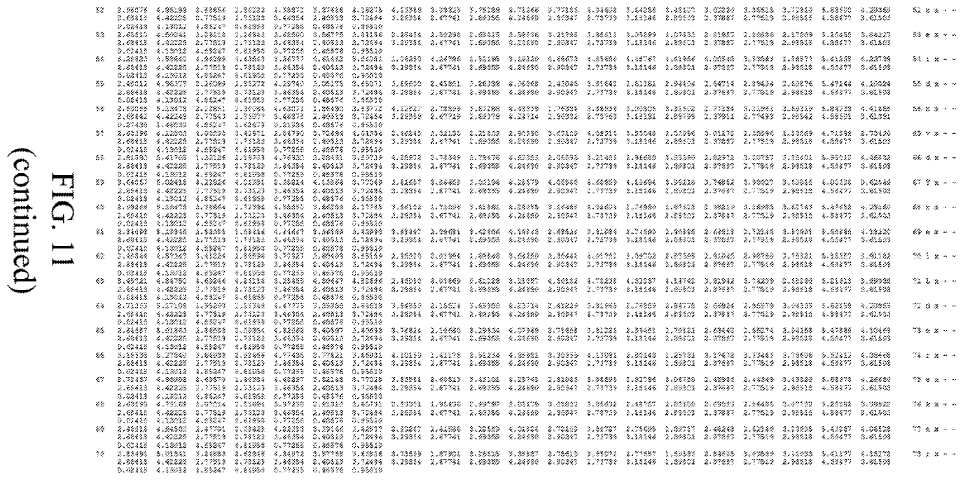

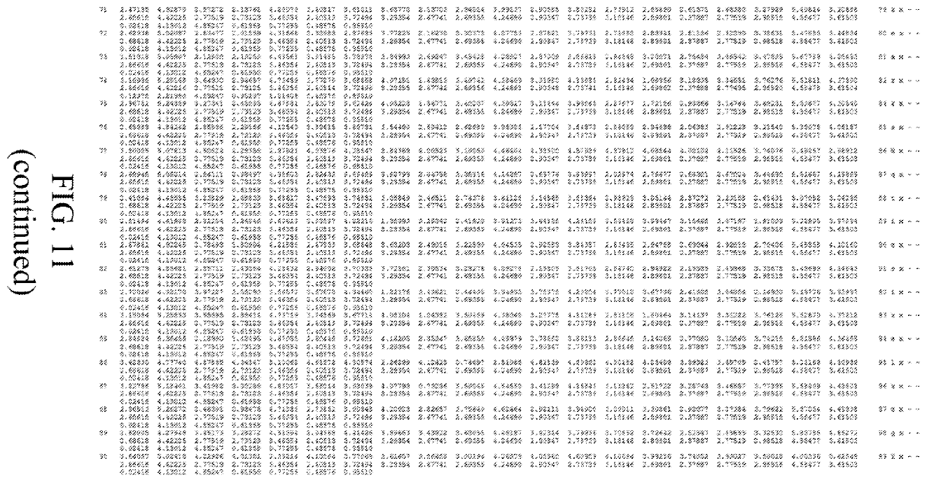

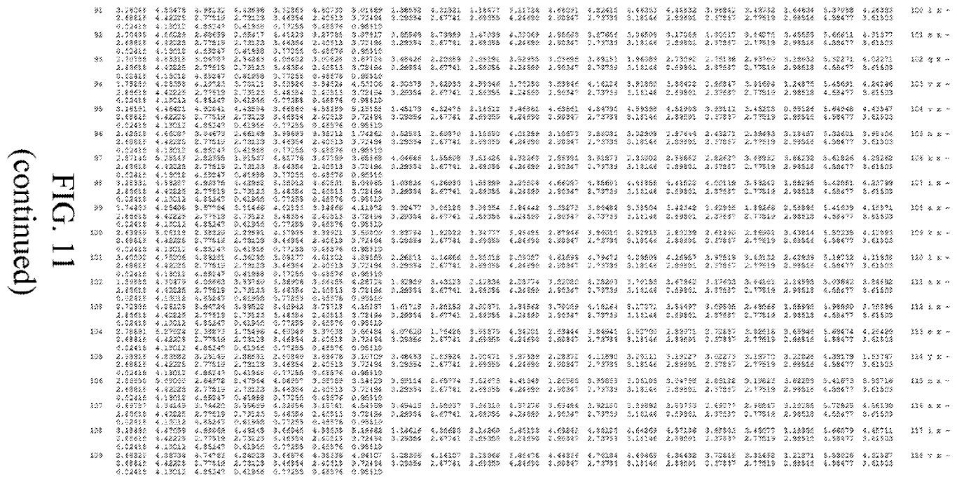

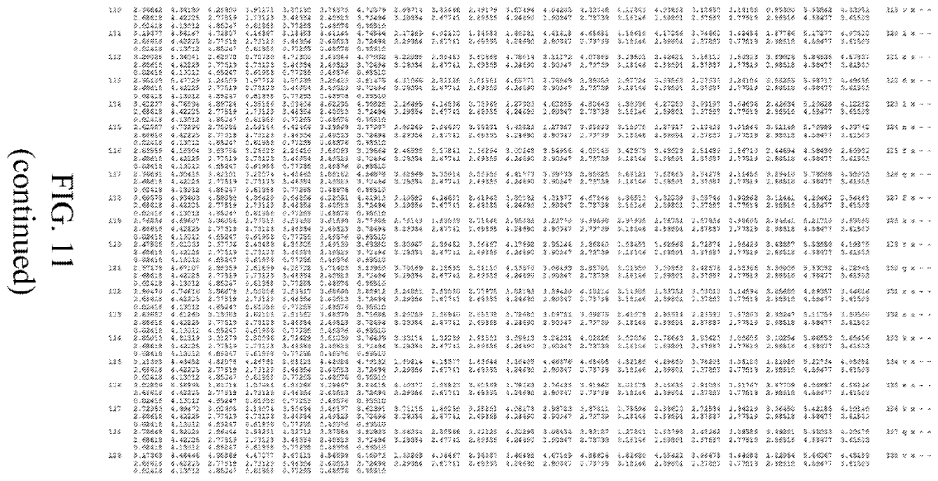

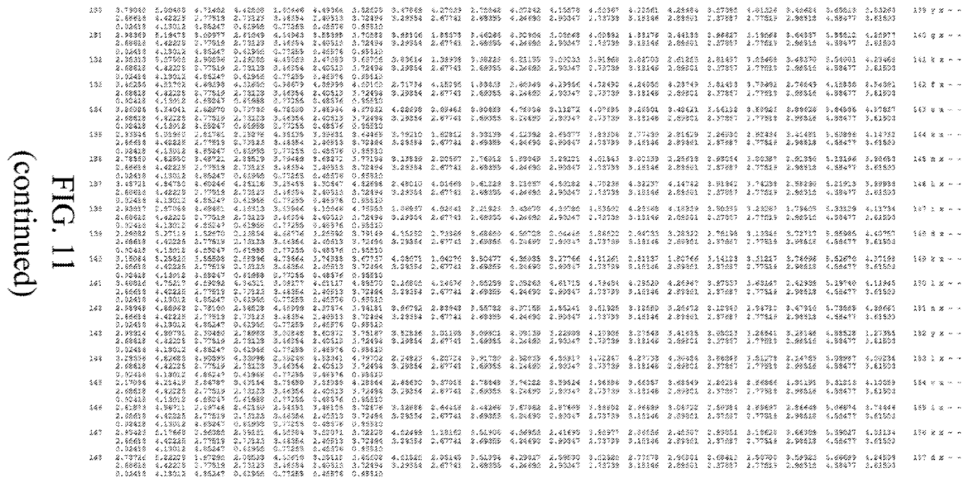

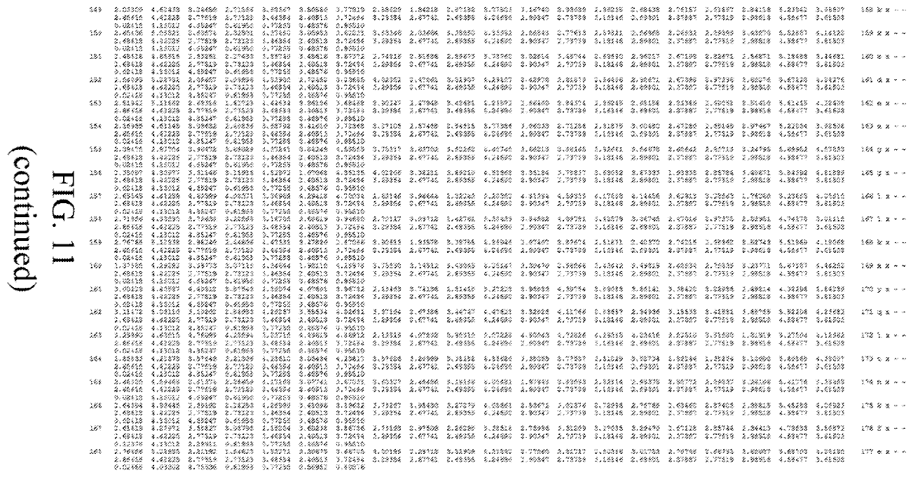

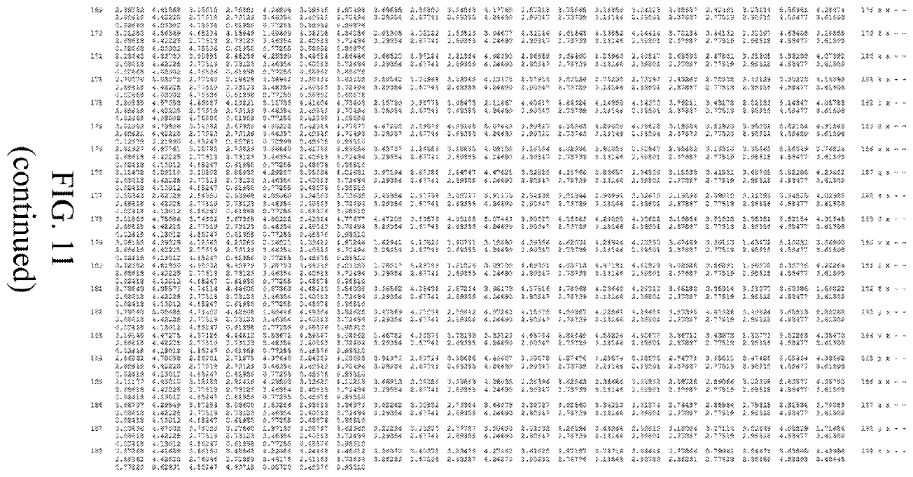

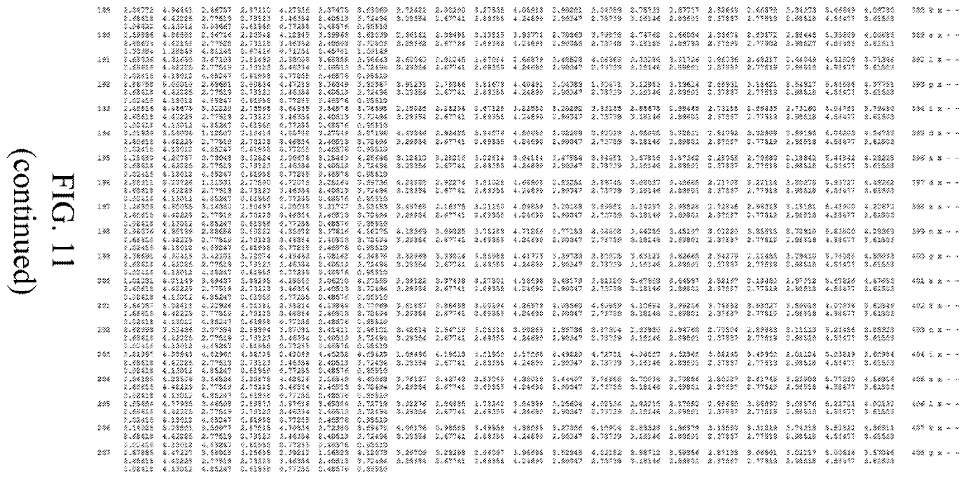

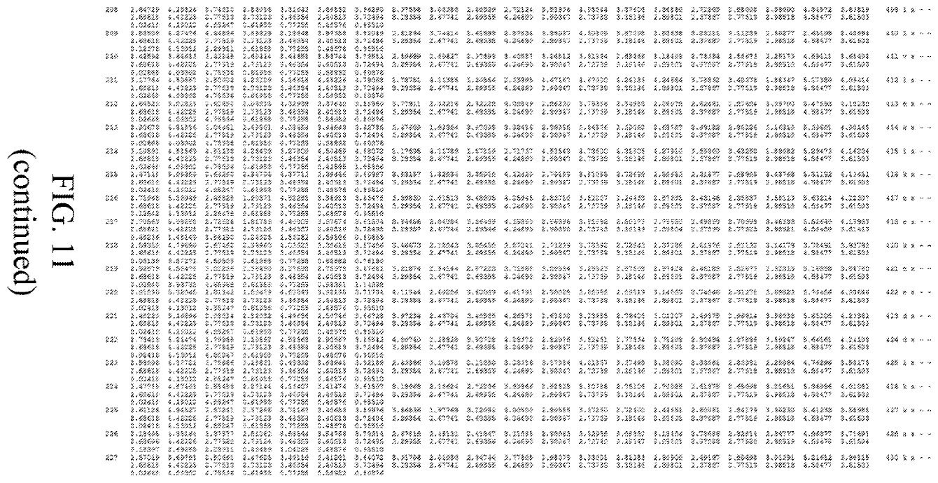

Results are shown in FIG. 9. FIG. 9A shows results from assays in which the R1490 guide was used. FIG. 9B shows results from assays in which the R1488 guide was used. FIG. 9C shows results from assays in which the R1491 guide was used. In FIG. 9A-9C, the trace appearing more linear from about 1000 to about 2000 AU of raw fluorescence corresponds to assays with no target ssDNA (shown as 0 μM). In FIG. 9A-9C, the trace appearing more curved corresponds to assays with 2 μM of ssDNA. As demonstrated in FIG. 9, the results indicated that the Cas13a DNA-activated programmable RNA nuclease is able to detect long, genome-sized ssDNA products, and not just short ssDNA oligonucleotides (as shown in EXAMPLE 11).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A composition comprising:
a) a Type VI Clustered Regularly Interspaced Short Palindromic Repeats (CRISPR)-Cas nuclease; and
b) an engineered guide nucleic acid comprising a first segment that is reverse complementary to a segment of a target single-stranded deoxyribonucleic acid, wherein the engineered guide nucleic acid comprises a second segment that binds to the Type VI CRISPR-Cas nuclease nuclease to form a complex.

2. The composition of claim 1, further comprising an RNA reporter or a DNA reporter.

3. (canceled)

4. (canceled)

5. The composition of claim 1, wherein the composition further comprises the target single-stranded deoxyribonucleic acid.

6. The composition of claim 5, wherein the target single-stranded deoxyribonucleic acid is an amplicon of a nucleic acid.

7. The composition of claim 6, wherein the nucleic acid is a deoxyribonucleic acid.

8. The composition of claim 6, wherein the nucleic acid is a ribonucleic acid.

9. (canceled)

10. (canceled)

11. The composition of claim 1, wherein the Type VI CRISPR-Cas nuclease is a Cas13 protein.

12. The composition of claim 11, wherein the Cas13 protein comprises a Cas13a polypeptide, a Cas13b polypeptide, a Cas13c polypeptide, a Cas13c polypeptide, a Cas13d polypeptide, or a Cas13e polypeptide.

13. (canceled)

14. The composition of claim 12, wherein the Cas13 protein comprises the Cas13a polypeptide, and wherein the Cas13a polypeptide is LbuCas13a or LwaCas13a.

15. The composition of claim 1, wherein the Type VI CRISPR-Cas nuclease comprises an amino acid sequence having at least 80% sequence identity to any one of SEQ ID NO: 18-SEQ ID NO: 35.

16. The composition of claim 15, wherein the Type VI CRISPR-Cas nuclease comprises an amino acid sequence having at least 80% sequence identity to SEQ ID NO: 19.

17. The composition of claim 1, wherein the composition has a pH from pH 6.8 to pH 8.2.

18. The composition of claim 5, wherein the target single-stranded deoxyribonucleic acid lacks a guanine at the 3′ end.

19. The composition of claim 5, wherein the terminal 3′ nucleotide in the segment of the target single-stranded deoxyribonucleic acid is A, C or T.

20. The composition of claim 15, wherein the Type VI CRISPR-Cas nuclease comprises an amino acid sequence having at least 80% sequence identity to SEQ ID NO: 25.

21. (canceled)

22. (canceled)

23. The composition of claim 1, wherein the target single-stranded deoxyribonucleic acid has a length of from 18 to 100 nucleotides.

24. (canceled)

25. (canceled)

26. The composition of claim 1, wherein the composition is comprised within a support medium.

27. The composition of claim 1, further comprising a second engineered guide nucleic acid comprising a first segment that is reverse complementary to a segment of a second target deoxyribonucleic acid; and
a Type V CRISPR-Cas nuclease, wherein the second engineered guide nucleic acid comprises a second segment that binds to the Type V CRISPR-Cas nuclease to form a complex.

28. (canceled)

29. (canceled)

30. (canceled)

31. The composition of claim 1, wherein the target single-stranded deoxyribonucleic acid is a reverse transcribed ribonucleic acid.

32. The composition of claim 1, wherein the composition further comprises a reagent for reverse transcription, amplification, in vitro transcription, or a combination thereof.

33. (canceled)

34. (canceled)

35. The composition of claim 32, wherein the composition further comprises the reagent for reverse transcription, and wherein the reagent for reverse transcription comprises a reverse transcriptase, an oligonucleotide primer, dNTPs, or any combination thereof.

36. The composition of claim 32, wherein the composition further comprises the reagent for amplification, and wherein the reagent for amplification comprises a primer, a polymerase, dNTPs, or any combination thereof.

37. The composition of claim 32, wherein the composition further comprises the reagent for in vitro transcription, and wherein the reagent for in vitro transcription comprises an RNA polymerase, NTPs, a primer, or any combination thereof.

38. A method of assaying for a target single-stranded deoxyribonucleic acid in a sample, the method comprising:
contacting the sample to the composition of any one of claims 1-37; and
assaying for a signal produced by cleavage of at least some RNA reporters of a plurality of RNA reporters by the Type VI CRISPR-Cas nuclease upon hybridization of the first segment of the engineered guide nucleic acid to the segment of the target single-stranded deoxyribonucleic acid.

39. A method of assaying for a target ribonucleic acid in a sample, the method comprising:
amplifying the target ribonucleic acid in a sample to produce a target single-stranded deoxyribonucleic acid;
contacting the target single-stranded deoxyribonucleic acid to the composition of any one of claims 1-37; and
assaying for a signal produced by cleavage of at least some RNA reporters of a plurality of RNA reporters by the Type VI CRISPR-Cas nuclease upon hybridization of the first segment of the engineered guide nucleic acid to the segment of the target single-stranded deoxyribonucleic acid.

40. (canceled)

41. (canceled)

42. (canceled)

43. A kit comprising:
(a) a Type VI Clustered Regularly Interspaced Short Palindromic Repeats (CRISPR)-Cas nuclease;
(b) an engineered guide nucleic acid comprising a first segment that is reverse complementary to a segment of a target single-stranded deoxyribonucleic acid; and
(c) a detector nucleic acid comprising a detection moiety.

44. The kit of claim 43, further comprising a second engineered guide nucleic acid.

45. A method of determining the presence of a target single-stranded deoxyribonucleic acid in a sample, the method comprising:
assaying for a signal produced by cleavage of a detection moiety from an RNA reporter, wherein the cleavage occurs when the target single-stranded deoxyribonucleic acid is bound to a complex comprising a Type VI Clustered Regularly Interspaced Short Palindromic Repeats (CRISPR)-Cas nuclease and an engineered guide nucleic acid comprising a first segment that is reverse complementary to a segment of the target single-stranded deoxyribonucleic acid, wherein if the target single-stranded deoxyribonucleic acid is present in the sample, the signal is detected, thereby determining the presence of the single-stranded deoxyribonucleic acid in the sample.
